(19) United States
(12) Patent Application Publication
(10) Pub. No.: US 2025/0026840 A1
(43) Pub. Date: Jan. 23, 2025

(54) CHIMERIC ANTIGEN RECEPTORS AGAINST AXL OR ROR2 AND METHODS OF USE THEREOF

(71) Applicants: Exuma Biotech Corp., West Palm Beach, FL (US); BioAtla, Inc., San Diego, CA (US)

(72) Inventors: Gregory Ian FROST, West Palm Beach, FL (US); James Joseph ONUFFER, Jr., Alameda, CA (US); Jay M. SHORT, Jackson, WY (US); Gerhard FREY, San Diego, CA (US); Hwai Wen CHANG, San Marcos, CA (US)

(73) Assignees: Exuma Biotech Corp., West Palm Beach, FL (US); BioAtla, Inc., San Diego, CA (US)

(21) Appl. No.: 18/907,457

(22) Filed: Oct. 4, 2024

Related U.S. Application Data
(63) Continuation of application No. 16/478,360, filed on Jul. 16, 2019.
(60) Provisional application No. 62/447,898, filed on Jan. 18, 2017.
(60) Provisional application No. 62/467,059, filed on Mar. 3, 2017.
(60) Provisional application No. 62/530,193, filed on Jul. 8, 2017.

(51) Int. Cl. C07K 16/28; A61K 39/00; A61P 35/00; C07K 14/705; C07K 14/71; C07K 14/725; C12N 5/0783

(52) U.S. Cl. CPC C07K 16/2863; A61K 39/4611; A61K 39/4631; A61K 39/464402; A61K 2239/31; A61K 2239/38; A61K 2239/59; A61P 35/00; C07K 14/7051; C07K 14/70517; C07K 14/70521; C07K 14/70578; C07K 14/71; C07K 16/2803; C12N 5/0636; C12N 5/0646; C07K 2317/622; C07K 2317/73; C07K 2319/03

(57) ABSTRACT

The present disclosure provides chimeric antigen receptors that bind to Axl and Ror2, and conditionally active chimeric antigen receptors (CARs) that recognize Axl and Ror2. Furthermore, provided herein are nucleic acids encoding these CARs and methods of making and using the CARs, including methods of treating cancer, especially cancers that express Axl and/or Ror2, such as renal cell carcinoma. The present disclosure provides cells genetically modified to produce the CARs.

### CROSS-REFERENCE TO RELATED APPLICATIONS

This application is a continuation of U.S. patent application Ser. No. 16/478,360, filed Jul. 16, 2019. U.S. patent application Ser. No. 16/478,360 is a national stage entry of International Patent Application No. PCT/US2018/014122, filed Jan. 17, 2018. International Patent Application No. PCT/US2018/014122 claims the benefit of U.S. Provisional Application No. 62/447,898, filed Jan. 18, 2017, U.S. Provisional Application No. 62/467,059, filed Mar. 3, 2017, and U.S. Provisional Application No. 62/530,193, filed Jul. 8, 2017. These applications cited in this paragraph are incorporated by reference herein in their entirety.

### SEQUENCE LISTING

This application hereby incorporates by reference the material of the electronic Sequencing Listing filed concurrently herewith. The materials in the electronic Sequence Listing is submitted as an xml (.xml) file entitled “F1.002.US.01CON1 Sequence Listing.xml” created on 10/4/2024, which has a file size of 231 KB, and is herein incorporated by reference in its entirety.

### JOINT RESEARCH AGREEMENT

Exuma Biotech, Corp. and BioAtla, Inc. are parties to a joint research agreement that relates to the subject matter disclosed herein.

### FIELD OF DISCLOSURE

This disclosure relates to chimeric antigen receptors and uses of the chimeric antigen receptors in diagnostic and therapeutic methods.

### BACKGROUND OF THE DISCLOSURE

In cell-based adoptive immunotherapy, immune cells isolated from a patient can be modified to express synthetic proteins that enable the cells to perform new therapeutic functions after they are subsequently transferred back into the patient. An example of such a synthetic protein is a chimeric antigen receptor (CAR). An example of a currently used CAR is a fusion of an extracellular recognition domain (e.g., an antigen-specific targeting region or ASTR), a transmembrane domain, and one or more intracellular signaling domains. Upon antigen engagement, the intracellular signaling portion of the CAR can initiate an activation-related response in an immune cell, such as release of cytolytic molecules to induce tumor cell death, etc. Although CARs and CAR-T therapy has been very effective for certain types of blood cancers, there remains a need for CARs and CAR-T therapy against solid tumors, which have proven thus far to be much more elusive.

While CARs represent an impressive method to treat various diseases, the safety of CARs has recently come into question through adverse events during clinical trials. One method to decrease these adverse events is by reducing the on-target off-tumor binding of the ASTRs. CARs with conditionally active ASTRs only bind to antigen under certain conditions, such as those that exist in the tumor microenvironment, and provide a reduction in on-target off-tumor binding such that the CARs do not bind the antigen in normal physiological conditions. Thus, the side effects of these CARs are reduced and treatment can proceed more safely.

Receptor tyrosine kinases (RTKs) are a family of cell surface receptors that regulate a range of normal cellular processes through ligand-controlled tyrosine kinase activity. Over the past 20 years, deregulation of RTKs has been shown to play a critical role in cancer development and progression. RTKs are now recognized as prognostic molecular biomarkers and as targets of oncology therapeutics.

Axl protein (also known as Ark, UFO, Tyro-7) is an RTK in the Tyro-3 family of kinases. The Tyro-3 receptor kinases are characterized by a combination of two immunoglobin-like domains and dual fibronectin type III repeats in the extracellular region and a cytoplasmic kinase domain. The ligands for Tyro-3 receptor kinases are Gas6 (growth-arrest-specific 6) and protein S, two vitamin-K dependent proteins that show 43% amino acid sequence identity and share similar domain structures. Each protein has an N-terminal GIa domain containing 11 g-carboxyglutamic acid residues, followed by four epidermal growth factor (EGF)-like modules, and a C-terminal sex hormone-binding globlin (SHBG)-like structure consisting of two tandem laminin G domains. The SHBG domain is both necessary and sufficient for Tyro-3 receptor kinase binding and activation, whereas the GIa domain binds the negatively charged membrane phospholipids and plays an important role in Tyro-3 kinase-mediated phagocytosis of apoptotic cells.

Axl activation leads to signaling through PI-3-kinase/Akt and other major pathways like Ras/Erk and β-catenin/TCF. Axl is weakly expressed in a range of normal tissues, including brain, heart, skeletal muscle, the organ capsules and connective tissues of several other organs, and in monocytes, but not lymphocytes. Akt phosphorylation induced by Axl has been described in survival of fibroblasts, endothelial cells, vascular smooth muscle cells and neurons. Furthermore, Axl plays a role in cell-adhesion and chemotaxis because Axl knockout animals display impaired platelet aggregate stabilization and thrombus formation as a result of reduced activation of the platelet integrin IIb3.

Dysregulation of Axl or its ligand Gas6 is implicated in the pathogenesis of a variety of human cancers. Axl overexpression has been demonstrated in various cancer types, e.g. breast (Meric et al., Clin. Cancer Res., vol. 8, pp. 361-367, 2002; Berclaz et al., Ann. Oncol., vol. 12, pp. 819-824, 2001), colon (Chen et al., Int. J. Cancer, vol. 83, pp. 579-584, 1999; Craven et al., Int. J. Cancer, vol. 60, pp. 791-797, 1995), prostate (Jacob et al., Cancer Detect. Prey., vol. 23, pp. 325-332, 1999), lung (Wimmel et al., Eur J Cancer, vol. 37, pp. 2264-2274, 2001), gastric (Wu et al., Anticancer Res., vol. 22, pp. 1071-1078, 2002), ovarian (Sun et al., Oncology, vol. 66, pp. 450-457, 2004), endometrial (Sun et al., Ann. Oncol., vol. 14, pp. 898-906, 2003), renal (Chung et al., DNA Cell Biol., vol. 22, pp. 533-540, 2003), hepatocellular (Tsou et al., Genomics, vol. 50, pp. 331-340, 1998), thyroid (Ito et al., Thyroid, vol. 12, pp. 971-975, 2002; Ito et al., Thyroid, vol. 9, pp. 563-567, 1999), osteosarcoma (Nakano et al., J Biol. Chem., vol. 270, pp. 5702-5705, 2003), melanoma (van Ginkel et al., Cancer Res., vol. 64, pp. 128-134, 2004), in head and neck squamous cell carcinoma (Green et al., Br J Cancer., vol. 94, pp. 1446-51, 2006), ovarian cancer, renal cancer, glioma, endocrine, pancreas, lymphoma, brain, liver, renal cell carcinoma, renal clear cell, bladder, rectum, cervical squamous cell carcinoma, and furthermore in lymphoma and various leukemias, including chronic myelogenous leukemia (Janssen et al., Oncogene, vol. 6, pp. 2113-2120, 1991; Braunger et al., Oncogene, vol. 14, pp. 2619-2631 1997; O'Bryan et al., Mol. Cell. Biol., vol. 11, pp. 5016-5031, 1991), and acute myeloid leukemia (Rochlitz et al., Leukemia, vol. 13, pp. 1352-1358, 1999).

Axl expression is induced by targeted chemotherapy drugs and drug-induced Axl expression confers resistance to chemotherapy in acute myeloid leukemia (Hong et al, Cancer Letters, vol. 268, pp. 314-324, 2008), as well as resistance to imatinib and Lapatinib/Herceptin in gastrointestinal stromal tumors (Mehadevan, et al, Oncogene, vol. 26, pp. 3909-3919, 2007) and breast cancer (Liu et al, Cancer Research, vol. 281, pp. 6871-6878, 2009), respectively.

Moreover, Axl has been identified to be related to tumor metastasis because Axl is upregulated in aggressive breast cancer cell lines compared to non-invasive cells. In vitro, Axl activity was found to be required for migration and invasion, and this activity could be inhibited by antibody treatment (WO 04/008147). Similarly, abrogation of Axl activity in vivo, either via expression of a dominant negative version of Axl (Vajkoczy, P., et al., Proc. Natl. Acad. Science U.S.A., vol. 103, pp. 5799-5804, 2005) or by siRNA mediated downregulation of Axl (Holland et al., Cancer Res., vol. 65, pp. 9294-9303, 2005) prevented subcutaneous and orthotopic cell growth in murine xenograft experiments.

Accordingly, anti-Axl monoclonal antibodies have been suggested for use in the treatment of cancers. For example, publications relating to anti-Axl antibodies include WO 2009/063965, WO 2009/062690, WO 2011/014457, US 2014/0227283, and U.S. Pat. No. 8,853,369. US 2014/0227283 discloses monoclonal anti-Axl antibodies and uses thereof in diagnostic and therapeutic methods. WO 2009/062690 discloses antibodies that bind to the extracellular domain of the Axl protein and can at least partially inhibit Axl activity. However, even for targets such as Axl, where monoclonal antibodies have been identified and are available as reagents, many challenges exist that make it very difficult to create a conditionally active CAR against Axl or Ror2.

Another RTK, Ror2, also called receptor tyrosine kinase-like orphan receptor 2, is a membrane-bound that is activated by non-canonical Wnt signaling through its association with the Wnt5A glycoprotein during normal bone and cartilage development. Ror2 has only one transmembrane domain, which separates its extracellular and intracellular domains. Ror2 is known to play crucial roles in the normal development of various organs and tissues. In mammals, Ror2- and Wnt5A-deficient mice exhibit similar abnormalities during developmental morphogenesis, reflecting their defects in convergent extension movements and planar cell polarity. Furthermore, mutations of the human Ror2 gene are responsible for the genetic skeletal disorders dominant brachydactyly type B and recessive Robinow syndrome. Ror2 has been found to mediate polarized cell migration and malfunction of Ror2 results in heritable skeletal disorders and tumor invasion (Minami et al., “Ror-family receptor tyrosine kinases in noncanonical Wnt signaling: their implications in developmental morphogenesis and human diseases,” Dev Dyn., vol. 239, pp. 1-15, 2010). Further, Debebe et al., (“Ror2 as a therapeutic target in cancer,” Pharmacol. Ther., vol. 50, pp. 143-148, 2015) discloses that Ror2 mediates both canonical and non-canonical signaling pathways.

Ror2 has also been reported to have pro-tumorigenic effects. US 2014/0322234 discloses that the expression and activity of Ror2 in various cancers is different from normal tissues. Thus, it is suggested that dysregulation of Ror2 plays a role in the pathogenesis of a variety of human cancers. US 2014/0322234 also contemplates that antibodies against Ror2 may be used in diagnosis of cancers and inhibition of cancer cell growth. For example, such antibodies may be conjugated to a cytotoxic agent that has a high degree of cytotoxicity for cancer cells expressing Ror2, such that the cytotoxic agent can effectively kill the cancer cells. The Ror2 gene may also be used in classification of cancers according to the Ror2 expression pattern in the cancers.

Ror2 is involved in the development and progression of cancers (“The dual role of the novel Wnt receptor tyrosine kinase, Ror2, in human carcinogenesis,” _{International Journal of Cancer}, vol. 133, pp. 779-787, 2013). Specifically, Ror2 has been found to play a pivotal role in carcinogenesis of numerous cancers including colon cancer, hepatocellular carcinoma, metastatic melanoma, and renal cell carcinoma. For example, Ror2 is over-expressed in osteosarcoma, melanoma, renal cell carcinoma, prostate carcinoma, squamous cell carcinomas of the head and neck, colon cancer, breast cancer, lymphoma, leukemia, thyroid, endocrine pancreas, brain, ovarian, renal papillary, lung, pancreas, liver, renal clear cell, bladder, endometrial, rectum, cervical squamous cell carcinoma, and stromal tumors. In the majority of these cancer types, Ror2 expression is associated with more aggressive cancer states. Ror2 thus has the potential of being a drug target for cancer treatments by inhibition of the Wnt signaling pathway.

There remains a need for an effective treatment that harnesses the power of the immune system to fight cancer, but that has reduced or eliminated on-target off-tumor as well as off-target effects. Though monoclonal antibodies against Ror2 and Axl are commercially available, there is a need for CARS that include antibody fragments targeting Ror2 or Axl that are conditionally active, that effectively target cells expressing Ror2 or Axl only in certain environments, such as a cancer microenvironment. Creating such conditionally active CARs presents numerous challenges. For example, antibody fragments must be created and identified, that not only bind Axl or Ror2 when they are expressed on the surface of T cells or NK cells as part of CARs, but that additionally have the ability to recognize an epitope that is exposed on cancer cells. Furthermore, such CARs much bind to their targets in a conditionally active manner, especially under the acidic pH of a tumor compared to a normal physiological pH. Additionally, such candidate CARs, when bound to their target, must activate a T cell or NK cell expressing the CAR to express a cytotoxic function. Thus, there are many requirements for a CAR containing such an antibody fragment, to help solve problems posed by current CAR-T methods. Such a conditionally active CAR against Axl or Ror2 would hold promise for treating solid cancers using CAR-T therapy, thus overcoming a major limitation of current CAR-T therapies.

### SUMMARY OF THE DISCLOSURE

The present disclosure provides chimeric antigen receptors (CARs), and nucleic acids comprising nucleotide sequences encoding the CARs, that bind to Axl and/or Ror2, and conditionally active biologic (CAB) CARs that bind to Axl and Ror2. The present disclosure provides cells genetically modified to produce the CARs, and methods for making such cells. The CARs of the present disclosure can be used in various methods, which are also provided, including methods for activating immune cells under certain conditions, such as a pH below a threshold value, methods for performing adoptive cell therapy such as CAR therapy, for example CAR therapy against cancer, for example renal cell carcinoma.

Details of aspects and embodiments provided herein are provided throughout this disclosure. For the sake of clarity, this Summary section is not intended to be, and should not be construed to limit the scope of the disclosure provided herein.

### BRIEF DESCRIPTION OF THE DRAWINGS

FIG. 1 provides a bar graph showing percent transduction of CD3⁺ cells based on FACS analysis of eTag expression.

FIGS. 2A-2F show the results from real-time killing assays of CHO-Axl cells at pH 6.7 and pH 7.4 by T cells expressing a control CAR with a wild-type ASTR that recognizes Axl (FIG. 2A) and T cells expressing candidate CAB-CARs that recognize Axl, which have conditionally active ASTRs that recognize Axl (FIGS. 2B-2F). The effector to target ratio was 3:1 in FIGS. 2A and 2B, and 1:1 in FIGS. 2C-2F.

FIGS. 3A-3J show the results from real-time killing assays of CHO-Ror2 cells at pH 6.7 and pH 7.4 by T cells expressing a control CAR with a wild-type ASTR that recognizes Ror2 (FIG. 3A) and T cells expressing candidate CAB-CARs that recognize Ror2, which have conditionally active ASTRs that recognize Ror2 (FIGS. 3B-J). The effector to target ratio was 1:1.

FIG. 4A shows representative results from a real-time killing assay of CHO-Axl cells by T cells expressing one of the conditionally active CARs against Axl provided herein, at various pHs. FIG. 4B provides lysis results at the 20 hour time point at various pHs as noted, for the same CAB-CAR as FIG. 4A.

FIG. 5A shows representative results from a real-time killing assay of CHO-Ror2 cells by T cells expressing one of the conditionally active CAB-CARs against Ror2 provided herein, at various pHs. FIG. 5B provides lysis results at the 20 hour time point at various pHs as noted, for the same CAB-CAR as FIG. 5A.

FIG. 6A shows the results from real-time killing assays of human Caki-1 cells at pH 6.7 and pH 7.4 by T cells expressing one of the conditionally active CAB-CARs against Axl. FIG. 6B shows the results from real-time killing assays of human HEK293 cells at pH 6.7 and pH 7.4 by T cells expressing one of the conditionally active CAB-CARs against Ror2.

FIG. 7A shows cell depletion results for PBMCs transduced with a lentivirus expression vector expressing eTag after treatment with Cetuximab at various concentrations, or a control (isotope). FIG. 7B provides cell depletion results for PBMCs transduced with a lentivirus expression vector expressing eTag after treatment with Cetuximab or a control (isotope) at various ratios of transduced (E) to non-transduced (T) cells.

FIGS. 8A-8D show bar graphs of the cytokine levels in media with or without co-incubation of different cells at pH 6.7 or pH 7.4. The various cells used were T cells expressing the GMCSF signal sequence and eTag (F1-0-01), T cells expressing an Axl CAB-CAR (F1-2-15), T cells expressing a Ror2 CAB-CAR (F1-1-15), CHO cells, CHO cells expressing Axl (CHO-AXL), and CHO cells expressing Ror2 (CHO-ROR2). FIG. 8A shows the levels of IL-2 in media from F1-0-01 or F1-2-15 T cells alone or when co-incubated with CHO or CHO-Axl. FIG. 8B shows the levels of IFN-γ in media from F1-0-01 or F1-2-15 T cells alone or when co-incubated with CHO or CHO-Axl. Cytokine levels in media when CHO or CHO-Axl cells are incubated in the absence of effector T cells are also shown as a control in FIGS. 8A and 8B. FIG. 8C shows the levels of IL-2 in media from F1-0-01 or F1-1-15 T cells alone or when co-incubated with CHO or CHO-Ror2. FIG. 8D shows the levels of IFN-γ in media from F1-0-01 or F1-1-15 T cells alone or when co-incubated with CHO or CHO-Ror2. Cytokine levels in media when CHO or CHO-Ror2 cells are incubated in the absence of effector T cells are also shown as a control in FIGS. 8C and 8D.

FIGS. 9A-9C show representative results from a real-time killing assay of CHO-Axl cells by T cells expressing one of the conditionally active CAB-CARs against Axl provided herein, with and without treatment with a pH-modulating pharmacologic agent. In FIG. 9A, the media was initially at pH 6.7 and experimental wells (solid line) and control cells (dashed line) were treated with or without NaHCO₃, respectively, at the time indicated by the arrow. In FIG. 9B, the media was initially at pH 6.7 and experimental wells (solid line) and control cells (dashed line) were treated with or without NaOH, respectively, at the time indicated by the arrow. In FIG. 9C, the media was initially at pH 7.4 and experimental wells (solid line) and control cells (dashed line) were treated with or without HCl, respectively.

FIG. 10 is a graph showing the RFU percentage from ProSense FAST probe in CHO-xenograft tumor bearing mice before and after administration of PBS or bicarbonate.

FIG. 11A shows the mean tumor volume of CHO tumors in B-NSG mice dosed intravenously with PBS or human T cells transduced with either a lentiviral particle containing a nucleic acid encoding F1-0-01 (eTag) or F1-1-15, one of the conditionally active CAB-CARs against Ror2. FIG. 11B shows the mean tumor volume of CHO-Ror2 tumors in B-NSG mice dosed intravenously with PBS or human T cells transduced with either a lentiviral particle containing a nucleic acid encoding F1-0-01 or F1-1-15.

FIG. 12A shows the mean tumor volume of CHO tumors in B-NSG mice dosed intratumorally with PBS or human T cells transduced with either a lentiviral particle containing a nucleic acid encoding F1-0-01 (eTag) or F1-2-15 or F1-2-22. F1-2-15 and F1-2-22 encode two of the conditionally active CAB-CARs against Axl. FIG. 12B shows the mean tumor volume of CHO-Axl tumors in B-NSG mice dosed intratumorally with PBS or human T cells transduced with a lentiviral particle containing a nucleic acid encoding either F1-0-01, F1-2-15, or F1-2-22.

FIG. 13A shows the mean tumor volume of CHO tumors in B-NSG mice dosed intravenously with PBS or human T cells transduced with a lentiviral particle containing a nucleic acid encoding either F1-0-01 (eTag) or F1-2-15 or F1-2-22. F1-2-15 and F1-2-22 encode two of the conditionally active CAB-CARs against Axl. FIG. 13B shows the mean tumor volume of CHO-Axl tumors in B-NSG mice dosed intravenously with PBS or human T cells transduced with a lentiviral particle containing a nucleic acid encoding either F1-0-01, F1-2-15, or F1-2-22.

FIG. 14A shows the mean tumor volume of CHO tumors in B-NSG mice dosed intravenously with PBS or human T cells transduced with a lentiviral particle containing a nucleic acid encoding either F1-0-01 (eTag) or F1-2-15 or F1-2-22. F1-2-15 and F1-2-22 encode two of the conditionally active CAB-CARs against Axl. FIG. 14B shows the mean tumor volume of CHO-Axl tumors in B-NSG mice dosed intravenously with PBS or human T cells transduced with a lentiviral particle containing a nucleic acid encoding either F1-0-01, F1-2-15, or F1-2-22. Mice dosed with T cells in FIGS. 14A and 14B also received subcutaneous injections of IL-2 (50,000 IU) in the contralateral site away from the tumor every day for the first 3 days.

### DEFINITIONS

The terms “chimeric antigen receptor” or “CAR” or “CARs” as used herein refer to engineered receptors, which graft an antigen specificity onto cells, for example T cells, NK cells, macrophages, and stem cells. The CARs of the invention may include at least one antigen-specific targeting region (ASTR), a hinge or stalk domain (i.e. extracellular stalk domain (ESD)), a transmembrane domain (TM), one or more co-stimulatory domains (CSDs), and an intracellular activating domain (IAD). In certain embodiments, the ESD and/or CSD are optional. CARs provided herein in some embodiments have specifically recited domains (e.g. a CD3Z intracellular activating domain). For such specially recited domains, it is intended that the domain retains an activity of a wild-type domain such that it can be effectively employed in a CAR (i.e. the CAR retains the ability to bind a target and in response, transmit a signal through an intracellular activating domain found in the CAR), and has at least 80% sequence identity to a known human sequence for that specific domain, over the portion of the domain providing such activity. For example, a CD3Z intracellular domain has at least 80% sequence identity at the amino acid level, to a known human CD3Z sequence, and when found on a CAR retains the ability to transmit a signal upon binding of an ASTR of the CAR to it target. In another embodiment, the CAR is a bispecific CAR, which is specific to two different antigens or epitopes. After the ASTR binds specifically to a target antigen, the IAD activates intracellular signaling. For example, the IAD can redirect T cell specificity and reactivity toward a selected target in a non-MHC-restricted manner, exploiting the antigen-binding properties of antibodies. The non-MHC-restricted antigen recognition gives T cells expressing the CAR the ability to recognize an antigen independent of antigen processing, thus bypassing a major mechanism of tumor escape. Moreover, when expressed in T cells, CARs advantageously do not dimerize with endogenous T cell receptor (TCR) alpha and beta chains.

The term “conditionally active” as used herein with respect to a CAR or an ASTR refers to a CAR or an ASTR that has a lower binding affinity or, in illustrative embodiments, a higher binding affinity to one or more target antigens under a condition(s) in a target microenvironment than under a condition in a normal physiological environment. In illustrative embodiments, conditionally active CARs provided herein are more active under a condition in a tumor microenvironment or an in vitro tumor surrogate assay compared to under a condition in a non-tumor microenvironment or a normal condition. The conditions in the tumor microenvironment include lower pH, higher concentrations of lactate and pyruvate, hypoxia, lower concentration of glucose, and slightly higher temperature in comparison with non-tumor microenvironment. For example, a conditionally active CAR, in certain embodiments, is virtually inactive at normal body temperature, but is active at a higher temperature in a tumor microenvironment. In yet another embodiment, a conditionally active CAR is less active in normal oxygenated blood, but more active under a less oxygenated environment that exists in a tumor. In an illustrative embodiments provided herein, a conditionally active CAR is less active in normal physiological pH 7.2-7.8, but more active under an acidic pH 6.0-6.8 that exists in a tumor microenvironment. There are other conditions in the tumor microenvironment known to a person skilled in the field that may also be used as the condition in the present invention under which conditionally active CARs and ASTRs have different binding affinities. Conditionally active CARs can be referred to herein as “conditionally active biologic CARs” or “CAB-CARs” and “microenvironment restricted biologic CARs” or “MRB-CARs”.

The term “microenvironment” as used herein means any portion or region of a tissue or body that has constant or temporal, physical or chemical differences from other regions of the tissue or regions of the body. For tumors, the term “tumor microenvironment” as used herein refers to the environment in which a tumor exists, which is the non-cellular area within the tumor and the area directly outside the tumorous tissue but does not pertain to the intracellular compartment of the cancer cell itself. The tumor and the tumor microenvironment are closely related and interact constantly. A tumor can change its microenvironment, and the microenvironment can affect how a tumor grows and spreads. Typically, the tumor microenvironment has a low pH in the range of 5.8 to 7.0, more commonly in the range of 6.0 to 6.8, in the range of 6.2-6.8. On the other hand, a normal physiological pH is in the range of 7.2-7.8. The tumor microenvironment is also known to have lower concentration of glucose and other nutrients, but higher concentration of lactic acid, in comparison with blood plasma. Furthermore, the tumor microenvironment can have a temperature that is 0.3 to 1° C. higher than the normal physiological temperature. The tumor microenvironment has been discussed in Gillies et al., “MRI of the Tumor Microenvironment,” Journal of Magnetic Resonance Imaging, vol. 16, pp. 430-450, 2002, hereby incorporated by reference herein its entirety. The term “non-tumor microenvironment” refers to a microenvironment at a site other than a tumor.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “antibodies” and “immunoglobulin” include antibodies or immunoglobulins of any isotype, as well as fragments of antibodies which retain specific binding to an epitope, typically the same epitope as the full antibody when both a heavy and light chain of the antibody are present for the assay, including, but not limited to, Fab, Fab′, Fab′-SH, (Fab′)₂ Fv, scFv, divalent scFv, and Fd fragments, chimeric antibodies, humanized antibodies, single-chain antibodies, and fusion proteins comprising an antigen-specific targeting region of an antibody and a non-antibody protein.

“Antibody fragments” include a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fe” fragment, a designation reflecting the ability to crystallize readily. Pepsin treatment yields an F(ab′)₂ fragment that has two antigen combining sites and is still capable of cross-linking antigen.

“Single-chain Fv,” “scFv,” or “sFv” antibody fragments include the V_{H} and V_{L} domains of antibody, wherein these domains are present in a single polypeptide chain. In some embodiments, the Fv polypeptide further includes a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg andMoore eds., Springer-Verlag, New York, pp. 269-315 (1994).

As used herein, the term “affinity” refers to the equilibrium constant for the reversible binding of two agents and is expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. Non-specific binding would refer to binding with an affinity of less than about 10⁻⁷ M, e.g., binding with an affinity of 10⁻⁶ M, 10⁻⁵ M, 10⁻⁴ M, etc.

As used herein, the term “hinge region” refers to a flexible polypeptide connector region (also referred to herein as “hinge” or “spacer”) providing structural flexibility and spacing to flanking polypeptide regions and can consist of natural or synthetic polypeptides. A “hinge region” derived from an immunoglobulin (e.g., IgG1) is generally defined as stretching from Glu216 to Pro230 of human IgG1 (Burton (1985) Molec. Immunol., 22:161-206). Hinge regions of other IgG isotypes may be aligned with the IgG 1 sequence by placing the first and last cysteine residues forming inter-heavy chain disulfide (S-S) bonds in the same positions. The hinge region may be of natural occurrence or non-natural occurrence, including but not limited to an altered hinge region as described in U.S. Pat. No. 5,677,425. The hinge region can include a complete hinge region derived from an antibody of a different class or subclass from that of the CHl domain. The term “hinge region” can also include regions derived from CD8, CD28, or other receptors that provide a similar function in providing flexibility and spacing to flanking regions.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In some embodiments, the polypeptide will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated polypeptide includes the polypeptide in situ within recombinant cells since at least one component of the polypeptide's natural environment will not be present. In some instances, isolated polypeptide will be prepared by at least one purification step.

The term “immune cells” as used herein generally includes white blood cells (leukocytes) which are derived from hematopoietic stem cells (HSC) produced in the bone marrow. “Immune cells” includes, e.g., lymphocytes (T cells (i.e. T lymphocytes), B cells, natural killer (NK) (CD3− CD56+) cells) and myeloid-derived cells (neutrophil, eosinophil, basophil, monocyte, macrophage, dendritic cells). “T cells” include all types of immune cells expressing CD3 including T-helper cells (CD4⁺ cells), cytotoxic T-cells (CD8⁺ cells), T-regulatory cells (Treg) and gamma-delta T cells, and NK T cells (CD3+ and CD56+). A skilled artisan will understand T cells and/or NK cells, as used throughout the disclosure, can include only T cells, only NK cells, or both T cells and NK cells. In certain illustrative embodiments and aspects provided herein, T cells are activated and transduced. Furthermore, T cells are provided in certain illustrative composition embodiments and aspects provided herein. A “cytotoxic cell” includes CD8⁺ T cells, natural-killer (NK) cells, NK-T cells, γδ T cells, and neutrophils, which are cells capable of mediating cytotoxicity responses.

As used herein, the term “stem cell” generally includes pluripotent or multipotent stem cells. “Stem cells” includes, e.g., embryonic stem cells (ES); mesenchymal stem cells (MSC); induced-pluripotent stem cells (iPS); and committed progenitor cells (hematopoeitic stem cells (HSC); bone marrow derived cells, etc.).

The term “Axl” as used herein, refers to any native Axl from any vertebrate source, including mammals such as primates (e.g. humans) and rodents (e.g., mice and rats), unless otherwise indicated. The term encompasses “full-length,” unprocessed Axl as well as any form of Axl that results from processing in the cell. The term also encompasses naturally occurring variants of Axl, e.g., splice variants or allelic variants. The amino acid sequence of human Axl is well-known in the art and available from public databases such as GenBank.

The term “Axl activation” as used herein refers to activation, or phosphorylation, of the Axl receptor. Generally, Axl activation results in signal transduction (e.g. that caused by an intracellular kinase domain of an Axl receptor phosphorylating tyrosine residues in Axl or a substrate polypeptide). Axl activation may be mediated by Axl ligand (Gas6) binding to an Axl receptor of interest. Gas6 binding to Axl may activate a kinase domain of Axl and thereby result in phosphorylation of tyrosine residues in the Axl and/or phosphorylation of tyrosine residues in additional substrate polypeptides(s).

The term “Axl mediated anti-apoptosis” as used herein refers to all Axl-involving processes that prevent human cells, preferably but not limited to human cancer cells from programmed cell death (apoptosis). In particular, it refers to processes that prevent human cells, preferably but not limited to human cancer cells from induction of apoptosis through growth factor withdrawal, hypoxia, exposure to chemotherapeutic agents or radiation, or initiation of the Fas/Apo-1 receptor-mediated signaling, and are stimulated or mediated by non-catalytic or catalytic activities of Axl, preferably including Axl phosphorylation and/or Axl-mediated signal transduction.

The term “Ror2” as used herein, refers to receptor tyrosine kinase-like orphan receptor 2, which is a predicted 943-amino acid protein with in vitro protein kinase activity, shown in Genbank accession number AAI30523. Many lineage-restricted receptor tyrosine kinases were initially identified as ‘orphans’ homologous to known receptors, and only subsequently used to identify their unknown growth factors. DeChiara et al. (2000) identified one such orphan, encoded by Ror2.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, humans, murines (e.g., rats, mice), lagomorphs (e.g., rabbits), non-human primates, humans, canines, felines, ungulates (e.g., equines, bovines, ovines, porcines, caprines), etc.

A “therapeutically effective amount” or “efficacious amount” refers to the amount of an agent, or combined amounts of two agents, that, when administered to a mammal or other subject for treating a disease, is sufficient to affect such treatment for the disease. The “therapeutically effective amount” will vary depending on the agent(s), the disease and its severity and the age, weight, etc., of the subject to be treated.

As used herein, the term “evolution”, or “evolving”, refers to using one or more methods of mutagenesis to generate a different polynucleotide encoding a different polypeptide, which is itself an improved biological molecule and/or contributes to the generation of another improved biological molecule.

“Physiological” or “normal” or “normal physiological” conditions are conditions such as, but not limited to, temperature, pH, osmotic pressure, osmolality, oxidative stress and electrolyte concentration, as well as other parameters, that would be considered within a normal range at the site of administration, or at the tissue or organ at the site of action, to a subject.

It is to be understood that the present disclosure and the aspects and embodiments provided herein, are not limited to particular examples disclosed, as such may, of course, vary. It is also to be understood that the terminology used herein is for the purpose of disclosing particular examples and embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a chimeric antigen receptor” includes a plurality of such chimeric antigen receptors and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

### DETAILED DESCRIPTION

The aspects and embodiments disclosure herein overcome the problem of off-tumor effects of current therapies by providing in one aspect, a conditionally active chimeric antigen receptor (CAR) for binding Axl and/or Ror2. The CARs for binding Axl and/or Ror2 are active in a tumor environment but not normal physiological tissue/organs. In addition to various embodiments of CARs that bind Axl and/or Ror2, provided herein are nucleic acid embodiments that include a nucleotide sequence encoding any of the CARs provided herein, as well as viral constructs for expressing any of the CARs, cells infected with at least one of the viral constructs, as well as recombinant cells expressing the CARs. A CAR of the present disclosure can be used in various methods, which are also provided, along with methods of infecting T-cells and other cytotoxic cells with expression vectors, such as recombinant viral vectors, that encode the CARs of the present disclosure.

### Chimeric Antigen Receptors

The present disclosure provides a chimeric antigen receptor, which, for simplicity, is referred to herein as a “CAR.” In illustrative embodiments, a CAR of the present disclosure binds to Axl or Ror2 and in further illustrative embodiments, the CAR binds to Axl or Ror2 in a conditionally active manner. In certain illustrative embodiments, a CAR provided herein includes: a) at least one conditionally active antigen-specific targeting region (ASTR) that exhibits an increased binding at pH 6.7 compared to a pH of 7.4; b) a transmembrane domain; and c) an intracellular activating domain. In illustrative embodiments, the antigen-specific targeting region of the CAR is a conditionally active scFv portion of an anti-Axl or anti-Ror2 antibody. Furthermore, in illustrative embodiments the ASTR exhibits an increase in activity in a tumor environment or an in vitro tumor surrogate assay condition compared to a normal physiological environment.

A CAR of the present disclosure can be present in the plasma membrane of a eukaryotic cell, e.g., a mammalian cell, where suitable mammalian cells include, but are not limited to, a cytotoxic cell, a T lymphocyte, a stem cell, a progeny of a stem cell, a progenitor cell, a progeny of a progenitor cell, and an NK cell, an NK-T cell, and a macrophage. When present in the plasma membrane of a eukaryotic cell, a CAR of the present disclosure is active in the presence of Axl and/or Ror2 that, in certain conditions, binds the ASTR. Axl and Ror2 are a second member of the specific binding pair. Axl and/or Ror2 of the specific binding pair can be a soluble (e.g., not bound to a cell) factor; a factor present on the surface of a cell such as a target cell; a factor presented on a solid surface; a factor present in a lipid bilayer; and the like. Where the ASTR is an antibody, and the second member of the specific binding pair is an antigen, the antigen can be a soluble (e.g., not bound to a cell) antigen; an antigen present on the surface of a cell such as a target cell; an antigen presented on a solid surface; an antigen present in a lipid bilayer; and the like.

In some instances, a CAR of the present disclosure, when present in the plasma membrane of a eukaryotic cell, and when activated by Axl and/or Ror2, increases expression of at least one nucleic acid in the cell. For example, in some cases, a CAR of the present disclosure, when present in the plasma membrane of a eukaryotic cell, and when activated by Axl and/or Ror2, increases expression of at least one nucleic acid in the cell by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared with the level of transcription of the nucleic acid in the absence of Axl and/or Ror2.

As an example, the CAR of the present disclosure can include an immunoreceptor tyrosine-based activation motif (ITAM)-containing intracellular signaling polypeptide; in such cases, a CAR of the present disclosure, when present in the plasma membrane of a eukaryotic cell, and when activated by Axl and/or Ror2, increases nuclear factor of activated T cells (NFAT)-dependent transcription. NFAT-dependent transcription includes transcription induced by any member of the NFAT family, including, e.g., NFATe1, NFATc2, NFATc3, NFATc4, NFAT5; AP-1; Spl; NKKB; and the like.

A CAR of the present disclosure, when present in the plasma membrane of a eukaryotic cell, and when activated by Axl or Ror2, can, in some instances, result in increased production of one or more cytokines by the cell. For example, a CAR of the present disclosure, when present in the plasma membrane of a eukaryotic cell, and when activated by Axl or Ror2, can increase production of a cytokine by the cell by at least 10%, at least 15%, at least 20%, at least 25%, at least 30%, at least 40%, at least 50%, at least 75%, at least 2-fold, at least 2.5-fold, at least 5-fold, at least 10-fold, or more than 10-fold, compared with the amount of cytokine produced by the cell in the absence of Axl and/or Ror2. In some embodiments, a CAR of the present disclosure, when present in the membrane of a eukaryotic cell, and when activated by Axl and/or Ror2, can increase secretion of a cytokine by the cell by at least 10%, at least 15%, at least 20%, at least 25%, at least 30%, at least 40%, at least 50%, at least 75%, at least 2-fold, at least 2.5-fold, at least 5-fold, at least 10-fold, or more than 10-fold, compared with the amount of cytokine secreted by the cell in the absence of Axl and/or Ror2. Cytokines whose production can be increased include, but are not limited to interferon gamma (IFN-γ), tumor necrosis factor-alpha (TNF-α), IL-2, IL-15, IL-12, IL-4, IL-5, IL-10; a chemokine; a growth factor; and the like.

In some cases, a CAR of the present disclosure, when present in the plasma membrane of a eukaryotic cell, and when activated by Axl and/or Ror2, can result in an increase in transcription of a nucleic acid in the cell, an increase in production of a cytokine, and an increase in secretion of the cytokine by the cell.

In some instances, a CAR of the present disclosure, when present in the plasma membrane of a eukaryotic cell, and when activated by Axl and/or Ror2, results in cytotoxic activity by the cell toward a target cell that expresses on its cell surface an antigen to which the antigen-binding domain of the first polypeptide of the CAR binds. For example, where the eukaryotic cell is a cytotoxic cell (e.g., an NK cell or a cytotoxic T lymphocyte), a CAR of the present disclosure, when present in the plasma membrane of the cell, and when activated by Axl and/or Ror2, increases cytotoxic activity of the cell toward a target cell that expresses on its cell surface Axl and/or Ror2. For example, where the eukaryotic cell is an NK cell or a T lymphocyte, a CAR of the present disclosure, when present in the plasma membrane of the cell, and when activated by Axl and/or Ror2, increases cytotoxic activity of the cell by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared to the cytotoxic activity of the cell in the absence of Axl and/or Ror2.

In some cases, a CAR of the present disclosure, when present in the plasma membrane of a eukaryotic cell, and when activated by Axl and/or Ror2, can result in other CAR activation related events such as proliferation and expansion (either due to increased cellular division or anti-apoptotic responses).

In some cases, a CAR of the present disclosure, when present in the plasma membrane of a eukaryotic cell, and when activated by Axl and/or Ror2, can result in other CAR activation related events such as intracellular signaling modulation, cellular differentiation, or cell death.

A CAR of the present disclosure can be present in a eukaryotic cell membrane, where the first and second polypeptides of the CAR are not covalently linked to one another. A CAR of the present disclosure can be present in a eukaryotic cell membrane as a single heterodimer that is not covalently linked to any other polypeptide in the membrane. Alternatively, a first CAR of the present disclosure can be present in a eukaryotic cell membrane as a heterodimer that is covalently or non-covalently linked to a second CAR of the present disclosure. In some cases, the first and the second CAR are covalently linked via a disulfide bond formed between cysteines present in a hinge region present in both the first polypeptide of the first CAR and the first polypeptide of the second CAR.

In some cases, a CAR of the present disclosure can be present in a eukaryotic cell membrane, where the first polypeptides of the CAR include an antibody fragment and the second polypeptides of the CAR include a signal transducing domain derived from a cytokine receptor, such that, upon dimerization, the CAR may represent a heterodimeric-signalobody CAR, e.g., a signalobody composed of at least two independent polypeptides. A “signalobody”, as it is known in the art, is a single chimeric macromolecule composed of an antibody fragment and a signal transduction domain derived from a cytokine receptor. In certain instances, a heterodimeric-signalobody CAR of the present disclosure, when present in the cell membrane of a eukaryotic cell, dimerized by a dimerizer, and activated by an antigen, e.g., an oligomerized antigen, may induce the oligomerization of the heterodimeric-signalobody CAR. Such ligand-induced oligomerization of a heterodimeric-signalobody CAR may activate, e.g., increase, or perpetuate, e.g., maintain, signal transduction, e.g., ligand-induced oligomerization of a heterodimeric-signalobody CAR may transmit a signal eliciting a cellular response. In some instances, a plurality of heterodimeric-signalobody CARs may be utilized combinatorially to elicit a desired cellular response.

### Antigen-Specific Targeting Regions

A CAR of the present disclosure includes a member of a specific binding pair, which is typically an ASTR. Specific binding pairs include, but are not limited to, antigen-antibody binding pairs; ligand-receptor binding pairs; and the like. Thus, a member of a specific binding pair suitable for use in a CAR of the present disclosure includes an ASTR that is an antibody, an antigen, a ligand, a receptor binding domain of a ligand, a receptor, a ligand binding domain of a receptor, and an affibody.

An ASTR suitable for use in a CAR of the present disclosure can be any antigen-binding polypeptide. In certain embodiments, the ASTR is an antibody such as a full-length antibody, a single-chain antibody, an Fab fragment, an Fab′ fragment, an (Fab′)2 fragment, an Fv fragment, and a divalent single-chain antibody or a diabody.

In some embodiments, the ASTR is a single chain Fv (scFv). In some embodiments, the heavy chain is positioned N-terminal of the light chain in the CAR. In other embodiments, the light chain is positioned N-terminal of the heavy chain in the CAR. In any of the disclosed embodiments, the heavy and light chains can be separated by a linker as discussed in more detail herein. In any of the disclosed embodiments, the heavy or light chain can be at the N-terminus of the CAR and is typically C-terminal of another domain, such as a signal sequence or peptide.

Other antibody-based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable for use with the CARs and methods using the CARs of the present disclosure. In some instances, T-cell receptor (TCR) based recognition domains such as single chain TCR (scTv, single chain two-domain TCR containing VatVp) are also suitable for use.

### Conditionally Active Biologic CARs (CAB-CARs)

CARs of the present disclosure are typically conditionally active. This property is typically the result of the conditionally active nature of the ASTR domain of the CAR. In illustrative embodiments, CAB-CARs of the present disclosure have a higher binding affinity to Axl or Ror2 under a condition(s) in a tumor microenvironment than under a condition in a non-tumor microenvironment. In some embodiments, the condition in the tumor microenvironment and the condition in a non-tumor microenvironment are both pH. Thus, the CAB-CARs can selectively bind to Axl or Ror2 in a conditionally active manner typically because they have a higher binding affinity for Axl or Ror2 at a pH of about 6.0-6.8, a pH that is encountered in a tumor microenvironment, compared to a pH of 7.2-7.8, a pH that is encountered in a normal physiological environment. For example, CAB-CARs can have a higher binding affinity to Axl or Ror2 at pH 6.7 than at pH 7.4. Additionally, or alternatively, CAB-CARs can have a higher binding affinity to Axl or Ror2 at pH 6.0 than at pH 7.4. Such conditions can be tested in an in vitro tumor surrogate assay that for example, tests for antigen binding and/or CAR activity (e.g. cell lysis) under one or more conditions found in an in vivo tumor environment, as set out in more detail below, which differ from the corresponding condition(s) in normal physiological tissue. For example, an in vitro tumor surrogate assay condition can be a low pH (e.g. 6.0-6.8) compared to a physiological pH (7.2-7.8). In an illustrative example, a tumor surrogate assay condition is a pH of 6.7 whereas a corresponding physiological pH is 7.4.

In some embodiments, CAB-CARs can be obtained by identifying a VH and/or VL of an antibody that was identified under physiologic conditions (i.e. parent, “wild type” or “wt” antibody). Antibodies can then mutated and tested (evolved). A skilled artisan can utilize the method for identifying conditionally active antibodies disclosed in U.S. Pat. No. 8,709,755 to identify additional conditionally active antibodies and antibody fragments that can be used in ASTRs for CABCARs of the present disclosure. The complementarity determining region (CDR) is a 3-dimensional structure that is formed by the interaction of the VH and VL. To alter the binding specificity of a starting point (“wt” antibody), it is reasonable to expect that mutating either or both the VH/VL could lead to CAB activity in a CAB-CAR. To generate a conditionally active antibody, both the VH and the VL are typically identified under physiologic conditions, and then either the VH or the VL or more typically both the VH and VL are mutated and tested in non-physiologic conditions, such as a pH of 6.0 to 6.7 or another condition of a tumor microenvironment, to generate a conditionally active antibody.

Nucleic acid encoding VH and/or VL regions of wild-type antibodies can be cloned using known methods. Variants of such wild-type VH and VL regions can then be prepared by introducing modifications into the nucleotide sequence encoding the heavy and light chain variable regions. Such modifications include, for example, deletions from, and/or insertions into and/or substitutions of residues within the amino acid sequences of the antibody or antibody fragment. Any combination of deletion(s), insertion(s), and substitution(s) can be made to arrive at a conditionally active antibody fragment.

More detailed methods for making and/or isolating conditionally active ASTRs are provided in a separate section herein.

### Conditionally Active ASTRs Targeting Axl

Illustrative embodiments of any of the various aspects provided herein, include a CAR with a conditionally active ASTR that specifically binds to an Axl protein at a pH of 6.7 compared to a pH of 7.4 Examples of such ASTRs and CARs containing such ASTRs, are provided in the Examples herein. In certain embodiments, the ASTR binds to the same epitope of Axl as an antibody that includes the heavy chain of SEQ ID NO:79 and the light chain of SEQ ID NO:80. In illustrative embodiments, the ASTR binds to the same epitope of Axl as a single-chain variable antibody fragment|comprising an antibody heavy chain of SEQ ID NO:79 and an antibody light chain of SEQ ID NO:80.

The ASTR can be a single-chain antibody, an Fab fragment, an Fab′ fragment, an (Fab′)2 fragment, an Fv fragment, a divalent single-chain antibody, or a diabody. In illustrative embodiments, the conditionally active ASTR that binds Axl is a single-chain variable fragment comprising a heavy chain and a light chain.

In some embodiments where the ASTR binds to Axl, and in illustrative embodiments binds to the same epitope of Axl as a single-chain variable antibody fragment comprising an antibody heavy chain of SEQ ID NO:79 and an antibody light chain of SEQ ID NO:80, the heavy chain variable region can include three complementarity determining regions, said regions having sequences H1, H2, and H3, wherein:
the H1 sequence is X₁GX₂TMN (SEQ ID NO:87);
the H2 sequence is LIKPSNGGTSYNQKFKG (SEQ ID NO:88); and the H3 sequence is GX₃YX₄SYX₅AMDY (SEQ ID NO:89), wherein X₁ is T or W; X₂ is H or A, X₃ is H or D; X₄ is E or H; and X₅ is E or F.

In some embodiments where the ASTR binds to Axl, and in illustrative embodiments binds to the same epitope of Axl as a single-chain variable antibody fragment comprising an antibody heavy chain of SEQ ID NO:79 and an antibody light chain of SEQ ID NO:80, including the heavy chain embodiments immediately above, the ASTR can include a light chain variable region that includes three complementarity determining regions, said regions having sequences L1, L2, and L3, wherein: the L1 sequence is KASQDVX₆SAVA (SEQ ID NO:90);
the L2 sequence is WX₇X₈TRX₉T (SEQ ID NO:91); and
the L3 sequence is QEHFSX₁₀PLX₁₁ (SEQ ID NO: 92),
wherein X₆ is S or V; X₇ is A or Q; X₈ is S or D; X₉ is H or D; X₁₀ is T or P; and X₁₁ is T or R.

In some embodiments where the ASTR binds to Axl, and in illustrative embodiments binds to the same epitope of Axl as a single-chain variable antibody fragment comprising an antibody heavy chain of SEQ ID NO:79 and an antibody light chain of SEQ ID NO:80, the heavy chain variable region can include three complementarity determining regions, said regions having sequences H1, H2, and H3, wherein:
the H1 sequence is X₁GX₂X₃MX₄ (SEQ ID NO: 134);
the H2 sequence is LIKX₅SNGGTX₆YNQKFKG (SEQ ID NO: 135); and the H3 sequence is GX₇X₈X₉X₁₀X₁₁X₁₂X₁₃X₁₄DYX₁₅X₁₆ (SEQ ID NO: 136),
wherein X₁ is T, A, or W; X₂ is H or A; X₃ is T or I; X₄ is N or I; X₅ is P or N; X₆ is S, I, or T; X₇ is H, D, E, P, R, or W; X₈ is Y or N; X₉ is E, A, D, F, G, H, I, L, M, N, R, V, or Y; X₁₀ is S, D, M, N, or Q; X₁₁ is Y, C, E, or P; X₁₂ is F, E, N, S, T, or V; X₁₃ is A, D, G, L, or Y; X₁₄ is M, E, or F; X₁₅ is W, A, D, H, L, N, P, R, or T; and X₁₆ is G or H.

In some embodiments where the ASTR binds to Axl, and in illustrative embodiments binds to the same epitope of Axl as a single-chain variable antibody fragment comprising an antibody heavy chain of SEQ ID NO:79 and an antibody light chain of SEQ ID NO:80, including the heavy chain embodiments immediately above, the ASTR can include a light chain variable region that includes three complementarity determining regions, said regions having sequences L1, L2, and L3, wherein: the L1 sequence is KASQDX₁₇X₁₈SX₁₉VX₂₀ (SEQ ID NO: 137);
the L2 sequence is X₂₁X₂₂X₂₃TRX₂₄T (SEQ ID NO: 138); and
the L3 sequence is QEX₂₅X₂₆SX₂₇X₂₈X₂₉X₃₀ (SEQ ID NO: 139), wherein X₁₇ is V, D, G, N, or W; X₁₈ is S or V; X₁₉ is A, L, or M; X₂₀ is A, D, N, or Q; X₂₁ is W or F; X₂₂ is A, I, N, P, or Q; X₂₃ is S or D; X₂₄ is H or D; X₂₅ is H, C, F, I, L, Q, S, T, V, or Y; X₂₆ is F, C, D, E, G, N, or S; X₂₇ is T, C, or P; X₂₈ is P, A, C, D, E, H, K, S, T, V, or W; X₂₉ is L, G, or R; and X₃₀ is T, I, or R.

In certain illustrative embodiments, the ASTR includes the light chain variable region of SEQ ID NO:80 and/or the heavy chain variable region of SEQ ID NO:79. These illustrative embodiments can include the heavy chain N-terminal to the light chain or the light chain N-terminal to the heavy chain. In an illustrative embodiment, the anti-Axl ASTR can include the sequence of any of SEQ ID NO: 128, SEQ ID NO: 129, SEQ ID NO:159, SEQ ID NO: 160, or SEQ ID NO:161.

The heavy and light chains of any of these anti-Axl embodiments that include two variable regions, the variable regions are typically separated by a linker. The linker can be between 6 and 100 amino acids in length. In some embodiments, the linker is linker 1 (SEQ ID NO:53), linker 2 (SEQ ID NO:54), or linker 3 (SEQ ID NO:55). In illustrative embodiments, the two variable regions are a heavy chain variable region and a light chain variable region. Either the heavy chain or light chain can be located N-terminal to the other on the ASTR. In certain illustrative embodiments, the heavy chain is N-terminal to the light chain.

Exemplary conditionally active CARS (CAB-CARs) that have increased binding to Axl at pH 6.7 compared to ph7.4 are found in Example 1 herein. In illustrative embodiments, the CAR or ASTR can bind to the same epitope of Axl as a single-chain variable antibody fragment comprising an antibody heavy chain of SEQ ID NO:79 and an antibody light chain of SEQ ID NO:80. In further embodiments of such illustrative embodiments, the anti-Axl CAR or ASTR comprises or is a single chain variable fragment (scFv). In further illustrative examples, the anti-Axl scFv comprises either a heavy chain that is N-terminal to a light chain or a light chain that is N-terminal to a heavy chain. In any of the embodiments herein that includes a CAR, and in illustrative embodiments binds to the same epitope of Axl as an antibody that includes the antibody heavy chain of SEQ ID NO:79 and the antibody light chain of SEQ ID NO:80, the ASTR can include any of SEQ ID NOs:128, 129, 159, 160, or 161. Furthermore, anti-Axl CARs of any of the embodiments herein can include any of the CAR components provided herein. In certain exemplary embodiments, the anti-Axl CARs can include the CAR components listed in Table 1 and can be any of the CARs in Table 1. More typically for any embodiments herein that include an anti-Axl CAR, the CAR is a CAB-CAR, and in non-limiting illustrative embodiments, can include, for example, any of the CAB-CAR components and CAB-CARs provided in Table 1 that demonstrated cytotoxic activity. For example, the anti-Axl CAB-CAR can include a CD8 signal peptide, a CD8 or CD28 stalk/transmembrane domain, a CD137, ICA, or both a ICA co-stimulatory domain and a CD137 co-stimulatory domain, and/or a CD3Z activation domain. Furthermore, illustrative CARs for any of the embodiments herein that includes an anti-Axl CAR, and especially an anti-Axl CAB-CAR, in non-limiting illustrative embodiments include any of the anti-Axl CAB-CARs that demonstrated conditional cytotoxic activity in Table 1. Such illustrative CAB-CARs include F1-2-1, F1-2-2, F1-2-3, F1-2-6, F1-2-8, F1-2-10, F1-2-13, F1-2-14, F1-2-15, F1-2-22, or F1-2-23 of Table 1. In any of the embodiments herein that includes an ASTR, the ASTR can include the ASTR of F1-2-1, F1-2-2, F1-2-3, F1-2-6, F1-2-8, F1-2-10, F1-2-13, F1-2-14, F1-2-15, F1-2-22, or F1-2-23. Furthermore, illustrative CARs for any of the embodiments herein that includes an anti-Axl CAR, and especially an anti-Axl CAB-CAR, in non-limiting illustrative embodiments include any of the anti-Axl CAB-CARs that demonstrated high conditional cytotoxic activity in Table 1. Such illustrative CAB-CARs include F1-2-13, F1-2-15, F1-2-22, or F1-2-23. Accordingly, in any of the embodiments herein that includes an ASTR, the ASTR can include the ASTR of F1-2-1, F1-2-2, F1-2-3, F1-2-6, F1-2-8, F1-2-10, F1-2-13, F1-2-14, F1-2-15, F1-2-22, or F1-2-23.

The heavy chain variable region polypeptides and light chain variable region polypeptides disclosed herein were identified from a parent antibody heavy chain variable region (SEQ ID NO:93) and a parent antibody light chain variable region (SEQ ID NO:94) using a method disclosed in U.S. Pat. No. 8,709,755. A skilled artisan can utilize the method for identifying conditionally active antibodies disclosed in U.S. Pat. No. 8,709,755 to identify additional conditionally active antibodies and antibody fragments that can be used in ASTRs for CAB-CARs of the present disclosure.

In some embodiments, the heavy chain variable regions can be SEQ ID NOs:112-114. In some embodiments, the light chain variable regions can be SEQ ID NOs:108-111. These heavy and light chain variable regions can specifically bind to Axl. Antibodies comprising any one of these heavy and light chain variable regions have been found to have a higher binding affinity to Axl at a pH 6.7 than at a pH 7.4. A pH 6.7 is a pH found in the tumor microenvironment. A pH 7.4 is a pH found in a non-tumor, normal physiological microenvironment.

The CAR can also include variants of the heavy and light chain variable regions of the sequences of SEQ ID NOs:108-114 that can specifically bind to Axl. In order to derive these variants, it was determined that the complementarity determining regions (CDRs) of the heavy chain variable regions (H1-H3) and the CDRs of the light chain variable regions (L1-L3) should remain intact. The variants of these heavy and light chain variable regions may be prepared by introducing appropriate modifications into the nucleotide sequence encoding the heavy and light chain variable regions, or by peptide synthesis. Such modifications include, for example, deletions from, and/or insertions into and/or substitutions of residues within the amino acid sequences of the antibody or antibody fragment. Any combination of deletion(s), insertion(s), and substitution(s) can be made to arrive at the final construct, provided that the final construct possesses at least one of the desired characteristics, e.g., antigen-binding.

### Conditionally Active ASTRs Targeting Ror2

Illustrative embodiments of any of the various aspects provided herein, include a CAR with a conditionally active ASTR that specifically binds to a Ror2 protein at a pH of 6.7 compared to a pH of 7.4. Examples of such ASTRs and CARs containing such ASTRs, are provided in the Examples herein. In certain embodiments, the ASTR binds to the same epitope of Ror2 as an antibody that includes the heavy chain of SEQ ID NO:82 or SEQ ID NO:83 or the ASTR binds to the same epitope of Ror2 as an antibody that includes the heavy chain of SEQ ID NO:151. In illustrative embodiments, the ASTR binds to the same epitope of Ror2 as a single-chain variable antibody fragment comprising an antibody heavy chain of SEQ ID NO:82 or SEQ ID NO:83 or the ASTR binds to the same epitope of Ror2 as a single-chain variable antibody fragment comprising an antibody heavy chain of SEQ ID NO:151. In illustrative embodiments, the ASTR binds to the same epitope of Ror2 as a single-chain variable antibody fragment that includes the antibody heavy chain of SEQ ID NO:82 or SEQ ID NO:83 and an antibody light chain of SEQ ID NO:84 or the ASTR binds to the same epitope of Ror2 as a single-chain variable antibody fragment that includes the antibody heavy chain of SEQ ID NO:151 and the antibody light chain of SEQ ID NO: 152.

The ASTR can be a single-chain antibody, an Fab fragment, an Fab′ fragment, an (Fab′)2 fragment, an Fv fragment, a divalent single-chain antibody, or a diabody. In illustrative embodiments, the conditionally active ASTR that binds Ror2 is a single-chain variable fragment comprising a heavy chain and a light chain.

In some embodiments where the ASTR binds to Ror2, the ASTR can include a heavy chain variable region with three complementarity determining regions, said regions having H1, H2, and H3 sequences, wherein:
the H1 sequence is GYTX₁TEX₂TX₃H (SEQ ID NO:95) or X₄GYSITTGYYWN (SEQ ID NO:96);
the H2 sequence is GX₅NX₆NNGGTGYNQKFKG (SEQ ID NO:97) or YITYDGSKNYNPSLKN (SEQ ID NO:98); and
the H3 sequence is GSLYSYGNSYFDY (SEQ ID NO:99) or FEGVWX₇GLDY (SEQ ID NO: 100),
wherein X₁ is F or E; X₂ is Y or D, X₃ is M or D; X₄ is T or S; X₅ is E or I; X₆ is T or D; and X₇ is Y or G.

In some embodiments where the ASTR binds to Ror2, and in illustrative embodiments binds to the same epitope of Ror2 as an antibody that includes the antibody heavy chain of SEQ ID NO: 151 and the antibody light chain of SEQ ID NO: 152, the ASTR can include a heavy chain variable region with three complementarity determining regions, said regions having H1, H2, and H3 sequences, wherein:
the H1 sequence is GYTX₁TEX₂TX₃H (SEQ ID NO:95);
the H2 sequence is GX₅NX₆NNGGTGYNQKFKG (SEQ ID NO:97); and
the H3 sequence is GSLYSYGNSYFDY (SEQ ID NO:99),
wherein X₁ is F or E; X₂ is Y or D, X₃ is M or D; X₅ is E or I; and X₆ is T or D.

In some embodiments where the ASTR binds to Ror2, and in illustrative embodiments binds to the same epitope of Ror2 as an antibody that includes the antibody heavy chain of SEQ ID NO:82 or SEQ ID NO:83 and the antibody light chain of SEQ ID NO:84, the ASTR can include a heavy chain variable region with three complementarity determining regions, said regions having H1, H2, and H3 sequences, wherein:
the H1 sequence is X₄GYSITTGYYWN (SEQ ID NO:96);
the H2 sequence is YITYDGSKNYNPSLKN (SEQ ID NO:98); and
the H3 sequence is FEGVWX₇GLDY (SEQ ID NO: 100),
wherein X₄ is T or S; and X₇ is Y or G.

In some embodiment where the ASTR binds to Ror2, including but not limited to those having a heavy chain with the H1, H2, and H3 sequences above, the ASTR includes a light chain variable region that includes three complementarity determining regions, said regions having L1, L2, and L3 sequences, wherein:
the L1 sequence is SATSSX₈SYMH (SEQ ID NO: 101) or RASESVDRYGNSFIH (SEQ ID NO: 102);
the L2 sequence is X₉TSNLAS (SEQ ID NO: 103) or RTYNLES (SEQ ID NO: 104); and
the L3 sequence is QQRSSYPFT (SEQ ID NO: 105) or QQTNEDPWT (SEQ ID NO: 106),
wherein X₈ is E or V; and X₉ is G or H.

In some embodiments where the ASTR binds to Ror2, and in illustrative embodiments binds to the same epitope of Ror2 as an antibody that includes the antibody heavy chain of SEQ ID NO: 151 and the antibody light chain of SEQ ID NO: 152, the ASTR can include a light chain variable region with three complementarity determining regions, said regions having L1, L2, and L3 sequences, wherein:
the L1 sequence is SATSSX₈SYMH (SEQ ID NO:101);
the L2 sequence is X₉TSNLAS (SEQ ID NO: 103); and
the L3 sequence is QQRSSYPFT (SEQ ID NO: 105),
wherein X₈ is E or V; and X₉ is G or H.

In some embodiments where the ASTR binds to Ror2, and in illustrative embodiments binds to the same epitope of Ror2 as an antibody that includes the antibody heavy chain of SEQ ID NO:82 or SEQ ID NO:83 and the antibody light chain of SEQ ID NO:84, the ASTR can include a light chain variable region with three complementarity determining regions, said regions having L1, L2, and L3 sequences, wherein:
the L1 sequence is RASESVDRYGNSFIH (SEQ ID NO: 102);
the L2 sequence is RTYNLES (SEQ ID NO: 104); and
the L3 sequence is QQTNEDPWT (SEQ ID NO: 106).

In some embodiments where the ASTR binds to Ror2, the ASTR can include a heavy chain variable region including three complementarity determining regions, said regions having H1, H2, and H3 sequences, wherein:
the H1 sequence is GYTX₁TEX₂X₃X₄H (SEQ ID NO: 140) or GYSITTGX₂₉YWN (SEQ ID NO:141);
the H2 sequence is X₅X₆X₇X₈NNGGTGYNQKFKG (SEQ ID NO: 142) or YITYDGSX₃₀NYNPSLKN (SEQ ID NO: 143); and
the H3 sequence is X₉X₁₀X₁₁SX₁₂YX₁₃YX₁₄X₁₅SYFX₁₆X₁₇X₁₈ (SEQ ID NO: 144) or CSX₃₁X₃₂X₃₃X₃₄VX₃₅X₃₆X₃₇LDX₃₈ (SEQ ID NO: 145),
wherein X₁ is F or E; X₂ is Y or D; X₃ is T or C; X₄ is M, D, E, or Y; X₅ is G or S; X₆ is I or E; X₇ is N, C, L, or V; X₈ is T, D or E; X₉ is A, M, or T; X₁₀ is Ror H; X₁₁ is G or E; X₁₂ is L or F; X₁₃ is S or G; X₁₄ is G or D; X₁₅ is N or E; X₁₆ is D or L; X₁₇ is Y, C, or T; X₁₈ is W or L; X₂₉ is Y, E, R, or T; X₃₀ is K or N; X₃₁ is R, G, H, W, or Y; X₃₂ is F, C, N, or Q; X₃₃ is E or S; X₃₄ is G, E, F, H, M, Q, or S; X₃₅ is W, A, I, P, Q, T, or V; X₃₆ is Y, G, N, or Q; X₃₇ is G, S, or T; and X₃₈ is Y or I.

In some embodiments where the ASTR binds to Ror2, and in illustrative embodiments binds to the same epitope of Ror2 as an antibody that includes the antibody heavy chain of SEQ ID NO: 151 and the antibody light chain of SEQ ID NO: 152, the ASTR can include a heavy chain variable region including three complementarity determining regions, said regions having H1, H2, and H3 sequences, wherein:
the H1 sequence is GYTX₁TEX₂X₃X₄H (SEQ ID NO: 140);
the H2 sequence is X₅X₆X₇X₈NNGGTGYNQKFKG (SEQ ID NO: 142); and
the H3 sequence is X₉X₁₀X₁₁SX₁₂YX₁₃YX₁₄X₁₅SYFX₁₆X₁₇X₁₈ (SEQ ID NO: 144),
wherein X₁ is F or E; X₂ is Y or D; X₃ is T or C; X₄ is M, D, E, or Y; X₅ is G or S; X₆ is I or E; X₇ is N, C, L, or V; X₈ is T, D or E; X₉ is A, M, or T; X₁₀ is Ror H; X₁₁ is G or E; X₁₂ is L or F; X₁₃ is S or G; X₁₄ is G or D; X₁₅ is N or E; X₁₆ is D or L; X₁₇ is Y, C, or T; and X₁₈ is W or L.

In some embodiments where the ASTR binds to Ror2, and in illustrative embodiments binds to the same epitope of Ror2 as an antibody that includes the antibody heavy chain of SEQ ID NO:82 or SEQ ID NO:83 and the antibody light chain of SEQ ID NO:84, the ASTR can include a heavy chain variable region including three complementarity determining regions, said regions having H1, H2, and H3 sequences, wherein:
the H1 sequence is GYSITTGX₂₉YWN (SEQ ID NO:141);
the H2 sequence is YITYDGSX₃₀NYNPSLKN (SEQ ID NO: 143); and
the H3 sequence is CSX₃₁X₃₂X₃₃X₃₄VX₃₅X₃₆X₃₇LDX₃₈ (SEQ ID NO: 145),
wherein X₂₉ is Y, E, R, or T; X₃₀ is K or N; X₃₁ is R, G, H, W, or Y; X₃₂ is F, C, N, or Q; X₃₃ is E or S; X₃₄ is G, E, F, H, M, Q, or S; X₃₅ is W, A, I, P, Q, T, or V; X₃₆ is Y, G, N, or Q; X₃₇ is G, S, or T; and X₃₈ is Y or I.

In some embodiments where the ASTR binds to Ror2, the ASTR can include a light chain variable region that includes three complementarity determining regions, said regions having L1, L2, and L3 sequences, wherein:
the L1 sequence is SATSSX₁₉X₂₀X₂₁MX₂₂ (SEQ ID NO: 146) or RASESVDRYGNSX₃₉IH (SEQ ID NO: 147);
the L2 sequence is X₂₃TSNLAS (SEQ ID NO: 148) or X₄₀TYX₄₁LES (SEQ ID NO: 149); and
the L3 sequence is QX₂₄X₂₅SX₂₆YPFX₂₇X₂₈ (SEQ ID NO: 150) or QQX₄₂NX₄₃DPX₄₄TX₄₅ (SEQ ID NO:85),
wherein X₁₉ is V or E; X₂₀ is S or D; X₂₁ is Y, C, or D; X₂₂ is H, G, or L; X₂₃ is G, C, H, or P; X₂₄ is Q or E; X₂₅ is R or H; X₂₆ is S, D, G, I, Q, or V; X₂₇ is T or D; X₂₈ is F, D, or E; X₃₉ is F, S, or T; X₄₀ is R, C, D, E, or W; X₄₁ is N or D; X₄₂ is T, I, or P; X₄₃ is E or V; X₄₄ is W or T; and X₄₅ is F or T.

In some embodiments where the ASTR binds to Ror2, and in illustrative embodiments binds to the same epitope of Ror2 as an antibody that includes the antibody heavy chain of SEQ ID NO: 151 and the antibody light chain of SEQ ID NO: 152, the ASTR can include a light chain variable region that includes three complementarity determining regions, said regions having L1, L2, and L3 sequences, wherein:
the L1 sequence is SATSSX₁₉X₂₀X₂₁MX₂₂ (SEQ ID NO: 146);
the L2 sequence is X₂₃TSNLAS (SEQ ID NO: 148); and
the L3 sequence is QX₂₄X₂₅SX₂₆YPFX₂₇X₂₈ (SEQ ID NO: 150),
wherein X₁₉ is V or E; X₂₀ is S or D; X₂₁ is Y, C, or D; X₂₂ is H, G, or L; X₂₃ is G, C, H, or P; X₂₄ is Q or E; X₂₅ is R or H; X₂₆ is S, D, G, I, Q, or V; X₂₇ is T or D; and X₂₈ is F, D, or E.

In some embodiments where the ASTR binds to Ror2, and in illustrative embodiments binds to the same epitope of Ror2 as an antibody that includes the antibody heavy chain of SEQ ID NO:82 or SEQ ID NO:83 and the antibody light chain of SEQ ID NO:84, the ASTR can include a light chain variable region that includes three complementarity determining regions, said regions having L1, L2, and L3 sequences, wherein:
the L1 sequence is RASESVDRYGNSX₃₉IH (SEQ ID NO: 147);
the L2 sequence is X₄₀TYX₄₁LES (SEQ ID NO: 149); and
the L3 sequence is QQX₄₂NX₄₃DPX₄₄TX₄₅ (SEQ ID NO:85),
wherein X₃₉ is F, S, or T; X₄₀ is R, C, D, E, or W; X₄₁ is N or D; X₄₂ is T, I, or P; X₄₃ is E or V; X₄₄ is W or T; and X₄₅ is F or T.

In some embodiments, the conditionally active ASTR that binds Ror2, and in illustrative embodiments the conditionally active ASTR that binds to the same epitope of Ror2 as an antibody comprising an antibody heavy chain of SEQ ID NO: 151 and an antibody light chain of SEQ ID NO: 152, includes a heavy chain variable region having an amino acid sequence selected from the sequences of SEQ ID NOs: 115-119 and SEQ ID NO:151. In these illustrative embodiments where the conditionally active ASTR binds to the same epitope of Ror2 as an antibody comprising an antibody heavy chain of SEQ ID NO: 151 and an antibody light chain of SEQ ID NO: 152, optionally including the heavy chains listed in the previous sentence, the light chain can include the light chain of SEQ ID NO:81, SEQ ID NOs: 122-124, or SEQ ID NO: 152.

In certain illustrative embodiments, the conditionally active ASTR binds to the same epitope of Ror2 as an antibody comprising an antibody heavy chain of SEQ ID NO:82 or SEQ ID NO:83 and an antibody comprising an antibody light chain of SEQ ID NO:84, and includes an antibody heavy chain variable region of any one of SEQ ID NOs: 120-121 and 82-83. In these illustrative embodiment where the conditionally active ASTR binds to the same epitope of Ror2 as an antibody comprising an antibody heavy chain of SEQ ID NO:82 or SEQ ID NO:83 and an antibody comprising an antibody light chain of SEQ ID NO:84, optionally including the heavy chains listed in the previous sentence, the light chance can include the light chain of SEQ ID NOs:84 or 86.

Exemplary conditionally active CARs (CAB-CARs) that have increased binding to Ror2 at pH 6.7 compared to pH 7.4 are found in Example 1 herein. In illustrative embodiments, the CAR or ASTR can bind to the same epitope of Ror2 as a single-chain variable antibody fragment comprising an antibody heavy chain of SEQ ID NO:82 or SEQ ID NO:83 and an antibody light chain of SEQ ID NO:84 or the CAR or ASTR can bind to the same epitope of Ror2 as a single-chain variable antibody fragment comprising an antibody heavy chain of SEQ ID NO: 151 and an antibody light chain of SEQ ID NO: 152. In further embodiments of such illustrative embodiments, the anti-Ror2 CAR or ASTR comprises or is a single chain variable fragment (scFv) and in further illustrative examples, comprises a light chain that is N terminal to a heavy chain, or comprises a heavy chain that is N-terminal to a light chain. In any of the embodiments herein that includes a CAR or ASTR, and in illustrative embodiments binds to the same epitope of Ror2 as a single-chain variable antibody fragment comprising an antibody heavy chain of SEQ ID NO:82 or SEQ ID NO:83 and an antibody light chain of SEQ ID NO:84 or binds to the same epitope of Ror2 as a single-chain variable antibody fragment comprising an antibody heavy chain of SEQ ID NO: 151 and an antibody light chain of SEQ ID NO: 152, the ASTR can include any of SEQ ID NOs:130-132, or 153-158. Furthermore, anti-Ror2 CARs of any of the embodiments herein can include any of the CAR components provided herein. In certain exemplary embodiments, the anti-Ror2 CARS can include the CAR components listed in Table 2 and can be any of the CARs in Table 2. More typically for any embodiments herein that include an anti-Ror2 CAR, the CAR is a CAB-CAR, and in non-limiting illustrative embodiments, can include, for example, any of the CAB-CAR components and CAB-CARs provided in Table 2 that demonstrated cytotoxic activity. For example, the anti-Ror2 CAB-CAR can include a CD8 signal peptide, a CD8 or CD28 stalk/transmembrane domain, a CD137 co-stimulatory domain, and/or a CD3Z activation domain. Furthermore, illustrative CARs for any of the embodiments herein that includes an anti-Ror2 CAR and especially an anti-Ror2 CAB-CAR, in non-limiting illustrative embodiments include any of the anti-Ror2 CAB-CARs that demonstrated conditional cytotoxic activity in Table 2. Such illustrative CAB-CARs include F1-1-9, F1-1-10, F1-1-11, F1-1-12, F1-1-15, F1-1-17, F1-1-18, F1-1-19, F1-1-20, F1-1-21, F1-1-23, F1-1-25, or F1-1-26. In any of the embodiments herein that includes an anti-Ror2 ASTR, the ASTR can include the ASTR of F1-1-9, F1-1-10, F1-1-11, F1-1-12, F1-1-15, F1-1-17, F1-1-18, F1-1-19, F1-1-20, F1-1-21, F1-1-23, F1-1-25, or F1-1-26 of Table 2. Furthermore, illustrative CARs for any of the embodiments herein that includes an anti-Ror2 CAR and especially an anti-Ror2 CAB-CAR, in non-limiting illustrative embodiments include any of the anti-Ror2 CAB-CARs that demonstrated high conditional cytotoxic activity in Table 2. Such illustrative CAB-CARs include F1-1-11, F1-1-12, F1-1-15, F1-1-17, F1-1-19, F1-1-20, or F1-1-23. Accordingly, in any of the embodiments herein that includes an anti-Ror2 ASTR, the ASTR can include the ASTR of F1-1-11, F1-1-12, F1-1-15, F1-1-17, F1-1-19, F1-1-20, F1-1-23.

More generally, with respect to any of the embodiments provided herein, whether directed to Axl or Ror2, the ASTR can be a single-chain antibody, an Fab fragment, an Fab′ fragment, an (Fab′)2 fragment, an Fv fragment, a divalent single-chain antibody, or a diabody. In illustrative embodiments, the conditionally active ASTR that binds Ror2 is a single-chain variable fragment comprising a heavy chain and a light chain.

The heavy chain variable region polypeptides and light chain variable region polypeptides disclosed herein were identified from a parent antibody through the method disclosed in U.S. Pat. No. 8,709,755. A skilled artisan can utilize the method for identifying conditionally active antibodies disclosed in U.S. Pat. No. 8,709,755 to identify additional conditionally active antibodies and antibody fragments that can be used in ASTRs for CAB-CARs of the present disclosure.

Amino acid sequences of the heavy chain variable regions of some exemplary ASTRs are shown in SEQ ID NOs:115-121. The amino acid sequences of the light chain variable regions of these exemplary ASTRs are shown in SEQ ID NOs:81, 86, and 122-124. These heavy chain variable regions and light chain variable regions can specifically bind to human Ror2. Antibodies or antibody fragments including any one of these heavy chain variable regions and light chain variable regions have been found to have higher binding affinity to Ror2 at a pH in the tumor microenvironment than at a pH in a non-tumor microenvironment or in physiological conditions. For example, the antibodies and antibody fragments have a higher binding affinity to Ror2 at pH 6.0 than at pH 7.4. In some embodiments, the antibodies and antibody fragments have a higher binding affinity to Ror2 at pH 6.7 than at pH 7.4.

The anti-Ror2 antibodies or antibody fragments have a higher binding affinity to Ror2 in a tumor in comparison with their binding affinity to Ror2 in a normal tissue. These anti-Ror2 antibodies or antibody fragments are believed to have a longer half-life and reduced side-effects, as well as comparable efficacy, in comparison with monoclonal anti-Ror2 antibodies known in the art. These features permit use of a higher dosage of these anti-Ror2 antibodies or antibody fragments to be delivered to a patient thus being a more effective therapeutic option.

Though the ASTR can include the heavy chain variable regions and light chain variable regions having amino acid sequences with SEQ ID NOs:81, 86, and 115-124, the present invention also provides variants thereof that can specifically bind to human Ror2. In order to derive these variants, the complementarity determining regions (CDRs) of the heavy chain variable regions (H1-H3) and the complementarity determining regions of the light chain variable regions (L1-L3) should remain intact. However, the amino acid sequence of the heavy chain variable regions and light chains variable regions outside of the complementarity determining regions may be mutated in accordance with the principles of substitution, insertion and deletion. The variants of the heavy chain variable regions and light chain variable regions may be prepared by introducing appropriate modifications into the nucleotide sequence encoding the heavy chain variable regions and light chain variable regions, or by peptide synthesis. Such modifications include, for example, deletions from, and/or insertions into and/or substitutions of residues within the amino acid sequences of the heavy chain variable regions and light chain variable regions. Any combination of deletion, insertion, and substitution can be made to arrive at the ASTR for the CARs, provided that they possess the desired characteristics, e.g., conditional antigen-binding to human Ror2.

### Multispecific ASTRs

In some embodiments, the ASTR can be multispecific, e.g. bispecific antibodies. Multispecific antibodies have binding specificities for at least two different sites or targets. In certain embodiments, one of the binding specificities is for Axl or Ror2 and the other is for another antigen. In certain embodiments, bispecific antibodies may bind to two different epitopes of Axl or Ror2. Bispecific antibodies may also be used to localize cytotoxic agents to cells which express Axl or Ror2. Bispecific antibodies can be prepared as full length antibodies or antibody fragments. In certain embodiments, one of the binding specifities binds to Axl and the other binds to Ror2, where neither, either or both can be conditionally active.

An ASTR suitable for use in a CAR of the present disclosure can have a variety of antigen-binding specificities. In illustrative embodiments, the ASTR binds to Axl or Ror2, which are known to be expressed on certain cancer cells (i.e. cancer-specific antigens). In some cases, the ASTR is bispecific, and in addition to an antigen-binding domain that binds Axl or Ror2, typically in a conditionally active manner, the ASTR can include a second antigen-binding domain that is specific for a second antigen that is expressed by (synthesized by) a cancer cell, i.e., a cancer cell associated antigen. The cancer cell associated antigen can be an antigen associated with, e.g., a breast cancer cell, a B cell lymphoma, a Hodgkin lymphoma cell, an ovarian cancer cell, a prostate cancer cell, a mesothelioma, a lung cancer cell (e.g., a small cell lung cancer cell), a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a chronic lymphocytic leukemia cell, an acute lymphocytic leukemia cell, a neuroblastoma cell, a glioma, a glioblastoma, a medulloblastoma, a colorectal cancer cell, etc. A cancer cell associated antigen may also be expressed by a non-cancerous cell.

Non-limiting examples of antigens to which a bispecific ASTR of a CAR can bind in addition to Axl or Ror2, include, e.g., CD19, CD20, CD38, CD30, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like.

In some cases, a member of a specific binding pair suitable for use in a subject bispecific ASTR of a CAR in addition to a specific binding pair member that binds Axl or Ror2, binds a ligand for a receptor. Ligands include, but are not limited to, cytokines (e.g., IL-13, etc.); growth factors (e.g., heregulin; vascular endothelial growth factor (VEGF); and the like); an integrin-binding peptide (e.g., a peptide comprising the sequence Arg-Gly-Asp); and the like.

Where the member of a specific binding pair in a subject bispecific CAR is a ligand, the CAR can be activated in the presence of a second member of the specific binding pair, where the second member of the specific binding pair is a receptor for the ligand. For example, where the ligand is VEGF, the second member of the specific binding pair can be a VEGF receptor, including a soluble VEGF receptor.

As noted above, in some cases, the member of a specific binding pair that is included in a subject bispecific CAR is an ASTR that is a receptor, e.g., a receptor for a ligand, a co-receptor, etc. The receptor can be a ligand-binding fragment of a receptor. Suitable receptors include, but are not limited to, a growth factor receptor (e.g., a VEGF receptor); a killer cell lectin-like receptor subfamily K, member 1 (NKG2D) polypeptide (receptor for MICA, MICB, and ULB6); a cytokine receptor (e.g., an IL-13 receptor; an IL-2 receptor; etc.); CD27; a natural cytotoxicity receptor (NCR) (e.g., NKP30 (NCR3/CD337) polypeptide (receptor for HLA-B-associated transcript 3 (BAT3) and B7-H6); etc.); etc.

### Stalk Region

In some cases, the CAR includes a hinge domain (also referred to herein as a “spacer” or a “stalk”) which is located in the CAR portion outside the cell and interposed between the ASTR and the transmembrane domain. In illustrative embodiments, the hinge domain is a CD8 stalk domain or a CD28 stalk domain. In some cases, the stalk domain has at least 85, 90, 95, 96, 97, 98, 99, or 100% identity to a wild-type CD8 stalk region (TTTPAPRPPTPAPTIASQPLSLRPEACRPAAGG AVHTRGLDFA (SEQ ID NO: 125)), has at least 85, 90, 95, 96, 97, 98, 99, or 100% identity to a wild-type CD28 stalk region (FCKIEVMYPPPYLDNEKSNGTIIHVKGKHLCPSPLFPGPSKP (SEQ ID NO: 126)) or has at least 85, 90, 95, 96, 97, 98, 99, or 100% identity to a wild-type immunoglobulin heavy chain hinge/stalk region. In some cases, the stalk domain is a hinge region polypeptide derived from a receptor (e.g., a CDS-derived hinge region). In a CAR, the stalk employed allows the conditionally active antigen-specific targeting region, and typically the entire CAR, to retains its increased binding to Ror2 or Axl property, in an in vitro tumor surrogate assay condition relative to a corresponding physiological condition.

The stalk region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa.

In some cases, the hinge region of a subject CAR includes at least one cysteine. For example, in some cases, the hinge region can include the sequence Cys-Pro-Pro-Cys (SEQ ID NO:62). If present, a cysteine in the hinge region of a first CAR can be available to form a disulfide bond with a hinge region in a second CAR.

Immunoglobulin hinge/stalk region amino acid sequences are known in the art; see, e.g., Tan et al. (1990) Proc. Natl. Acad. Sci. USA 87:162; and Huck et al. (1986) Nucl. Acids Res. 14:1779. As non-limiting examples, an immunoglobulin hinge region can include a domain with at least 50, 60, 70, 75, 80, 85, 90, 95, 96, 97, 98, 99 or 100% sequence identity to a stretch of at least 10, 15, 20, or all of the amino acids of any of the following amino acid sequences: DKTHT (SEQ ID NO:63); CPPC (SEQ ID NO:62); CPEPKSCDTPPPCPR (SEQ ID NO:64) (see, e.g., Glaser et al. (2005) J Biol. Chem. 280:41494); ELKTPLGDTTHT (SEQ ID NO:65); KSCDKTHTCP (SEQ ID NO:66); KCCVDCP (SEQ ID NO:67); KYGPPCP (SEQ ID NO:68); EPKSCDKTHTCPPCP (SEQ ID NO:69) (human IgGl hinge); ERKCCVECPPCP (SEQ ID NO:70) (human IgG2 hinge); ELKTPLGDTTHTCPRCP (SEQ ID NO:71) (human IgG3 hinge); SPNMVPHAH1HAQ (SEQ ID NO:72) (human IgG4 hinge); and the like. The hinge region can include an amino acid sequence of a human IgG1, IgG2, IgG3, or IgG4, hinge region. The hinge region can include one or more amino acid substitutions and/or insertions and/or deletions compared to a wild-type (naturally-occurring) hinge region. For example, His229 of human IgG 1 hinge can be substituted with Tyr, so that the hinge region includes the sequence EPKSCDKTYTCPPCP; see, e.g., Yan et al. (2012) J. Biol. Chem. 287:5891. The hinge region can include an amino acid sequence derived from human CD8; e.g., the hinge region can include the amino acid sequence: TTTPAPRPPTPAPTIASQPLSLRPEACRPAAGGAVHTRGLDFACD (SEQ ID NO:73), or a variant thereof.

### Transmembrane Domain

The CAR of the present disclosure includes transmembrane domains for insertion into a eukaryotic cell membrane. The transmembrane domain can be interposed between the ASTR and the co-stimulatory domain. The transmembrane domain can be interposed between the hinge region and the co-stimulatory domain, such that the chimeric antigen receptor includes, in order from the amino terminus (N-terminus) to the carboxyl terminus (C-terminus): an ASTR; a hinge region; a transmembrane domain; and an activating domain.

Any transmembrane (TM) domain that provides for insertion of a polypeptide into the cell membrane of a eukaryotic (e.g., mammalian) cell is suitable for use in aspects and embodiments disclosed herein. In certain embodiments provided herein, the TM domain for any aspect provided herein that includes a CAR, is a C* alpha TM domain, a CD8 TM domain, a CD4 TM domain, a C3Z TM domain, a C28 TM domain, a C134 TM domain, a CD7 TM domain, a CD8 TM domain, or a CD28 TM domain. Illustrative embodiments of CARs provided herein include a CD8 TM domain or a CD28 TM domain. Further non-limiting examples of TM domains suitable for any of the aspects or embodiments provided herein, include a domain with at least 50, 60, 70, 75, 80, 85, 90, 95, 96, 97, 98, 99 or 100% sequence identity to a stretch of at least 10, 15, 20, or all of the amino acids of any of the following TM domains:

a) CD8 alpha
(IYIWAPLAGTCGVLLLSLVITLYC (SEQ ID NO: 46));
b) CD8 beta
(LGLLVAGVLVLLVSLGVAIHLCC (SEQ ID NO: 47));
c) CD4
(ALIVLGGVAGLLLFIGLGIFFCVRC (SEQ ID NO: 48));
d) CD3Z
(LCYLLDGILFIYGVILTALFLRV (SEQ ID NO: 49);
e) CD28
(FWVLVVVGGVLACYSLLVTVAFIIFWV (SEQ ID NO: 50));
f) CD134 (OX40):
(VAAILGLGLVLGLLGPLAILLALYLL (SEQ ID NO: 51));
g) CD7
(ALPAALAVISFLLGLGLGVACVLA (SEQ ID NO: 52)),
h) CD8 alpha stalk and TM
(TTTPAPRPPTPAPTIASQPLSLRPEACRPAAGGAVHTRGLDFACDIYIW
APLAGTCGVLLLSLVITLYC (SEQ ID NO: 75)),
and
i) CD28 stalk and TM
(IEVMYPPPYLDNEKSNGTIIHVKGKHLCPSPLFPGPSKPFWVLVVVGGV
LACYSLLVTVAFIIFWV (SEQ ID NO: 76)).

As non-limiting examples, a transmembrane domain of an aspect of the invention can have at least 80, 90, or 95% sequence identity to the SEQ ID NO:46 transmembrane domain, the CD8 beta transmembrane domain, the CD4 transmembrane domain, the CD3 zeta transmembrane domain, the CD28 transmembrane domain, the CD134 transmembrane domain, or the CD7 transmembrane domain.

### CAR Linkers

In some cases, the CAR includes a linker between any two adjacent domains. For example, a linker can be between the transmembrane domain and the first co-stimulatory domain. As another example, the ASTR can be an antibody and a linker can be between the heavy chain and the light chain. As another example, a linker can be between the ASTR and the transmembrane domain and a co-stimulatory domain. As another example, a linker can be between the co-stimulatory domain and the intracellular activating domain of the second polypeptide.

The linker peptide may have any of a variety of amino acid sequences. Proteins can be joined by a spacer peptide, generally of a flexible nature, although other chemical linkages are not excluded. A linker can be a peptide of between about 1 and about 100 amino acids in length, or between about 1 and about 25 amino acids in length. These linkers can be produced by using synthetic, linker-encoding oligonucleotides to couple the proteins. Peptide linkers with a degree of flexibility can be used.

The linking peptides may have virtually any amino acid sequence, bearing in mind that suitable linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)ₙ, glycine-serine polymers (including, for example, (GS)ₙ, GSGGSₙ, GGGSₙ, and GGGGSₙ where n is an integer of at least one), glycine-alanine polymers, alanine-serine polymers, and other flexible linkers known in the art. Glycine and glycine-serine polymers are of interest since both of these amino acids are relatively unstructured, and therefore may serve as a neutral tether between components. Glycine polymers are of particular interest since glycine accesses significantly more phi-psi space than even alanine, and is much less restricted than residues with longer side chains (see Scheraga, Rev. Computational Chem. 11173-142 (1992)). Exemplary flexible linkers include, but are not limited GGGGSGGGGSGGGGS (SEQ ID NO:53), GGGGSGGGGSGGGGSGGGGSGGGGSGGGGS (SEQ ID NO:54), GGGGSGGGSGGGGS (SEQ ID NO:55), GGSG (SEQ ID NO:56), GGSGG (SEQ ID NO:57), GSGSG (SEQ ID NO:58), GSGGG (SEQ ID NO:59), GGGSG (SEQ ID NO:60), GSSSG (SEQ ID NO:61), and the like. The ordinarily skilled artisan will recognize that design of a peptide conjugated to any elements described above can include linkers that are all or partially flexible, such that the linker can include a flexible linker as well as one or more portions that confer less flexible structure.

### Modulatory Domains

Modulatory domains can change the effect of the activating domain in the CAR, including enhancing or dampening the downstream effects of the activating domain or changing the nature of the response. Modulatory domains suitable for use in a CAR of the present disclosure, and included in certain illustrative embodiments of any aspect herein that includes a CAR, include co-stimulatory domains. In some embodiments, a CAR can have more than one modulatory domain (e.g. co-stimulatory domain) or a modulatory domain (e.g. co-stimulatory domain) of a CAR can be derived from more than one polypeptide. A modulatory domain (e.g. co-stimulatory domain) suitable for inclusion in the CAR can have a length of from about 30 amino acids to about 70 amino acids (aa), e.g., a modulatory domain (e.g. co-stimulatory domain) can have a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa. In other cases, modulatory domain can have a length of from about 70 aa to about 100 aa, from about 100 aa to about 200 aa, or greater than 200 aa.

Co-stimulatory domains typically enhance and/or change the nature of the response to activation of an activation domain. Co-stimulatory domains suitable for use in a CAR of the present disclosure are generally polypeptides derived from receptors. In some embodiments, co-stimulatory domains homodimerize. A subject co-stimulatory domain can be an intracellular portion of a transmembrane protein (i.e., the co-stimulatory domain can be derived from a transmembrane protein). Non-limiting examples of suitable co-stimulatory polypeptides include, but are not limited to, 4-1BB (CD137), B7-H3, CD2, CD7, CD27, CD28, CD28 deleted for Lek binding (ICA), ICOS, OX40, BTLA, CD27, CD30, CD40, GITR, HVEM, LFA-1, LIGHT, NKG2C, PD-1, TILR2, TILR4, TILR7, TILR9, Fc receptor gamma chain, Fc receptor E chain, or a ligand that specifically binds with CD83. For example, a co-stimulatory domain of an aspect of the invention can have at least 80%, 90%, or 95% sequence identity to the co-stimulatory domain of 4-1BB (CD137), CD27, CD28, CD28 deleted for Lek binding (ICA), ICOS, OX40, BTLA, CD27, CD30, GITR, or HVEM. In some embodiments, a CAR can include more than one co-stimulatory domain, for example, a CAR can include a co-stimulatory domain from ICA and a co-stimulatory domain from 4-1BB (CD137),

In some cases, the co-stimulatory domain is derived from an intracellular portion of the transmembrane protein CD137 (also known as TNFRSF9; CD137; 4-1BB; CDwl37; ILA; etc.). For example, a suitable co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in the following amino acid sequence:

(SEQ ID NO: 1)
KRGRKKLLYIFKQPFMRPVQTTQEEDGCSCRFPEEEEGGCEL.

In some of these embodiments, the co-stimulatory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

In some cases, the co-stimulatory domain is derived from an intracellular portion of the transmembrane protein CD28 (also known as Tp44). For example, a suitable co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in the following amino acid sequence: RSKRSRLLHSDYMNMTPRRPGPTRKHYQPYAPPRDFAAYRS (SEQ ID NO:2). In some of these embodiments, the co-stimulatory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

In some cases, the co-stimulatory domain is derived from an intracellular portion of the transmembrane protein CD28 deleted for Lek binding (ICA). For example, a suitable co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in the following amino acid sequence: RSKRSRLLHSDYMNMTPRRPGPTRKHYQAYAAARDFAAYRS (SEQ ID NO:3). In some of these embodiments, the co-stimulatory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

In some cases, the co-stimulatory domain is derived from an intracellular portion of the transmembrane protein ICOS (also known as AILIM, CD278, and CVIDI). For example, a suitable co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in the following amino acid sequence: TKKKYSSSVHDPNGEYMFMRAVNTAKKSRLTDVTL (SEQ ID NO:4). In some of these embodiments, the co-stimulatory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

In some cases, the co-stimulatory domain is derived from an intracellular portion of the transmembrane protein OX40 (also known as TNFRSF4, RP5-902P8.3, ACT35, CD134, OX-40, TXGPIL). For example, a suitable co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in the following amino acid sequence:

(SEQ ID NO: 5)
RRDQRLPPDAHKPPGGGSFRTPIQEEQADAHSTLAKI.

In some of these embodiments, the co-stimulatory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

In some cases, the co-stimulatory domain is derived from an intracellular portion of the transmembrane protein CD27 (also known as S 152, T 14, TNFRSF7, and Tp55). For example, a suitable co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in the following amino acid sequence: HQRRKYRSNKGESPVEPAEPCRYSCPREEEGSTIPIQEDYRKPEPACSP (SEQ ID NO:6).

(SEQ ID NO: 6)
HQRRKYRSNKGESPVEPAEPCRYSCPREEEGSTIPIQEDYRKPEPACSP.

In some of these embodiments, the co-stimulatory domain has a length of from about 30 aa to about 35 aa, from about 35 aa to about 40 aa, from about 40 aa to about 45 aa, or from about 45 aa to about 50 aa.

In some cases, the co-stimulatory domain is derived from an intracellular portion of the transmembrane protein BTLA (also known as BTLA1 and CD272). For example, a suitable co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in the following amino acid sequence:

(SEQ ID NO: 7)
CCLRRHQGKQNELSDTAGREINLVDAHLKSEQTEASTRQNSQVLLSETGI
YDNDPDLCFRMQEGSEVYSNPCLEENKPGIVYASLNHSVIGPNSRLARNV
KEAPTEYASICVRS.

In some cases, the co-stimulatory domain is derived from an intracellular portion of the transmembrane protein CD30 (also known as TNFRSF8, DlS166E, and Ki-1). For example, a suitable co-stimulatory domain can include a domain with at least 50%, 60%, 70%, 75%, 80%, 85%, 90%, 95%, 96%, 97%, 98%, 99% or 100% sequence identity to a stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, from about 150 aa to about 160 aa, or from about 160 aa to about 185 aa of the following amino acid sequence:

(SEQ ID NO: 8)
RRACRKRIRQKLHLCYPVQTSQPKLELVDSRPRRSSTQLRSGASVTEPVA
EERGLMSQPLMETCHSVGAAYLESLPLQDASPAGGPSSPRDLPEPRVSTE
HTNNKIEKIYIMKADTVIVGTVKAELPEGRGLAGPAEPELEEELEADHTP
HYPEQETEPPLGSCSDVMLSVEEEGKEDPLPTAASGK.

In some cases, the co-stimulatory domain is derived from an intracellular portion of the transmembrane protein GITR (also known as TNFRSF18, RP5-902P8.2, AITR, CD357, and GITR-D). For example, a suitable co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in the following amino acid sequence:

(SEQ ID NO: 9)
HIWQLRSQCMWPRETQLLLEVPPSTEDARSCQFPEEERGERSAEEKGRLG
DLWV.

In some of these embodiments, the co-stimulatory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

In some cases, the co-stimulatory domain derived from an intracellular portion of the transmembrane protein HVEM (also known as TNFRSF14, RP3-395M20.6, ATAR, CD270, HVEA, HVEM, LIGHTR, and TR2). For example, a suitable co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in the following amino acid sequence: CVKRRKPRGDVVKVIVSVQRKRQEAEGEATVIEALQAPPDVTTVAVEETIPSFTGRSPNH (SEQ ID NO: 10). In some of these embodiments, the co-stimulatory domain of both the first and the second polypeptide has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

### Intracellular Activating Domain

Intracellular activating domains suitable for use in a CAR of the present disclosure when activated, typically induce the production of one or more cytokines; increased cell death; and/or increased proliferation of CD8⁺ T cells, CD4⁺ T cells, natural killer T cells, γδ T cells, and/or neutrophils. In some embodiments, the intracellular activating domain includes at least one (e.g., one, two, three, four, five, six, etc.) ITAM motifs as described below. Intracellular activating domains can be referred to herein as activating domains or activation domains. The intracellular activating domains for use in an engineered signaling polypeptide can include intracellular signaling domains of several types of immune signaling receptors, including T cell signaling proteins such as CD3, B7 family co-stimulatory, and Tumor Necrosis Factor receptor (TNFR) superfamily receptors; signaling domains used by NK and NKT cells such as NKp30 (B7-H6), DAP12, NKG2D, NKp44, NKp46, DAP10, and CD3z; and the signaling domains of human immunoglobulin receptors that contain immunoreceptor tyrosine based activation motifs (ITAM) such as FcgammaRI, FcgammaRIIA, FcgammaRIIC, FcgammaRIIIA, and FcRL5. As such, in certain embodiments of CARs for any of aspects of the present disclosure, the intracellular activating domain is a signaling domain from NKp30 (B7-H6), DAP12, NKG2D, NKp44, NKp46, DAP10, CD3z, FcgammaRI, FcgammaRIIA, FcgammaRIIC, FcgammaRIIIA, or FcRL5. These are referred to herein as an NKp30 (B7-H6) activating domain, a DAP12 activating domain, an NKG2D activating domain, an NKp44 activating domain, an NKp46 activating domain, a DAP10 activating domain, a CD3z activating domain, a FcgammaRI activating domain, a FcgammaRIIA activating domain, an FcgammaRIIC activating domain, an FcgammaRIIIA activating domain, or an FcRL5 activating domain, respectively.

In some embodiments, the intracellular activating domain includes DAP10/CD28 type signaling chains. In some embodiments, the intracellular activating domain is not covalently attached to the membrane bound CAR, but is instead diffused in the cytoplasm. As non-limiting examples, an intracellular activating domain of any aspect of the invention that includes a CAR can be a CD3Z activating domain, a CD3D activating domain, a CD3E activating domain, a CD3G activating domain, a CD79A activating domain, a DAP12 activating domain, a FCERIG activating domain, a DAP10/CD28 activating domain, or a ZAP70 activating domain. As non-limiting examples, an intracellular activating domain of any aspect of the invention that includes a CAR can have at least 80%, 90%, or 95% sequence identity to the CD3Z, CD3D, CD3E, CD3G, CD79A, DAP12, FCERIG, DAP10/CD28, or ZAP70 domains as described below.

### ITAMs

Intracellular activating domains suitable for use in a CAR of the present disclosure include immunoreceptor tyrosine-based activation motif (ITAM)-containing intracellular signaling polypeptides. An ITAM motif is YX₁X₂L/I, where X₁ and X₂ are independently any amino acid. In some cases, the intracellular activating domain of a subject CAR includes 1, 2, 3, 4, or 5 ITAM motifs. In some cases, an ITAM motif is repeated twice [in an intracellular activating domain, where the first and second instances of the ITAM motif are separated from one another by 6 to 8 amino acids, e.g., (YX₁X₂L/I)(X₃) (YX₁X₂L/I), where n is an integer from 6 to 8, and each of the 6-8 X₃ can be any amino acid. In some cases, the intracellular activating domain of a subject CAR includes 3 ITAM motifs.

A suitable intracellular activating domain can be an ITAM motif-containing portion that is derived from a polypeptide that contains an ITAM motif. For example, a suitable intracellular activating domain can be an ITAM motif-containing domain from any ITAM motif-containing protein. Thus, a suitable intracellular activating domain need not contain the entire sequence of the entire protein from which it is derived. Examples of suitable ITAM motif-containing polypeptides include, but are not limited to: CD3Z (CD3 zeta); CD3D (CD3 delta); CD3E (CD3 epsilon); CD3G (CD3 gamma); CD79A (antigen receptor complex-associated protein alpha chain); DAP12; and FCERIG (Fc epsilon receptor I gamma chain).

In some cases, the intracellular activating domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). For example, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequences or to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of either of the following amino acid sequences (2 isoforms):

(SEQ ID NO: 11)
MKWKALFTAAILQAQLPITEAQSFGLLDPKLCYLLDGILFIYGVILTALF
LRVKFSRSADAPAYQQGQNQL[YNELNLGRREEYDVL]DKRRGRDPEMGG
KPRRKNPQEGL[YNELQKDKMAEAYSEI]GMKGERRRGKGHDGL[YQGLS
TATKDTYDAL]HMQALPPR
or
(SEQ ID NO: 12)
MKWKALFTAAILQAQLPITEAQSFGLLDPKLCYLLDGILFIYGVILTALF
LRVKFSRSADAPAYQQGQNQL[YNELNLGRREEYDVL]DKRRGRDPEMGG
KPQRRKNPQEGL[YNELQKDKMAEAYSEI]GMKGERRRGKGHDGL[YQGL
STATKDTYDAL]HMQALPPR,

where the ITAM motifs are set out with brackets.

Likewise, a suitable intracellular activating domain polypeptide can include an ITAM motif-containing a portion of the full length CD3 zeta amino acid sequence. Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequences or to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of either of the following amino acid sequences:

(SEQ ID NO: 13)
RVKFSRSADAPAYQQGQNQL[YNELNLGRREEYDVL]DKRRGRDPEMGGK
PRRKNPQEGL[YNELQKDKMAEAYSEI]GMKGERRRGKGHDGL[YQGLST
ATKDTYDAL]HMQALPPR;
(SEQ ID NO: 127)
RVKFSRSADAPAYQQGQNQL[YNELNLGRREEYDVL]DKRRGRDPEMGGK
PQRRKNPQEGL[YNELQKDKMAEAYSEI]GMKGERRRGKGHDGL[YQGLS
TATKDTYDAL]HMQALPPR;
SEQ ID NO: 14)
NQL[YNELNLGRREEYDVL]DKR;
(SEQ ID NO: 15)
EGL[YNELQKDKMAEAYSEI]GMK;
or
(SEQ ID NO: 16)
DGL[YQGLSTATKDTYDAL]HMQ,

where the ITAM motifs are set out with brackets.

In some cases, the intracellular activating domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequences or to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of either of the following amino acid sequences:

(SEQ ID NO: 17)
MEHSTFLSGLVLATLLSQVSPFKIPIEELEDRVFVNCNTSITWVEGTVGT
LLSDITRLDLGKRILDPRGIYRCNGTDIYKDKESTVQVHYRMCQSCVELD
PATVAGIIVTDVIATLLLALGVFCFAGHETGRLSGAADTQALLRNDQV[Y
QPLRDRDDAQYSHL]GGNWARNK
or
(SEQ ID NO: 18)
MEHSTFLSGLVLATLLSQVSPFKIPIEELEDRVFVNCNTSITWVEGTVGT
LLSDITRLDLGKRILDPRGIYRCNGTDIYKDKESTVQVHYRTADTQALLR
NDQV[YQPLRDRDDAQYSHL]GGNWARNK,

, where the ITAM motifs are set out with brackets.

Likewise, a suitable intracellular activating domain polypeptide can comprise an ITAM motif-containing portion of the full length CD3 delta amino acid sequence. Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequence: DQV[YQPLRDRDDAQYSHL]GGN (SEQ ID NO: 19), where the ITAM motifs are set out with brackets.

In some cases, the intracellular activating domain is derived from T-cell surface glycoprotein CD3 epsilon chain (also known as CD3e, T-cell surface antigen T3/Leu-4 epsilon chain, T-cell surface glycoprotein CD3 epsilon chain, A1504783, CD3, CD3epsilon, T3e, etc.). Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequences or to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of the following amino acid sequence:

(SEQ ID NO: 20)
MQSGTHWRVLGLCLLSVGVWGQDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDMSVATIVIVDICITGGLLLLVYYW
SKNRKAKAKPVTRGAGAGGRQRGQNKERPPPVPNPD[YEPIRKGQRDLYS
GL]NQRRI,

where the ITAM motifs are set out with brackets.

Likewise, a suitable intracellular activating domain polypeptide can comprise an ITAM motif-containing portion of the full length CD3 epsilon amino acid sequence. Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequence: NPD[YEPIRKGQRDLYSGL]NQR (SEQ ID NO:21), where the ITAM motifs are set out with brackets.

In some cases, the intracellular activating domain is derived from T-cell surface glycoprotein CD3 gamma chain (also known as CD3G, T-cell receptor T3 gamma chain, CD3-GAMMA, T3G, gamma polypeptide (TiT3 complex), etc.). Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequences or to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of the following amino acid sequence:

(SEQ ID NO: 22)
MEQGKGLAVLILAIILLQGTLAQSIKGNHLVKVYDYQEDGSVLLTCDAEA
KNITWFKDGKMIGFLTEDKKKWNLGSNAKDPRGMYQCKGSQNKSKPLQVY
YRMCQNCIELNAATISGFLFAEIVSIFVLAVGVYFIAGQDGVRQSRASDK
QTLLPNDQL[YQPLKDREDDQYSHL]QGNQLRRN,

where the ITAM motifs are set out with brackets.

Likewise, a suitable intracellular activating domain polypeptide can comprise an ITAM motif-containing portion of the full length CD3 gamma amino acid sequence. Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequence: DQL[YQPLKDREDDQYSHL]QGN (SEQ ID NO:23), where the ITAM motifs are set out with brackets.

In some cases, the intracellular activating domain is derived from CD79A (also known as B-cell antigen receptor complex-associated protein alpha chain; CD79a antigen (immunoglobulin-associated alpha); MB-1 membrane glycoprotein; Ig-alpha; membrane-bound immunoglobulin-associated protein; surface IgM-associated protein; etc.). Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequences or to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of either of the following amino acid sequences:

(SEQ ID NO: 24)
MPGGPGVLQALPATIFLLFLLSAVYLGPGCQALWMHKVPASLMVSLGEDA
HFQCPHNSSNNANVTWWRVLHGNYTWPPEFLGPGEDPNGTLIIQNVNKSH
GGIYVCRVQEGNESYQQSCGTYLRVRQPPPRPFLDMGEGTKNRIITAEGI
ILLFCAVVPGTLLLFRKRWQNEKLGLDAGDEYEDENL[YEGLNLDDCSMY
EDI]SRGLQGTYQDVGSLNIGDVQLEKP
or
(SEQ ID NO: 25)
MPGGPGVLQALPATIFLLFLLSAVYLGPGCQALWMHKVPASLMVSLGEDA
HFQCPHNSSNNANVTWWRVLHGNYTWPPEFLGPGEDPNEPPPRPFLDMGE
GTKNRIITAEGIILLFCAVVPGTLLLFRKRWQNEKLGLDAGDEYEDENL
[YEGLNLDDCSMYEDI]SRGLQGTYQDVGSLNIGDVQLEKP,

where the ITAM motifs are set out with brackets.

Likewise, a suitable intracellular activating domain polypeptide can comprise an ITAM motif-containing portion of the full length CD79A amino acid sequence. Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequence: ENL[YEGLNLDDCSMYEDI]SRG (SEQ ID NO:26), where the ITAM motifs are set out with brackets.

In some cases, the intracellular activating domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). For example, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequences or to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of either of the following amino acid sequences (4 isoforms):

(SEQ ID NO: 27)
MGGLEPCSRLLLLPLLLAVSGLRPVQAQAQSDCSCSTVSPGVLAGIVMGD
LVLTVLIALAVYFLGRLVPRGRGAAEAATRKQRITETESP[YQELQGQRS
DVYSDL]NTQRPYYK,
(SEQ ID NO: 28)
MGGLEPCSRLLLLPLLLAVSGLRPVQAQAQSDCSCSTVSPGVLAGIVMGD
LVLTVLIALAVYFLGRLVPRGRGAAEATRKQRITETESP[YQELQGQRSD
VYSDL]NTQ,
(SEQ ID NO: 29)
MGGLEPCSRLLLLPLLLAVSDCSCSTVSPGVLAGIVMGDLVLTVLIALAV
YFLGRLVPRGRGAAEAATRKQRITETESP[YQELQGQRSDVYSDL]NTQR
PYYK,
or
(SEQ ID NO: 30)
MGGLEPCSRLLLLPLLLAVSDCSCSTVSPGVLAGIVMGDLVLTVLIALAV
YFLGRLVPRGRGAAEATRKQRITETESP[YQELQGQRSDVYSDL]NTQRP
YYK,

where the ITAM motifs are set out with brackets.

Likewise, a suitable intracellular activating domain polypeptide can comprise an ITAM motif-containing portion of the full length DAP12 amino acid sequence. Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequence: ESP[YQELQGQRSDVYSDL]NTQ (SEQ ID NO:31), where the ITAM motifs are set out with brackets.

In some cases, the intracellular activating domain is derived from FCERIG (also known as FCRG; Fc epsilon receptor I gamma chain; Fc receptor gamma-chain; fc-epsilon RI-gamma; fcRgamma; fceRI gamma; high affinity immunoglobulin epsilon receptor subunit gamma; immunoglobulin E receptor, high affinity, gamma chain; etc.). For example, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequences or to a contiguous stretch of from about 50 amino acids to about 60 amino acids (aa), from about 60 aa to about 70 aa, from about 70 aa to about 80 aa, or from about 80 aa to about 88 aa, of the following amino acid sequence:

(SEQ ID NO: 32)
MIPAVVLLLLLLVEQAAALGEPQLCYILDAILFLYGIVLTLLYCRLKIQV
RKAAITSYEKSDGV[YTGLSTRNQETYETL]KHEKPPQ,

where the ITAM motifs are set out with brackets.

Likewise, a suitable intracellular activating domain polypeptide can comprise an ITAM motif-containing portion of the full length FCER1G amino acid sequence. Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequence: DGV[YTGLSTRNQETYETL]KHE (SEQ ID NO:33), where the ITAM motifs are set out with brackets.

Intracellular activating domains suitable for use in a CAR of the present disclosure include a DAP10/CD28 type signaling chain. An example of a DAP10 signaling chain is the amino acid sequence is: RPRRSPAQDGKV[YINM]PGRG (SEQ ID NO:34). In some embodiments, a suitable intracellular activating domain includes a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequence: RPRRSPAQDGKV[YINM]PGRG (SEQ ID NO:34), where a noteworthy motif is set out in brackets.

An example of a CD28 signaling chain is the amino acid sequence is FWVLVVVGGVLACYSLLVTVAFIIFWVRSKRSRLLHSD[YMNM]TPRRPGPTRKHYQPYAPPRDF AAYRS (SEQ ID NO:35). In some embodiments, a suitable intracellular domain includes a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequence:

(SEQ ID NO: 35)
FWVLVVVGGVLACYSLLVTVAFIIFWVRSKRSRLLHSD[YMNM]TPRRPG
PTRKHYQPYAPPRDFAAYRS.

Intracellular activating domains suitable for use in a CAR of the present disclosure include a ZAP70 polypeptide, For example, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequences or to a contiguous stretch of from about 300 amino acids to about 400 amino acids, from about 400 amino acids to about 500 amino acids, or from about 500 amino acids to 619 amino acids, of the following amino acid sequence:

(SEQ ID NO: 36)
MPDPAAHLPFFYGSISRAEAEEHLKLAGMADGLFLLRQCLRSLGGYVLSL
VHDVRFHHFPIERQLNGTYAIAGGKAHCGPAELCEFYSRDPDGLPCNLRK
PCNRPSGLEPQPGVFDCLRDAMVRDYVRQTWKLEGEALEQAIISQAPQVE
KLIATTAHERMPWYHSSLTREEAERKLYSGAQTDGKFLLRPRKEQGTYAL
SLIYGKTVYHYLISQDKAGKYCIPEGTKFDTLWQLVEYLKLKADGLIYCL
KEACPNSSASNASGAAAPTLPAHPSTLTHPQRRIDTLNSDGYTPEPARIT
SPDKPRPMPMDTSVYESPYSDPEELKDKKLFLKRDNLLIADIELGCGNFG
SVRQGVYRMRKKQIDVAIKVLKQGTEKADTEEMMREAQIMHQLDNPYIVR
LIGVCQAEALMLVMEMAGGGPLHKFLVGKREEIPVSNVAELLHQVSMGMK
YLEEKNFVHRDLAARNVLLVNRHYAKISDFGLSKALGADDSYYTARSAGK
WPLKWYAPECINFRKFSSRSDVWSYGVTMWEALSYGQKPYKKMKGPEVMA
FIEQGKRMECPPECPPELYALMSDCWIYKWEDRPDFLTVEQRMRACYYSL
ASKVEGPPGSTQKAEAACA.

### Additional Domains

The CAR can further include one or more additional polypeptide domains, where such domains include, but are not limited to, a signal sequence; an epitope tag; an affinity domain; and a polypeptide that produces a detectable signal. Non-limiting examples of additional domains for any of the aspects or embodiments provided herein, include a domain with at least 50%, 60%, 70%, 75%, 80%, 85%, 90%, 95%, 96%, 97%, 98%, 99% or 100% sequence identity to any of the following sequences as described below: a signal sequence, an epitope tag, an affinity domain, or a polypeptide that produces a detectable signal.

Signal sequences that are suitable for use in a subject CAR, e.g., in the first polypeptide of a subject CAR, include any eukaryotic signal sequence, including a naturally-occurring signal sequence, a synthetic (e.g., man-made) signal sequence, etc. In some embodiments, for example, the signal sequence can be the CD8 signal sequence MALPVTALLLPLALLLHAARP (SEQ ID NO:74).

Suitable epitope tags include, but are not limited to, hemagglutinin (HA; e.g., YPYDVPDYA; SEQ ID NO:37); FLAG (e.g., DYKDDDDK; SEQ ID NO:38); c-myc (e.g., EQKLISEEDL; SEQ ID NO:39), and the like.

Affinity domains include peptide sequences that can interact with a binding partner, e.g., such as one immobilized on a solid support, useful for identification or purification. DNA sequences encoding multiple consecutive single amino acids, such as histidine, when fused to the expressed protein, may be used for one-step purification of the recombinant protein by high affinity binding to a resin column, such as nickel sepharose. Exemplary affinity domains include His5 (HHHHH; SEQ ID NO:40), HisX6 (HHHHHH; SEQ ID NO:41), c-myc (EQKLISEEDL; SEQ ID NO:39), Flag (DYKDDDDK; SEQ ID NO:38), Strep Tag (WSHPQFEK; SEQ ID NO:42), hemagluttinin, e.g., HA Tag (YPYDVPDYA; SEQ ID NO:37), GST, thioredoxin, cellulose binding domain, RYIRS (SEQ ID NO:43), Phe-His-His-Thr (SEQ ID NO:44), chitin binding domain, S-peptide, T7 peptide, SH2 domain, C-end RNA tag, WEAAAREACCRECCARA (SEQ ID NO:45), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100proteins, parvalbumin, calbindin D9K, calbindin D28K, and calretinin, inteins, biotin, streptavidin, MyoD, Id, leucine zipper sequences, and maltose binding protein.

Suitable detectable signal-producing proteins include, e.g., fluorescent proteins; enzymes that catalyze a reaction that generates a detectable signal as a product; and the like.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilized EGFP (dEGFP), destabilized ECFP (dECFP), destabilized EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFPl, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrapel, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

Recognition and/or Elimination Domains

Any of the CARs disclosed herein can include recognition or elimination domain. In some embodiments, the recognition or elimination domain can be derived from herpes simplex virus-derived enzyme thymidine kinase (HSV-tk) or inducible caspase-9 or can be the FLAG epitope (SEQ ID NO:38). In some embodiments, the recognition or elimination domain is recognized by an antibody that is approved by a government regulatory agency for use in humans, such as but limited to, cetuximab, rituximab, or Herceptin. In some embodiments, the recognition or elimination domain can include a modified endogenous cell-surface molecule as described in U.S. Pat. No. 8,802,374. The modified endogenous cell-surface molecule can be any cell-surface related receptor, ligand, glycoprotein, cell adhesion molecule, antigen, integrin, or cluster of differentiation (CD) that is modified. In some embodiments, the modified endogenous cell-surface molecule is a truncated tyrosine kinase receptor. In one aspect, the truncated tyrosine kinase receptor is a member of the epidermal growth factor receptor family (e.g., ErbB1, ErbB2, ErbB3, ErbB4), for example SEQ ID NO:78. The recognition or elimination domain can be expressed as part of a single polypeptide that also includes the CAR. In some embodiments, the recognition or elimination domain can be at or near the N-terminus of the single polypeptide, such as after an N-terminal signal peptide. In some embodiments, the recognition or elimination domain can be at or near the C-terminus of the polypeptide. In some embodiments, the recognition or elimination domain at or near the C-terminus can be separated from the amino acid sequence encoding the CAR by a cleavage signal or a ribosomal skip sequence. The cleavage signal can be any cleavage signal known in the art. The ribosomal skip sequence can be any ribosomal skip sequence known in the art, for example 2A-1 with amino acid sequence GSGEGRGSLLTCGDVEENPGP (SEQ ID NO:77). In some embodiments, the recognition or elimination domain can be between any of the domains of the CAR, for example, between the stalk and transmembrane domains or the recognition or elimination domain can be in a linker, for example the linker between the heavy and light chains of a single chain antibody.

Epidermal growth factor receptor, also known as EGFR, ErbB1 and HER1, is a cell-surface receptor for members of the epidermal growth factor family of extracellular ligands. Alterations in EGFR activity have been implicated in certain cancers. In some embodiments, a gene encoding an EGFR polypeptide including human epidermal growth factor receptor (EGFR) that is constructed by removal of nucleic acid sequences that encode polypeptides including the membrane distal EGF-binding domain and the cytoplasmic signaling tail, but retains the extracellular membrane proximal epitope recognized by an anti-EGFR antibody. In illustrative embodiments, the antibody is a known, commercially available anti-EGFR monoclonal antibody, such as cetuximab, matuzumab, necitumumab or panitumumab.

Application of biotinylated-cetuximab to immunomagnetic selection in combination with anti-biotin microbeads successfully enriches T cells that have been lentivirally transduced with EGFRt-containing constructs from as low as 2% of the population to greater than 90% purity without observable toxicity to the cell preparation. Constitutive expression of this inert EGFR molecule does not affect T cell phenotype or effector function as directed by the coordinately expressed chimeric antigen receptor (CAR), CD19R. Through flow cytometric analysis, EGFR was successfully utilized as an in vivo tracking marker for T cell engraftment in mice. Furthermore, EGFR was demonstrated to have suicide gene potential through Erbitux® mediated antibody dependent cellular cytotoxicity (ADCC) pathways. Thus, EGFR may be used as a non-immunogenic selection tool, tracking marker, and suicide gene for transduced T cells that have immunotherapeutic potential. The EGFR nucleic acid may also be detected by means well known in the art.

In some embodiments, EGFR is expressed as part of a single polypeptide that also includes the CAR. In some embodiments, the amino acid sequence encoding the EGFR recognition domain can be separated from the amino acid sequence encoding the CAR by a cleavage signal or ribosomal skip sequence. The cleavage signal can be any cleavage signal known in the art. The ribosomal skip sequence can be any ribosomal skip sequence known in the art, for example 2A-1 with amino acid sequence GSGEGRGSLLTCGDVEENPGP (SEQ ID NO:77). In some embodiments, the polynucleotide sequence encoding the recognition domain can be on the same transcript as the CAR but separated from the polynucleotide sequence encoding the CAR by an internal ribosome entry site.

### Recombination of Sequences

In certain instances, sequences of the polypeptides of a CAR, e.g., CAR domains, may be rearranged or deleted in a cell through the use of site-specific recombination technology. In certain embodiments, the cellular activation-related response to a particular CAR can be changed by site-specific recombination, e.g., a first intracellular activating domain of a CAR eliciting a first activation-related response may be exchanged for a second intracellular activating domain eliciting a second activation-related response. As will be clear to one skilled in the art, site-specific recombination can be used in a cell to exchange any domain or sequence of a CAR with any other domain or sequence as disclosed herein. As will also be clear to one skilled in the art, site-specific recombination can be used in a cell to delete any domain or sequence of a CAR. Such exchange and excision of sequences and domains is known in the art, see, e.g., domain switching in signalobodies as described in Tone et al. (2013) Biotechnology and Bioengineering, 3219-3226, the disclosure of which is disclosed herein by reference. Mechanisms and requirements for performing site-specific recombination in vivo are also well known in the art, see, e.g., Grindley et al. (2006) Annual Review of Biochemistry, 567-605 and Tropp (2012) Molecular Biology (Jones & Bartlett Publishers, Sudbury, MA), the disclosures of which are incorporated herein by reference.

CARs are chimeric proteins that are generated by fusing all the different domains discussed above together to form a fusion protein. The CAR is typically generated by an expression vector comprising polynucleotide sequences that encode the different domains of the CAR as discussed herein. The ASTR of the present invention, which functions to recognize and bind with an antigen on target cells, is conditionally active. Specifically, the ASTR is less active or inactive at a normal physiological condition and active at an in vitro tumor surrogate assay condition for binding with the target antigen, in comparison with an ASTR of the corresponding wild-type protein.

The wild-type or native protein that is suitable to be used in whole or in part for at least its binding domain for the target antigen, as an ASTR in the present invention may be discovered by generating a protein library and screening the library for a protein with a desired binding affinity to the target antigen. The wild-type protein may be discovered by screening a cDNA library. A cDNA library is a combination of cloned cDNA (complementary DNA) fragments inserted into a collection of host cells, which together constitute some portion of the transcriptome of the organism. cDNA is produced from fully transcribed mRNA and therefore contains the coding sequence for expressed proteins of an organism. The information in cDNA libraries is a powerful and useful tool for discovery of proteins with desired properties by screening the libraries for proteins with the desired binding affinity to the target antigen.

### Tumor Microenvironment

Cancer cells in a solid tumor are able to form a tumor microenvironment in their surroundings to support the growth and metastasis of the cancer cells. A tumor microenvironment is the cellular environment in which the tumor exists, including surrounding blood vessels, immune cells, fibroblasts, other cells, soluble factors, signaling molecules, an extracellular matrix, and mechanical cues that can promote neoplastic transformation, support tumor growth and invasion, protect the tumor from host immunity, foster therapeutic resistance, and provide niches for dormant metastases to thrive. The tumor and its surrounding microenvironment are closely related and interact constantly. Tumors can influence their microenvironment by releasing extracellular signals, promoting tumor angiogenesis and inducing peripheral immune tolerance, while the immune cells in the microenvironment can affect the growth and evolution of cancerous cells. See Swarts et al. “Tumor Microenvironment Complexity: Emerging Roles in Cancer Therapy,” Cancer Res, vol., 72, pages 2473-2480, 2012.

The tumor microenvironment is often hypoxic. As the tumor mass increases, the interior of the tumor grows farther away from existing blood supply, which leads to difficulties in fully supplying oxygen to the tumor microenvironment. The partial oxygen pressure in the tumor environment is below 5 mm Hg in more than 50% of locally advanced solid tumors, in comparison with a partial oxygen pressure at about 40 mm Hg in blood plasma. In contrast, other parts of the body are not hypoxic. The hypoxic environment leads to genetic instability, which is associated with cancer progression, via downregulating nucleotide excision repair and mismatch repair pathways. Hypoxia also causes the upregulation of hypoxia-inducible factor I alpha (HIF1-α), which induces angiogenesis, and is associated with poorer prognosis and the activation of genes associated with metastasis. See Weber et al., “The tumor microenvironment,” Surgical Oncology, vol. 21, pages 172-177, 2012 and Blagosklonny, “Antiangiogenic therapy and tumor progression,” Cancer Cell, vol. 5, pages 13-17, 2004.

In addition, tumor cells tend to rely on energy generated from lactic acid fermentation, which does not require oxygen. So tumor cells are less likely to use normal aerobic respiration that does require oxygen. A consequence of using lactic acid fermentation is that the tumor microenvironment is acidic (pH 6.5-6.9), in contrast to other parts of the body which are typically either neutral or slightly basic. For example, human blood plasma has a pH of about 7.4. See Estrella et al., “Acidity Generated by the Tumor Microenvironment Drives Local Invasion,” Cancer Research, vol. 73, pages 1524-1535, 2013. The nutrient availability in the tumor microenvironment is also low due to the relatively high nutrient demand of the proliferating cancer cells, in comparison with cells located in other parts of the body.

Further, the tumor microenvironment also contains many distinct cell types not commonly found in other parts of the body. These cell types include endothelial cells and their precursors, pericytes, smooth muscle cells, fibroblasts, carcinoma-associated fibroblasts, myofibroblasts, neutrophils, eosinophils, basophils, mast cells, T and B lymphocytes, natural killer cells and antigen presenting cells (APC) such as macrophages and dendritic cells (Lorusso et al., “The tumor microenvironment and its contribution to tumor evolution toward metastasis,” Histochem Cell Biol, vol. 130, pages 1091-1103, 2008).

Accordingly, the tumor microenvironment has at least several physiological conditions that are different from those of other parts of body, such as the physiological conditions in blood plasma. The tumor microenvironment has a pH (acidic) that is lower than other parts of the body, especially the blood plasma (pH 7.4). The tumor microenvironment has a lower concentration of oxygen than other parts of the body, such as blood plasma. Also, the tumor microenvironment has a lower nutrient availability than other parts of the body, especially the blood plasma. The tumor microenvironment also has some distinct cell types that are not commonly found in other parts of the body, especially the blood plasma.

In illustrative embodiments, CARs of the present invention include a conditionally active ASTR generated from a wild-type biological (i.e. native) protein, such as a wild-type or native antibody isolated from a mammalian organism such as a mouse or a human, for example, that may be a candidate for tumor treatment. The conditionally active ASTR in such illustrative embodiments has lower activity under at least one physiological condition in parts of the body other than the tumor microenvironment. such as blood plasma, than the native or wild-type biological protein, while it has higher activity under at least one physiological condition in the tumor microenvironment than the native or wild-type biological protein. Such conditionally active native or biological proteins can preferentially act upon cancer cells in the tumor microenvironment for treating tumors, and thus will be less likely to cause side effects. In embodiments where the native or biological protein is an antibody against an antigen on the surface of the tumor cells where the antigen is exposed to the tumor microenvironment, the conditionally active antibody has lower affinity to the antigen than the native or wild-type antibody in other parts of the body, e.g. a non-tumor microenvironment, while it has higher affinity to the antigen than the native or wild-type antibody in the tumor microenvironment. Such conditionally active antibodies can bind weakly or not at all to the antigen in other parts of the body, but have greater binding, or bind strongly and tightly, to the antigen in the tumor microenvironment.

### In Vitro Tumor Surrogate Assay

ASTRs used in CARs or the present disclosure, and typically the CARS herein, which include such ASTRs, in illustrative embodiments are conditionally active in a tumor environment and/or in an in vitro tumor surrogate assay condition. The in vitro tumor surrogate assay condition can be any condition that is tested in vitro by comparing that condition at values or levels found in at least some cancers in vivo, against that condition at values or levels found in physiological tissue under physiological conditions. Provided in the Examples herein, is a non-limiting in vitro tumor surrogate assay for cell lysis at low pH (e.g. 6.0 or 6.7) as compared to physiological pH (e.g. 7.4). In vitro tumor surrogate assay conditions under which a CAR of the present disclosure can be active include but are not limited to high hyaluronan, lactic acid, and/or albumin, wherein the normal condition is low levels of lactic acid, hyaluronan, and albumin. Another in vitro tumor surrogate assay condition is pH, especially specific conditions where the pH in the in vitro tumor surrogate assay condition is less than normal physiological pH. For example, the tumor surrogate assay condition can be a pH of between 6.0, 6.1, 6.2, 6.3, 6.4, and 6.5 on the low end of the range, and 6.6, 6.7, 6.8, and 6.9 on the high end of the range. Whereas physiological pH can be between 7.2, 7.3, and 7.4 on the low end of the range and 7.5, 7.6, 7.7 and 7.8 on the high end of the range. In illustrative embodiments, low pH in the in vitro tumor surrogate assay condition is between 6.5 and 6.9, and can specifically be 6.7. Physiological pH can be pH 7.2 to 7.6, or can specifically be set at pH 7.4. In another embodiment, the pH is unchanged but the in vitro tumor surrogate assay condition differs only in concentrations of lactic acid. In another embodiment, the in vitro tumor surrogate assay condition includes elevated levels of adenosine compared to the physiologic environment. In another embodiment, the in vitro tumor surrogate assay condition includes elevated levels of R-2-hydroxyglutarate as compared to the physiologic environment. In other embodiments, the scFv is grafted from the heavy and light chains of a monoclonal antibody that has been selected for microenvironmental specificity through molecular evolution, such as those described in U.S. Pat. No. 8,709,755 B2 and application WO/2016/033331A1.

### Nucleic Acids

The present disclosure provides a nucleic acid that includes a nucleotide sequence encoding the polypeptide of a conditionally active CAR of the present disclosure. A nucleic acid including a nucleotide sequence encoding the conditionally active CAR of the present disclosure will in some embodiments be DNA, including, e.g., a recombinant expression vector. A nucleic acid including a nucleotide sequence encoding the conditionally active CAR of the present disclosure will in some embodiments be RNA, e.g., in vitro synthesized RNA.

In some cases, a nucleic acid provides for production of a CAR of the present disclosure, e.g., in a mammalian cell. In other cases, a subject nucleic acid provides for amplification of the CAR-encoding nucleic acid.

A nucleotide sequence encoding the CAR of the present disclosure can be operably linked to a transcriptional control element, e.g., a promoter, and enhancer, etc.

Suitable promoter and enhancer elements are known in the art. For expression in a bacterial cell, suitable promoters include, but are not limited to, lacl, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some instances, the locus or construct or trans gene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., PNAS (2000) 28:e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. (2006) Annual Review of Biochemistry, 567-605 and Tropp (2012) Molecular Biology (Jones & Bartlett Publishers, Sudbury, MA), the disclosures of which are incorporated herein by reference.

In some cases, the promoter is a CD8 cell-specific promoter, a CD4 cell-specific promoter, a neutrophil-specific promoter, or an NK-specific promoter. For example, a CD4 gene promoter can be used; see, e.g., Salmon et al. (1993) Proc. Natdl. Acad. Sci. USA 90:7739; and Marodon et al. (2003) Blood 101:3416. As another example, a CD8 gene promoter can be used. NK cell-specific expression can be achieved by use of an Neri (p46) promoter; see, e.g., Eckelhart et al. (2011) Blood 117:1565.

In some embodiments, e.g., for expression in a yeast cell, a suitable promoter is a constitutive promoter such as an ADHl promoter, a PGKl promoter, an ENO promoter, a PYKl promoter and the like; or a regulatable promoter such as a GALl promoter, a GALlO promoter, an ADH2 promoter, a PH05 promoter, a CUPl promoter, a GAL7 promoter, a MET25 promoter, a MET3 promoter, a CYC1 promoter, a HIS3 promoter, an ADHl promoter, a PGK promoter, a GAPDH promoter, an ADCl promoter, a TRPl promoter, a URA3 promoter, a LEU2 promoter, an ENO promoter, a TPl promoter, and AOXl (e.g., for use in Pichia). Selection of the appropriate vector and promoter is well within the level of ordinary skill in the art.

Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacterial., 1991: 173(1): 86-93; Alpuche-Aranda et al., PNAS, 1992; 89(21): 10079-83), a nirB promoter (Harborne et al. (1992) Mal. Micro. 6:2805-2813), and the like (see, e.g., Dunstan et al. (1999) Infect. Immun. 67:5133-5141; McKelvie et al. (2004) Vaccine 22:3243-3255; and Chatfield et al. (1992) Biotechnol. 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spv promoter, and the like; a promoter derived from the pathogenicity island SPI-2 (see, e.g., WO96/17951); an actA promoter (see, e.g., Shetron-Rama et al. (2002) Infect. Immun. 70:1087-1096); an rpsM promoter (see, e.g., Valdivia and Falkow (1996). Mal. Microbial. 22:367); a tet promoter (see, e.g., Hillen, W. and Wissmann, A. (1989) In Saenger, W. and Heinemann, U. (eds), Topics in Molecular and Structural Biology, Protein-Nucleic Acid Interaction. Macmillan, London, UK, Vol. 10, pp. 143-162); an SP6 promoter (see, e.g., Melton et al. (1984) Nucl. Acids Res. 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (Laci repressor protein changes conformation when contacted with lactose, thereby preventing the Laci repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, for example, deBoer et al. (1983) Proc. Natl. Acad. Sci. U.S.A. 80:21-25).

A nucleotide sequence encoding a CAR can be present in an expression vector and/or a cloning vector. Where a CAR includes two separate polypeptides, nucleotide sequences encoding the two polypeptides can be cloned in the same or separate vectors. An expression vector can include a selectable marker, an origin of replication, and other features that provide for replication and/or maintenance of the vector. Suitable expression vectors include, e.g., plasmids, viral vectors, and the like.

Large numbers of suitable vectors and promoters are known to those of skill in the art; many are commercially available for generating a subject recombinant constructs. The following vectors are provided by way of example. Bacterial: pBs, phagescript, PsiX174, pBluescript SK, pBs KS, pNH8a, pNH16a, pNH18a, pNH46a (Stratagene, La Jolla, Calif, USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90: 10613-10617); SV40; herpes simplex virus; gamma retrovirus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

As noted above, in some embodiments, a nucleic acid including a nucleotide sequence encoding the conditionally active CAR of the present disclosure will in some embodiments be RNA, e.g., in vitro synthesized RNA. Methods for in vitro synthesis of RNA are known in the art; any known method can be used to synthesize RNA including a nucleotide sequence encoding the conditionally active CAR of the present disclosure. Methods for introducing RNA into a host cell are known in the art. See, e.g., Zhao et al. (2010) Cancer Res. 15:9053. Introducing RNA including a nucleotide sequence encoding the conditionally active CAR of the present disclosure into a host cell can be carried out in vitro or ex vivo or in vivo. For example, a host cell (e.g., an NK cell, a cytotoxic T lymphocyte, etc.) can be electroporated in vitro or ex vivo with RNA comprising a nucleotide sequence encoding the conditionally active CAR of the present disclosure.

### Cells

Some aspects of the present disclosure include or are cells, in illustrative examples, mammalian cells, that are used as packaging cells to make viruses, such as lentiviruses, for transduction of T cells and/or NK cells. Any of a wide variety of cells can be selected for in vitro production of a virus, such as a pseudotyped retroviris, according to the invention. Eukaryotic cells are typically used, particularly mammalian cells including human, simian, canine, feline, equine and rodent cells. In illustrative examples, the cells are human cells. In further illustrative embodiments, the cells reproduce indefinitely, and are therefore immortal. Examples of cells that may be advantageously used in the present invention include NIH 3T3 cells, COS cells, Madin-Darby canine kidney cells, human embryonic 293T cells and any cells derived from such cells, such as gpnislacZ (pNX cells, which are derived from 293T cells. Highly transfectable cells, such as human embryonic kidney 293T cells, can be used. By “highly transfectable” it is meant that at least about 50%, more preferably at least about 70% and most preferably at least about 80% of the cells can express the genes of the introduced DNA.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCLlO), PC12 cells (ATCC No. CRL1721), COS cells, COS-7 cells (ATCC No. CRL1651), RATI cells, mouse L cells (ATCC No. CCLI.3), human embryonic kidney (HEK) cells (ATCC No. CRL1573), HLHepG2 cells, Hut-78, Jurkat, HL-60, NK cell lines (e.g., NKL, NK92, and YTS), and the like.

### Methods of Activating an Immune Cell

The present disclosure provides methods of activating an immune cell in vitro, in vivo, or ex vivo. The methods generally involve contacting an immune cell (in vitro, in vivo, or ex vivo) with Axl and/or Ror2, where the immune cell has been genetically modified to produce (i.e. express) a conditionally active CAR of the present disclosure. In the presence of Axl and/or Ror2, the conditionally active CAR activates the immune cell, thereby producing an activated immune cell. Immune cells include, e.g., a cytotoxic T lymphocyte, an NK cell, a CD4⁺ T cell, a T regulatory (Treg) cell, a γδ T cell, an NK-T cell, neutrophils, etc. In illustrative embodiments, the immune cell is a T cell or NK cell, in particularly illustrative embodiments, the immune cell is a T cell, which include NK-T cells. In such illustrative embodiments the activating is typically activating the cytotoxic activity of the T cell or NK cell. Such methods can be performed using a plurality of immune cells (e.g. T cells or NK cells). In further illustrative embodiments, the contacting involves contacting a target mammalian cell expressing Axl and/or Ror2 with the immune cell. Such methods for activation of the T cells or NK cells can be detected by detecting the release of cytokines by the T cells or NK cells such as the release of IFN-γ or IL-2, increases in the cytotoxic activity of T cells and/or NK cells against cells expressing Axl or Ror2, increases in the intracellular expression of IFNγ and/or IL-2 in the T cell or NK cells, and increases in the expression of CD107a and/or CD69 by the T cell or NK cells as measured by fluorescence-activated cell sorting (FACS) analysis. Examples 1, 3, and 4 herein provide details for some of these methods of detecting the activation of T cells and/or NK cells.

Further aspects provided herein, include methods for binding an immune cell (e.g. a T cell or NK cell) to a target mammalian cell, that include contacting the target mammalian cell with the immune cell in vitro, in vivo, or ex vivo, wherein the target mammalian cell expresses Axl and/or Ror2, and the immune cell expresses any of the CARs provided herein that bind to Axl or Ror2. Such binding can activate the immune cell. Such methods can be performed using a plurality of immune cells (e.g. T cells or NK cells). Such methods for binding, as detected by detecting activation of the T cells or NK cells by release of cytokines and increase in cytotoxic activity are provided in Example 1, Example 3, and Example 4 herein.

The contacting in methods for binding or activating an immune cell, in illustrative embodiments herein involves contacting the immune cell (e.g. T cell or NK cell) in a microenvironment at a pH of less than 7.4. For example, the pH can be less than 5.8, 5.9, 6.0, 6.1, 6.2, 6.3, 6.4, 6.5, 6.6, 6.7, 6.8, or 6.9, or in the range of 5.8 to 7.0, in illustrative embodiments in the range of 6.0 to 6.8, in the range of 6.1 to 6.9, in the range of 6.2 to 6.8, or between 6.0, 6.1, 6.2, 6.3, 6.4, and 6.5 on the low end of the range, and 6.6, 6.7, 6.8, and 6.9 on the high end of the range. In such illustrative embodiments, the CAR is any of the CAB-CARs disclosed herein, that recognizes Axl or Ror2 provided herein.

Contacting the genetically modified immune cell (e.g., a T lymphocyte, an NK cell) with Axl and/or Ror2 can increase production of a cytokine by the immune cell by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared with the amount of cytokine produced by the immune cell in the absence of Axl and/or Ror2. Contacting the genetically modified immune cell (e.g., a T lymphocyte or an NK cell) with Axl and/or Ror2 can increase secretion of a cytokine by the immune cell by at least 10%, at least 15%, at least 20%, at least 25%, at least 30%, at least 40%, at least 50%, at least 75%, at least 2-fold, at least 2.5-fold, at least 5-fold, at least 10-fold, or more than 10-fold, compared with the amount of cytokine secreted by the immune cell in the absence of Axl and/or Ror2. Cytokines whose production can be increased include, but are not limited to, IL-2 and IFN-γ.

Contacting a genetically modified cytotoxic cell (e.g., cytotoxic T lymphocyte) with AAR can increase cytotoxic activity of the cytotoxic cell by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared to the cytotoxic activity of the cytotoxic cell in the absence of Axl and/or Ror2.

Contacting a genetically modified cytotoxic cell (e.g., cytotoxic T lymphocyte) with Axl and/or Ror2 can increase cytotoxic activity of the cytotoxic cell by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared to the cytotoxic activity of the cytotoxic cell in the absence of Axl and/or Ror2.

In other embodiments, e.g., depending on the host immune cell, contacting a genetically modified host cell with an antigen can increase or decrease cell proliferation, cell survival, cell death, and the like.

### Methods for Making/Isolating a Conditionally Active Antigen-Specific Targeting Region

In illustrative embodiments, the anti-Axl and anti-Ror2 antigen receptors disclosed herein are conditionally active such that they exhibit an increase in binding to Axl or Ror2 at a pH 6.7 (an exemplary pH of a tumor environment and/or in an in vitro tumor surrogate assay condition) compared to 7.4 (a normal physiological condition). In some illustrative embodiments of any aspect disclosed herein, the conditionally active anti-Axl or anti-Ror2 ASTR is identified from an initial polypeptide library without mutating/evolving members of the library before screening/evolving and/or without mutating during or between optional repeated rounds of screening. In other embodiments, the conditionally active anti-Axl or anti-Ror2 ASTR is identified by a method that includes mutating/evolving, and in some embodiments starts with a wild-type antibody. Exemplary transmembrane domains and intracellular activating domains can be any of those disclosed herein for CARs.

In one aspect, provided herein is a method for selecting a conditionally active anti-Axl or anti-Ror2 ASTR, comprising panning a polypeptide display library by:
a. subjecting polypeptides of the polypeptide display library to an Axl or Ror2 binding assay at a pH 7.4 (or other normal physiological condition) and an Axl or Ror2 binding assay at a pH 6.7 (or other in vitro tumor surrogate assay condition); and
b. selecting a polypeptide which exhibits an increase in Axl or Ror2 binding activity at pH 6.7 compared to pH7.4, or other in vitro tumor surrogate assay condition compared to the normal physiological condition, thereby selecting the conditionally active antigen specific targeting region.

In some embodiments, a single round of selection is performed to obtain the conditionally active anti-Axl or anti-Ror2 targeting region. In certain embodiments, the screening or panning method is repeated after identifying free antibodies that bound antigen under in vitro tumor surrogate assay conditions and did not bind under physiological conditions, or cells expressing a test antibody that had these properties, or phage coated with a test antibody that has such properties in an initial or previous round. In some methods, phage that are collected are used to infect cells, which can be infected with helper phage as well, in order to amplify the collected phage. In other methods where antibodies on the surface of cells are tested, collected cells can be grown to “amplify” the antibodies expressed by the cells by amplifying antibodies in the cells that encode the polypeptides. In some embodiments, the amplifying is done by growing cells that express the identified antibodies without performing a process to mutate the antibodies encoding the identified antibodies between rounds. Thus, antibodies that are collected in a previous round are then enriched by amplifying cells that contain antibodies encoding these collected antibodies.

The panning or screening method can be performed a single time, or repeated for 1 to 1000 times. In illustrative embodiments, the panning is repeated 1 to 20 times or 2 to 10 times or 2 to 5 times.

In other methods, conditionally active anti-Axl or anti-Ror2 ASTRs are generated using one or more rounds of mutation/evolution between rounds of panning. In one method, a wild-type protein (e.g. antibody) is identified for example by generating a polypeptide or protein library and screening the polypeptide or protein library for a polypeptide or protein with a desired binding affinity to a target antigen. In some embodiments where the wild-type proteins are antibodies, the wild-type antibodies can be discovered by generating and screening polyclonal or monoclonal antibody libraries, including phage display antibody libraries, for example phage display humanized antibody libraries.

Evolved anti-Axl or Ror2 ASTRs can be generated by subjecting the wild-type protein, or a nucleic acid sequence encoding the wild-type protein, to a process of mutagenesis to produce a population of mutant polypeptides that can be screened to identify a mutant ASTR with an increased activity (e.g. enhanced binding affinity to the target antigen) in a tumor environment and/or in an in vitro tumor surrogate assay condition, compared to a normal physiological environment. Examples of such methods are provided in WO2016033331 (“CONDITIONALLY ACTIVE CHIMERIC ANTIGEN RECEPTORS FOR MODIFIED T-CELLS”) or U.S. Pat. No. 8,709,755.

Conditionally active anti-Axl or anti-Ror2 ASTRs identified using methods provided herein are typically polypeptides and more specifically polypeptide antibodies, and in illustrative embodiments, single chain antibodies, as discussed in more detail herein. These polypeptides can bind to Axl or Ror2 with higher or lower affinity under in vitro tumor surrogate assay conditions vs. normal physiologic conditions, but in illustrative embodiments, bind with higher affinity under in vitro tumor surrogate assay conditions than normal conditions. In some embodiments, these polypeptides can bind to their cognate antigen with a 10%, 20%, 25%, 50%, 75%, 90%, 95% or 99% greater affinity under in vitro tumor surrogate assay conditions than physiological (i.e. normal) conditions. In some embodiments, the ASTRs identifying using methods provided herein do not bind to their cognate antigens under normal physiological conditions to any detectable level above background levels obtained using negative controls, such as negative control antibodies.

The nucleotide sequence encoding a conditionally active anti-Axl or anti-Ror2 ASTR isolated by the method provided herein, can be determined by sequencing nucleotides of the collected cell expressing the conditionally active anti-Axl or anti-Ror2 antigen-specific targeting. This nucleotide sequence information can then be used to make a conditionally active anti-Axl or anti-Ror2 biologic chimeric antigen receptor (CAB-CAR) by generating a polynucleotide that encodes a polypeptide comprising the conditionally active anti-Axl or anti-Ror2 antigen-specific targeting region, a transmembrane domain, and an intracellular activating domain. Conditionally active anti-Axl or anti-Ror2 antigen-specific targeting regions can be cloned into a CAR construct expression system, which can be used to generate recombinant lentiviruses that include the CAR in their genome, and then the recombinant lentiviruses can be used to transduce T cells for testing for CAR-mediated Axl or Ror2-expressing target cell killing in a tumor-selective environment compared to normal physiologic conditions, as illustrated in Example 1 herein.

### Methods for Generating a Conditionally Activatable Cell

The present disclosure provides a method of generating a conditionally activatable cell.

The method generally involves genetically modifying a mammalian cell with an expression vector (e.g. a plasmid or a virus), or an RNA (e.g., in vitro transcribed RNA), including nucleotide sequences encoding a conditionally active CAR of the present disclosure. The genetically modified cell is conditionally activatable in the presence of Axl and/or Ror2. The genetic modification can be carried out in vivo, in vitro, or ex vivo. The cell is typically an immune cell (e.g., a T lymphocyte, a T-helper cell, or an NK cell), a stem cell, a progenitor cell, etc. In illustrative embodiments, the cell is a T cell.

In some cases, the genetic modification is carried out ex vivo. For example, a T lymphocyte, a stem cell, a T-helper cell, or an NK cell is obtained from an individual; and the cell obtained from the individual is genetically modified to express a CAR of the present disclosure. The genetically modified cell is conditionally activatable in the presence of Axl and/or Ror2. In some cases, the genetically modified cell is activated ex vivo. In other cases, the genetically modified cell is introduced into an individual (e.g., the individual from whom the cell was obtained); and the genetically modified cell is activated in vivo. For example, where Axl and/or Ror2 are present on the surface of a cell in the individual, there is no need to administer the antigen. The genetically modified cell comes into contact with the antigen present on the surface of a cell in the individual and the genetically modified cell is activated. For example, where the genetically modified cell is a T lymphocyte, the genetically modified cell can exhibit cytotoxicity toward a cell that expresses Axl and/or Ror2 on its surface to which the CAR binds.

In one aspect, provided herein is an ex vivo method for making conditionally activatable T cells and/or NK cells comprising a chimeric antigen receptor (CAR) for conditionally binding Axl or Ror2, wherein the method comprises:
a) enriching peripheral blood mononuclear cells (PBMCs) to isolate PBMCs comprising T cells and/or NK cells from isolated blood;
b) activating T cells and/or NK cells of the enriched PBMCs under effective conditions;
c) transducing the activated T cells and/or NK cells with replication incompetent recombinant retroviral particles under effective conditions, thereby producing genetically modified T cells and/or NK cells, wherein the replication incompetent recombinant retroviral particles each comprise a retroviral genome comprising one or more nucleic acid sequences operatively linked to a promoter active in T cells and/or NK cells, wherein a first nucleic acid sequence of the one or more nucleic acid sequences encodes a CAB-CAR according to any embodiment provided herein; and
d) expanding the genetically modified T cells and/or NK cells, thereby making the conditionally activatable T cells and/or NK cells.

In some embodiments of the above aspect, the method further includes harvesting the expanded genetically modified T cells and/or NK cells. In some embodiments of the above aspect, the method further includes collecting blood from a subject, before enriching PBMCs. In further embodiments, the method further includes introducing the harvested, expanded genetically modified T cells and/or NK cells into the subject. In further embodiments, the genetically modified T cells and/or NK cells are present in the subject 1, 2, 3, 4, 5, 6, 7, or 14 days after they are introduced into the subject.

### Blood Collection

Blood containing PBMCs can be collected or obtained from a subject by any suitable method known in the art. For example, the blood can be collected by venipuncture or any other blood collection method by which a sample of blood and/or PBMCs is collected. In some embodiments, PBMCs can be obtained by apheresis as discussed below.

### Enrichment of PBMCs

In ex vivo methods for making conditionally activatable T cells and/or NK cells, peripheral blood mononuclear cells (PBMCs) including T cells and/or NK cells, are isolated away from other components of a blood sample in an enrichment step. Enrichment of PBMCs from other blood components and blood cells can be performed using any methods known in the art, for example, using apheresis, and/or density gradient centrifugation. In some embodiments, Ficoll-Paque (GE Healthcare) can be used. In some embodiments, an automated apheresis separator is used which takes blood from the subject, passes the blood through an apparatus that sorts out a particular cell type (such as, for example, PBMCs), and returns the remainder back into the subject. Density gradient centrifugation can be performed after apheresis. In some embodiments, the PBMCs can be enriched and isolated using a leukoreduction filter device. In some embodiments, magnetic bead activated cell sorting is then used for purifying a specific cell population from PBMCs, such as, for example, T cells and/or NK cells, according to a cellular phenotype (i.e. positive selection). In some embodiments, monocytes and/or macrophages can be removed from the PBMCs using methods known in the art. With reference to a subject to be treated, the cells can be allogeneic and/or autologous. During the PBMC enrichment process, one or more washes can be performed as is known in the art, before the enriched PBMCs are isolated and then activated. The wash solution can any solution suitable for washing blood and/or PBMCs. According to methods known in the art, the isolated PBMCs can be resuspended in any suitable base culture medium used for culturing T cells and/or NK cells. In some embodiments, the media can be supplemented with HSA, human AB+serum, serum derived from the subject and/or serum replacement.

### Activation of PBMCs

Ex vivo methods for making conditionally activatable T cells and/or NK cells provided herein typically include a step of activating or stimulating the isolated PBMCs with one or more activating agents to generate activated T cells and/or NK cells. Activating can be performed on either freshly isolated PBMCs or previously cryopreserved PBMCs. In the event that cryopreserved cells are used, the cells may be thawed using developed protocols prior to use.

Media is typically present during the activating, such as those known in the art for ex vivo processes (as non-limiting examples, X-VIVO 15 (Lonza) or CTS media (Thermo Fisher)). In some embodiments, the media can be supplemented with HSA, human AB+serum, serum derived from the subject, and/or serum replacement. In illustrative embodiments, the media can be supplemented with serum replacement, such as CTS Serum Replacement (Thermo Fisher). In some embodiments, the media can be supplemented with HSA, human AB+serum, serum derived from the subject and/or serum replacement.

Any combination of one or more activating agents can be added to the media to produce activated T cells and/or NK cells. A reaction mixture is typically formed to perform the activating. In some embodiments, the reaction mixture can be formed by adding one or more activating agents to the media. In any of the embodiments disclosed herein, the one or more activating agents are used in effective amounts such that activated T cells and/or NK cells are produced.

It is noteworthy that such activation in embodiments for making conditionally activatable T cells and/or NK cells can involve activating the cells with Axl or Ror2, such as isolated soluble Axl or Rors, at a pH below 7.0. However, activation in methods for making conditionally activatable T cells and/or NK cells typically utilizes more generic activating agents. Accordingly, in some embodiments, the activating agent can be a polypeptide or an antibody (e.g. anti-CD2, anti-CD3, and/or anti-CD28) or functional fragments thereof that target or bind to a T-cell stimulatory or co-stimulatory molecule, a T cell cytokine, or any other suitable mitogen (e.g., tetradecanoyl phorbol acetate (TPA), phytohaemagglutinin (PHA), concanavalin A (conA), lipopolysaccharide (LPS), pokeweed mitogen (PWM)), a natural ligand to a T-cell stimulatory or co-stimulatory molecule, phospho-antigens, or aminobisphosphonates, such as zoledronate. Various antibodies and functional fragments thereof are known in the art to activate or stimulate T cells and/or NK cells. In some embodiments, the one or more antibodies or functional fragments thereof can be immobilized on a solid surface, such as a bead.

Transduction of T Cells and/or NK Cells

Ex vivo methods for making conditionally activatable T cells and/or NK cells provided herein typically include a step of transforming or transducing activated T cells and/or NK cells. In some embodiments of such methods, T cells and/or NK cells are contacted ex vivo with expression vectors such as replication incompetent recombinant retroviral particles to genetically modify the T cells and/or NK cells. Not to be limited by theory, during the period of contact the replication incompetent recombinant retroviral particles bind to T cells and/or NK cells at which point the retroviral and host cell membranes start to fuse. Then, through the process of transduction, genetic material from the replication incompetent recombinant retroviral particles enters the T cells and/or NK cells and typically is incorporated into the host cell DNA. Accordingly, such methods include genetically modifying T cells and/or NK cells by transduction. Methods are known in the art for transducing T cells and/or NK cells ex vivo with replication incompetent recombinant retroviral particles, such as replication incompetent recombinant lentiviral particles. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505. In some embodiments, the T cells and/or NK cells can be contacted with replication incompetent recombinant retroviral particles. In illustrative embodiments, the T cells and/or NK cells can be contacted with replication incompetent recombinant lentiviral particles.

Expansion of Transduced T Cells and/or NK Cells

In illustrative embodiments of ex vivo methods for making conditionally activatable T cells and/or NK cells provided herein, transduced T cells and/or NK cells are expanded before harvesting. In any of the embodiments disclosed herein, media is present for the activating and transducing and can be further added or exchanged after transducing, to perform the expansion. In some embodiments, media can be added to the reaction mixture formed during the activating. The media used for the expanding typically includes the same base media used in the activating and transducing, such as those known in the art for ex vivo processes, especially for T cells and/or NK cells (as non-limiting examples, X-VIVO 15 (Lonza) or Optimizer CTS media (Thermo Fisher)). In some embodiments, the media can be supplemented with HSA, human AB+serum, serum derived from the subject, and/or serum replacement, such as CTS Serum Replacement (Thermo Fisher). Cytokines, such as IL-2, IL-7, or IL-15, or those found in HSA can be added to the media before, during, and/or after activation, transduction, and expansion. Cell expanding can be performed for a certain number days. In some embodiments, expanding can be performed for 4, 5, 6, 7, 8, 9, 10, 11, 12, 13, 14, 15, 16, 17, 18, 19, 20, or 21 days. In some embodiments, expanding can be performed for between 4, 5, 6, 7, or 8 days on the low end of the range and 9, 10, 11, 12, 13, 14, 15, 16, 17, 18, 19, 20, or 21 days on the high end of the range. In certain illustrative embodiments, expanding is performed for between 6 and 12 days, or between 8 and 10 days.

### Cell Harvesting

In ex vivo methods for making conditionally activatable T cells and/or NK cells provided herein typically include harvesting the genetically modified T cells and/or NK cells after expanding. In some embodiments, the transduced T cells and/or NK cells can be concentrated or collected during harvesting using methods known in the art. In some embodiments, the T cells and/or NK cells can be washed one or more times during the harvesting using any suitable wash solution known in the art. At the end of the harvesting, the T cells and/or NK cells can be resuspended in any suitable media known in the art. In any of the embodiments disclosed herein, harvesting of the expanded T cells and/or NK cells can be performed based on an expansion completion criteria. In some embodiments, the expansion completion criteria can be lactate concentration, cell density, or a number of days in expansion.

In some embodiments, the harvested cells can be introduced, introduced back, reintroduced, infused, or reinfused into a subject. In some embodiments, harvested cells can be cryopreserved as described below before reintroduction into a subject. In illustrative embodiments, harvested cells are introduced, introduced back, reintroduced, infused, or reinfused into a subject without first cryopreserving the cells. The subject is typically the same subject the blood was collected from.

Throughout this disclosure, a transduced T cell and/or NK cell includes progeny of the transduced cells that retain at least one of the nucleic acids that are incorporated into the cell during the ex vivo transduction. In methods herein that recite “reintroducing” a transduced cell, it will be understood that such a cell is typically not in a transduced state when it is collected from the blood of a subject.

### Cell Introduction/Reintroduction

In certain embodiments of the ex vivo methods for making conditionally activatable T cells and/or NK cells disclosed herein, the harvested T cells and/or NK cells can be introduced, introduced back, reintroduced, infused, or reinfused in a subject for a therapeutic effect. The number of T cells and/or NK cells to be reintroduced can be a predetermined dose, which can be a therapeutically effective dose. In some embodiments, the predetermined dose can depend on the CAR that is expressed on the cells (e.g., the affinity and density of the antigen-specific targeting region expressed on the transduced T cell and/or NK cell), the type of target cell, the nature of the disease or pathological condition being treated, or a combination. In some embodiments, the predetermined dose of harvested cells can be based on the mass of a subject, for example, cells per kilogram of the subject (cells/kg).

### Cell Cryopreservation

In ex vivo methods for making conditionally activatable T cells and/or NK cells provided herein, the harvested cells produced by the methods described herein can be cryopreserved at a predetermined dose for use at a later time. Methods and reagents for cryopreserving cells are well-known in the art. Cryopreservation can include one or more washes and/or a step of concentrating the T cells and/or NK cells. The method can also include a step of forming a cryopreservation mixture, which includes the T cells and/or NK cells in the diluent solution and a suitable cryopreservative solution. In some embodiments, the method can include a step of freezing the cryopreservation mixture as is known in the art. Methods of thawing cryopreserved T cells and/or NK cells are known in the art.

Methods for Modulating Cab-Car-Expressing T Cell and/or Nk Cell Activity by Changing pH

Provided herein in certain aspects, are methods for modulating activation of an immune cell (e.g. T cell or NK cell) by contacting the immune cell with Axl or Ror2 in a microenvironment at a pH below 7.0 (e.g below 6.9 or 6.8) and then changing the pH of the microenvironment, such that it is at or above 7.0 (e.g. above 7.1, 7.2, or 7.3), wherein the immune cell expresses any of the CAB-CARs provided herein. In illustrative embodiments, the Axl or Ror2 is expressed on the surface of a target mammalian cell. In certain embodiments, such methods for modulating activation are the identical to methods for activating an immune cell provided herein, further comprising the additional step of increasing the pH of the microenvironment to a pH at or above 7.0 (e.g. above 7.1, 7.2, or 7.3), thereby decreasing the activation of the immune cell. In illustrative embodiments, such increase in pH deactivates the immune cell.

Provided herein in certain aspects, are methods for modulating binding and resulting lysis/killing of a target cell by a CAB-CAR-expressing T cell or NK cell by causing a change or shift in pH within a microenvironment that includes a target cell either within a target tissue or within one or more non-target (e.g. healthy/normal) tissues, by modulating binding of the CAB-CAR to its cognate antigen on a target cell(s), wherein the cognate antigen is an Axl polypeptide or an epitope thereof or a Ror2 polypeptide or an epitope thereof. Such methods typically include contacting a target cell, such as a mammalian cell (e.g. a human cell) with a CAB-CAR-expressing T cell or NK cell in a microenvironment and then changing the pH of the microenvironment, either by decreasing or more typically increasing the pH. The microenvironment can be a target microenvironment, for example a tumor, or an off-target microenvironment, where off-target binding can cause side-effects. In some embodiments, such methods can provide a transient reduction of tumor microenvironment sensitive CAR-T target binding.

Accordingly, in one aspect, provided herein is a method for modulating binding of a conditionally active biologic chimeric antigen receptor (CAB-CAR)-expressing T cell or NK cell to a cell expressing a cognate antigen of the CAB-CAR in a subject, that includes the following:
a. introducing a T cell and/or NK cell comprising a nucleic acid encoding the CAB-CAR into the subject, wherein after (and optionally and/or during) the introducing, the T cell and/or the NK cell comprising the nucleic acid encoding the CAB-CAR expresses the CAB-CAR and binds to the cell expressing the cognate antigen in the subject, wherein the cognate antigen is an Axl polypeptide or an epitope thereof or a Ror2 polypeptide or an epitope thereof; and
b. administering a pharmacologic agent to the subject in sufficient amount to increase blood pH and/or pH of a tissue and/or pH of a microenvironment, wherein the administering is performed before, during, or after the introducing, and wherein the increased pH of the blood, the tissue, and/or the microenvironment modulates binding of the CAB-CAR expressing T cell and/or NK cell to the cell expressing the cognate antigen in the blood, the tissue, or the microenvironment with the increased pH.

The change/shift in pH in aspects that include a step of administering a pH-modulating pharmacologic agent of the present disclosure can be accomplished by exposing target or non-target cells/tissue to a pH-modulating pharmacologic agent, such as by administering the pH modulating pharmacologic agent to a subject. Non-limiting examples of pH-modulating pharmacologic agents are provided herein. In certain aspects, provided herein is a pharmacologic agent for use in a method for modulating binding of a CAB-CAR to its cognate antigen or for modulating binding of a CAB-CAR-expressing T cell and/or NK cell to a cell that expresses its cognate antigen or for reducing or alleviating on target off tumor toxicity in a subject. Such aspects in certain embodiments, relate to treating tumor growth, cancer, hyperplasia, or cell proliferative disorders.

In other aspects, provided herein is use of a pH-modulating pharmacologic agent for use in the manufacture of a medicament or a kit for controlling binding of a genetically engineered T cell and/or NK cell to a target mammalian cell in a subject in vivo, wherein the target cell expresses an Axl polypeptide or an epitope thereof or a Ror2 polypeptide or an epitope thereof. In other aspects, provided herein is a kit that includes a container containing a replication incompetent recombinant retroviral particle, and instructions for use thereof for performing a method for treating tumor growth, wherein the instructions instruct a method for controlling binding of a T cell and/or NK cell to a target mammalian cell by modulating pH, wherein the target mammalian cell expresses an Axl polypeptide or an epitope thereof or a Ror2 polypeptide or an epitope thereof. Such method can be any of the methods provided herein this section for modulating CAB-CAR-expressing T cell and/or NK cell binding and/or activity by changing pH. The container that contains the recombinant retroviral particles can be a tube, vial, well of a plate, or other vessel for storage of a recombinant retroviral particle and/or a pH-modulating pharmacologic agent. Any of these can be of industrial strength and grade. The kit can include two or more containers in certain embodiments. One container/vessel can include the recombinant retroviral particles and another container/vessel can include a pH-modulating pharmacologic agent. In such methods the pharmacologic agent is delivered/administered in sufficient amount to increase blood pH and/or a tissue pH and/or a microenvironment pH to modulate binding of the CAB-CAR of a modified/recombinant T cell and/or NK cell expressing the CAB-CAR, to its cognate antigen in the blood and/or the tissue with the increased pH. Non-limiting exemplary details are provided herein for administering a pH modulating pharmacologic agent in sufficient amount and for a sufficient time.

Target cells, whether on target or off target with respect to a tissue, can be contacted with a pH modulating agent, such as a pH modulating pharmacologic agent, after introducing the CAB-CAR into a subject. Accordingly, exemplary aspects provided herein for modulating binding and/or cytotoxic activity of a CAB-CAR-expressing T cell that is capable of binding to (i.e. recognizes) an Axl polypeptide or an epitope thereof, or a Ror2 polypeptide or an epitope thereof, for example for alleviating on target off tumor activity and/or for inhibiting target cell proliferation, such as tumor cell proliferation, can include the following steps:
a. introducing a T cell and/or NK cell comprising a nucleic acid encoding a CAB-CAR into a subject wherein after the introducing, the T cell and/or the NK cell comprising the nucleic acid encoding the CAB-CAR expresses the CAB-CAR, wherein the CAB-CAR is capable of binding to Axl polypeptide or an epitope thereof, or a Ror2 polypeptide or an epitope thereof, and optionally binds to the cell expressing the cognate antigen in the subject; and
b. administering a pharmacologic agent to the subject in sufficient amount to increase blood pH and/or a tissue pH and/or a microenvironment pH to modulate binding of the CAB-CAR-expressing T cell and/or NK cell to cells expressing the cognate antigen of the CAB-CAR, in the blood, the tissue, or the microenvironment with the increased pH. It will be understood that depending on the specific method used to introduce the nucleic acid encoding the CAB-CAR into the T cell and/or NK cell, the T cell and/or NK cell may or may not express the CAB-CAR before it is introduced into the subject. However, at some timepoint after introduction into the subject, e.g. 2 hours, 4 hours, 8 hours, 12 hours, 1 day, 2 days, 4 days and/or 7 days, or longer, the T cell and/or NK cell that include the nucleic acid encoding the CAB-CAR, express the CAB-CAR. Then such cells typically bind to a target cell expressing the cognate antigen for the CAB-CAR.

Methods provided herein for genetically modifying and optionally expanding lymphocytes of a subject can be used to introduce a nucleic acid sequence that encodes a CAB-CAR into the genome of a T cell and/or NK cell of the subject to produce an T cell and/or NK cell capable of expressing the CAB-CAR, and then to introduce the T cell and/or NK cell capable of expressing the CAB-CAR into the subject, wherein after introducing the T cell and/or NK cell expresses the CAB-CAR in order to contact the CAB-CAR with a target cells/tissue. The present disclosure provides details of how to perform such methods, along with various alternatives for different CAR components, any of which can be used in aspects of the disclosure that include changing pH to modulate binding of a CAB-CAR-expressing T cell and/or NK cell to a target cell expressing a cognate antigen for the CAB-CAR.

Such methods for genetically modifying and expanding lymphocytes typically involve contacting T cells and/or NK cells, with a replication incompetent recombinant retroviral particle to transduce the T cells and/or NK cells. Such contacting typically occurs ex vivo after removing the lymphocytes from the subject. The T cells and/or NK cells are then introduced/reintroduced into the subject, typically from whom they were removed. The replication incompetent recombinant retroviral particle includes a genome with a polynucleotide that encodes the CAB-CAR. Many alternative embodiments and further details regarding such a replication incompetent recombinant retroviral particle are provided in other sections herein and can be used in methods provided herein for regulating binding and resulting lysis/killing of T cells expressing CAB-CARs that are capable of binding to an Axl polypeptide or an epitope thereof, or a Ror2 polypeptide or an epitope thereof, by modulating pH in a microenvironment of a cell expressing a cognate target polypeptide recognized by the CAB-CAR in a pH-dependent manner.

Such methods for modulating binding of a target cell by a CAB-CAR-expressing T cell and/or NK cell can be used, for example, to reduce on target, off-tumor toxicity by increasing the pH of blood and/or a non-tumor tissue(s) within the subject. For example, in a situation where a “normal” tissue pH within a subject becomes transiently lower, a pH modulating agent can be delivered in a manner where pH of the normal tissue is increased while pH of the tumor remains lower and still at a pH where the CAB-CAR-expressing T cell and/or NK cell binds a target tumor cell. In these embodiments, the pH modulating agent can be delivered at a lower concentration or in a targeted manner to the normal tissue.

In some embodiments, this can be accomplished while allowing the pH within the tumor microenvironment to remain low enough for a CAB-CAR T cell and/or NK cell to bind to its cognate target-expressing cells within the tumor. In illustrative aspects of methods provided herein, the pH of a tissue remains at a pH under which a CAB-CAR-expressing T cell and/or NK cell binds its target for a period of time sufficient for a CAB-CAR-expressing T cell and/or NK cell to contact and bind to a cell expressing its cognate antigen (e.g. 2, 4, 8, 12, or 24 hours, or 2, 4, 7, 14, 28, or 30 days, or 1, 2, 3, 4, 5, 6, 12, 24 months, or longer), and then the pH is shifted/changed, for example by increasing the pH of the tissue to such a magnitude as to affect binding of the CAB-CAR-expressing T cell and/or NK cell to a target cell.

Accordingly, provided herein, in one aspect, is a method for transient reduction of tumor microenvironment sensitive CAR-T cell target binding through pharmacologic modification of vascular and tissue pH, wherein the CAR-T cell expresses a CAB-CAR that is capable of binding to an Axl polypeptide or an epitope thereof, or a Ror2 polypeptide or an epitope thereof. These microenvironmentally controlled ASTRs in CAR-T cells provide an additional level of protection against on-target off tumor toxicity, requiring tumor local environmental conditions to enable T cell engagement. While attractive for some monoclonal antibody therapies, adoptive cellular therapy may create local environments that are transiently permissive for their CAR-T targets. For example, CAR-T cells activated in tissues with a low pH may further reduce the pH of the microenvironment, depending on cytoplasmic domains present in the CAR construct. In other instances, cytokine release syndrome and other morbidity associated with adoptive cellular therapy may result in loss of the bicarbonate buffering capacity of blood, leading to lactic acidosis. It has been established that adoptive cellular therapies administered by intravenous infusion result in temporary pulmonary entrapment. For some cellular therapies, infusion rate requires constant monitoring of dissolved oxygen (Fischer et al. Stem Cells Dev. 2009 June; 18(5): 683-691). The extent of pulmonary entrapment is dependent upon cell size, activation state, cell dose, and infusion rate. Cruz et al (Cytotherapy. 2010 October; 12(6): 743-749) report the adverse findings from over 300 T cell infusions, that low doses and slow infusion may reduce pulmonary entrapment. However, with certain high potency CAR-T cells, targets present even in low levels on lung endothelium, such as Her2 (Morgan et al. Mol Ther. 2010 April; 18(4): 843-851), can result in immediate toxicity that cannot be controlled, and results in rapid patient deterioration due to the initial high CAR-T cellular concentration in the lung following infusion and the presence of the T cell target in these tissues. In other cases, the presence of T cell targets in other off target tissues such as bile duct may create on target off tumor toxicities that cannot be controlled (Lamers Mol Ther. 2013 April; 21(4):904-12) and result in severe organ toxicity before other agents such as steroids or cell elimination epitopes can be utilized. While venous and arterial plasma have strong buffering capacity against acidosis, conditions of respiratory acidosis, shock, metabolic acidosis and ischemic acidosis can occur in patients with cancer treated with adoptive cellular therapy.

In some aspects provided herein, the binding of a CAB-CAR in a subject can be modulated by administering a pharmacologic agent to the subject to increase or decrease the pH of the blood, a tissue and/or a microenvironment. In some aspects, on-target off tumor toxicity can be alleviated in a subject by administering a pharmacologic agent to the subject to increase or decrease the blood pH and/or the pH of a tissue and/or the pH of a microenvironment. In some aspects, the binding of a T cell and/or NK cell to a target mammalian cell can be controlled by introducing a pharmacologic agent to increase or decrease the blood pH and/or the pH of a tissue and/or the pH of a microenvironment. In some aspects, the binding of a genetically engineered T cell and/or NK cell to a target mammalian cell in a subject in vivo can be controlled by administering a pH-modulating pharmacologic agent to the subject. In illustrative embodiments, the pharmacologic agent can increase the blood pH and/or the pH of a tissue and/or the pH of a microenvironment. In some embodiments, the microenvironment can be an in vivo microenvironment. In illustrative embodiments, the microenvironment can be a tumor microenvironment. In some embodiments, the microenvironment can include a target mammalian cell, wherein the target mammalian cell expressed the target antigen on its surface. In some embodiments, administering a pharmacologic agent to a subject can increase the pH of blood, a tissue, and/or a microenvironment from a pH of less than 5.8, 5.9, 6.0, 6.1, 6.2, 6.3, 6.4, 6.5, 6.6, 6.7, 6.8, or 6.9 to a pH of at least 6.6, 6.7, 6.8, 6.9, 7.0, 7.1, 7.2, 7.3, 7.4, 7.5, or 7.6, wherein the pH of the blood, tissue, and/or microenvironment is lower before administering the pharmacologic agent than after administering the pharmacologic agent. In some embodiments, administering a pharmacologic agent to a subject can decrease the pH of blood, a tissue, or a microenvironment from a pH of more than 6.6, 6.7, 6.8, 6.9, 7.0, 7.1, 7.2, 7.3, 7.4, 7.5, or 7.6 to a pH of less than 5.8, 5.9, 6.0, 6.1, 6.2, 6.3, 6.4, 6.5, 6.6, 6.7, 6.8, 6.9, or 7.0, wherein the pH of the blood, tissue, and/or microenvironment is higher before administering the pharmacologic agent than after administering the pharmacologic agent. In some embodiments, administering a pharmacologic agent to a subject can cause a pH shift in the subject in the blood, a tissue, and/or a microenvironment. In some embodiments, the pH shift can be at least 0.1, 0.2, 0.3, 0.4, 0.5, 0.6, 0.7, 0.8, 0.9, 1.0, 1.1, 1.2, 1.3, 1.4, 1.5, 1.6, 1.7, or 1.8 pH units in either direction, i.e. an increase or decrease in pH after administering the pharmacologic agent relative to the pH before administering the pharmacologic agent. In illustrative embodiments, the pH shift is an increase in pH.

The CAB-CARs of the present disclosure can have reduced binding to its cognate antigen at one pH than at a different pH. In illustrative embodiments where illustrative pH values for differential binding of a CAB-CAR are not already provided in the broadest aspect and alternatively for other embodiments in place of those values for such aspects, the CAB-CAR can have reduced binding at a higher pH than at a lower pH. For example, the CAB-CAR can have reduced binding to its cognate antigen at a pH above 7.0, 7.1, 7.2, 7.3, 7.4, or 7.5 than at a pH below 6.4, 6.5, 6.6, 6.7, 6.8, 6.9, or 7.0. In other embodiments, the CAB-CAR can have reduced binding at a higher pH than at a lower pH. For example, the CAB-CAR can have reduced binding to its cognate antigen at a pH below 6.4, 6.5, 6.6, 6.7, 6.8, 6.9, or 7.0 than at a pH above 7.0, 7.1, 7.2, 7.3, 7.4, or 7.5. In some illustrative embodiments, the CAB-CAR exhibits increased binding at a pH of 6.5 to 6.7 compared to pH 7.4 to 7.6. In other illustrative embodiments, the CAB-CAR exhibits increased binding at a pH of 6.7 compared to a pH of 7.4. In other embodiments, the CAB-CAR exhibits increased binding in the pH of a tumor compared to the pH of blood. In some embodiments, the CAB-CAR can include an antigen-specific targeting region, a stalk, and an intracellular activating domain. In some embodiments, the CAB-CAR can also include a co-stimulatory domain. In some embodiments, the CAB-CAR can bind to a tumor associated antigen. In some embodiments, the CAB-CAR binds to an Axl polypeptide or an epitope thereof or a Ror2 polypeptide or an epitope thereof.

In methods that include modulating the pH of the blood, a tissue, or a microenvironment, the pH of the microenvironment can be increased from a pH below 7.0 to a pH above 7.0. For example, the pH can be increased from a pH below 6.4, 6.5, 6.6, 6.7, 6.8, 6.9, or 7.0 to a pH above 7.0, 7.1, 7.2, 7.3, or 7.4. In some embodiments, the CAB-CAR can bind to the cognate antigen at the increased pH but not a pH of the microenvironment before introducing the pharmacologic agent. In certain embodiments, the pH can be increased from below 7.0 to a pH of 7.1 to 8.0 or to a pH of 7.1 to 7.8 or to a pH of 7.2 to 7.8 or a pH of 7.2 to 7.6 or a pH of 7.3 to 7.6 or to a pH of 7.4 to 7.8 or to a pH of 7.4 to 7.6. Such an increase in pH can occur for less than 1, 2, 4, 6, 8, 12, or 24 hours or for more than 1, 2, 4, 6, 8, 12 or 24 hours depending on the type and dose of pharmacologic agent administered. In certain embodiments, the pharmacologic agent is administered such that the pH remains above 7.0, 7.1, 7.2, 7.3, 7.4, or 7.5; or between 7.0, 7.1, 7.2, 7.3 on the low end of the range and 7.4, 7.5, 7.6, 7.7, or 7.8 on the high end of the range, in the target tissue, such as a tumor, and for example in at least a surface of a target tissue (e.g. tumor) microenvironment, in at least a portion of a target tissue (e.g. tumor) microenvironment, and in illustrative embodiments throughout a target tissue (e.g. tumor) microenvironment. The microenvironment can be an in vivo microenvironment, such as a tumor, a tissue, a non-tumor tissue, a normal tissue, or a tissue that has undergone a transient shift in pH. For example, tissues that typically undergo transient shifts in pH include a muscle tissue in anaerobic conditions or muscle tissue undergoing exercise or an inflamed tissue or a tissue experiencing inflammation. In some embodiments that include a target mammalian cell, the target mammalian cell can be a tumor cell or a non-tumor or normal cell.

In some aspects, methods for transiently increasing vascular pH to reduce affinity of microenvironmentally controlled CAB-CARs that recognize, are capable of binding, and in some embodiments bind an Axl polypeptide or an epitope thereof, or a Ror2 polypeptide or an epitope thereof are provided. A 0.4U shift in blood pH can reduce the affinity of certain scFvs that form a portion of a CAB-CAR, for their cognate antigen by greater than 10-fold. In some embodiments, therapeutic pH control can be achieved via IV or oral administration routes of various pharmacologic agents. For example, in some embodiments, inactivation of binding affinity can be achieved with bicarbonate or sodium bicarbonate. In other embodiments, Tris-hydroxymethyl aminomethane (also known as tromethamine, trometamol, and THAM) and/or Carbicarb™ (an equimolar hypertonic solution of sodium bicarbonate and sodium carbonate) can be utilized to increase the pH of the blood in a sufficient amount to alleviate on-target off tumor toxicities. In still other embodiments, small molecule proton pump inhibitors can be utilized to increase blood pH and/or tissue pH in a sufficient amount to alleviate on-target off tumor toxicities. Proton pump inhibitors that can be used in methods that include modulating pH include, but are not limited to, esomeprazole (Nexium), esomeprazole and naproxen (Vimovo), lansoprazole (Prevacid), omeprazole (Prilosec and Zegerid), and rabeprazole (Aciphex). Administration of proton pump inhibitors can be used effectively over longer time periods to modulate the binding affinity of the antigen biding domain to its cognate antigen for days, weeks, months, or years. In other embodiments, the affinity of the antigen binding domain for its cognate antigen can be modulated by altering the blood pH and/or tissue pH by controlling the transcription, translation, membrane expression, and stability of transporters and pumps. Examples of such transporters and pumps whose altered expression can be to modulate pH include, but are not limited to, proton pumps, members of the sodium proton exchange family (NHE), bicarbonate transporter family (BCT), and monocarboxylate transporter family.

In certain embodiments, a pH-modulating pharmacologic agent, such as, for example, bicarbonate, THAM, or Caricarb™ are administered prior to or concurrent with infusion of a patient's CAR-T cells expressing conditionally active biologic ASTRs (e.g. scFvs or scFvFcs). Such treatment will alleviate the immediate cytoxicity that is otherwise associated with the temporary pulmonary entrapment of CAR-T cell infusions. Accordingly, in certain aspects provided herein is a method for reducing cytotoxicity caused to normal, healthy tissue of a subject by administering a pharmacologic agent to the subject in sufficient amount to increase blood pH and/or a tissue pH and/or a microenvironment pH; and either concomitantly or subsequently (e.g. 1, 2, 4, 6, 8, 12, or 24 hours, or 1, 2, 3, 4, or 7 days later) introducing a CAB-CAR-expressing T cell or NK cell into the subject. In certain embodiments, at a target time after such introducing (e.g. 1, 2, 4, 6, 8, 12, or 24 hours, or 1, 2, 3, 4, or 7 days later), administration of the pharmacologic agent is terminated for a period of time or indefinitely, in order to change the pH of the blood, a tissue, or a microenvironment of the subject and modulate binding/activity of the CAB-CAR-expressing T cell.

Various effective dosing regimens for administering the pharmacologic agents capable of modulating pH (e.g. increasing blood pH and/or a tissue pH and/or the pH of a microenvironment in a subject) can be used, as will be understood by a skilled artisan. Herein, administering can refer to giving a pharmacologic agent to a subject including injecting a pharmacologic agent through an IV into a subject or providing an oral dose of a pharmacologic agent to a subject or a subject taking a pharmacologic agent. The pharmacologic agents can be administered to the subject or patient for various lengths of time, for example, at least 1, 2, 3, 4, 5, or 6 days; 1, 2, 3, 4, 5, 6, 7, 8, 9, 10, or 11 weeks; 3, 4, 5, 6, 7, 8, 9, 10, 12, 15, or 18 months; or 2, 2.5, 3, 3.5, 4, 4.5, or 5 years or indefinitely. In some embodiments, the pharmacologic agent can be bicarbonate, sodium bicarbonate (NaHCO₃), or a solution of sodium bicarbonate and sodium carbonate and a parenteral or IV dosage can be: 0.2×weight of subject (kg)×base deficit of the subject; HCO₃ (mEq) required=0.5×weight (kg)×[24−serum HCO₃ (mEq/L)]; or 2 to 5 mEq/kg IV infusion over 4 to 8 hours. In some embodiments, standard dosing regimens of bicarbonate, sodium bicarbonate, or a solution of sodium bicarbonate can be used depending on the severity of the acidosis. For example, 50 to 150 mEq bicarbonate diluted in 1 L of 5% dextrose in water can be administered via IV at a rate of 1 to 1.5 L/hour. In another non-limiting example, 90 to 180 mEq bicarbonate diluted in 1 L of 5% dextrose in water can be administered via IV at a rate of 1 to 1.5 L/hour. In some embodiments where the pharmacologic agent is bicarbonate or sodium bicarbonate (NaHCO₃), an enteral or oral dosage can be, for example, 325 to 2000 mg sodium bicarbonate given to a subject 1 to 4 times/day.

In some embodiments, the pharmacologic agent can be tris-hydroxymethyl aminomethane (also known as tromethamine, trometamol, and THAM) and a parenteral or IV dosage can be estimated as: Tromethamine solution (mL of 0.3 M) required=Body Weight (kg)×Base Deficit (mEq/liter)×1.1. In some embodiments, the IV dosage of tris-hydroxymethyl aminomethane can be estimated from the buffer base deficit of the extracellular fluid in mEq/L as determined by means of the Siggaard-Andersen nomogram. In some embodiments, the initial dose can be 500 ml (150 mEq) of tris-hydroxymethyl aminomethane injected by slow IV infusion with up to 1000 mL, wherein the maximum dose is 500 mg/kg (227 mg/lb) over a period of not less than one hour.

In some embodiments, the pharmacologic agent can be a small molecule proton pump inhibitor and can be administered for extended treatment lengths. For example, the small molecule proton pump inhibitor can be administered for at least 1, 2, 3, 4, 5, or 6 days; 1, 2, 3, 4, 5, 6, 7, 8, 9, 10, or 11 weeks; 3, 4, 5, 6, 7, 8, 9, 10, 12, 15, or 18 months; or 2, 2.5, 3, 3.5, 4, 4.5, or 5 years or indefinitely. In some embodiments, the proton pump inhibitor can be esomeprazole (Nexium) and 20 mg or 40 mg esomeprazole can be administered orally once or twice daily. In some embodiments, the proton pump inhibitor can be a combination of esomeprazole and naproxen (Vimovo) and 20 mg esomeprazole with 375 or 500 mg naproxen can be administered orally twice daily. In some embodiments, the proton pump inhibitor can be lansoprazole (Prevacid) and 15, 30, or 60 mg lansoprazole can be administered orally once or twice daily. In some embodiments, lansoprazole can be administered by IV with 30 mg lansoprazole injected over 30 minutes once daily for up to 7 days. The subject can then switch to oral lansoprazole and continue treatment. In some embodiments, the proton pump inhibitor can be omeprazole (Prilosec and Zegerid) and 10, 20, or 40 mg omeprazole can be administered orally once or twice daily. In some embodiments, the proton pump inhibitor can be rabeprazole (Aciphex) and 20 or 60 mg rabeprazole can be administered orally once or twice daily or 100 mg rabeprazole can be administered orally once daily. In any of the embodiments disclosed herein, the pharmacologic agents can be used in combination with each other.

In any of the embodiments disclosed herein, the pH of the blood, a tissue, and/or a microenvironment of a subject can be measured before, during, or after the administration of a pharmacologic agent. In some embodiments, the decision to administer or to continue to administer, to a subject the pharmacologic agent to increase or decrease the pH can be based on the pH measurement of the blood, a tissue, and/or a microenvironment of the subject. Methods to measure the blood pH and/or bicarbonate levels of the blood of a subject are well-known in the art. In some embodiments, positron emission tomography (PET), magnetic resonance spectroscopy (MRS), magnetic resonance imaging (MRI), and optical imaging can be used to measure in vivo pH in microenvironments, for example, in tumors (for details of measuring tumor pH, see: Zhang X, Lin Y, Gillies R J. Tumor pH and its measurement. J Nucl Med. 2010 August; 51(8):1167-70).

In another aspect, provided herein is a method for alleviating on target off tumor toxicity in a subject, that includes the following:
a. introducing a polynucleotide encoding a conditionally active biologic chimeric antigen receptor (CAB-CAR) into a T cell or NK cell of the subject to produce a T cell and/or NK cell capable of expressing the CAB-CAR, wherein the CAB-CAR is capable of binding to an Axl polypeptide or an epitope thereof, or a Ror2 polypeptide or an epitope thereof;
b. introducing the T cell and/or NK cell capable of expressing the CAB-CAR into the subject, wherein the T cell and/or NK cell express the CAB-CAR in the subject; and
c. administering a pharmacologic agent to the subject in sufficient amount to increase blood pH and/or pH of a tissue and/or pH of a microenvironment to modulate binding of the CAB-CAR to its cognate antigen in the blood, the tissue, and/or the microenvironment with the increased pH, thereby alleviating on target off tumor toxicity in the subject.

In the introducing step, the T cell or NK cell is capable of expressing the CAB-CAR that is capable of binding to an Axl polypeptide or an epitope thereof, or a Ror2 polypeptide or an epitope thereof because it is genetically modified to contain the nucleic acid that encodes the CAB-CAR. This genetic modification can be the presence of the CAB-CAR coding sequence on a vector that has been introduced inside the T cell or NK cell by transfection or transduction. In illustrative embodiments the nucleic acid encoding the CAB-CAR is integrated into the genome of the T cell or NK cell.

It is envisioned that various methods known in the art for introducing a polynucleotide into a T cell and/or NK cell could be used with methods provided herein for aspects that include changing pH to affect binding of a CAB-CAR T cell or NK cell to its cognate antigen on a cell using an agent such as a pH-modulating pharmacologic agent (sometimes referred to herein as “pH Switch aspects”). Typically, a vector, in illustrative examples an expression vector, is used to deliver the polynucleotide. Such vectors can include various vectors known in the art for delivery nucleic acids to T cells and/or NK cells. Illustrative aspects of the invention utilize retroviral vectors and retroviral particles, and in some particularly illustrative embodiments lentiviral vectors and in illustrative embodiments, recombinant lentiviral particles.

Other suitable expression vectors can be used in pH switch aspects provided herein. Such expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90: 10613-10617); SV40; herpes simplex virus; or a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus), for example a gamma retrovirus; or human immunodeficiency virus (see, e.g., Miyoshi et al., PNAS 94:10319 23, 1997; Takahashi et al., J Virol 73:7812 7816, 1999); and the like.

In some embodiments, DNA-containing viral particles are utilized instead of recombinant retroviral particles. Such viral particles can be adenoviruses, adeno-associated viruses, herpesviruses, cytomegaloviruses, poxviruses, avipox viruses, influenza viruses, vesicular stomatitis virus (VSV), or Sindbis virus. A skilled artisan will appreciate how to modify the methods disclosed herein for use with different viruses and retroviruses. Where viral particles are used that include a DNA genome, a skilled artisan will appreciate that functional units can be included in such genomes to induce integration of all or a portion of the DNA genome of the viral particle into the genome of a T cell and/or NK cell transduced with such virus. Alternatively, functional DNA can be delivered to a T cell and/or NK cell that is expressed in the cell but is not integrated into the genome of the T cell and/or NK cell.

In illustrative embodiments, the vector used in a pH switch aspect of the present disclosure is a recombinant retroviral particle and in certain embodiments, a recombinant lentiviral particle. Such retroviral particle typically includes a retroviral genome within a capsid which is located within a viral envelope. The present disclosure in various sections herein, provide various embodiments of recombinant retroviral particles that disclose elements that can be included on the surface or within, and/or in the genome of a recombinant retroviral particle. Any of these recombinant retroviral particle embodiments can be used in the pH switch aspects provided herein.

In any of the embodiments disclosed above, the cognate antigen to which the CAB-CAR binds can be an Axl polypeptide or an epitope thereof, or a Ror2 polypeptide or an epitope thereof. In some embodiments, the cognate antigen can be a polypeptide with at least 50%, 55%, 60%, 65%, 70%, 75%, 80%, 85%, 90%, 95%, 96%, 97%, 98%, 99%, or 100% sequence identity to a stretch of at least 10, 15, 20, or all of the amino acids of an Axl polypeptide or an epitope thereof or to a Ror2 polypeptide or an epitope thereof. As disclosed herein, the CAB-CAR capable of binding to an Axl polypeptide or an epitope thereof, or a Ror2 polypeptide or an epitope thereof typically binds its cognate antigen with a higher binding affinity at a pH of 6.7 than a pH of 7.4. Thus, such anti-ROR2 and anti-AXL CAB-CARs of the present disclosure typically bind to their cognate antigen with a higher binding affinity in a tumor microenvironment than a normal tissue with a physiological pH.

### Treatment Methods

The present disclosure provides various methods for treating a disorder that include an anti-Axl or anti-Ror2 CAB-CAR provided herein. In some embodiments, the methods take advantage of the fact that a CAB-CAR of the present disclosure, when present in and expressed by a T lymphocyte or an NK cell, can mediate cytotoxicity toward a target cell. A CAB-CAR of the present disclosure binds to an antigen present on a target cell under certain target conditions, thereby mediating killing of a target cell by a T lymphocyte or an NK cell genetically modified to produce the CAB-CAR. The ASTR of the CAB-CAR typically binds to an antigen present on the surface of a target cell.

Target cells include, but are not limited to, cancer cells. Thus, the present disclosure provides methods of killing, or inhibiting the growth of, a target cancer cell, the method involving contacting a cytotoxic immune effector cell (e.g., a cytotoxic T cell, or an NK cell) that is genetically modified to produce a subject CAR, such that the T lymphocyte or NK cell recognizes an antigen present on the surface of a target cancer cell, and mediates killing of the target cell. Illustrative aspects of such methods provide methods for treating cancer. CAB-CARs are not limited to uses for treating cancer or targeting tumor or cancer cells, but rather could be appropriate for use in one or more indication including the treatment of circulatory disorders, arthritis, multiple sclerosis, autoimmune disorders, dermatologic disorders, viral diseases and disorders and use in various diagnostic formats.

In certain aspects, the present disclosure provides a method of treating cancer in a subject having a cancer. As such the present disclosure provides methods for adoptive cellular therapy against cancer, especially a cancer that expresses Axl or Ror2, that use the anti-Axl and Anti-Ror2 CAB-CARs provided herein. Accordingly, in one aspect the method includes the following: A. introducing an expression vector configured to express a polynucleotide sequence encoding a CAB-CAR directed to Axl or Ror2 as provided herein, into peripheral blood cells obtained from the subject to produce a genetically engineered cytotoxic cell (such as a T cell or NK cell); and B. administering the genetically engineered cytotoxic cell to the subject. Detailed methods for processing T cells to activate, transduce and typically expand such cells that provide illustrative embodiments of step A above are provided herein.

The cancer typically expresses Ror2 or Axl, respectively, and in illustrative embodiments the cancer is any cancer in which cells of such cancer express Ror2 and/or Axl, such as renal cell carcinoma. The CAR can be any of the CAB-CARs that recognize Axl or Ror2 disclosed herein, especially those that are cytotoxic to cancer cells expressing these antigens. The expression vector encoding an anti-Axl CAB-CAR or anti-Ror2 CAB-CAR can be introduced into peripheral blood cells by transducing peripheral blood leucocytes that include T cell and/or NK cells with the vector. In certain illustrative embodiments, the vector is a recombinant virus, such as a recombinant retrovirus that in some embodiments is a recombinant lentivirus. In some embodiments, the cancer is a soft tissue sarcoma or mesothelioma that expresses Ror2 and T cells and/or NK cells of the subject (e.g. soft tissue sarcoma patient or mesothelioma patient) are transduced with an anti-Ror2 CAR, for example an anti-Ror2 CAB-CAR disclosed herein.

Methods for treating a disorder provided herein typically include administering a genetically modified T cells or NK cells that express anti-Axl or anti-Ror2 CAB-CARs provided herein, to a subject. The administration can be, for example, intravenous administration, subcutaneous administration, or intratumor administration. In methods in which genetically modified T cells and/or NK cells are intravenously administered, typically between 1×10⁴ cells/kg and 1×10⁸ cells/kg cells are delivered in a suitable buffer for parenteral administration. In methods in which genetically modified T cells and/or NK cells are administered intratumorally, typically between 1×10⁶ cells and 5×10⁸ cells are delivered in an isotonic solution.

In some embodiments, the administration is preceded, accompanied by, and/or followed by administration of an interleukin or a modified version thereof. For example, some embodiments provided herein include co-administration of IL-2, or a modified version of IL-2 that has sustained release and/or binds to certain IL-2 receptors that are biased toward activating proliferation and/or killing activity of T cells. For example, the modified IL-2 in certain embodiments is a pegylated IL-2, and can be NKTR-214 (Nektar Therapeutics, San Francisco, CA). In other embodiments, the modified IL-2 is ALKS 4230 (Alkermes, Inc.).

Carcinomas that can be amenable to therapy by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

Sarcomas that can be amenable to therapy by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be amenable to therapy by a method disclosed herein include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Leukemias that can be amenable to therapy by a method disclosed herein include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; non-Hodgkin's lymphoma, and the like.

Other cancers that can be amenable to treatment according to the methods disclosed herein include atypical meningioma (brain), islet cell carcinoma (pancreas), medullary carcinoma (thyroid), mesenchymoma (intestine), hepatocellular carcinoma (liver), hepatoblastoma (liver), clear cell carcinoma (kidney), and neurofibroma mediastinum.

### Combination Therapy

In some embodiments, a CAR cell is administered as an adjuvant therapy to a standard cancer therapy. Standard cancer therapies include surgery (e.g., surgical removal of cancerous tissue), radiation therapy, bone marrow transplantation, chemotherapeutic treatment, antibody treatment, biological response modifier treatment, and certain combinations of the foregoing.

Radiation therapy includes, but is not limited to, x-rays or gamma rays that are delivered from either an externally applied source such as a beam, or by implantation of small radioactive sources.

Suitable antibodies for use in cancer treatment include, but are not limited to, naked antibodies, e.g., trastuzumab (Herceptin), bevacizumab (Avastin™), cetuximab (Erbitux™) panitumumab (Vectibix™), Ipilimumab (Yervoy™), rituximab (Rituxan), alemtuzumab (Lemtrada™) Ofatumumab (Arzerra™), Oregovomab (OvaRex™), Lambrolizumab (MK-3475), pertuzumab (Perjeta™), ranibizumab (Lucentis™) etc., and conjugated antibodies, e.g., gemtuzumab ozogamicin (Mylortarg™), Brentuximab vedotin ⁹⁰Y-labelled ibritumomab tiuxetan (Zevalin™), ¹³¹I-labelled tositumoma (Adcetris™), (Bexxarr®), etc. Suitable antibodies for use in cancer treatment include, but are not limited to, antibodies raised against tumor-associated antigens. Such antigens include, but are not limited to, CD20, CD30, CD33, CD52, EpCAM, CEA, gpA33, Mucins, TAG-72, CAIX, PSMA, Folate-binding protein, Gangliosides (e.g., GD2, GD3, GM2, etc.), Le^{y}, VEGF, VEGFR, Integrin alpha-V-beta-3, Integrin alpha-5-beta-1, EGFR, ERBB2, ERBB3, MET, IGFlR, EPHA3, TRAILR1, TRAILR2, RANKL, PAP, Tenascin, etc.

Biological response modifiers suitable for use in connection with the methods of the present disclosure include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) interferon-α; (7) interferon-γ; (8) colony-stimulating factors; (9) inhibitors of angiogenesis; and (10) antagonists of tumor necrosis factor.

Chemotherapeutic agents are non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB37 1 7), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysteine, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and progestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 1 7a-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation, therefore compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

### Subjects Suitable for Treatment

A variety of subjects are suitable for treatment with a method of treating cancer. Suitable subjects include any individual, e.g., a human or non-human animal who has cancer, who has been diagnosed with cancer, who is at risk for developing cancer, who has had cancer and is at risk for recurrence of the cancer, who has been treated with an agent for the cancer and failed to respond to such treatment, or who has been treated with an agent for the cancer but relapsed after initial response to such treatment.

Subjects suitable for treatment with an immunomodulatory method include individuals who have an autoimmune disorder; individuals who are organ or tissue transplant recipients; and the like; individuals who are immunocompromised; and individuals who are infected with a pathogen.

### Exemplary Embodiments

The present disclosure provides chimeric antigen receptors (CARs), and nucleic acids comprising the nucleotide sequences encoding the CARs, that bind to Axl and/or Ror2, and conditionally active CARs that bind to Axl and Ror2. The present disclosure provides cells genetically modified to produce the CARs, and methods for making such cells. The CARs of the present disclosure can be used in various methods, which are also provided, including methods for performing adoptive cell therapy such as CAR therapy, for example CAR therapy against cancer, for example renal cell carcinoma.

Some non-limiting exemplary embodiments that are aspects of the present disclosure are provided in the following Embodiments:

Embodiment 1. A chimeric antigen receptor (CAR) for binding Axl or Ror2, comprising:
a) a conditionally active antigen-specific targeting region (ASTR) that exhibits an increased binding to Axl or Ror2 at a pH of 6.7 compared to a pH of 7.4;
b) a transmembrane domain; and
c) an intracellular activating domain.

Embodiment A1. A chimeric antigen receptor (CAR) for binding Axl or Ror2, comprising:
a) a conditionally active antigen-specific targeting region (ASTR) that exhibits an increase in (i.e. a greater) activity in a tumor microenvironment and/or in an in vitro tumor surrogate assay condition, compared to a normal physiological environment, wherein the ASTR binds to Axl or Ror2;
b) a transmembrane domain; and
c) an intracellular activating domain.

Embodiment A2. The CAR of any one of Embodiments 1 or A1, or according to any other Embodiment provided herein unless explicitly recited otherwise, wherein the ASTR is selected from an antibody, an antigen, a ligand, a receptor binding domain of a ligand, a receptor, a ligand binding domain of a receptor, and an affibody.

Embodiment A3. The CAR of Embodiment A2, or according to any other Embodiment provided herein unless explicitly recited otherwise, wherein the ASTR is an antibody fragment.

Embodiment A4. The CAR of any one of Embodiments 1 or A1 to A3, or according to any other Embodiment provided herein unless explicitly recited otherwise, wherein the conditionally active ASTR exhibits an increase in antigen binding in a tumor microenvironment and/or in an in vitro tumor surrogate assay condition relative to a corresponding physiological condition, wherein the tumor microenvironment and/or the in vitro tumor surrogate assay conditions are selected from the group consisting of hypoxia, an acidic pH, a higher concentration of lactic acid, a higher concentration of hyaluronan, a higher concentration of albumin, a higher concentration of adenosine, a higher concentration of R-2-hydroxyglutarate, and a lower nutrient availability.

Embodiment A5. The CAR of any one of Embodiments A1 to A4, or according to any other Embodiment provided herein unless explicitly recited otherwise, wherein the conditionally active ASTR exhibits an increase in (or a higher) antigen binding at a pH of 6.7 as compared to a pH of 7.4.

Embodiment A6. The CAR of any one of Embodiments 1 or A1 to A5, or according to any other Embodiment provided herein unless explicitly recited otherwise, wherein the intracellular activating domain is a human CD3Z activating domain, a human CD3D activating domain, a human CD3E activating domain, a human CD3G activating domain, a human CD28 activating domain, a human CD79A activating domain, a human DAPIO activating domain, a human DAP12 activating domain, a human FCERIG activating domain, a human CD137 activating domain, or a human ZAP70 activating domain.

Embodiment A7. The CAR of any one of Embodiments 1 or A1 to A6, or according to any other Embodiment provided herein unless explicitly recited otherwise, further comprising a first, second, third, or fourth co-stimulatory domain that has a different amino acid sequence than the intracellular activating domain.

Embodiment A8. The CAR of Embodiment A7, or according to any other Embodiment provided herein unless explicitly recited otherwise, wherein the first, second, third, and/or fourth co-stimulatory domain comprise a co-stimulatory domain of 4-1BB (CD137), B7-H3, CD2, CD7, CD27, CD28, CD28 deleted for Lek binding (ICA), ICOS, OX40, BTLA, CD27, CD30, CD40, GITR, HVEM, LFA-1, LIGHT, NKG2C, PD-1, TILR2, TILR4, TILR7, TILR9, Fc receptor gamma chain, Fc receptor E chain, or a ligand that specifically binds with CD83.

Embodiment A9. The CAR of Embodiment A7, or according to any other Embodiment provided herein unless explicitly recited otherwise, wherein the first co-stimulatory domain retains a co-stimulating activity and is a human CD137 co-stimulatory domain, a human CD28 co-stimulatory domain, a human ICA co-stimulatory domain, a human ICOS co-stimulatory domain, a human OX40 co-stimulatory domain, a human BTLA co-stimulatory domain, a human CD27 costimulatory domain, a human CD30 co-stimulatory domain, a human GITR co-stimulatory domain, or a human HVEM co-stimulatory domain.

Embodiment B1. A replication incompetent recombinant retroviral particle, comprising a retroviral genome comprising one or more nucleic acid sequences operably linked to a promoter active in T cells and/or NK cells, wherein the one or more nucleic acid sequences encode the CAR of any one of Embodiments 1 or A1 to A9, or of any other Embodiment provided herein unless explicitly recited otherwise.

Embodiment B2. An isolated recombinant T cell or NK cell genetically modified with the replication incompetent recombinant retroviral particle of Embodiment B1.

Embodiment B3. An isolated recombinant T cell genetically modified with the replication incompetent recombinant retroviral particle of Embodiment B1.

Embodiment B4. A reaction mixture comprising the replication incompetent recombinant retroviral particle of Embodiment B1, and a T cell and/or an NK cell.

Embodiment B5. A reaction mixture comprising the replication incompetent recombinant retroviral particle of Embodiment B1, and a T cell.

Embodiment C1. An isolated (e.g. recombinant or genetically modified) T cell or NK cell, comprising a genome comprising one or more nucleic acid sequences (for example, two or more, three or more, four or more, five or more, or six or more nucleic acid sequences) operably linked to a promoter active in T cells and/or NK cells, wherein the one or more (or two or more, three or more, four or more, five or more, or six or more) nucleic acid sequences encode the CAR of any one of Embodiments 1 or A1 to A8, or of any other Embodiment provided herein unless explicitly recited otherwise.

Embodiment C2. An isolated (e.g. recombinant or genetically modified) T cell or NK cell of Embodiment C1, wherein the isolated cell is a T cell.

Embodiment C3. An isolated (e.g. recombinant or genetically modified) T cell or NK cell (in certain illustrative embodiments, a T cell) comprising one or more nucleic acid sequences operably linked to a promoter active in T cells and/or NK cells, wherein the one or more nucleic acid sequences encode a chimeric antigen receptor (CAR) for binding Axl or Ror2, comprising:
a) a conditionally active antigen-specific targeting region (ASTR) that exhibits an increased binding to Axl or Ror2 at a pH of 6.7 compared to a pH of 7.4;
b) a transmembrane domain; and
c) an intracellular activating domain.

Embodiment D1. A method for binding a T cell and/or NK cell to a target mammalian cell, comprising contacting the target mammalian cell with the T cell and/or the NK cell in a microenvironment at a pH of less than 7.4 (for example, less than 5.8, 5.9, 6.0, 6.1, 6.2, 6.3, 6.4, 6.5, 6.6, 6.7, 6.8, or 6.9, or in the range of 5.8 to 7.0, in illustrative embodiments in the range of 6.0 to 6.8, in the range of 6.1 to 6.9, in the range of 6.2 to 6.8, or between 6.0, 6.1, 6.2, 6.3, 6.4, and 6.5 on the low end of the range, and 6.6, 6.7, 6.8, and 6.9 on the high end of the range), wherein the T cell and/or NK cell expresses the CAR of any one of Embodiments 1 or A1 to A8, or of any other Embodiment provided herein unless explicitly recited otherwise, and the target mammalian cell expresses Axl and/or Ror2.

Embodiment D2. A method for binding a T cell or NK cell to a target mammalian cell, comprising contacting the target mammalian cell with the T cell and/or the NK cell in a microenvironment at a pH of less than 7.4 (for example, less than 5.8, 5.9, 6.0, 6.1, 6.2, 6.3, 6.4, 6.5, 6.6, 6.7, 6.8, or 6.9, or in the range of 5.8 to 7.0, in illustrative embodiments in the range of 6.0 to 6.8, in the range of 6.2 to 6.8, or between 6.0, 6.1, 6.2, 6.3, 6.4, and 6.5 on the low end of the range, and 6.6, 6.7, 6.8, and 6.9 on the high end of the range), wherein the target mammalian cell expresses Axl or Ror2, and wherein the T cell or NK cell expresses a chimeric antigen receptor (CAR) for binding Axl or Ror2, respectively, wherein the CAR comprises:
a) a conditionally active antigen-specific targeting region (ASTR) that exhibits an increased binding to Axl or Ror2 at a pH of 6.7 compared to a pH of 7.4;
b) a transmembrane domain; and
c) an intracellular activating domain.

Embodiment D3. The method of any one of Embodiments D1 to D3, wherein the binding activates the T cell and/or NK cell.

Embodiment D4. A method for activating a T cell or NK cell, comprising contacting a target mammalian cell with the T cell and/or the NK cell in a microenvironment at a pH of less than 7.4 (for example, less than 5.8, 5.9, 6.0, 6.1, 6.2, 6.3, 6.4, 6.5, 6.6, 6.7, 6.8, or 6.9, or in the range of 5.8 to 7.0, in illustrative embodiments in the range of 6.0 to 6.8, in the range of 6.2 to 6.8, or between 6.0, 6.1, 6.2, 6.3, 6.4, and 6.5 on the low end of the range, and 6.6, 6.7, 6.8, and 6.9 on the high end of the range), wherein the target mammalian cell expresses Axl or Ror2, and wherein the T cell or NK cell expresses a chimeric antigen receptor (CAR) for binding Axl or Ror2, respectively, wherein the CAR comprises:
a) a conditionally active antigen-specific targeting region (ASTR) that exhibits an increased binding to Axl or Ror2 at a pH of 6.7 compared to a pH of 7.4;
b) a transmembrane domain; and
c) an intracellular activating domain.

Embodiment D5. The method of Embodiment D5, wherein the CAR is any of the CARs provided in another Embodiment. in non-limiting illustrative examples, Embodiments 1 or A1 to A8.

Embodiment D6. The method of Embodiment D5, wherein the CAR is a CAR provided in any one of Embodiments 1 or A1 to A8.

Embodiment D7. The method of any one of Embodiments D3 to D6, wherein activation comprises increased expression and/or production and/or secretion of a cytokine.

Embodiment D8. The method of any one of Embodiments D3 to D7, wherein upon activation, the T cell and/or NK cell increases expression of IL-2 or IFN-γ.

Embodiment D9. The method of Embodiment D8, wherein expression of IL-2 or IFN-γ is increased by at least 10%, at least 15%, at least 20%, at least 25%, at least 30%, at least 40%, at least 50%, at least 75%, at least 2-fold, at least 2.5-fold, at least 5-fold, at least 10-fold, or more than 10-fold, compared with the amount of IL-2 or IFN-γ produced by the T cell or NK cell before the contacting.

Embodiment D10. The method of Embodiment D8, wherein secretion of IL-2 or IFN-γ is increased by at least 10%, at least 15%, at least 20%, at least 25%, at least 30%, at least 40%, at least 50%, at least 75%, at least 2-fold, at least 2.5-fold, at least 5-fold, at least 10-fold, or more than 10-fold, compared with the amount of IL-2 or IFN-γ secreted by the T cell or NK cell before the contacting.

Embodiment D11. The method of any one of Embodiments D3 to D7, wherein upon activation, cytotoxic activity of the T cell or NK cell is increased by at least 10%, at least 15%, at least 20%, at least 25%, at least 30%, at least 40%, at least 50%, at least 75%, at least 2-fold, at least 2.5-fold, at least 5-fold, at least 10-fold, or more than 10-fold, compared to the cytotoxic activity of the T cell or NK cell before the contacting.

Embodiment D12. The method of any one of embodiments D1 to D11, wherein the target mammalian cell is lysed after activation of the T cell or NK cell.

Embodiment D13. The method of any one of Embodiments D1 to D12, further comprising before the contacting, transducing the T cell or the NK cell with the replication incompetent recombinant retroviral particle of any one of Embodiments B1 or B2 to genetically modify the T cell or NK cell to express the CAR.

Embodiment D14. The method of Embodiment D13, wherein the transducing is performed ex vivo.

Embodiment D15. The method of any one of Embodiments D1 to D14, further comprising, increasing the pH of the microenvironment to a pH at or above 7.0 (e.g. above 7.1, 7.2, or 7.3), thereby decreasing the activation of the T cell or NK cell.

Embodiment D16. The method of any one of Embodiments D1 to D14, further comprising, increasing the pH of the microenvironment to a pH at or above 7.0 (e.g. above 7.1, 7.2, or 7.3), thereby deactivating the T cell or NK cell.

Embodiment D17. The method of any one of Embodiments D1 to D16, wherein the microenvironment is a tumor.

Embodiment D18. The method of claim D17, wherein the tumor is in a human subject.

Embodiment D19. The method of any one of Embodiments D1 to D16, wherein the microenvironment is in vitro or ex vivo.

Embodiment E1. A method for genetically modifying a lymphocyte, comprising contacting a T cell and/or NK cell with a replication incompetent recombinant retroviral particle comprising in its genome a polynucleotide comprising one or more nucleic acid sequences (for example, two or more, three or more, four or more, five or more, or six or more nucleic acid sequences) operatively linked to a promoter active in T cells and/or NK cells, wherein the one or more (or two or more, three or more, four or more, five or more, or six or more) nucleic acid sequences encode the chimeric antigen receptor of any one of Embodiments 1 or A1 to A8, or of any other Embodiment provided herein unless explicitly recited otherwise, wherein said contacting facilitates transduction of the T cell and/or NK cell by the replication incompetent recombinant retroviral particle, thereby producing a genetically modified T cell and/or NK cell.

Embodiment F1. A replication incompetent recombinant retroviral particle for use in a method for genetically modifying a lymphocyte, wherein the replication incompetent recombinant retroviral particle comprises in its genome a polynucleotide comprising one or more nucleic acid sequences (for example, two or more, three or more, four or more, five or more, or six or more nucleic acid sequences) operatively linked to a promoter active in T cells and/or NK cells, wherein the one or more (or two or more, three or more, four or more, five or more, or six or more) nucleic acid sequences encode the chimeric antigen receptor of any one of Embodiments 1 or A1 to A8, or of any other Embodiment provided herein unless explicitly recited otherwise, wherein the method comprises contacting a T cell and/or NK cell, and said contacting facilitates transduction of the T cell and/or NK cell by the replication incompetent recombinant retroviral particles, thereby producing a genetically modified T cell and/or NK cell.

Embodiment G1. A replication incompetent recombinant retroviral particle for use in a method for genetically modifying a T cell and/or NK cell, for treating tumor growth, wherein the method comprises contacting the T cell and/or NK cell with a replication incompetent recombinant retroviral particle comprising in its genome a polynucleotide comprising one or more nucleic acid sequences (for example, two or more, three or more, four or more, five or more, or six or more nucleic acid sequences) operatively linked to a promoter active in T cells and/or NK cells, wherein the one or more (or two or more, three or more, four or more, five or more, or six or more) nucleic acid sequences encode the chimeric antigen receptor of any one of Embodiments 1 or A1 to A8, or of any other Embodiment provided herein unless explicitly recited otherwise, wherein said contacting facilitates transduction of the T cell and/or NK cell by the replication incompetent recombinant retroviral particles, thereby producing a genetically modified T cell and/or NK cell.

Embodiment H1. Use of a replication incompetent recombinant retroviral particle in the manufacture of a kit for genetically modifying a T cell and/or NK cell, wherein the use of the kit comprises contacting the T cell and/or NK cell with a replication incompetent recombinant retroviral particle comprising in its genome a polynucleotide comprising one or more nucleic acid sequences (for example, two or more, three or more, four or more, five or more, or six or more nucleic acid sequences) operatively linked to a promoter active in T cells and/or NK cells, wherein the one or more (or two or more, three or more, four or more, five or more, or six or more) nucleic acid sequences encode the chimeric antigen receptor of any one of Embodiments 1 or A1 to A8, or of any other Embodiment provided herein unless explicitly recited otherwise, wherein said contacting facilitates transduction of the T cell and/or NK cell by the replication incompetent recombinant retroviral particles, thereby producing a genetically modified T cell and/or NK cell.

Embodiment I1. A commercial container containing a replication incompetent recombinant retroviral particle and instructions for the use thereof, wherein the replication incompetent recombinant retroviral particle comprises in its genome a polynucleotide comprising one or more nucleic acid sequences (for example, two or more, three or more, four or more, five or more, or six or more nucleic acid sequences) operatively linked to a promoter active in T cells and/or NK cells, wherein the one or more (or two or more, three or more, four or more, five or more, or six or more) nucleic acid sequences encode the chimeric antigen receptor of any one of Embodiments 1 or A1 to A8, or of any other Embodiment provided herein unless explicitly recited otherwise.

Embodiment J1. A kit containing a container containing a replication incompetent recombinant retroviral particle, and instructions for use thereof, wherein the instructions instruct a method for binding a T cell and/or NK cell to a target mammalian cell, in a method comprising:
a) transducing the T cell and/or NK cell with the replication incompetent recombinant retroviral particle, comprising in its genome a polynucleotide comprising one or more nucleic acid sequences (for example, two or more, three or more, four or more, five or more, or six or more nucleic acid sequences) operatively linked to a promoter active in T cells and/or NK cells, wherein the one or more (or two or more, three or more, four or more, five or more, or six or more) nucleic acid sequences encode the CAR of any one of Embodiments 1 or A1 to A8, or of any other Embodiment provided herein unless explicitly recited otherwise; and
b) contacting the target mammalian cell with the transduced T cell and/or the NK cell in a microenvironment at a pH of less than 7.4 (for example, less than 5.8, 5.9, 6.0, 6.1, 6.2, 6.3, 6.4, 6.5, 6.6, 6.7, 6.8, or 6.9, or in the range of 5.8 to 7.0, more commonly in the range of 6.0 to 6.8, in the range of 6.2-6.8, or between 6.0, 6.1, 6.2, 6.3, 6.4, and 6.5 on the low end of the range, and 6.6, 6.7, 6.8, and 6.9 on the high end of the range), wherein the T cell and/or NK cell expresses the CAR of any one of Embodiments 1 or A1 to A8, and the target mammalian cell expresses Axl and/or Ror2.

Embodiment K1. A method for activating a T cell and/or NK cell, comprising contacting a target mammalian cell with the T cell and/or the NK cell in a microenvironment at a pH of less than 7.4 (for example, less than 5.8, 5.9, 6.0, 6.1, 6.2, 6.3, 6.4, 6.5, 6.6, 6.7, 6.8, or 6.9, or in the range of 5.8 to 7.0, more commonly in the range of 6.0 to 6.8, in the range of 6.2-6.8, or between 6.0, 6.1, 6.2, 6.3, 6.4, and 6.5 on the low end of the range, and 6.6, 6.7, 6.8, and 6.9 on the high end of the range), wherein the T cell and/or NK cell expresses the chimeric antigen receptor of any one of Embodiments 1 or A1 to A8, or of any other Embodiment provided herein unless explicitly recited otherwise, and the target mammalian cell expresses Axl and/or Ror2.

Embodiment K2. The method of Embodiment K1, further comprising before the contacting, transducing the T cell and/or the NK cell with the replication incompetent recombinant retroviral particle of any one of Embodiments B1 or B2.

Embodiment K3. The method of Embodiment K1, wherein after the activating, the T cell and/or NK cell induces expression and/or production and/or secretion of a cytokine.

Embodiment K4. The method of Embodiment K3, wherein the cytokine is chosen from the group consisting of IL-2 or IFN-γ.

Embodiment L1. A method for inducing expression and/or production of a cytokine in a T cell and/or NK cell and/or inducing secretion of a cytokine from a T cell and/or NK cell, comprising contacting a target mammalian cell with the T cell and/or the NK cell in a microenvironment at a pH of less than 7.4 (for example, less than 5.8, 5.9, 6.0, 6.1, 6.2, 6.3, 6.4, 6.5, 6.6, 6.7, 6.8, or 6.9, or in the range of 5.8 to 7.0, more commonly in the range of 6.0 to 6.8, in the range of 6.2-6.8, or between 6.0, 6.1, 6.2, 6.3, 6.4, and 6.5 on the low end of the range, and 6.6, 6.7, 6.8, and 6.9 on the high end of the range), wherein the T cell and/or NK cell expresses the chimeric antigen receptor of any one of Embodiments 1 or A1 to A8, or of any other Embodiment provided herein unless explicitly recited otherwise, and the target mammalian cell expresses Axl and/or Ror2.

Embodiment L2. The method of Embodiment L1, further comprising before the contacting, transducing the T cell and/or the NK cell with the replication incompetent recombinant retroviral particle of any one of Embodiments B1 or B2.

Embodiment M1. An isolated nucleic acid encoding a chimeric antigen receptor for binding Axl or Ror2 according to any of Embodiments 1 or A1 to A8, or of any other Embodiment provided herein unless explicitly recited otherwise.

Embodiment M2. An isolated nucleic acid of Embodiment M1, wherein the nucleic acid further comprises a promoter active in T cells and/or NK cells, and wherein nucleic acid sequences encoding the CAR are operably linked to the promoter.

Embodiment M3. The isolated nucleic acid of Embodiment M2, wherein the isolated nucleic acid sequence further encodes a recognition domain.

Embodiment M4. The isolated nucleic acid of Embodiment M3, wherein nucleic acids encoding the recognition domain are separated from nucleic acids encoding the CAR by a ribosomal skip sequence.

Embodiment M5. The isolated nucleic acid of Embodiment M4, wherein the ribosomal skip sequence is 2A-1.

Embodiment M6. A replication incompetent recombinant retroviral particle comprising any of the nucleic acids of Embodiments M1 to M5.

Embodiment M7. The replication incompetent recombinant retroviral particle of Embodiment M6, wherein the replication incompetent recombinant retroviral particle is a lentiviral particle.

Embodiment N1. An expression vector comprising a nucleic acid encoding a chimeric antigen receptor according to any one of Embodiments 1 or A1 to A8, or of any other Embodiment provided herein unless explicitly recited otherwise, operably linked to a promoter.

Embodiment O1. A mammalian cell infected with any one of the expression vectors of Embodiment N1.

Embodiment P1. A mammalian cell expressing any of the chimeric antigen receptors of Embodiments 1 or A1 to A8, or of any other Embodiment provided herein unless explicitly recited otherwise.

Embodiment Q1. A method for making a conditionally activatable cell comprising a chimeric antigen receptor (CAR) for conditionally binding Axl or Ror2, wherein the method comprises genetically modifying a mammalian cell with an expression vector comprising a promoter operably linked to nucleotide sequences encoding the CAR, wherein the CAR comprises:
a) a conditionally active antigen-specific targeting region (ASTR) that exhibits an increase in activity in a tumor environment and/or in an in vitro tumor surrogate assay condition, compared to a normal physiological environment, wherein the ASTR binds to Axl or Ror2;
b) a transmembrane domain; and
c) an intracellular activating domain.

Embodiment R1. A method for treating a cancer in a subject, comprising the steps of:
a) introducing an expression vector comprising a nucleic acid encoding any of the CARs of Embodiments 1 or A1 to A8, or of any other Embodiment provided herein unless explicitly recited otherwise, into cytotoxic cells obtained from the subject to produce genetically modified cytotoxic cells; and
b) administering the genetically modified cytotoxic cells to the subject.

Embodiment S1. An ex vivo method for making conditionally activatable T cells and/or NK cells comprising a chimeric antigen receptor (CAR) for conditionally binding Axl or Ror2, wherein the method comprises:
a) enriching peripheral blood mononuclear cells (PBMCs) to isolate PBMCs comprising T cells and/or NK cells from isolated blood;
b) activating T cells and/or NK cells of the enriched PBMCs under effective conditions;
c) transducing the activated T cells and/or NK cells with replication incompetent recombinant retroviral particles under effective conditions, thereby producing genetically modified T cells and/or NK cells, wherein the replication incompetent recombinant retroviral particles each comprise a retroviral genome comprising one or more nucleic acid sequences operatively linked to a promoter active in T cells and/or NK cells, wherein a first nucleic acid sequence of the one or more nucleic acid sequences encodes a CAR of any of Embodiments 1 or A1 to A8, or of any other Embodiment provided herein unless explicitly recited otherwise; and
d) expanding the genetically modified T cells and/or NK cells, thereby making the conditionally activatable T cells and/or NK cells.

Embodiment T1. A modified T cell produced by a method of any of Embodiments D3, E1, K2, Q1, R1, or Sl.

Embodiment U1. A modified NK cell produced by a method of any of claims Embodiments D3, E1, K2, Q1, R1, or Sl.

In any of the embodiments herein that includes a chimeric antigen receptor (CAR), the antigen-specific targeting region (ASTR) can be selected from an antibody, an antigen, a ligand, a receptor binding domain of a ligand, a receptor, a ligand binding domain of a receptor, and an affibody. In any of the embodiments herein that includes a CAR, the ASTR can be an antibody selected from a full-length antibody, a single-chain antibody, an Fab fragment, an Fab′ fragment, an (Fab′)2 fragment, an Fv fragment, and a divalent single-chain antibody or a diabody. In any of the embodiments herein that includes a CAR, the ASTR can include a heavy chain and a light chain from an antibody. In any of the embodiments herein that includes a CAR with an ASTR that includes an antibody, the antibody can be a single-chain variable fragment with a heavy chain and a light chain. In any of the embodiments herein that includes a CAR with an ASTR that includes a heavy chain and a light chain from an antibody, the heavy and light chains can be separated by a linker, and the linker can be between 3, 4, 5, 6, 7, 8, 9, or 10 amino acids in length on the low end of the range and 20, 25, 30, 40, 50, 60 70, 80, 90, 100, 125, 150, 175, or 200 amino acids in length on the high end of the range. In some embodiments, the ASTR can be a single-chain variable fragment comprising a heavy chain and a light chain, wherein the heavy and light chains are separated by a linker, wherein the linker is between 6 and 100 amino acids in length. In any of the embodiments herein that includes a CAR with an ASTR that includes a single-chain variable fragment including a heavy chain and a light chain, the heavy and light chains can be separated by a linker and the linker can be between 6 and 100 amino acids in length, and the ASTR can include a conditionally active antibody heavy chain or a conditionally active antibody light chain and the other of the antibody heavy chain or antibody light chain can be wild type.

In any of the embodiments herein that includes a CAR with an ASTR that includes a heavy chain and a light chain from an antibody, the heavy chain can be positioned N-terminal to the light chain on the chimeric antigen receptor or the light chain can be positioned N-terminal to the heavy chain on the chimeric antigen receptor. In any of the embodiments herein that includes a CAR with an ASTR that includes a heavy chain and a light chain from an antibody, the heavy chain and the light chain can be from a conditionally active antibody. In any of the embodiments herein that includes a CAR, the CAR can include a bispecific ASTR. In any of the embodiments herein that includes a CAR, the ASTR can be a conditionally active ASTR. In any of the embodiments herein that includes a CAR with a conditionally active ASTR, the conditionally active ASTR can exhibit an increase in antigen binding in a tumor microenvironment and/or in an in vitro tumor surrogate assay condition relative to a corresponding physiological condition, wherein the tumor microenvironment and/or in vitro tumor surrogate assay conditions can be selected from the group consisting of hypoxia, an acidic pH, a higher concentration of lactic acid, a higher concentration of hyaluronan, a higher concentration of albumin, a higher concentration of adenosine, a higher concentration of R-2-hydroxyglutarate, and a lower nutrient availability. In any of the embodiments herein that includes a CAR with a conditionally active ASTR, the conditionally active ASTR can exhibit an increase in antigen binding at a pH of 6.7 as compared to a pH of 7.4. In any of the embodiments herein that includes a CAR with a conditionally active ASTR, the conditionally active ASTR can exhibit an increase in antigen binding at a pH of 6.0 (instead of or in addition to at a pH of 6.7) as compared to a pH of 7.4.

In any of the embodiments herein that includes a CAR, the ASTR can bind to Axl. In any of the embodiments herein that includes a CAR with an ASTR that binds to Axl, the ASTR can bind to the same epitope of Axl as an antibody comprising the antibody heavy chain of SEQ ID NO:79 and an antibody light chain of SEQ ID NO:80.

In any of the embodiments herein that includes a CAR with an ASTR that binds to Axl, and in illustrative embodiments binds to the same epitope of Axl as an antibody that includes the antibody heavy chain of SEQ ID NO:79 and the antibody light chain of SEQ ID NO:80, the ASTR can include an antibody heavy chain variable region with three complementarity determining regions having H1, H2, and H3 sequences, wherein: a) the H1 sequence is X₁GX₂TMN (SEQ ID NO:87); b) the H2 sequence is LIKPSNGGTSYNQKFKG (SEQ ID NO:88); and c) the H3 sequence is GX₃YX₄SYX₅AMDY (SEQ ID NO:89), wherein X₁ is T or W; X₂ is H or A; X₃ is H or D; X₄ is E or H; and X₅ is E or F. In any of the embodiments herein that includes a CAR with an ASTR that binds to Axl, and in illustrative embodiments binds to the same epitope of Axl as an antibody that includes the antibody heavy chain of SEQ ID NO:79 and the antibody light chain of SEQ ID NO:80, the ASTR can include an antibody light chain variable region with three complementarity determining regions having L1, L2, and L3 sequences, wherein: d) the L1 sequence is KASQDVX₆SAVA (SEQ ID NO:90); e) the L2 sequence is WX₇X₈TRX₉T (SEQ ID NO:91); and f) the L3 sequence is QEHFSX₁₀PLX₁₁ (SEQ ID NO:92), wherein X₆ is S or V; X₇ is A or Q; X₈ is S or D; X₉ is H or D; X₁₀ is T or P; and X₁₁ is T or R. In any of the embodiments herein that includes a CAR with an ASTR that binds to Axl, and in illustrative embodiments binds to the same epitope of Axl as an antibody that includes the antibody heavy chain of SEQ ID NO:79 and the antibody light chain of SEQ ID NO:80, the ASTR can include an antibody heavy chain variable region with three complementarity determining regions having H1, H2, and H3 sequences, wherein: a) the H1 sequence is X₁GX₂X₃MX₄ (SEQ ID NO: 134); b) the H2 sequence is LIKX₅SNGGTX₆YNQKFKG (SEQ ID NO: 135); and c) the H3 sequence is GX₇X₈X₉X₁₀X₁₁X₁₂X₁₃X₁₄DYX₁₅X₁₆ (SEQ ID NO: 136), wherein X₁ is T, A, or W; X₂ is H or A; X₃ is T or I; X₄ is N or I; X₅ is P or N; X₆ is S, I, or T; X₇ is H, D, E, P, R, or W; X₈ is Y or N; X₉ is E, A, D, F, G, H, I, L, M, N, R, V, or Y; X₁₀ is S, D, M, N, or Q; X₁₁ is Y, C, E, or P; X₁₂ is F, E, N, S, T, or V; X₁₃ is A, D, G, L, or Y; X₁₄ is M, E, or F; X₁₈ is W, A, D, H, L, N, P, R, or T; and X₁₆ is G or H. In any of the embodiments herein that includes a CAR with an ASTR that binds to Axl, and in illustrative embodiments binds to the same epitope of Axl as an antibody that includes the antibody heavy chain of SEQ ID NO:79 and the antibody light chain of SEQ ID NO:80, the ASTR can include an antibody light chain variable region with three complementarity determining regions having L1, L2, and L3 sequences, wherein: d) the L1 sequence is KASQDX₁₇X₁₈SX₁₉VX₂₀ (SEQ ID NO: 137); e) the L2 sequence is X₂₁X₂₂X₂₃TRX₂₄T (SEQ ID NO: 138); and f) the L3 sequence is QEX₂₅X₂₆SX₂₇X₂₈X₂₉X₃₀ (SEQ ID NO: 139), wherein X₁₇ is V, D, G, N, or W; X₁₈ is S or V; X₁₉ is A, L, or M; X₂₀ is A, D, N, or Q; X₂₁ is W or F; X₂₂ is A, I, N, P, or Q; X₂₃ is S or D; X₂₄ is H or D; X₂₅ is H, C, F, I, L, Q, S, T, V, or Y; X₂₆ is F, C, D, E, G, N, or S; X₂₇ is T, C, or P; X₂₈ is P, A, C, D, E, H, K, S, T, V, or W; X₂₉ is L, G, or R; and X₃₀ is T, I, or R. In any of the embodiments herein that includes a CAR with an ASTR that binds to Axl and includes a light chain variable region, the light chain variable region can be selected from SEQ ID NOs: 108-111. In any of the embodiments herein that includes a CAR with an ASTR that binds to Axl and includes a heavy chain variable region, the heavy chain variable region can be selected from SEQ ID NOs: 112-114.

In any of the embodiments herein that include a CAB-CAR that binds to Axl, the heavy chain can be N-terminal to the light chain. In these embodiments, the ASTR can include an amino acid sequence of SEQ ID NO: 128, SEQ ID NO: 129, or SEQ ID NO: 159.

In any of the embodiments herein that include a CAB-CAR that binds to Axl, the light chain can be N-terminal to the heavy chain. In these embodiments, the ASTR can comprise an amino acid sequence of SEQ ID NO:160 or SEQ ID NO:161.

In any of the embodiments herein that includes a CAR, the ASTR can bind to Ror2. In any of the embodiments herein that includes a CAR with an ASTR that binds to Ror2, the ASTR can bind to the same epitope of Ror2 as an antibody that includes the antibody heavy chain of SEQ ID NO:82 or SEQ ID NO:83 and the antibody light chain of SEQ ID NO:84 and/or the ASTR can bind to the same epitope of Ror2 as a single-chain variable antibody fragment comprising the antibody heavy chain of SEQ ID NO: 151 and the antibody light chain of SEQ ID NO: 152. In any of the embodiments herein that includes a CAR with an ASTR that binds to Ror2, the ASTR can include a heavy chain variable region with three complementarity determining regions, said regions having H1, H2, and H3 sequences, wherein: a) the H1 sequence is GYTX₁TEX₂TX₃H (SEQ ID NO:95) or X₄GYSITTGYYWN (SEQ ID NO:96); b) the H2 sequence is GX₅NX₆NNGGTGYNQKFKG (SEQ ID NO:97) or YITYDGSKNYNPSLKN (SEQ ID NO:98); and c) the H3 sequence is GSLYSYGNSYFDY (SEQ ID NO:99) or FEGVWX₇GLDY (SEQ ID NO: 100), wherein X₁ is F or E; X₂ is Y or D, X₃ is M or D; X₄ is T or S; X₈ is E or I; X₆ is T or D; and X₇ is Y or G. In any of the embodiments herein that includes a CAR with an ASTR that binds to Ror2, and in illustrative embodiments binds to the same epitope of Ror2 as an antibody that includes the antibody heavy chain of SEQ ID NO: 151 and the antibody light chain of SEQ ID NO: 152, the ASTR can include a heavy chain variable region with three complementarity determining regions, said regions having H1, H2, and H3 sequences, wherein: a) the H1 sequence is GYTX₁TEX₂TX₃H (SEQ ID NO:95); b) the H2 sequence is GX₅NX₆NNGGTGYNQKFKG (SEQ ID NO:97); and c) the H3 sequence is GSLYSYGNSYFDY (SEQ ID NO:99), wherein X₁ is F or E; X₂ is Y or D, X₃ is M or D; X₅ is E or I; and X₆ is T or D. In any of the embodiments herein that includes a CAR with an ASTR that binds to Ror2, and in illustrative embodiments binds to the same epitope of Ror2 as an antibody that includes the antibody heavy chain of SEQ ID NO:82 or SEQ ID NO:83 and the antibody light chain of SEQ ID NO:84, the ASTR can include a heavy chain variable region with three complementarity determining regions, said regions having H1, H2, and H3 sequences, wherein: a) the H1 sequence is X₄GYSITTGYYWN (SEQ ID NO:96); b) the H2 sequence is YITYDGSKNYNPSLKN (SEQ ID NO:98); and c) the H3 sequence is FEGVWX₇GLDY (SEQ ID NO: 100), wherein X₄ is T or S; and X₇ is Y or G. In any of the embodiments herein that includes a CAR with an ASTR that binds to Ror2, the ASTR can include a light chain variable region with three complementarity determining regions, said regions having L1, L2, and L3 sequences, wherein: a) the L1 sequence is SATSSX₈SYMH (SEQ ID NO:101) or RASESVDRYGNSFIH (SEQ ID NO: 102); b) the L2 sequence is X₉TSNLAS (SEQ ID NO: 103) or RTYNLES (SEQ ID NO: 104); and c) the L3 sequence is QQRSSYPFT (SEQ ID NO: 105) or QQTNEDPWT (SEQ ID NO: 106), wherein X₈ is E or V; and X₉ is G or H. In any of the embodiments herein that includes a CAR with an ASTR that binds to Ror2, and in illustrative embodiments binds to the same epitope of Ror2 as an antibody that includes the antibody heavy chain of SEQ ID NO: 151 and the antibody light chain of SEQ ID NO: 152, the ASTR can include a light chain variable region with three complementarity determining regions, said regions having L1, L2, and L3 sequences, wherein: a) the L1 sequence is SATSSX₈SYMH (SEQ ID NO: 101); b) the L2 sequence is X₉TSNLAS (SEQ ID NO: 103); and c) the L3 sequence is QQRSSYPFT (SEQ ID NO: 105), wherein X₈ is E or V; and X₉ is G or H. In any of the embodiments herein that includes a CAR with an ASTR that binds to Ror2, and in illustrative embodiments binds to the same epitope of Ror2 as an antibody that includes the antibody heavy chain of SEQ ID NO:82 or SEQ ID NO:83 and the antibody light chain of SEQ ID NO:84, the ASTR can include a light chain variable region with three complementarity determining regions, said regions having L1, L2, and L3 sequences, wherein: a) the L1 sequence is RASESVDRYGNSFIH (SEQ ID NO: 102); b) the L2 sequence is RTYNLES (SEQ ID NO: 104); and c) the L3 sequence is QQTNEDPWT (SEQ ID NO: 106).

In any of the embodiments herein that includes a CAR with an ASTR that binds to Ror2, the ASTR can include a heavy chain variable region including three complementarity determining regions, said regions having H1, H2, and H3 sequences, wherein: a) the H1 sequence is GYTX₁TEX₂X₃X₄H (SEQ ID NO: 140) or GYSITTGX₂₉YWN (SEQ ID NO:141); b) the H2 sequence is X₅X₆X₇X₈NNGGTGYNQKFKG (SEQ ID NO: 142) or YITYDGSX₃₀NYNPSLKN (SEQ ID NO: 143); and c) the H3 sequence is X₉X₁₀X₁₁SX₁₂YX₁₃YX₁₄X₁₅SYFX₁₆X₁₇X₁₈ (SEQ ID NO: 144) or CSX₃₁X₃₂X₃₃X₃₄VX₃₅X₃₆X₃₇LDX₃₈ (SEQ ID NO: 145), wherein X₁ is F or E; X₂ is Y or D; X₃ is T or C; X₄ is M, D, E, or Y; X₅ is G or S; X₆ is I or E; X₇ is N, C, L, or V; X₈ is T, D or E; X₉ is A, M, or T; X₁₀ is R or H; Xᵤ is G or E; X₁₂ is L or F; X₁₃ is S or G; X₁₄ is G or D; X₁₅ is N or E; X₁₆ is D or L; X₁₇ is Y, C, or T; X₁₈ is W or L; X₂₉ is Y, E, R, or T; X₃₀ is K or N; X₃₁ is R, G, H, W, or Y; X₃₂ is F, C, N, or Q; X₃₃ is E or S; X₃₄ is G, E, F, H, M, Q, or S; X₃₅ is W, A, I, P, Q, T, or V; X₃₆ is Y, G, N, or Q; X₃₇ is G, S, or T; and X₃₈ is Y or I. In any of the embodiments herein that includes a CAR with an ASTR that binds to Ror2, and in illustrative embodiments binds to the same epitope of Ror2 as an antibody that includes the antibody heavy chain of SEQ ID NO: 151 and the antibody light chain of SEQ ID NO: 152, the ASTR can include a heavy chain variable region including three complementarity determining regions, said regions having H1, H2, and H3 sequences, wherein: a) the H1 sequence is GYTX₁TEX₂X₃X₄H (SEQ ID NO: 140); b) the H2 sequence is X₅X₆X₇X₈NNGGTGYNQKFKG (SEQ ID NO: 142); and c) the H3 sequence is X₉X₁₀X₁₁SX₁₂YX₁₃YX₁₄X₁₅SYFX₁₆X₁₇X₁₈ (SEQ ID NO: 144), wherein X₁ is F or E; X₂ is Y or D; X₃ is T or C; X₄ is M, D, E, or Y; X₅ is G or S; X₆ is I or E; X₇ is N, C, L, or V; X₈ is T, D or E; X₉ is A, M, or T; X₁₀ is R or H; X₁₁ is G or E; X₁₂ is L or F; X₁₃ is S or G; X₁₄ is G or D; X₁₅ is N or E; X₁₆ is D or L; X₁₇ is Y, C, or T; and X₁₈ is W or L. In any of the embodiments herein that includes a CAR with an ASTR that binds to Ror2, and in illustrative embodiments binds to the same epitope of Ror2 as an antibody that includes the antibody heavy chain of SEQ ID NO:82 or SEQ ID NO:83 and the antibody light chain of SEQ ID NO:84, the ASTR can include a heavy chain variable region including three complementarity determining regions, said regions having H1, H2, and H3 sequences, wherein: a) the H1 sequence is GYSITTGX₂₉YWN (SEQ ID NO:141); b) the H2 sequence is YITYDGSX₃₀NYNPSLKN (SEQ ID NO: 143); and c) the H3 sequence is CSX₃₁X₃₂X₃₃X₃₄VX₃₅X₃₆X₃₇LDX₃₈ (SEQ ID NO: 145), wherein X₂₉ is Y, E, R, or T; X₃₀ is K or N; X₃₁ is R, G, H, W, or Y; X₃₂ is F, C, N, or Q; X₃₃ is E or S; X₃₄ is G, E, F, H, M, Q, or S; X₃₅ is W, A, I, P, Q, T, or V; X₃₆ is Y, G, N, or Q; X₃₇ is G, S, or T; and X₃₈ is Y or I.

In any of the embodiments herein that includes a CAR with an ASTR that binds to Ror2, the ASTR can include a light chain variable region that includes three complementarity determining regions, said regions having L1, L2, and L3 sequences, wherein: a) the L1 sequence is SATSSX₁₉X₂₀X₂₁MX₂₂ (SEQ ID NO: 146) or RASESVDRYGNSX₃₉IH (SEQ ID NO: 147); b) the L2 sequence is X₂₃TSNLAS (SEQ ID NO: 148) or X₄₀TYX₄₁LES (SEQ ID NO: 149); and c) the L3 sequence is QX₂₄X₂₅SX₂₆YPFX₂₇X₂₈ (SEQ ID NO: 150) or QQX₄₂NX₄₃DPX₄₄TX₄₅ (SEQ ID NO:85), wherein X₁₉ is V or E; X₂₀ is S or D; X₂₁ is Y, C, or D; X₂₂ is H, G, or L; X₂₃ is G, C, H, or P; X₂₄ is Q or E; X₂₅ is R or H; X₂₆ is S, D, G, I, Q, or V; X₂₇ is T or D; X₂₈ is F, D, or E; X₃₉ is F, S, or T; X₄₀ is R, C, D, E, or W; X₄₁ is N or D; X₄₂ is T, I, or P; X₄₃ is E or V; X₄₄ is W or T; and X₄₅ is F or T. In any of the embodiments herein that includes a CAR with an ASTR that binds to Ror2, and in illustrative embodiments binds to the same epitope of Ror2 as an antibody that includes the antibody heavy chain of SEQ ID NO: 151 and the antibody light chain of SEQ ID NO: 152, the ASTR can include a light chain variable region that includes three complementarity determining regions, said regions having L1, L2, and L3 sequences, wherein: a) the L1 sequence is SATSSX₁₉X₂₀X₂₁MX₂₂ (SEQ ID NO: 146); b) the L2 sequence is X₂₃TSNLAS (SEQ ID NO: 148); and c) the L3 sequence is QX₂₄X₂₅SX₂₆YPFX₂₇X₂₈ (SEQ ID NO: 150), wherein X₁₉ is V or E; X₂₀ is S or D; X₂₁ is Y, C, or D; X₂₂ is H, G, or L; X₂₃ is G, C, H, or P; X₂₄ is Q or E; X₂₅ is R or H; X₂₆ is S, D, G, I, Q, or V; X₂₇ is T or D; and X₂₈ is F, D, or E. In any of the embodiments herein that includes a CAR with an ASTR that binds to Ror2, and in illustrative embodiments binds to the same epitope of Ror2 as an antibody that includes the antibody heavy chain of SEQ ID NO:82 or SEQ ID NO:83 and the antibody light chain of SEQ ID NO:84, the ASTR can include a light chain variable region that includes three complementarity determining regions, said regions having L1, L2, and L3 sequences, wherein: a) the L1 sequence is RASESVDRYGNSX₃₉IH (SEQ ID NO: 147); b) the L2 sequence is X₄₀TYX₄₁LES (SEQ ID NO: 149); and c) the L3 sequence is QQX₄₂NX₄₃DPX₄₄TX₄₅ (SEQ ID NO:85), wherein X₃₉ is F, S, or T; X₄₀ is R, C, D, E, or W; X₄₁ is N or D; X₄₂ is T, I, or P; X₄₃ is E or V; X₄₄ is W or T; and X₄₅ is F or T.

In any of the embodiments herein that includes a CAR with an ASTR that binds to Ror2, in illustrative embodiments wherein the ASTR binds to the same epitope of Ror2 as an antibody comprising an antibody heavy chain of SEQ ID NO:82 or SEQ ID NO:83 and an antibody light chain of SEQ ID NO:84, the heavy chain variable region can comprise an amino acid sequence of SEQ ID NO:82, SEQ ID NO:83, SEQ ID NO: 120 or SEQ ID NO:121.

In any of the embodiments herein that includes a CAR with an ASTR that binds to Ror2, in illustrative embodiments wherein the ASTR binds to the same epitope of Ror2 as an antibody comprising an antibody heavy chain of SEQ ID NO:82 or SEQ ID NO:83 and an antibody light chain of SEQ ID NO:84, the heavy chain variable region can comprise an amino acid sequence of SEQ ID NO:82 or SEQ ID NO:83.320. The chimeric antigen receptor of claim 2931, wherein the ASTR binds to the same epitope of Ror2 as an antibody comprising an antibody heavy chain of SEQ ID NO:82 or SEQ ID NO:83 and an antibody light chain of SEQ ID NO:84, and wherein the light chain variable region comprises an amino acid sequence of SEQ ID NO:86.

In any of the embodiments herein that includes a CAR with an ASTR that binds to Ror2, in illustrative embodiments wherein the ASTR binds to the same epitope of Ror2 as an antibody comprising an antibody heavy chain of SEQ ID NO:82 or SEQ ID NO:83 and an antibody light chain of SEQ ID NO:84, the light chain variable region can comprise an amino acid sequence of SEQ ID NO:84.

In any of the embodiments herein that includes a CAR with an ASTR that binds to Ror2, in illustrative embodiments wherein the ASTR binds to the same epitope of Ror2 as an antibody comprising an antibody heavy chain of SEQ ID NO:82 or SEQ ID NO:83 and an antibody light chain of SEQ ID NO:84, the heavy chain variable region can comprise an amino acid sequence of SEQ ID NO:82, SEQ ID NO:83, SEQ ID NO: 120, or SEQ ID NO:121.

In any of the embodiments herein that includes a CAR with an ASTR that binds to Ror2, in illustrative embodiments wherein the ASTR binds to the same epitope of Ror2 as an antibody comprising an antibody heavy chain of SEQ ID NO:82 or SEQ ID NO:83 and an antibody light chain of SEQ ID NO:84, the heavy chain variable region can comprise an amino acid sequence of SEQ ID NO:82 or SEQ ID NO:83.

In any of the embodiments herein that includes a CAR with an ASTR that binds to Ror2, in illustrative embodiments wherein the ASTR binds to the same epitope of Ror2 as an antibody comprising an antibody heavy chain of SEQ ID NO:82 or SEQ ID NO:83 and an antibody light chain of SEQ ID NO:84, the ASTR can comprise an amino acid sequence of SEQ ID NO: 130, SEQ ID NO:131, or SEQ ID NO: 132, SEQ ID NO: 153, SEQ ID NO: 154, SEQ ID NO: 157, or SEQ ID NO: 158.

In any of the embodiments herein that includes a CAR with an ASTR that binds to Ror2, in illustrative embodiments wherein the ASTR binds to the same epitope of Ror2 as an antibody comprising an antibody heavy chain of SEQ ID NO: 151 and an antibody light chain of SEQ ID NO: 152, the light chain variable region can comprise an amino acid sequence of SEQ ID NO: 81, SEQ ID NO: 122, SEQ ID NO: 123, SEQ ID NO: 124, or SEQ ID NO:152.

In any of the embodiments herein that includes a CAR with an ASTR that binds to Ror2, in illustrative embodiments wherein the ASTR binds to the same epitope of Ror2 as an antibody comprising an antibody heavy chain of SEQ ID NO: 151 and an antibody light chain of SEQ ID NO: 152, the light chain variable region can comprise an amino acid sequence of SEQ ID NO: 152.

In any of the embodiments herein that includes a CAR with an ASTR that binds to Ror2, in illustrative embodiments wherein the ASTR binds to the same epitope of Ror2 as an antibody comprising an antibody heavy chain of SEQ ID NO: 151 and an antibody light chain of SEQ ID NO: 152, the heavy chain variable region can comprise an amino acid sequence of SEQ ID NO: 115, SEQ ID NO:116, SEQ ID NO:117, SEQ ID NO:118, SEQ ID NO:119, or SEQ ID:151.

In any of the embodiments herein that includes a CAR with an ASTR that binds to Ror2, in illustrative embodiments wherein the ASTR binds to the same epitope of Ror2 as an antibody comprising an antibody heavy chain of SEQ ID NO: 151 and an antibody light chain of SEQ ID NO: 152, the heavy chain variable region can comprise an amino acid sequence of SEQ ID:151.

In any of the embodiments herein that includes a CAR with an ASTR that binds to Ror2, in illustrative embodiments wherein the ASTR binds to the same epitope of Ror2 as an antibody comprising an antibody heavy chain of SEQ ID NO: 151 and an antibody light chain of SEQ ID NO: 152, the ASTR can comprise an amino acid sequence of SEQ ID NO: 155 or SEQ ID NO:156.

In any of the embodiments herein that includes a CAR, the transmembrane domain can have at least 50%, 60%, 70%, 75%, 80%, 85%, 90%, 95%, 96%, 97%, 98%, 99%, or 100% sequence identity to the sequence of SEQ ID NO:46, at least 50%, 60%, 70%, 75%, 80%, 85%, 90%, 95%, 96%, 97%, 98%, 99%, or 100% sequence identity to the CD8 transmembrane domain, for example, SEQ ID NOs:47 or 75, at least 50%, 60%, 70%, 75%, 80%, 85%, 90%, 95%, 96%, 97%, 98%, 99%, or 100% sequence identity to the CD4 transmembrane domain, for example, SEQ ID NO:48, at least 50%, 60%, 70%, 75%, 80%, 85%, 90%, 95%, 96%, 97%, 98%, 99%, or 100% sequence identity to the CD3 zeta transmembrane domain, for example, SEQ ID NO:49, at least 50%, 60%, 70%, 75%, 80%, 85%, 90%, 95%, 96%, 97%, 98%, 99%, or 100% sequence identity to the CD28 transmembrane domain, for example, SEQ ID NOs:50 or 76, at least 50%, 60%, 70%, 75%, 80%, 85%, 90%, 95%, 96%, 97%, 98%, 99%, or 100% sequence identity to the OX40 transmembrane domain, for example, SEQ ID NO:51, at least 50%, 60%, 70%, 75%, 80%, 85%, 90%, 95%, 96%, 97%, 98%, 99%, or 100% sequence identity to the CD134 transmembrane domain, or at least 50%, 60%, 70%, 75%, 80%, 85%, 90%, 95%, 96%, 97%, 98%, 99%, or 100% sequence identity to the CD7 transmembrane domain, for example, SEQ ID NO:52, and the conditionally active ASTR can retain an increased binding to Ror2 or Axl in a tumor microenvironment and/or in an in vitro tumor surrogate assay condition relative to a corresponding physiological condition when the ASTR is part of the CAR. In any of the embodiments herein that includes a CAR, the transmembrane can be the CD8 transmembrane domain or the CD28 transmembrane domain. In any of the embodiments herein that includes a CAR, the transmembrane domain can be located between the ASTR and the intracellular activating domain. In any of the embodiments herein that includes a CAR, the intracellular activating domain can retain an activating activity and have at least 50%, 60%, 70%, 75%, 80%, 85%, 90%, 95%, 96%, 97%, 98%, 99%, or 100% sequence identity to the CD3Z activating domain, for example, SEQ ID NOs:11-16, at least 50%, 60%, 70%, 75%, 80%, 85%, 90%, 95%, 96%, 97%, 98%, 99%, or 100% sequence identity to the CD3D activating domain, for example, SEQ ID NOs:17-19, at least 50%, 60%, 70%, 75%, 80%, 85%, 90%, 95%, 96%, 97%, 98%, 99%, or 100% sequence identity to the CD3E activating domain, for example, SEQ ID NOs:20-21, at least 50%, 60%, 70%, 75%, 80%, 85%, 90%, 95%, 96%, 97%, 98%, 99%, or 100% sequence identity to the CD3G activating domain, for example, SEQ ID NOs:22-23, at least 50%, 60%, 70%, 75%, 80%, 85%, 90%, 95%, 96%, 97%, 98%, 99%, or 100% sequence identity to the CD28 activating domain, for example, SEQ ID NO:35, at least 50%, 60%, 70%, 75%, 80%, 85%, 90%, 95%, 96%, 97%, 98%, 99%, or 100% sequence identity to the CD79A activating domain, for example, SEQ ID NOs:24-26, at least 50%, 60%, 70%, 75%, 80%, 85%, 90%, 95%, 96%, 97%, 98%, 99%, or 100% sequence identity to the DAP10 activating domain, for example, SEQ ID NO:34, at least 50%, 60%, 70%, 75%, 80%, 85%, 90%, 95%, 96%, 97%, 98%, 99%, or 100% sequence identity to the DAP12 activating domain, for example, SEQ ID NOs:27-31, at least 50%, 60%, 70%, 75%, 80%, 85%, 90%, 95%, 96%, 97%, 98%, 99%, or 100% sequence identity to the FCER1G activating domain, for example, SEQ ID NOs:32-33, at least 50%, 60%, 70%, 75%, 80%, 85%, 90%, 95%, 96%, 97%, 98%, 99%, or 100% sequence identity to the CD137 activating domain, or at least 50%, 60%, 70%, 75%, 80%, 85%, 90%, 95%, 96%, 97%, 98%, 99%, or 100% sequence identity to the ZAP70 activating domain, for example, SEQ ID NO:36.

In any of the embodiments herein that includes a CAR, the CAR can further include a stalk domain. In any of the embodiments herein that includes a CAR, the CAR can further include a co-stimulatory domain. In some embodiments, the CAR can include a stalk domain and a co-stimulatory domain. In some embodiments, the CAR can include from amino terminus to carboxy terminus, an ASTR, a stalk domain, a transmembrane domain, a co-stimulatory domain, and an intracellular activating domain.

In any of the embodiments herein that includes a CAR with a stalk domain, the stalk domain can have at least 50%, 60%, 70%, 75%, 80%, 85%, 90%, 95%, 96%, 97%, 98%, 99%, or 100% sequence identity to any CD8 stalk domain known in the art, for example, SEQ ID NO: 125, or at least 50%, 60%, 70%, 75%, 80%, 85%, 90%, 95%, 96%, 97%, 98%, 99%, or 100% sequence identity to any CD28 stalk domain known in the art, for example, SEQ ID NO: 126, and the conditionally active antigen-specific targeting region can retain an increased binding to Ror2 or Axl in a tumor microenvironment and/or an in vitro tumor surrogate assay condition relative to a corresponding physiological condition when the ASTR is part of the CAR.

In any of the embodiments herein that includes a CAR, the CAR can further include a co-stimulatory domain. In any of the embodiments herein that includes a CAR with a co-stimulatory domain, the intracellular co-stimulatory domain can include a DAP10/CD28 type signaling chain or an immunoreceptor tyrosine-based activation motif (ITAM)-containing intracellular signaling polypeptide. In any of the embodiments herein that includes a CAR with a co-stimulatory domain, the CAR can include a co-stimulatory domain that has a different amino acid sequence than the intracellular activating domain. In any of the embodiments herein that includes a CAR with a co-stimulatory domain, the co-stimulatory domain can have at least 50%, 60%, 70%, 75%, 80%, 85%, 90%, 95%, 96%, 97%, 98%, 99%, or 100% sequence identity to the CD137 co-stimulatory domain, for example, SEQ ID NO: 1, at least 50%, 60%, 70%, 75%, 80%, 85%, 90%, 95%, 96%, 97%, 98%, 99%, or 100% sequence identity to the CD28 co-stimulatory domain, for example, SEQ ID NO:2, at least 50%, 60%, 70%, 75%, 80%, 85%, 90%, 95%, 96%, 97%, 98%, 99%, or 100% sequence identity to the ICA co-stimulatory domain, for example, SEQ ID NO:3, at least 50%, 60%, 70%, 75%, 80%, 85%, 90%, 95%, 96%, 97%, 98%, 99%, or 100% sequence identity to the ICOS co-stimulatory domain, for example, SEQ ID NO:4, at least 50%, 60%, 70%, 75%, 80%, 85%, 90%, 95%, 96%, 97%, 98%, 99%, or 100% sequence identity to the OX40 co-stimulatory domain, for example, SEQ ID NO:5, at least 50%, 60%, 70%, 75%, 80%, 85%, 90%, 95%, 96%, 97%, 98%, 99%, or 100% sequence identity to the BTLA co-stimulatory domain, for example, SEQ ID NO:7, at least 50%, 60%, 70%, 75%, 80%, 85%, 90%, 95%, 96%, 97%, 98%, 99%, or 100% sequence identity to the CD27 co-stimulatory domain, for example, SEQ ID NO:6, at least 50%, 60%, 70%, 75%, 80%, 85%, 90%, 95%, 96%, 97%, 98%, 99%, or 100% sequence identity to the CD30 co-stimulatory domain, for example, SEQ ID NO:8, at least 50%, 60%, 70%, 75%, 80%, 85%, 90%, 95%, 96%, 97%, 98%, 99%, or 100% sequence identity to the GITR co-stimulatory domain, for example, SEQ ID NO:9, or at least 50%, 60%, 70%, 75%, 80%, 85%, 90%, 95%, 96%, 97%, 98%, 99%, or 100% sequence identity to the HVEM co-stimulatory domain, for example, SEQ ID NO: 10. In any of the embodiments herein that includes a CAR with a co-stimulatory domain, the co-stimulatory domain can be the CD28 co-stimulatory domain or the CD137 co-stimulatory domain. In any of the embodiments herein that includes a CAR with a co-stimulatory domain, the co-stimulatory domain can be the ICA co-stimulatory domain. In any of the embodiments herein that includes a CAR with a co-stimulatory domain, the co-stimulatory domain can be an ICΔ co-stimulatory domain and the intracellular activating domain can be a CD3Z activating domain. In any of the embodiments herein that includes a CAR with a stalk domain and a co-stimulatory domain, the stalk domain can be a CD8 stalk domain or a CD28 stalk domain, the transmembrane domain can be a CD8 transmembrane domain or a CD28 transmembrane domain, the intracellular activating domain can be a CD3Z activating domain, and the co-stimulatory domain can be a CD137 co-stimulatory domain, an ICΔ co-stimulatory domain, or a CD28 co-stimulatory domain, or the co-stimulatory domain can include both a CD137 co-stimulatory domain and an ICΔ co-stimulatory domain.

In any of the embodiments herein that includes a CAR, the CAR can further include a recognition domain. In any of the embodiments that includes a CAR with a recognition domain, the recognition domain can be expressed on the same polypeptide as the CAR. In any of the embodiments that includes a CAR with a recognition domain on the same polypeptide as the CAR, the recognition domain can be at or near the N-terminus, such as within 10, 20, 30, 40, or 50 amino acids of the N-terminus. In any of the embodiments that includes a CAR with a recognition domain on the same polypeptide as the CAR, the recognition domain can be at or near the C-terminus, such as within 10, 20, 30, 40, or 50 amino acids of the C-terminus. In any of the embodiments that includes a CAR with a recognition domain on the same polypeptide as the CAR, the recognition domain can be between any of the domains of the CAR, such as between the stalk and transmembrane domains. In any of the embodiments herein that includes a CAR with a recognition domain, the recognition domain can be expressed such that it is covalently attached to the CAR and the recognition domain can be separated from the CAR by a cleavage signal or a ribosomal skip sequence. In any of the embodiments herein that includes a CAR with a recognition domain with a ribosomal skip sequence, the ribosomal skip sequence can be 2A-1, for example SEQ ID NO:77. In any of the embodiments herein that includes a CAR with a recognition domain, the recognition domain can be at least 10, 15, 20, 25, 30, 35, 40, 45, or 50 contiguous amino acids of EGFR. In any of the embodiments that includes a CAR with a recognition domain, the recognition domain can be the FLAG epitope (SEQ ID NO:38).

In any of the embodiments herein that includes a CAR, the CAR can further include a signal sequence. In any of the embodiments herein that includes a CAR with a signal sequence, the signal sequence can be an epitope tag, an affinity domain, and/or a polypeptide that produces a detectable signal. In any of the embodiments herein that includes a CAR with a signal sequence, the signal sequence can be at the amino terminus.

In any of the embodiments herein that includes an expression vector that includes a nucleic acid encoding a CAR, the expression vector can further include nucleotides encoding a recognition domain. In any of the embodiments herein that includes an expression vector that includes a nucleic acid encoding a CAR and nucleotides encoding a recognition domain, the nucleotides encoding the recognition domain can encode at least 10, 15, 20, 25, 30, 35, 40, 45, or 50 contiguous amino acids of EGFR. In any of the embodiments herein that includes an expression vector that includes a nucleic acid encoding a CAR and nucleotides encoding a recognition domain, the nucleotides encoding the recognition domain can be separated from the nucleotides encoding the CAR by an internal ribosome entry site. In any of the embodiments herein that includes an expression vector that includes a nucleic acid encoding a CAR and nucleotides encoding a recognition domain, the recognition domain can be expressed as part of a single polypeptide that further includes the CAR and the recognition domain on the single polypeptide can be separated from the CAR by a cleavage signal or a ribosomal skip sequence, which can be a 2A-1 sequence, for example SEQ ID NO:77. In any of the embodiments herein that includes an expression vector, the expression vector can be a viral vector. In any of the embodiments herein that includes an expression vector that is a viral vector, the viral vector can be replication incompetent recombinant retroviral particle, replication incompetent recombinant lentiviral particle, a vaccinia virus, a poliovirus, an adeno-associated virus, SV40, a herpes simplex virus, a gamma retrovirus, a human immunodeficiency virus, a lentivirus, or an adenovirus.

In any of the embodiments herein that includes a mammalian cell expressing a CAR, the cell can be a lymphocyte. In any of the embodiments herein that includes a lymphocyte expressing a CAR, the lymphocyte can be a T cell. In any of the embodiments herein that includes a T cell expressing a CAR, the T cell can be a CD4+ T cell or the T cell can be a CD8+ T cell. In any of the embodiments herein that includes a lymphocyte expressing a CAR, the lymphocyte can be a cytotoxic cell that preferentially kills antigen-expressing cells in a tumor microenvironment, and the lymphocyte can include a nucleic acid encoding any of the CARs of Embodiments 1 or A1 to A8.

In any of the embodiments herein that includes a kit containing a container containing a replication incompetent recombinant retroviral particle, and instructions for use thereof, the instructions can further include increasing the pH of the microenvironment after contacting the target mammalian cell by introducing a pH-modulating pharmacologic agent to the microenvironment in sufficient amount, thereby affecting the binding of the target mammalian cell with the T cell and/or NK cell. In any of the embodiments herein that includes a kit containing a container containing a replication incompetent recombinant retroviral particle, and instructions for use thereof, the kit can further include a pH-modulating pharmacologic agent. In any of the embodiments herein that includes a kit containing a container containing a replication incompetent recombinant retroviral particle, and instructions for use thereof, the instructions can include a step of introducing the T cell and/or NK cell capable of expressing the CAR into a subject before the contacting, wherein after the introducing, the T cell and/or the NK cell including the one or more nucleic acid sequences encoding the CAR expresses the CAR and binds to the target mammalian cell expressing the cognate antigen in the subject.

In any of the embodiments herein that includes treating a cancer, the cancer can be renal cell carcinoma. In any of the embodiments herein that includes treating a cancer, the cancer can be soft tissue sarcoma or mesothelioma. In any of the embodiments herein that includes treating a cancer and include a CAR with a recognition domain, the method can further include administering a dose of antibodies directed against the recognition domain after administering the genetically engineered cytotoxic cell to the subject. In any of the embodiments herein that includes treating a cancer, the cancer can express Axl and the CAR can include an ASTR that bind Axl. In any of the embodiments herein that includes treating a cancer, the cancer can express Ror2 and the CAR can include an ASTR that bind Ror2. In any of the embodiments herein that includes treating a cancer, the cytotoxic cells can be T cells and/or NK cells. In any of the embodiments herein that includes treating a cancer, the cytotoxic cells can be T cells. In any of the embodiments herein that includes expanding T cells and/or NK cells, the method can further include harvesting the genetically modified T cells and/or NK cells after the expanding. In any of the embodiments herein that includes harvesting T cells and/or NK cells, the method can further include cryopreserving the harvested genetically modified T cells and/or NK cells. In any of the embodiments herein that includes cryopreserving T cells and/or NK cells, the cryopreserved genetically modified T cells and NK cells can be thawed. In any of the embodiments herein that includes harvesting T cells and/or NK cells, the method can further include introducing the harvested genetically modified T cells and/or NK cells into a subject.

Exemplary conditionally active CARs (CAB-CARs) that have increased binding to Axl at pH 6.7 compared to ph7.4 are found in Example 1 herein. In illustrative embodiments, the CAR or ASTR can bind to the same epitope of Axl as a single-chain variable antibody fragment comprising an antibody heavy chain of SEQ ID NO:79 and an antibody light chain of SEQ ID NO:80. In further embodiments of such illustrative embodiments, the anti-Axl CAR or ASTR comprises or is a single chain variable fragment (scFv). In further illustrative examples, the anti-Axl scFv comprises either a heavy chain that is N-terminal to a light chain or a light chain that is N-terminal to a heavy chain. In any of the embodiments herein that includes a CAR, and in illustrative embodiments binds to the same epitope of Axl as an antibody that includes the antibody heavy chain of SEQ ID NO:79 and the antibody light chain of SEQ ID NO:80, the ASTR can include any of SEQ ID NOs:128, 129, 159, 160, or 161. Furthermore, anti-Axl CARs of any of the embodiments herein can include any of the CAR components provided herein. In certain exemplary embodiments, the anti-Axl CARs can include the CAR components listed in Table 1 and can be any of the CARs in Table 1. More typically for any embodiments herein that include an anti-Axl CAR, the CAR is a CAB-CAR, and in non-limiting illustrative embodiments, can include, for example, any of the CAB-CAR components and CAB-CARs provided in Table 1 that demonstrated cytotoxic activity. For example, the anti-Axl CAB-CAR can include a CD8 signal peptide, a CD8 or CD28 stalk/transmembrane domain, a CD137, ICA, or both a ICΔ co-stimulatory domain and a CD137 co-stimulatory domain, and/or a CD3Z activation domain. Furthermore, illustrative CARs for any of the embodiments herein that includes an anti-Axl CAR, and especially an anti-Axl CAB-CAR, in non-limiting illustrative embodiments include any of the anti-Axl CAB-CARs that demonstrated conditional cytotoxic activity in Table 1. Such illustrative CAB-CARs include F1-2-1, F1-2-2, F1-2-3, F1-2-6, F1-2-8, F1-2-10, F1-2-13, F1-2-14, F1-2-15, F1-2-22, or F1-2-23 of Table 1. In any of the embodiments herein that includes an ASTR, the ASTR can include the ASTR of F1-2-1, F1-2-2, F1-2-3, F1-2-6, F1-2-8, F1-2-10, F1-2-13, F1-2-14, F1-2-15, F1-2-22, or F1-2-23. Furthermore, illustrative CARs for any of the embodiments herein that includes an anti-Axl CAR, and especially an anti-Axl CAB-CAR, in non-limiting illustrative embodiments include any of the anti-Axl CAB-CARs that demonstrated high conditional cytotoxic activity in Table 1. Such illustrative CAB-CARs include F1-2-13, F1-2-15, F1-2-22, or F1-2-23. Accordingly, in any of the embodiments herein that includes an ASTR, the ASTR can include the ASTR of F1-2-1, F1-2-2, F1-2-3, F1-2-6, F1-2-8, F1-2-10, F1-2-13, F1-2-14, F1-2-15, F1-2-22, or F1-2-23.

Exemplary conditionally active CARs (CAB-CARs) that have increased binding to Ror2 at pH 6.7 compared to pH 7.4 are found in Example 1 herein. In illustrative embodiments, the CAR or ASTR can bind to the same epitope of Ror2 as a single-chain variable antibody fragment comprising an antibody heavy chain of SEQ ID NO:82 or SEQ ID NO:83 and an antibody light chain of SEQ ID NO:84 or the CAR or ASTR can bind to the same epitope of Ror2 as a single-chain variable antibody fragment comprising an antibody heavy chain of SEQ ID NO: 151 and an antibody light chain of SEQ ID NO: 152. In further embodiments of such illustrative embodiments, the anti-Ror2 CAR or ASTR comprises or is a single chain variable fragment (scFv) and in further illustrative examples, comprises a light chain that is N terminal to a heavy chain, or comprises a heavy chain that is N-terminal to a light chain. In any of the embodiments herein that includes a CAR or ASTR, and in illustrative embodiments binds to the same epitope of Ror2 as a single-chain variable antibody fragment comprising an antibody heavy chain of SEQ ID NO:82 or SEQ ID NO:83 and an antibody light chain of SEQ ID NO:84 or binds to the same epitope of Ror2 as a single-chain variable antibody fragment comprising an antibody heavy chain of SEQ ID NO: 151 and an antibody light chain of SEQ ID NO: 152, the ASTR can include any of SEQ ID NOs:130-132, or 153-158. Furthermore, anti-Ror2 CARs of any of the embodiments herein can include any of the CAR components provided herein. In certain exemplary embodiments, the anti-Ror2 CARS can include the CAR components listed in Table 2 and can be any of the CARs in Table 2. More typically for any embodiments herein that include an anti-Ror2 CAR, the CAR is a CAB-CAR, and in non-limiting illustrative embodiments, can include, for example, any of the CAB-CAR components and CAB-CARs provided in Table 2 that demonstrated cytotoxic activity. For example, the anti-Ror2 CAB-CAR can include a CD8 signal peptide, a CD8 or CD28 stalk/transmembrane domain, a CD137 co-stimulatory domain, and/or a CD3Z activation domain. Furthermore, illustrative CARs for any of the embodiments herein that includes an anti-Ror2 CAR and especially an anti-Ror2 CAB-CAR, in non-limiting illustrative embodiments include any of the anti-Ror2 CAB-CARs that demonstrated conditional cytotoxic activity in Table 2. Such illustrative CAB-CARs include F1-1-9, F1-1-10, F1-1-11, F1-1-12, F1-1-15, F1-1-17, F1-1-18, F1-1-19, F1-1-20, F1-1-21, F1-1-23, F1-1-25, or F1-1-26. In any of the embodiments herein that includes an anti-Ror2 ASTR, the ASTR can include the ASTR of F1-1-9, F1-1-10, F1-1-11, F1-1-12, F1-1-15, F1-1-17, F1-1-18, F1-1-19, F1-1-20, F1-1-21, F1-1-23, F1-1-25, or F1-1-26 of Table 2. Furthermore, illustrative CARs for any of the embodiments herein that includes an anti-Ror2 CAR and especially an anti-Ror2 CAB-CAR, in non-limiting illustrative embodiments include any of the anti-Ror2 CAB-CARs that demonstrated high conditional cytotoxic activity in Table 2. Such illustrative CAB-CARs include F1-1-11, F1-1-12, F1-1-15, F1-1-17, F1-1-19, F1-1-20, or F1-1-23. Accordingly, in any of the embodiments herein that includes an anti-Ror2 ASTR, the ASTR can include the ASTR of F1-1-11, F1-1-12, F1-1-15, F1-1-17, F1-1-19, F1-1-20, F1-1-23.

In another aspect, provided herein is a method for modulating binding of a conditionally active biologic chimeric antigen receptor (CAB-CAR)-expressing T cell or NK cell to a cell expressing a cognate antigen of the CAB-CAR in a subject, including:
a. introducing a T cell and/or NK cell including a nucleic acid encoding the CAB-CAR into the subject, wherein after the introducing, the T cell and/or the NK cell including the nucleic acid encoding the CAB-CAR expresses the CAB-CAR and binds to the cell expressing the cognate antigen in the subject, and wherein the cognate antigen is an Axl polypeptide or an epitope thereof, or a Ror2 polypeptide or an epitope thereof; and
b. administering a pharmacologic agent to the subject in sufficient amount to increase blood pH and/or pH of a tissue and/or pH of a microenvironment, wherein the administering is performed before, during, or after the introducing, and wherein the increased pH of the blood, the tissue, and/or the microenvironment modulates binding of the CAB-CAR expressing T cell and/or NK cell to the cell expressing the cognate antigen in the blood, the tissue, or the microenvironment with the increased pH.

In another aspect, provided herein is a method for alleviating on target off tumor toxicity in a subject, including:
a. introducing a nucleic acid encoding a conditionally active biologic chimeric antigen receptor (CAB-CAR) into a T cell or NK cell of the subject to produce a T cell and/or NK cell including a nucleic acid encoding the CAB-CAR;
b. introducing the T cell and/or NK cell including the nucleic acid encoding the CAB-CAR into the subject, wherein after the introducing, the T cell and/or the NK cell including the nucleic acid encoding the CAB-CAR expresses the CAB-CAR and binds to the cell expressing a cognate antigen in the subject, wherein the cognate antigen is an Axl polypeptide or an epitope thereof, or a Ror2 polypeptide or an epitope thereof; and
c. administering a pharmacologic agent to the subject in sufficient amount to increase blood pH and/or pH of a tissue and/or pH of a microenvironment to modulate binding of the CAB-CAR to its cognate antigen in the blood, the tissue, and/or the microenvironment with the increased pH, thereby alleviating on target off tumor toxicity in the subject.

In some embodiments, the nucleic acid can be a vector. In illustrative embodiments, the vector is a retroviral vector.

In another aspect, provided herein is a method for controlling binding of a T cell and/or NK cell to a target mammalian cell, including:
a. contacting the target mammalian cell with the T cell and/or NK cell in a microenvironment, wherein the target mammalian cell expresses a cognate antigen, and the T cell and/or NK cell expresses a conditionally active biologic chimeric antigen receptor (CAB-CAR) that binds to the cognate antigen differentially at pH 6.7 as compared to pH 7.4, wherein the cognate antigen is an Axl polypeptide or an epitope thereof, or a Ror2 polypeptide or an epitope thereof; and
b. increasing the pH of the microenvironment by introducing a pharmacologic agent to the microenvironment in sufficient amount, thereby controlling the binding of the T cell and/or NK cell to the target mammalian cell.

In another aspect, provided herein is a method for controlling the binding of a T cell and/or NK cell expressing a conditionally active biologic chimeric antigen receptor (CAB-CAR) to a target mammalian cell in a subject in vivo, including administering a pH-modulating pharmacologic agent to the subject through an effective dosing regimen that increases the pH of a microenvironment within the subject, wherein the subject includes the T cell and/or the NK cell expressing the CAB-CAR, wherein the CAB-CAR binds to its cognate antigen differentially at pH 6.7 as compared to pH 7.4, wherein the cognate antigen is an Axl polypeptide or an epitope thereof, or a Ror2 polypeptide or an epitope thereof, wherein the microenvironment include the target mammalian cell, wherein the target mammalian cell expresses the cognate antigen on its surface, and wherein the T cell and/or NK cell binds to the target mammalian cell differentially before versus after the pH of the microenvironment is increased, thereby controlling the binding of the T cell and/or NK cell to the target mammalian cell in a subject in vivo.

In any of the aspects provided immediately above that include a pharmacologic agent and a CAB-CAR, the CAB-CAR can have reduced binding to its cognate antigen at one pH than at a different pH. In illustrative embodiments where illustrative pH values for differential binding of a CAB-CAR are not already provided in the broadest aspect and alternatively for other embodiments in place of those values for such aspects, the CAB-CAR can have reduced binding at a higher pH than at a lower pH. For example, the CAB-CAR can have reduced binding to its cognate antigen at a pH above 7.0, 7.1, 7.2, 7.3, 7.4, or 7.5 than at a pH below 6.4, 6.5, 6.6, 6.7, 6.8, 6.9, or 7.0. In other embodiments, the CAB-CAR can have reduced binding at a higher pH than at a lower pH. For example, the CAB-CAR can have reduced binding to its cognate antigen at a pH below 6.4, 6.5, 6.6, 6.7, 6.8, 6.9, or 7.0 than at a pH above 7.0, 7.1, 7.2, 7.3, 7.4, or 7.5. In some illustrative embodiments, the CAB-CAR exhibits increased binding at a pH of 6.5 to 6.7 compared to pH 7.4 to 7.6. In other illustrative embodiments, the CAB-CAR exhibits increased binding at a pH of 6.7 compared to a pH of 7.4. In other embodiments, the CAB-CAR exhibits increased binding in the pH of a tumor compared to the pH of blood. In some embodiments, the CAB-CAR can include an antigen-specific targeting region, a stalk, and an intracellular activating domain. In some embodiments, the CAB-CAR can also include a co-stimulatory domain. In some embodiments, the CAB-CAR can bind to a tumor associated antigen.

In any of the aspects provided immediately above that include a pharmacologic agent and a CAB-CAR, the pH of the microenvironment can be increased from a pH below 7.0 to a pH above 7.0. For example, the pH can be increased from a pH below 6.4, 6.5, 6.6, 6.7, 6.8, 6.9, or 7.0 to a pH above 7.0, 7.1, 7.2, 7.3, or 7.4. In some embodiments, the CAB-CAR can bind to the cognate antigen at the increased pH but not a pH of the microenvironment before introducing the pharmacologic agent. In certain embodiments, the pH can be increased from below 7.0 to a pH of 7.1 to 8.0 or to a pH of 7.1 to 7.8 or to a pH of 7.2 to 7.8 or a pH of 7.2 to 7.6 or a pH of 7.3 to 7.6 or to a pH of 7.4 to 7.8 or to a pH of 7.4 to 7.6. Such an increase in pH can occur for less than 1, 2, 4, 6, 8, 12, or 24 hours or for more than 1, 2, 4, 6, 8, 12 or 24 hours depending on the type and dose of pharmacologic agent administered. In certain embodiments, the pharmacologic agent is administered such that the pH remains above 7.0, 7.1, 7.2, 7.3, 7.4, or 7.5; or between 7.0, 7.1, 7.2, 7.3 on the low end of the range and 7.4, 7.5, 7.6, 7.7, or 7.8 on the high end of the range, in the target tissue, such as a tumor, and for example in at least a surface of a target tissue (e.g. tumor) microenvironment, in at least a portion of a target tissue (e.g. tumor) microenvironment, and in illustrative embodiments throughout a target tissue (e.g. tumor) microenvironment.

In any of the aspects provided immediately above that include a pharmacologic agent and a CAB-CAR, the microenvironment can be an in vivo microenvironment, such as a tumor, a tissue, a non-tumor tissue, a normal tissue, or a tissue that has undergone a transient shift in pH. For example, tissues that typically undergo transient shifts in pH include a muscle tissue in anaerobic conditions or muscle tissue undergoing exercise or an inflamed tissue or a tissue experiencing inflammation. In some embodiments that include a target mammalian cell, the target mammalian cell can be a tumor cell or a non-tumor or normal cell.

In any of the aspects provided immediately above that include a pharmacologic agent and a CAB-CAR, the pharmacologic agent can be sodium bicarbonate, tris-hydroxylmethyl aminomethane, an equimolar hypertonic solution of sodium bicarbonate and sodium carbonate, or proton pump inhibitors such esomeprazole, esomeprazole and naproxen, lansoprazole, omeprazole, and rabeprazole.

In any embodiment provided immediately above that includes a replication incompetent recombinant retroviral particle in a method that includes a CAB-CAR and a pharmacologic agent, the polynucleotide that includes a transcriptional unit operatively linked to a promoter active in T cells and/or NK cells that encodes the CAB-CAR is taken up by the T cell(s) and/or NK cell(s) such that such the cell(s) is capable of expressing the CAB-CAR. In illustrative embodiments, the T cell(s) and/or NK cell(s) integrate the polynucleotide into their genome.

In any embodiment provided immediately above that includes a replication incompetent recombinant retroviral particle in a method that includes a CAB-CAR and a pharmacologic agent, the replication incompetent recombinant retroviral particle can include on its surface a recognition domain of a monoclonal antibody approved biologic. For example, the recognition domain can include a polypeptide that is recognized by an antibody that recognizes EGFR, or an epitope thereof.

In another aspect, provided herein is a pH-modulating pharmacologic agent for use in a method for controlling the binding of a T cell and/or NK cell to a target mammalian cell in a subject in vivo, including administering the pH-modulating pharmacologic agent to the subject through an effective dosing regimen that increases the pH of a microenvironment within the subject, wherein the subject includes the T cell and/or the NK cell, wherein the T cell and/or NK cell expresses a conditionally active biologic chimeric antigen receptor (CAB-CAR) that binds to its cognate antigen differentially at pH 6.7 as compared to pH 7.4, wherein the cognate antigen is an Axl polypeptide or an epitope thereof, or a Ror2 polypeptide or an epitope thereof, wherein the T cell and/or NK cell expresses the CAB-CAR, wherein the microenvironment includes the target mammalian cell, wherein the target mammalian cell expresses the cognate antigen on their surface, and wherein the T cell and/or NK cell binds to the target mammalian cell differentially before versus after the pH of the microenvironment is increased by administering the pH-modulating pharmacologic agent thereby controlling the binding of the T cell and/or NK cell to the target mammalian cell in a subject in vivo.

In another aspect, provided herein is a pharmacologic agent for use in a method for modulating the binding of a conditionally active biologic chimeric antigen receptor (CAB-CAR) expressing T cell or NK cell to a cell expressing a cognate antigen of the CAB-CAR in a subject, for treating tumor growth, wherein the method includes:
a. introducing a T cell and/or NK cell capable of expressing the CAB-CAR into the subject, wherein the cognate antigen is an Axl polypeptide or an epitope thereof, or a Ror2 polypeptide or an epitope thereof, wherein the CAB-CAR binds to the cell expressing the cognate antigen in the subject,
wherein after the introducing, the T cell and/or the NK cell including the nucleic acid encoding the CAB-CAR expresses the CAB-CAR and binds to the cell expressing the cognate antigen in the subject; and
b. administering the pharmacologic agent to the subject in sufficient amount to increase blood pH and/or a tissue pH and/or a microenvironment pH to modulate binding of the CAB-CAR expressing T cell and/or NK cell to the cell expressing the cognate antigen in the blood, the tissue, or the microenvironment with the increased pH.

In another aspect, provided herein is a pharmacologic agent for use in a method for alleviating on target off tumor toxicity in a subject, wherein the method includes:
a. introducing a nucleic acid encoding a conditionally active biologic chimeric antigen receptor (CAB-CAR) into a T cell or NK cell of the subject, to produce a T cell and/or NK cell capable of expressing the CAB-CAR;
b. introducing the T cell and/or NK cell capable of expressing the CAB-CAR into the subject, wherein after the introducing, the T cell and/or the NK cell including the nucleic acid encoding the CAB-CAR expresses the CAB-CAR and binds to the cell expressing the cognate antigen in the subject; and
c. administering the pharmacologic agent to the subject in sufficient amount to increase blood pH and/or a tissue pH and/or a microenvironment pH to modulate binding of the CAB-CAR to its cognate antigen in the blood, the tissue, and/or the microenvironment with the increased pH, wherein the cognate antigen is an Axl polypeptide or an epitope thereof, or a Ror2 polypeptide or an epitope thereof, thereby alleviating on target off tumor toxicity in the subject.

In another aspect, provided herein is a pharmacologic agent for use in a method for controlling the binding of a T cell and/or NK cell expressing a conditionally active biologic chimeric antigen receptor (CAB-CAR) to a target mammalian cell, for treating tumor growth, wherein the method includes:
a. contacting the target mammalian cell with the T cell and/or NK cell expressing the CAB-CAR in a microenvironment, wherein the target mammalian cell expresses a cognate antigen, wherein the cognate antigen is an Axl polypeptide or an epitope thereof, or a Ror2 polypeptide or an epitope thereof, and wherein the T cell and/or NK cell expresses the CAB-CAR that binds to the cognate antigen differentially at pH 6.7 as compared to pH 7.4; and
b. increasing the pH of the microenvironment by introducing the pharmacologic agent to the microenvironment in sufficient amount, thereby controlling the binding of the T cell and/or NK cell expressing the CAB-CAR to the target mammalian cell.

In another aspect, provided herein is a pharmacologic agent for use in a method for controlling the binding of a T cell and/or NK cell expressing a conditionally active biologic chimeric antigen receptor (CAB-CAR) to a target mammalian cell in a subject in vivo, for treating tumor growth, wherein the pharmacologic agent is a pH-modulating pharmacologic agent, and wherein the method includes administering the pH-modulating pharmacologic agent to the subject through an effective dosing regimen that increases the pH of a microenvironment within the subject, wherein the subject includes the T cell and/or NK cell expressing the CAB-CAR, wherein the CAB-CAR binds to its cognate antigen differentially at pH 6.7 as compared to pH 7.4, wherein the cognate antigen is an Axl polypeptide or an epitope thereof, or a Ror2 polypeptide or an epitope thereof, wherein the microenvironment includes the target mammalian cell, wherein the target mammalian cell expresses the cognate antigen on its surface, and wherein the T cell and/or NK cell binds to the target mammalian cell differentially before versus after the pH of the microenvironment is increased.

In another aspect, provided herein is a pH-modulating pharmacologic agent for use in a method for controlling the binding of a T cell and/or NK cell expressing a conditionally active biologic chimeric antigen receptor (CAB-CAR) to a target mammalian cell in a subject in vivo, for treating tumor growth, wherein the method includes administering the pH-modulating pharmacologic agent to the subject through an effective dosing regimen that increases the pH of a microenvironment within the subject, wherein the subject includes the T cell and/or NK cell expressing the CAB-CAR, wherein the CAB-CAR binds to its cognate antigen differentially at pH 6.7 as compared to pH 7.4, wherein the cognate antigen is an Axl polypeptide or an epitope thereof, or a Ror2 polypeptide or an epitope thereof, wherein the microenvironment includes the target mammalian cell, wherein the target mammalian cell expresses the cognate antigen on its surface, and wherein the T cell and/or NK cell binds to the target mammalian cell differentially before versus after the pH of the microenvironment is increased by administering the pH-modulating pharmacologic agent.

In another aspect, provided herein is a use of a pH-modulating pharmacologic agent for use in the manufacture of a medicament for controlling the binding of a T cell and/or NK cell expressing a conditionally active biologic chimeric antigen receptor (CAB-CAR) to a target mammalian cell in a subject in vivo, wherein the pH-modulating pharmacologic agent is to be administered to the subject through an effective dosing regimen that increases the pH of a microenvironment within the subject, wherein the subject includes the T cell and/or NK cell expressing the CAB-CAR, wherein the CAB-CAR binds to its cognate antigen differentially at pH 6.7 as compared to pH 7.4, wherein the cognate antigen is an Axl polypeptide or an epitope thereof, or a Ror2 polypeptide or an epitope thereof, wherein the microenvironment includes the target mammalian cell, wherein the target mammalian cell expresses the cognate antigen on their surface, and wherein the T cell binds to the target mammalian cell differentially before versus after the pH of the microenvironment is increased by administering the pH-modulating pharmacologic agent.

In any of the aspects provided immediately above that include a pH-modulating pharmacologic agent or a pharmacologic agent for use in a method and a CAB-CAR or include the use of a pH-modulating pharmacologic agent and a CAB-CAR, the CAB-CAR can have reduced binding to its cognate antigen at one pH than at a different pH. In illustrative embodiments where illustrative pH values for differential binding of a CAB-CAR are not already provided in the broadest aspect and alternatively for other embodiments in place of those values for such aspects, the CAB-CAR can have reduced binding at a higher pH than at a lower pH. For example, the CAB-CAR can have reduced binding to its cognate antigen at a pH above 7.0, 7.1, 7.2, 7.3, 7.4, or 7.5 than at a pH below 6.4, 6.5, 6.6, 6.7, 6.8, 6.9, or 7.0. In other embodiments, the CAB-CAR can have reduced binding at a higher pH than at a lower pH. For example, the CAB-CAR can have reduced binding to its cognate antigen at a pH below 6.4, 6.5, 6.6, 6.7, 6.8, 6.9, or 7.0 than at a pH above 7.0, 7.1, 7.2, 7.3, 7.4, or 7.5. In some illustrative embodiments, the CAB-CAR exhibits increased binding at a pH of 6.5 to 6.7 compared to pH 7.4 to 7.6. In other illustrative embodiments, the CAB-CAR exhibits increased binding at a pH of 6.7 compared to a pH of 7.4. In other embodiments, the CAB-CAR exhibits increased binding in the pH of a tumor compared to the pH of blood. In some embodiments, the CAB-CAR can include an antigen-specific targeting region, a stalk, and an intracellular activating domain. In some embodiments, the CAB-CAR can also include a co-stimulatory domain. In some embodiments, the CAB-CAR can bind to a tumor associated antigen.

In any of the aspects provided immediately above that include a pH-modulating pharmacologic agent or a pharmacologic agent for use in a method and a CAB-CAR or include the use of a pH-modulating pharmacologic agent and a CAB-CAR, the pH of the microenvironment can be increased from a pH below 7.0 to a pH above 7.0. For example, the pH can be increased from a pH below 6.4, 6.5, 6.6, 6.7, 6.8, 6.9, or 7.0 to a pH above 7.0, 7.1, 7.2, 7.3, or 7.4. In some embodiments, the CAB-CAR can bind to the cognate antigen at the increased pH but not a pH of the microenvironment before introducing the pharmacologic agent. In certain embodiments, the pH can be increased from below 7.0 to a pH of 7.1 to 8.0 or to a pH of 7.1 to 7.8 or to a pH of 7.2 to 7.8 or a pH of 7.2 to 7.6 or a pH of 7.3 to 7.6 or to a pH of 7.4 to 7.8 or to a pH of 7.4 to 7.6. Such an increase in pH can occur for less than 1, 2, 4, 6, 8, 12, or 24 hours or for more than 1, 2, 4, 6, 8, 12 or 24 hours depending on the type and dose of pharmacologic agent administered. In certain embodiments, the pharmacologic agent is administered such that the pH remains above 7.0, 7.1, 7.2, 7.3, 7.4, or 7.5; or between 7.0, 7.1, 7.2, 7.3 on the low end of the range and 7.4, 7.5, 7.6, 7.7, or 7.8 on the high end of the range, in the target tissue, such as a tumor, and for example in at least a surface of a target tissue (e.g. tumor) microenvironment, in at least a portion of a target tissue (e.g. tumor) microenvironment, and in illustrative embodiments throughout a target tissue (e.g. tumor) microenvironment.

In any of the aspects provided immediately above that include a pH-modulating pharmacologic agent or a pharmacologic agent for use in a method and a CAB-CAR or include the use of a pH-modulating pharmacologic agent and a CAB-CAR, wherein the cognate antigen is an Axl polypeptide or an epitope thereof, or a Ror2 polypeptide or an epitope thereof; the microenvironment can be an in vivo microenvironment, such as a tumor, a tissue, a non-tumor tissue, a normal tissue, or a tissue that has undergone a transient shift in pH. For example, tissues that typically undergo transient shifts in pH include a muscle tissue in anaerobic conditions or muscle tissue undergoing exercise or an inflamed tissue or a tissue experiencing inflammation. In some embodiments that include a target mammalian cell, the target mammalian cell can be a tumor cell or a non-tumor or normal cell.

In any of the aspects provided immediately above that include a pH-modulating pharmacologic agent or a pharmacologic agent for use in a method and a CAB-CAR or include the use of a pH-modulating pharmacologic agent and a CAB-CAR, wherein the cognate antigen is an Axl polypeptide or an epitope thereof, or a Ror2 polypeptide or an epitope thereof, the pharmacologic agent can be sodium bicarbonate, tris-hydroxylmethyl aminomethane, an equimolar hypertonic solution of sodium bicarbonate and sodium carbonate, or proton pump inhibitors such esomeprazole, esomeprazole and naproxen, lansoprazole, omeprazole, and rabeprazole.

In any of the aspects provided immediately above that include a pH-modulating pharmacologic agent or a pharmacologic agent for use in a method and a CAB-CAR or include the use of a pH-modulating pharmacologic agent and a CAB-CAR, wherein the cognate antigen is an Axl polypeptide or an epitope thereof, or a Ror2 polypeptide or an epitope thereof, the pharmacologic agent can be used in a method for the treatment of cancer, tumors, tumor growth, or a cell proliferative disorder.

In another aspect, provided herein is a kit containing a container containing a replication incompetent recombinant retroviral particle, and instructions for use thereof for treating tumor growth, wherein the instructions instruct a method for controlling the binding of a T cell and/or NK cell to a target mammalian cell, in a method including:
a. transducing the T cell and/or NK cell with the replication incompetent recombinant retroviral particle including in its genome a conditionally active biologic chimeric antigen receptor (CAB-CAR) that binds to the cognate antigen differentially at pH 6.7 as compared to pH 7.4 to produce a T cell and/or NK cell capable of expressing the CAB-CAR, wherein the cognate antigen is an Axl polypeptide or an epitope thereof, or a Ror2 polypeptide or an epitope thereof;
b. introducing the T cell and/or NK cell capable of expressing the CAB-CAR into the subject, wherein after the introducing, the T cell and/or the NK cell including the nucleic acid encoding the CAB-CAR expresses the CAB-CAR and binds to the cell expressing the cognate antigen in the subject;
c. contacting the target mammalian cell with the CAB-CAR expressing T cell and/or NK cell in a microenvironment, wherein the target mammalian cell expresses a cognate antigen of the CAB-CAR, and the T cell and/or NK cell expresses the CAB-CAR; and
d. increasing the pH of the microenvironment by introducing a pH-modulating pharmacologic agent to the microenvironment in sufficient amount, thereby affecting the binding of the target mammalian cell with the T cell and/or NK cell.

In some embodiments, the kit can further include a pH-modulating pharmacologic agent.

In some embodiments of the kit, the CAB-CAR can have reduced binding to an Axl polypeptide or an epitope thereof, or a Ror2 polypeptide or an epitope thereof at one pH than at a different pH. In illustrative embodiments where illustrative pH values for differential binding of a CAB-CAR are not already provided in the broadest aspect and alternatively for other embodiments in place of those values for such aspects, the CAB-CAR can have reduced binding at a higher pH than at a lower pH. For example, the CAB-CAR can have reduced binding to its cognate antigen at a pH above 7.0, 7.1, 7.2, 7.3, 7.4, or 7.5 than at a pH below 6.4, 6.5, 6.6, 6.7, 6.8, 6.9, or 7.0. In other embodiments, the CAB-CAR can have reduced binding at a higher pH than at a lower pH. For example, the CAB-CAR can have reduced binding to its cognate antigen at a pH below 6.4, 6.5, 6.6, 6.7, 6.8, 6.9, or 7.0 than at a pH above 7.0, 7.1, 7.2, 7.3, 7.4, or 7.5. In some illustrative embodiments, the CAB-CAR exhibits increased binding at a pH of 6.5 to 6.7 compared to pH 7.4 to 7.6. In other illustrative embodiments, the CAB-CAR exhibits increased binding at a pH of 6.7 compared to a pH of 7.4. In other embodiments, the CAB-CAR exhibits increased binding in the pH of a tumor compared to the pH of blood. In some embodiments, the CAB-CAR can include an antigen-specific targeting region, a stalk, and an intracellular activating domain. In some embodiments, the CAB-CAR can also include a co-stimulatory domain. In some embodiments, the CAB-CAR can bind to a tumor associated antigen.

In some embodiments of the kit, the pH of the microenvironment can be increased from a pH below 7.0 to a pH above 7.0. For example, the pH can be increased from a pH below 6.4, 6.5, 6.6, 6.7, 6.8, 6.9, or 7.0 to a pH above 7.0, 7.1, 7.2, 7.3, or 7.4. In some embodiments, the CAB-CAR can bind to the cognate antigen at the increased pH but not a pH of the microenvironment before introducing the pharmacologic agent. In certain embodiments, the pH can be increased from below 7.0 to a pH of 7.1 to 8.0 or to a pH of 7.1 to 7.8 or to a pH of 7.2 to 7.8 or a pH of 7.2 to 7.6 or a pH of 7.3 to 7.6 or to a pH of 7.4 to 7.8 or to a pH of 7.4 to 7.6. Such an increase in pH can occur for less than 1, 2, 4, 6, 8, 12, or 24 hours or for more than 1, 2, 4, 6, 8, 12 or 24 hours depending on the type and dose of pharmacologic agent administered. In certain embodiments, the pharmacologic agent is administered such that the pH remains above 7.0, 7.1, 7.2, 7.3, 7.4, or 7.5; or between 7.0, 7.1, 7.2, 7.3 on the low end of the range and 7.4, 7.5, 7.6, 7.7, or 7.8 on the high end of the range, in the target tissue, such as a tumor, and for example in at least a surface of a target tissue (e.g. tumor) microenvironment, in at least a portion of a target tissue (e.g. tumor) microenvironment, and in illustrative embodiments throughout a target tissue (e.g. tumor) microenvironment. In some embodiments, the microenvironment can be an in vivo microenvironment, such as a tumor, a tissue, a non-tumor tissue, a normal tissue, or a tissue that has undergone a transient shift in pH. For example, tissues that typically undergo transient shifts in pH include a muscle tissue in anaerobic conditions or muscle tissue undergoing exercise or an inflamed tissue or a tissue experiencing inflammation. In some embodiments that include a target mammalian cell, the target mammalian cell can be a tumor cell or a non-tumor or normal cell.

In some embodiments of the kit, the pharmacologic agent can be sodium bicarbonate, tris-hydroxylmethyl aminomethane, an equimolar hypertonic solution of sodium bicarbonate and sodium carbonate, or proton pump inhibitors such esomeprazole, esomeprazole and naproxen, lansoprazole, omeprazole, and rabeprazole.

As disclosed above, the cognate antigen to which the CAB-CAR binds can be an Axl polypeptide or an epitope thereof or a Ror2 polypeptide or an epitope thereof. In some embodiments, the cognate antigen can be a polypeptide with at least 50%, 55%, 60%, 65%, 70%, 75%, 80%, 85%, 90%, 95%, 96%, 97%, 98%, 99%, or 100% sequence identity to a stretch of at least 10, 15, 20, or all of the amino acids of an Axl polypeptide or an epitope thereof or to a Ror2 polypeptide or an epitope thereof. As disclosed herein, the CAB-CAR capable of binding to an Axl polypeptide or an epitope thereof, or a Ror2 polypeptide or an epitope thereof typically binds its cognate antigen with a higher binding affinity at a pH of 6.7 than a pH of 7.4.

A skilled will recognize that when the specification refers to “Axl CAB” or “Axl CAB-CAR” the “Axl” refers to an ASTR that recognizes Axl or an epitope thereof. Similarly, a skilled will recognize that when the specification refers to “Ror2 CAB” or “Ror2 CAB-CAR” the “Ror2” refers to an ASTR that recognizes Ror2 or an epitope thereof.

Any section headings used herein are for organizational purposes only and are not to be construed as limiting the described subject matter in any way.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); i.v., intravenous(ly); and the like.

### Example 1: Production and Analysis of Chimeric Antigen Receptors for Targeting Axl and Ror2

This example demonstrates methods for making and using conditionally active CARs of certain embodiments of the present invention. Nucleic acids encoding conditionally active ASTRs targeting Axl and Ror2 were obtained and used to generate lentiviral expression vectors encoding conditionally active CARs. T cells were transduced with lentivirus containing these expression vectors and the transduced cells were tested in various in vitro tumor surrogate assays against target cells.

### Generation of Lentiviral Expression Vectors Expressing Conditionally Active CAR

A nucleic acid encoding a conditionally active ASTR targeting Axl (an scFv construct containing SEQ ID NOs:79 and 80) and 3 nucleic acids encoding conditionally active ASTRs targeting Ror2 (an scFv construct containing SEQ ID NOs:83 and 84, an scFv construct containing SEQ ID NOs:82 and 84, and an scFv construct containing SEQ ID NOs:151 and 152) were obtained. The detailed description herein provides methods for making conditionally active ASTRs (sometimes referred to as conditionally active biologics (CABs)), including methods for making CABs by evolving and selecting antibody fragments that was used to make the CABs that were obtained. Minor modifications to the sequences were made, including codon optimization for expression in human cells and the removal of splice donor and acceptor sites. The heavy chains of the single-chain antibodies (SEQ ID NOs:79, 83, and 82) and the light chains of the single-chain antibodies (SEQ ID NOs:80 and 84) were joined by linkers of various lengths (SEQ ID NOs: 53, 54, and 55) and incorporated into lentiviral expression vectors with the other domains to make the candidate CARs listed in Tables 1 and 2.

### Production of Lentiviruses

Lentiviruses were produced by transient transfection of 293T cells (Lenti-X 293T, Clontech) with the lentiviral expression vectors. The cells were adapted to suspension culture by serial growth in Freestyle 293 Expression Medium (Thermo Fisher Scientific). The cells in suspension were transfected using PEI (Polysciences) dissolved in weak acid. Cells (30 mL) were grown to 1×10⁶ cells/mL in a 125 mL Erlenmeyer flask.

Plasmid DNA was diluted in 1.5 ml Opti-MEM media for 30 mL of cells. Total DNA (1 μg/mL of culture volume) was a mixture of 4 plasmids with the following molar ratios: 2× genomic plasmid, 1× Rev-containing plasmid, 1×VSVg-containing plasmid, and 1× Gagpol-containing plasmid. Separately, the PEI was diluted in 1.5 ml Opti-MEM to 2 μg/mL (culture volume, 2:1 ratio to DNA). After a 5 minute room temperature incubation, the two solutions were mixed together well and incubated at room temperature for 20 more minutes. The final volume (3 ml) was added to the cells. The cells were then incubated at 37° C. for 72 hours with rotation at 120 rpm and with 5-8% CO₂.

After 72 hours, the supernatant was harvested by centrifugation at 1,000 g for 10 minutes. The supernatant was decanted to a fresh tube and % of the supernatant volume in PEG solution (PEG-IT, System Biosciences) was added. The virus was precipitated by incubation overnight at 4° C. followed by centrifugation at 1,500 g for 20 minutes at 4° C. The supernatant was removed, and the virus was resuspended in 1:100 volume of Ex-Vivol5 media. Viruses were titered by GFP on HT1080 cells or by eTAG expression on Jurkat cells.

### T Cell Transduction/Expansion for Axl and Ror2

In an initial method for T cell transduction and expansion Pan-T cells were obtained from All Cells (Allcells, PB009-1F). Anti-Axl CAB-CAR and Anti-ROR2 CAB-CAR replication incompetent recombinant lentiviral particles were made as discussed above. Two days prior to lentiviral transduction, cells were thawed and cultured in human T cell medium, consisting of X-VIVO15 (Lonza #04-418Q), 5% human AB serum (Valley Biomedical Inc., #HP1022), and 10 mM N-acetyl L-Cysteine (Sigma-Aldrich #A9165). Recombinant human IL-2 (R&D 202-IL-010) was added to a final concentration of 100 IU/mL. Twenty-four hours prior to viral transduction, primary human T cells were seeded into 12-well plate at 0.5×10⁶ cells/well and activated using Dynabeads Human T-Activator CD3/CD28 (Thermo #1113ID) at a 1:3 cell:bead ratio. On the day of transduction, lentiviral particle solution was added to the wells at MOI 5 for Axl and MOI 20 for Ror2. Transduced T cells were maintained at ˜10⁶/mL in human T cell medium for 3 days, then transferred into 6-well-G-Rex with 30 mL/well of human T cell medium contain IL-2. Cells were cultured for at least 10 days before experiments were conducted and IL-2 was added every other day.

Certain follow-on experiments were performed with slightly modified transduction and expansion conditions. Where indicated, in place of Pan T cells from AllCells, T cells were enriched from buffy coats (San Diego Blood Bank) by density gradient centrifugation with Ficoll-Paque PREMIUM® (GE Healthcare Life Sciences) according to the manufacturer's instructions followed by lysis of red blood cells. To study conditional CAR dependent killing of the human kidney cell lines Caki-1 and HEK293, CD56+ cells were depleted from transduced Pan T cells (AllCells) using the EasySep Human CD56 Positive Selection Kit II (Stem Cell Technologies #17855) prior to expansion.

FACS Analysis of eTAG Expression

Transduced primary T cells (0.5×10⁶) were collected 8 days after transduction, washed and resuspended in FACS buffer (PBS+2% FBS+0.1% sodium azide). Cells were stained with 100 μL FACS buffer containing 0.9 μg/ml biotinylated-cetuximab (Promab) for 30 mins on ice. Stained cells were washed with FACS buffer and stained with Streptavidin PE (eBioscience, 12-4317-87, 0.2 mg/ml), CD3-BV421 (Biolegend, 317344), CD4-PE-Dazzle 594 (Biolegend, 300548), and CD8-BV570 (Biolegend, 301038) for 30 mins on ice. Cells were washed twice in FACS buffer, fixed in a 1:1 mixture of the FACS buffer and BD Cytofix (BD #554655), processed with Novocyte (ACEA), and the resulting data was analyzed with NovoExpress software (ACEA).

### Real-Time Cell Killing Assays

Cytotoxic activity of transduced T cells was measured by xCELLigence System (ACEA). Briefly, transduced primary T cells (effector cells) were produced as above, stored frozen, thawed and rested for 2 days in human T cell medium containing 100 IU/ML of IL-2. Target cells included CHO cells (ATCC) and CHO cells stably transfected to express human Axl (CHO-Axl) or ROR2 (CHO-ROR2) or in other experiments, human renal cell carcinoma lines Caki-1 (source) and HEK293 (Lenti-X 293T, Clontech) which tested positive by FACS for Axl and ROR2, respectively. Target cells were seeded to E-plates at 20K cells/well one day before the experiment with human T cell medium containing 40 mM HEPES and PIPES at pH 6.7 and 7.4 or at a range of pHs from pH 6.3 to pH 7.4 in 0.1 pH increments. On the day of the assay, rested effector cells were added into experimental wells at effector cell/target cell ratios (E/T) of 3:1, 1:1, and in some instances, 0.3:1. Impedance readings were taken every 5 minutes for approximately 40 hours after effector cell addition and impedance was reported as the Cell Index(CI). Percentage of specific cytolysis was calculated as follows ((CI Target+Control virus transduced effector T cells)−(CI Target+effector T cells transduced with CARs directed to Axl or Ror2))/(CI Target+Control virus transduced effector T cells)×100.

### Results

Chimeric antigen receptor (CARs) for binding Axl or Ror2 with increased activity at the reduced pH of a tumor environment compared to normal tissue (sometimes referred to herein as (CAB-CARs) were made by incorporating the heavy chains and light chains of conditionally active single-chain antibodies into lentiviral expression vectors along with other CAR domains and an eTag domain. The wild-type control CARS and conditionally active (CAB) CARs that were made and tested included various combinations of different CAR modules/components as indicated in Table 1 (Axl) and Table 2 (Ror2) with further detailed sequence information provided in Table 3. The various combinations of modules from amino to carboxy terminus are indicated in Table1 and Table 2. These modules included a CD8 signal sequence peptide (“sp”) (P1) (SEQ ID NO:74); Axl CAB VH (SEQ ID NO:79), Axl CAB VL (SEQ ID NO:80), Axl VH (SEQ ID NO:93), Axl VL (SEQ ID NO:94), Ror2 VH (SEQ ID NO: 107), Ror2 VL (SEQ ID NO:84), Ror2 CAB VL (SEQ ID NO: 152) Ror2 CAB1 VH (SEQ ID NO:82), Ror2 CAB2 VH (SEQ ID NO:83), Ror2 CAB3 VH (SEQ ID NO:151) (P2) and (P4); linker 1 (SEQ ID NO:53), linker 2 (SEQ ID NO:54), or linker 3 (SEQ ID NO:55) (P3); stalk/hinge and transmembrane domain from CD8 (SEQ ID NO:75) or CD28 (SEQ ID NO:76) (P5); co-stimulatory domain from CD137 (SEQ ID NO: 1), CD28 (SEQ ID NO:2), ICΔ (SEQ ID NO:3), or both ICΔ and CD137 (SEQ ID NO: 133) (P6); activation domain from CD3Z (SEQ ID NO: 13) (P7); a 2A-1 ribosomal skip sequence (SEQ ID NO:77) (P8); an eTAG (SEQ ID NO:78) (P9).

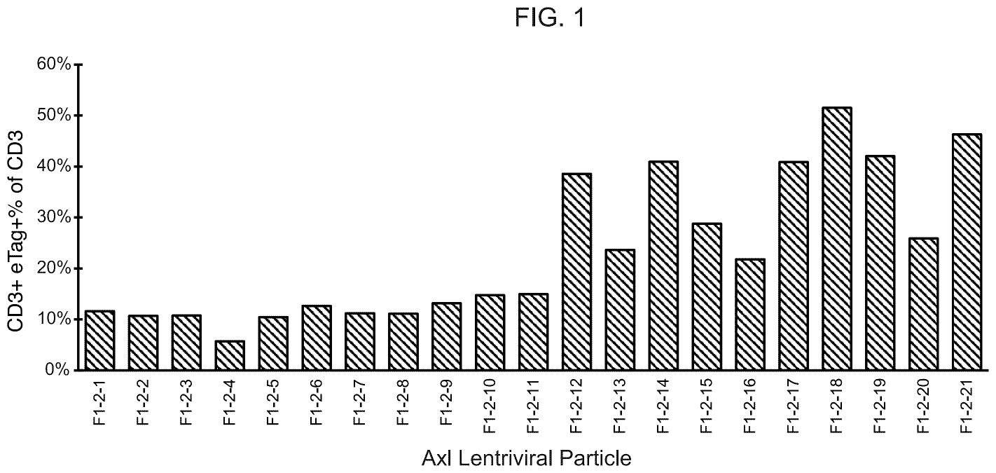

Primary T cells were transduced with the recombinant lentiviral particles expressing the candidate CARs of Table 1 and Table 2 and the percent transfected cells was determined by determining the percent of cells expressing the eTag using FACS. Primary T cells were successfully transduced with the recombinant lentiviral particles encoding the candidate CARs as shown for representative Axl CARs in FIG. 1.

The cytotoxic activity of the candidate CARs against target cells expressing either Axl or Ror2 was analyzed at a pH of 7.4 (physiological pH) or a pH of 6.7 (surrogate tumor assay condition). As explained in more detail below, many of the candidate CARs were more effective at lysing target cells at a pH of 6.7 than a pH of 7.4. CAB activity was not detected in control CARs that included fully wild-type heavy and light chains (i.e. both VH and VL chains that were identified under physiologic conditions and were not further evolved).

In certain initial experiments related to Axl, eighteen anti-Axl CAB-CARs (F1-2-1 to F1-2-18) were tested using a first conditionally active heavy chain and a first conditionally active light chain that were both evolved from wild type heavy and light chain, respectively. As shown in Table 1, nine of the eighteen anti-AXL CAB-CARs initially tested had detectable cytotoxic activity (indicated in the table as “CAB activity”). None of the CAB-CARs tested that had only wild type ASTR antibody heavy and light chains had conditional activity (identified in Table 1 as “Axl VH” and “Axl VL” (F1-2-19, F1-2-20)). In follow-on experiments using the same transduction and expansion conditions provided above, anti-Axl CAB-CARs that included different co-stimulatory domains exhibited conditional cytotoxic activity (F1-2-22 and F1-2-23).

In certain initial experiments related to Ror2, conditionally active ASTRs against two different epitopes were analyzed. With respect to Ror2 CAB-CARs against epitope 1 (F1-1-9 to F1-1-24), eleven of the sixteen anti-Ror2 CAB-CARs initially tested had conditional cytotoxic activity (Table 2). With respect to Ror2 CAB-CARs against epitope 2, two (F1-1-25 and F1-1-26) of six anti-Ror2 CAB-CARs that exhibited killing activity in initial experiments, had conditional cytotoxic activity (Table 2). None of the CAB-CARs tested that had only wild type ASTR antibody heavy and light chains had conditional activity in these killing assays (identified in Table 2 as “Ror2 VH” and “Ror2 VL” (F1-1-1 to F1-1-8)).

In summary, many of the candidate CAB-CARs made and tested had higher cytotoxic activity on the target cells at a pH of 6.7 than at a pH of 7.4. Exemplary CAB-CARs that were more effective at lysing target cells at a pH of 6.7 than at a pH of 7.4 included CAB-CAR F1-2-3, which included an anti-Axl ASTR (SEQ ID NO: 159), CAB-CAR F1-2-8, which included an anti-Axl ASTR (SEQ ID NO: 160), CAB-CAR F1-2-10, which included an anti-Axl ASTR (SEQ ID NO:161), CAB-CAR F1-2-13, which included an anti-Axl ASTR (SEQ ID NO: 129), CAB-CAR F1-2-15, which included an anti-Axl ASTR (SEQ ID NO: 128), CAB-CAR F1-1-9, which included an anti-Ror2 ASTR (SEQ ID NO:157), CAB-CAR F-1-11, which included an anti-Ror2 ASTR (SEQ ID NO: 153), CAB-CAR F-1-15, which included an anti-Ror2 ASTR (SEQ ID NO: 132), CAB-CAR F-1-17, which included an anti-Ror2 ASTR (SEQ ID NO: 154), CAB-CAR F-1-19, which included an anti-Ror2 ASTR (SEQ ID NO: 130), CAB-CAR F1-1-21, which included an anti-Ror2 ASTR (SEQ ID NO: 158), CAB-CAR F1-1-23, which included an anti-Ror2 ASTR (SEQ ID NO:131), CAB-CAR F-1-25, which included an anti-Ror2 ASTR (SEQ ID NO: 155), and CAB-CAR F-1-26, which included an anti-Ror2 ASTR (SEQ ID NO: 156). The ASTRs all included at least one module of either the VH or VL that was identified under physiologic conditions and was further evolved.

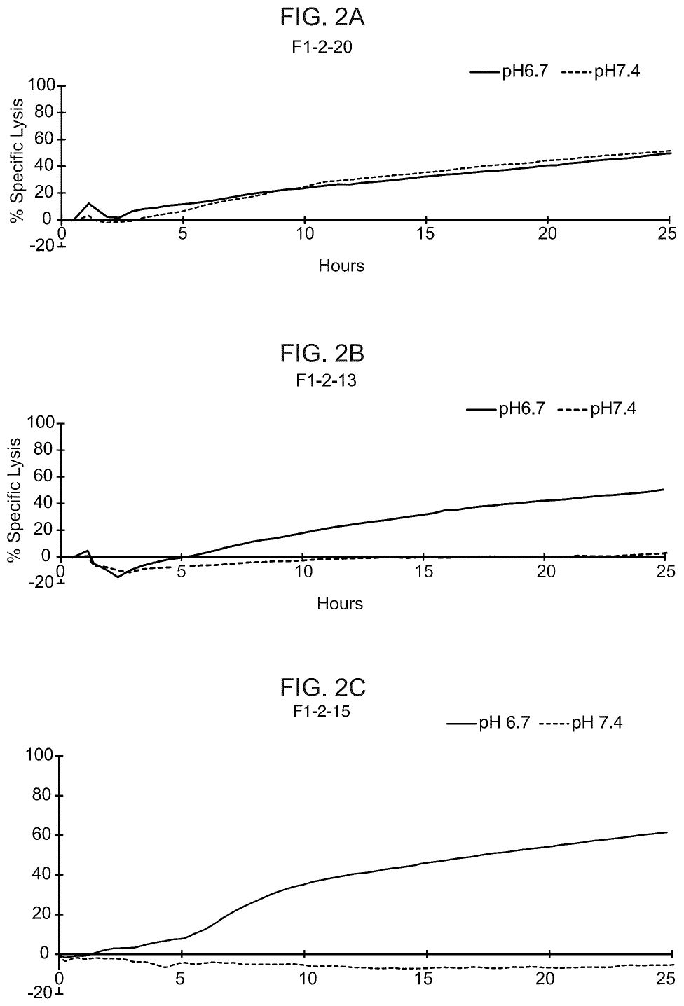

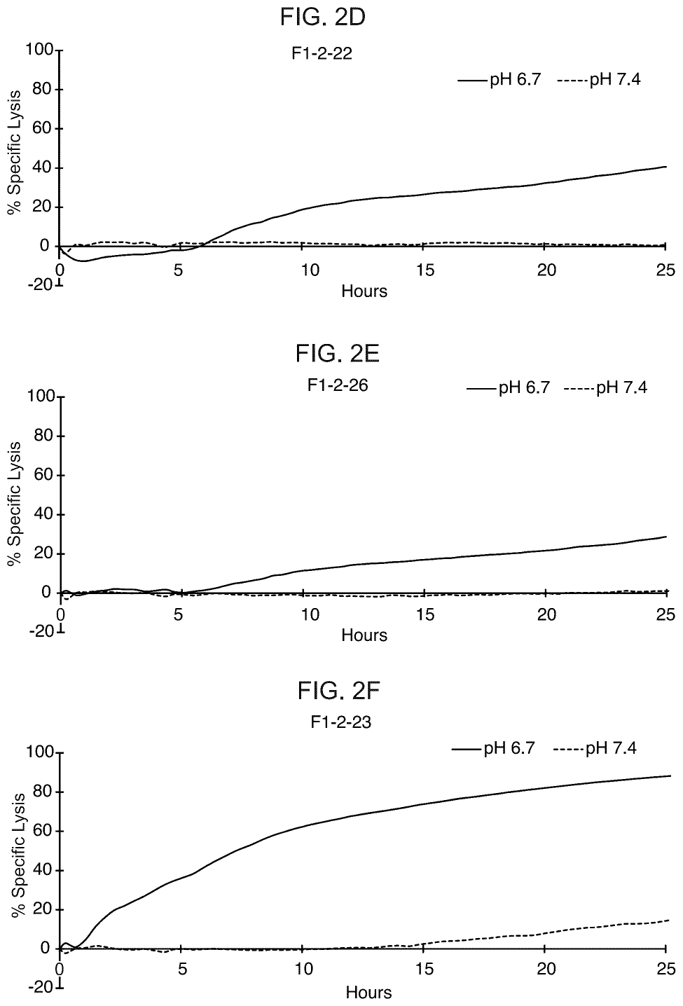

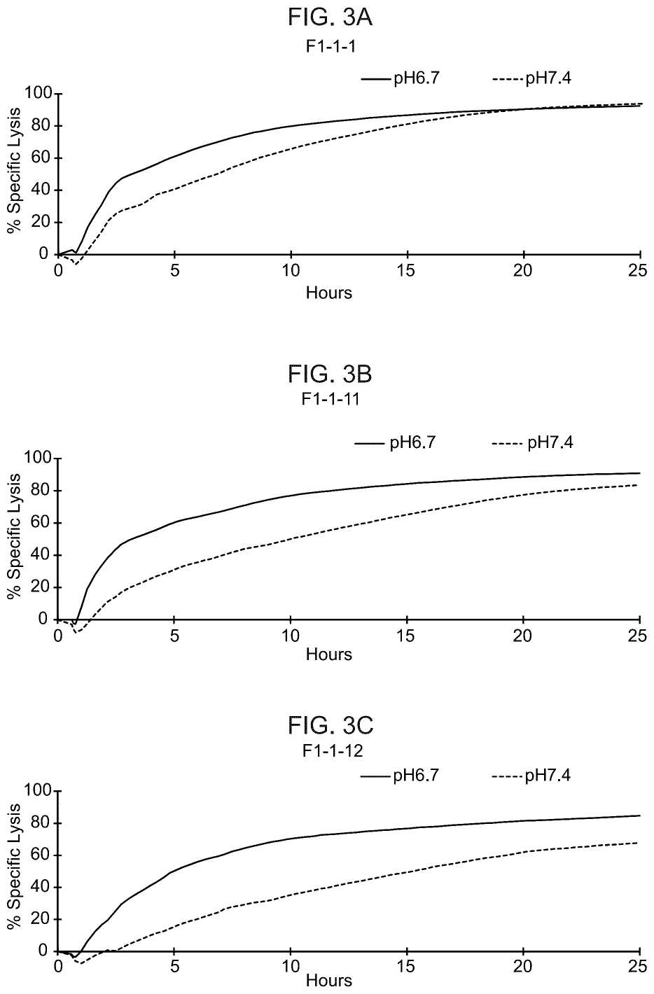

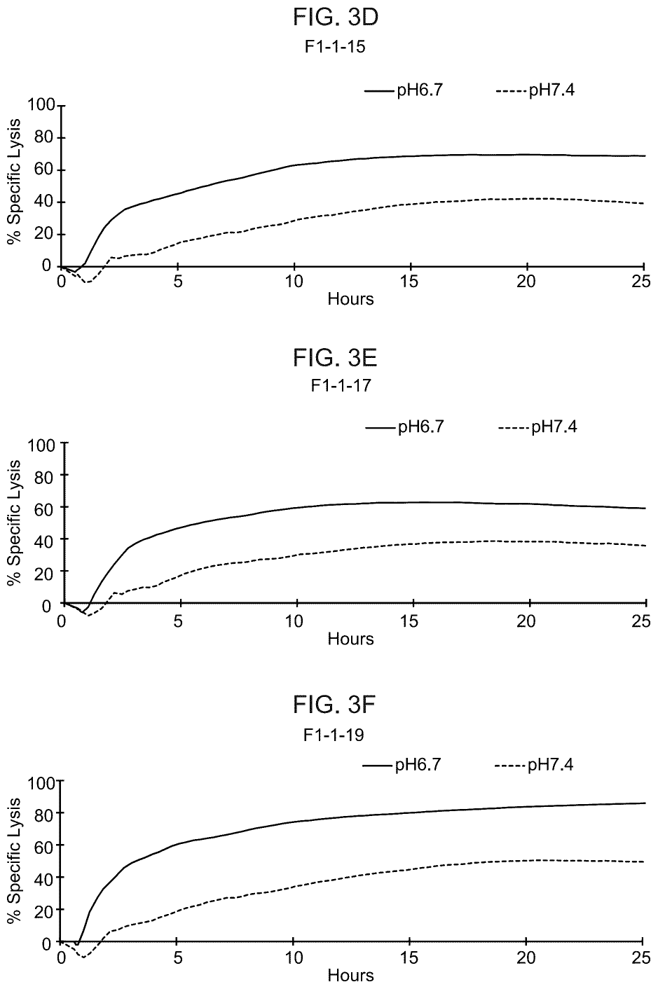

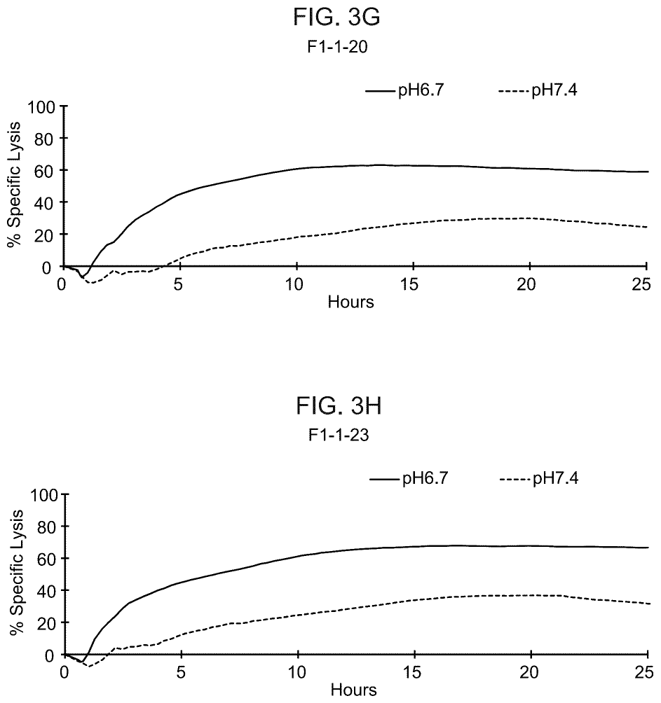

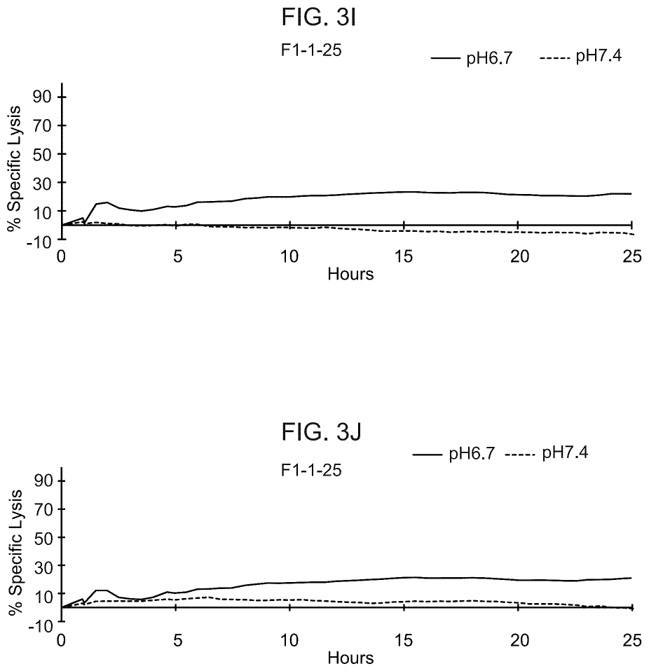

FIGS. 2B-2F show representative in vitro killing of CHO-Axl tumors over time for some of the more conditionally active CAB-CARs against Axl versus a control CAR having a wild-type ASTR that is not conditionally active (FIG. 2A). A 3:1 ratio of effector to target cells was used in FIGS. 2A and 2B. A 1:1 ratio of effector to target was used in FIGS. 2C-2F. Similarly, FIGS. 3B-3J show representative in vitro killing of CHO-Ror2 tumors over time at a 3:1 ratio of effector to target cells for some of the more conditionally active CAB-CARs against Ror2 versus a control CAR having a wild-type ASTR that is not conditionally active (FIG. 3A).

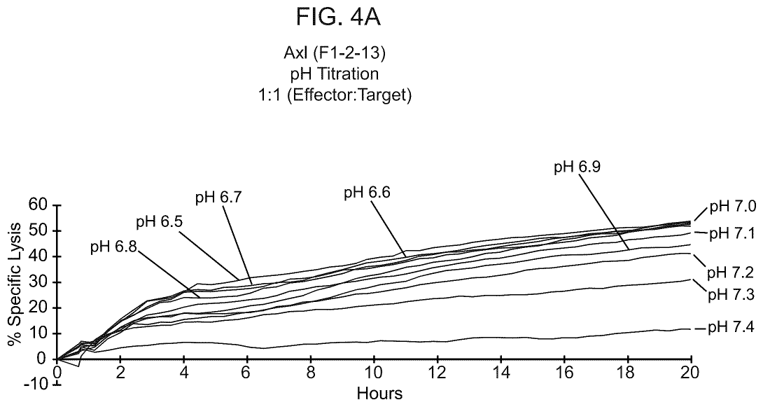

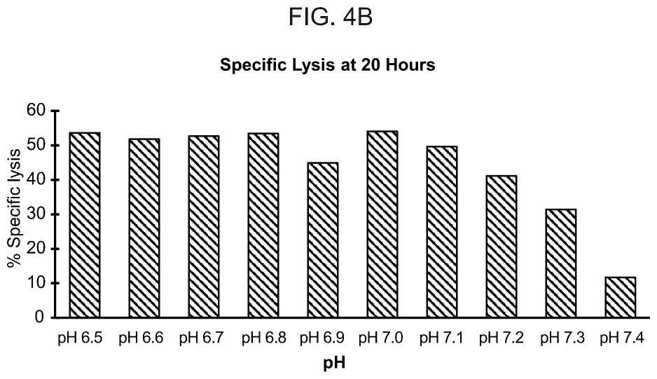

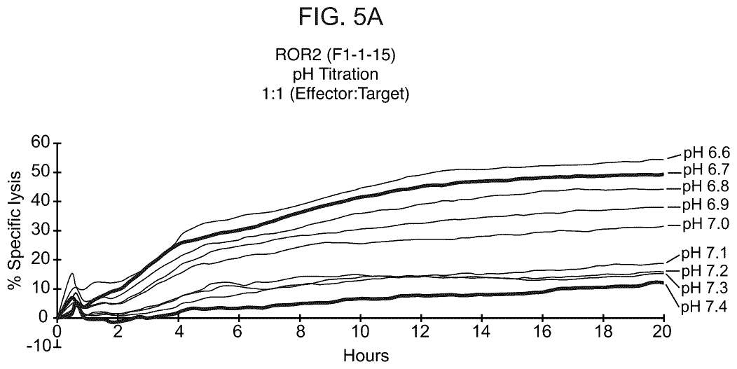

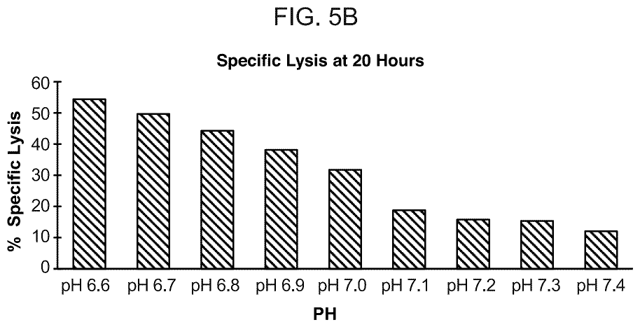

The conditional activity of some of the candidate CARs was analyzed at a range of pHs from 7.4 to 6.7 in pH increments of 0.1 pH (pH titration) over time. As shown in the representative curves for CAB-CAR F1-2-13 (AXL) in FIG. 4A and CAB-CAR F1-1-15 (Ror2) in FIG. 5A, all of the candidate CARs tested (5 CAB-CARs directed to each of Axl and Ror2) that included the conditionally active ASTRs (CAB-CARs) demonstrated pH-dependent killing of CHO cells expressing their cognate antigen, that was low at physiologic pH and increased as the culture became more acidic. FIG. 4B shows that the pH dependent killing by CAB-CAR F1-2-13 increased from pH 7.4 to pH 7.0 and was consistently high from pH 7.0 to pH 6.5. FIG. 5B shows that the pH dependent killing by CAB-CAR F1-1-15 increased gradually from pH 7.4 to pH 6.6.

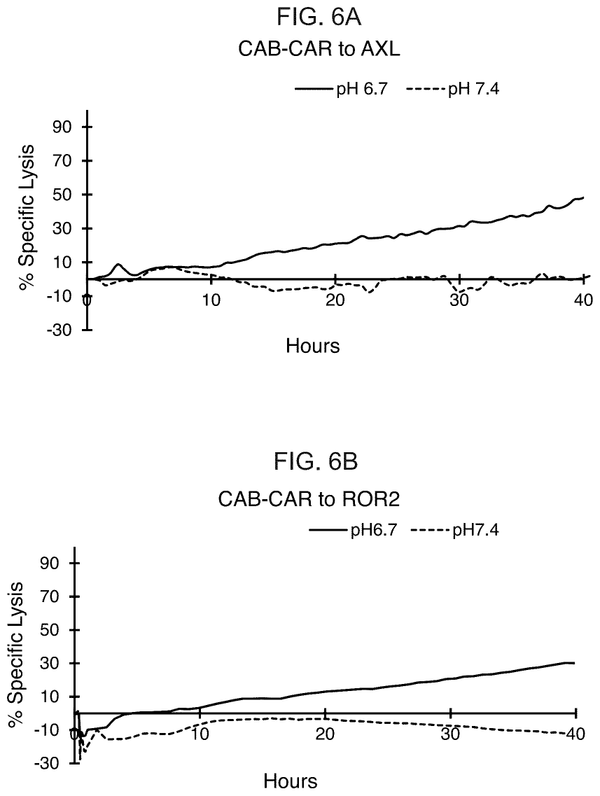

The conditional killing of candidate CAB-CARs was also tested on human kidney cells that express endogenous levels of Axl and Ror2 in the absence of transgene expression of these proteins. FIG. 6A shows conditional killing of Axl-expressing Caki-1 cells by T cells expressing F1-2-15. FIG. 6B shows conditional killing of Ror2-expressing HEK293 cells by T cells expressing F1-1-15.

Thus, the methods provided herein were effective at making and identifying conditionally active CARs against Axl and Ror2 that demonstrated conditional killing activity when expressed on T cells in vitro.

TABLE 1
Real-time cell analysis results for T cells expressing various anti-Axl CARs.

| | Modules | | | | | | | | | CAB |
|---|---|---|---|---|---|---|---|---|---|---|
| ID | P1 | P2 | P3 | P4 | P5 | P6 | P7 | P8 | P9 | activity |
| F1-2-1 | CD8 sp | Axl CAB VH | Linker 1 | Axl CAB VL | CD8 | CD137 | CD3Z | 2A-1 | eTAG | Medium |
| F1-2-2 | CD8 sp | Axl CAB VH | Linker 1 | Axl CAB VL | CD28 | CD137 | CD3Z | 2A-1 | eTAG | Medium |
| F1-2-3 | CD8 sp | Axl CAB VH | Linker 2 | Axl CAB VL | CD8 | CD137 | CD3Z | 2A-1 | eTAG | Medium |
| F1-2-4 | CD8 sp | Axl CAB VH | Linker 2 | Axl CAB VL | CD28 | CD137 | CD3Z | 2A-1 | eTAG | None |
| F1-2-5 | CD8 sp | Axl CAB VH | Linker 3 | Axl CAB VL | CD8 | CD137 | CD3Z | 2A-1 | eTAG | None |
| F1-2-6 | CD8 sp | Axl CAB VH | Linker 3 | Axl CAB VL | CD28 | CD137 | CD3Z | 2A-1 | eTAG | Medium |
| F1-2-7 | CD8 sp | Axl CAB VL | Linker 1 | Axl CAB VH | CD8 | CD137 | CD3Z | 2A-1 | eTAG | None |
| F1-2-8 | CD8 sp | Axl CAB VL | Linker 1 | Axl CAB VH | CD28 | CD137 | CD3Z | 2A-1 | eTAG | Medium |
| F1-2-9 | CD8 sp | Axl CAB VL | Linker 2 | Axl CAB VH | CD8 | CD137 | CD3Z | 2A-1 | eTAG | None |
| F1-2-10 | CD8 sp | Axl CAB VL | Linker 2 | Axl CAB VH | CD28 | CD137 | CD3Z | 2A-1 | eTAG | Medium |
| F1-2-11 | CD8 sp | Axl CAB VL | Linker 3 | Axl CAB VH | CD8 | CD137 | CD3Z | 2A-1 | eTAG | None |
| F1-2-12 | CD8 sp | Axl CAB VL | Linker 3 | Axl CAB VH | CD28 | CD137 | CD3Z | 2A-1 | eTAG | None |
| F1-2-13 | CD8 sp | Axl CAB VH | Linker 1 | Axl CAB VL | CD28 | ICΔ | CD3Z | 2A-1 | eTAG | High |
| F1-2-14 | CD8 sp | Axl CAB VH | Linker 2 | Axl CAB VL | CD28 | ICΔ | CD3Z | 2A-1 | eTAG | Medium |
| F1-2-15 | CD8 sp | Axl CAB VH | Linker 3 | Axl CAB VL | CD28 | ICΔ | CD3Z | 2A-1 | eTAG | High |
| F1-2-16 | CD8 sp | Axl CAB VL | Linker 1 | Axl CAB VH | CD28 | ICΔ | CD3Z | 2A-1 | eTAG | None |
| F1-2-17 | CD8 sp | Axl CAB VL | Linker 2 | Axl CAB VH | CD28 | ICΔ | CD3Z | 2A-1 | eTAG | None |
| F1-2-18 | CD8 sp | Axl CAB VL | Linker 3 | Axl CAB VH | CD28 | ICΔ | CD3Z | 2A-1 | eTAG | None |
| F1-2-19 | CD8 sp | Axl VH | Linker 3 | Axl VL | CD8 | CD137 | CD3Z | 2A-1 | eTAG | None |
| F1-2-20 | CD8 sp | Axl VH | Linker 3 | Axl VL | CD28 | CD137 | CD3Z | 2A-1 | eTAG | None |
| F1-2-21 | CD8 sp | Axl VH | Linker 3 | Axl VL | CD28 | ICΔ | CD3Z | 2A-1 | eTAG | None |
| F1-2-22 | CD8 sp | Axl CAB VH | Linker 3 | Axl CAB VL | CD28 | ICΔCD137 | CD3Z | 2A-1 | eTAG | High |
| F1-2-23 | CD8 sp | Axl CAB VH | Linker 3 | Axl CAB VL | CD28 | CD28 | CD3Z | 2A-1 | eTAG | High |

TABLE 2
Real-time cell analysis results for T cells expressing various anti Ror2 CARs.

| | Modules | | | | | | | | | CAB |
|---|---|---|---|---|---|---|---|---|---|---|
| ID | P1 | P2 | P3 | P4 | P5 | P6 | P7 | P8 | P9 | activity |
| F1-1-1 | CD8 sp | Ror2 VH | Linker 1 | Ror2 VL | CD8 | CD137 | CD3Z | 2A-1 | eTAG | none |
| F1-1-2 | CD8 sp | Ror2 VH | Linker 1 | Ror2 VL | CD28 | CD137 | CD3Z | 2A-1 | eTAG | none |
| F1-1-3 | CD8 sp | Ror2 VH | Linker 2 | Ror2 VL | CD8 | CD137 | CD3Z | 2A-1 | eTAG | none |
| F1-1-4 | CD8 sp | Ror2 VH | Linker 2 | Ror2 VL | CD28 | CD137 | CD3Z | 2A-1 | eTAG | none |
| F1-1-5 | CD8 sp | Ror2 VL | Linker 1 | Ror2 VH | CD8 | CD137 | CD3Z | 2A-1 | eTAG | none |
| F1-1-6 | CD8 sp | Ror2 VL | Linker 1 | Ror2 VH | CD28 | CD137 | CD3Z | 2A-1 | eTAG | none |
| F1-1-7 | CD8 sp | Ror2 VL | Linker 2 | Ror2 VH | CD8 | CD137 | CD3Z | 2A-1 | eTAG | none |
| F1-1-8 | CD8 sp | Ror2 VL | Linker 2 | Ror2 VH | CD28 | CD137 | CD3Z | 2A-1 | eTAG | none |
| F1-1-9 | CD8 sp | Ror2 CAB1 VH | Linker 1 | Ror2 VL | CD8 | CD137 | CD3Z | 2A-1 | eTAG | Medium |
| F1-1-10 | CD8 sp | Ror2 CAB1 VH | Linker 1 | Ror2 VL | CD28 | CD137 | CD3Z | 2A-1 | eTAG | Medium |
| F1-1-11 | CD8 sp | Ror2 CAB1 VH | Linker 2 | Ror2 VL | CD8 | CD137 | CD3Z | 2A-1 | eTAG | High |
| F1-1-12 | CD8 sp | Ror2 CAB1 VH | Linker 2 | Ror2 VL | CD28 | CD137 | CD3Z | 2A-1 | eTAG | High |
| F1-1-13 | CD8 sp | Ror2 VL | Linker 1 | Ror2 CAB1 VH | CD8 | CD137 | CD3Z | 2A-1 | eTAG | none |
| F1-1-14 | CD8 sp | Ror2 VL | Linker 1 | Ror2 CAB1 VH | CD28 | CD137 | CD3Z | 2A-1 | eTAG | none |
| F1-1-15 | CD8 sp | Ror2 VL | Linker 2 | Ror2 CAB1 VH | CD8 | CD137 | CD3Z | 2A-1 | eTAG | high |
| F1-1-16 | CD8 sp | Ror2 VL | Linker 2 | Ror2 CAB1 VH | CD28 | CD137 | CD3Z | 2A-1 | eTAG | none |
| F1-1-17 | CD8 sp | Ror2 CAB2 VH | Linker 1 | Ror2 VL | CD8 | CD137 | CD3Z | 2A-1 | eTAG | High |
| F1-1-18 | CD8 sp | Ror2 CAB2 VH | Linker 1 | Ror2 VL | CD28 | CD137 | CD3Z | 2A-1 | eTAG | Medium |
| F1-1-19 | CD8 sp | Ror2 CAB2 VH | Linker 2 | Ror2 VL | CD8 | CD137 | CD3Z | 2A-1 | eTAG | High |
| F1-1-20 | CD8 sp | Ror2 CAB2 VH | Linker 2 | Ror2 VL | CD28 | CD137 | CD3Z | 2A-1 | eTAG | High |
| F1-1-21 | CD8 sp | Ror2 VL | Linker 1 | Ror2 CAB2 VH | CD8 | CD137 | CD3Z | 2A-1 | eTAG | Medium |
| F1-1-22 | CD8 sp | Ror2 VL | Linker 1 | Ror2 CAB2 VH | CD28 | CD137 | CD3Z | 2A-1 | eTAG | none |
| F1-1-23 | CD8 sp | Ror2 VL | Linker 2 | Ror2 CAB2 VH | CD8 | CD137 | CD3Z | 2A-1 | eTAG | High |
| F1-1-24 | CD8 sp | Ror2 VL | Linker 2 | Ror2 CAB2 VH | CD28 | CD137 | CD3Z | 2A-1 | eTAG | none |
| F1-1-25 | CD8 sp | Ror2 CAB VL | Linker 1 | Ror2 CAB3 VH | CD28 | CD137 | CD3Z | 2A-1 | eTAG | Medium |
| F1-1-26 | CD8 sp | Ror2 CAB VL | Linker 2 | Ror2 CAB3 VH | CD28 | CD137 | CD3Z | 2A-1 | eTAG | Medium |

### Example 2: Elimination of eTAG Expressing Target Cells

### Effect of Cetuximab Concentration on Elimination

PBMCs were isolated from ACD-peripheral blood from a healthy donor by Ficoll-Paque Premium (GE Healthcare, 45-001-751). To prepare target cells, PBMCs were transduced with lentivirus, which contain coding regions for GFP and eTag operably linked to a promoter, to express GFP fluorescent protein and an eTag target. PBMCs activated and expanded at the same time without transduction were used as nontarget control cells. Expanded target cells and nontarget control cells were stored in freezing medium in liquid nitrogen until use. Freshly isolated PBMCs from the same donor were pre-stimulated overnight with 10 ng/mL huGM-CSF (R&D Systems, 215-GM-010) and used as effector cells. Target cells and nontarget cells were allowed to recover for one day from cryopreservation and then nontarget control cells were labeled with CT-Violet before use. Target cell and nontarget control cells were mixed at a 1:1 ratio and then were co-incubated with effector PBMCs at 50:1 effector cell:target cell ratio in a U-bottom 96 well plate, with different concentrations of cetuximab or isotype control antibody. After incubation for 22 hours, samples were centrifuged at 400 g for 5 minutes. Pelleted cells were resuspended in FACS wash buffer (PBS+2% FBS+0.1% sodium azide) and fixed with an equal volume of BD Cytofix (BD #554655) prior to flow cytometry. Control samples containing only the target cells were used to set FACS gating. The numbers of target cells and nontarget control cells were quantified. A ratio of the surviving target cells (GFP+) to non-target control cells (CT-Violet) was calculated and normalized to samples without Ab. Percentage of depletion was calculated as follows: 1-((Target/NonTarget)/(Target without Ab/Non-target without Ab)).

### Effect of Ratios of Effector Cells:Target Cells on Cetuximab Elimination

The experiment was performed as described above except the PBMCs used were previously frozen and the target cells and nontarget control cells were mixed at a 1:1 ratio and then co-incubated with effector PBMCs at 50:1, 25:1, 5:1, and 1:1 effector cell:target cell ratios in a U-bottom 96 well plate, with 1 μg/mL of cetuximab or isotype control.

### Results

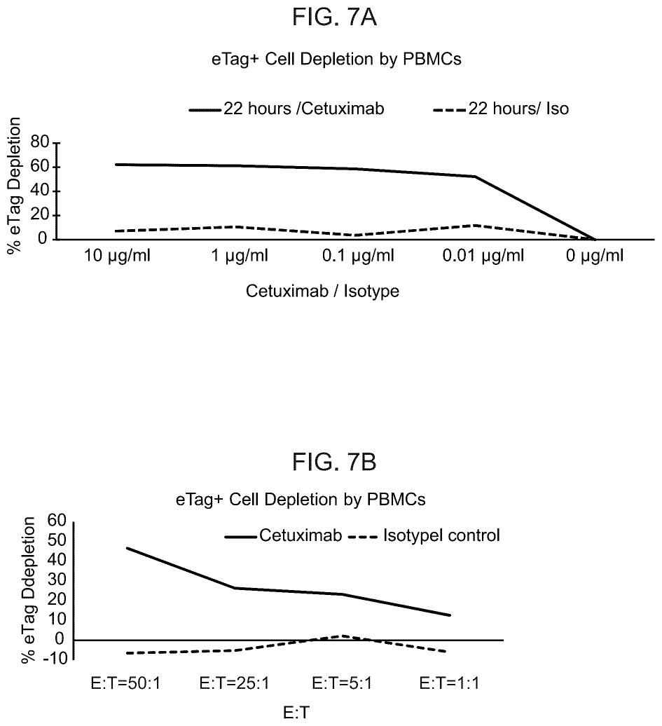

The effectiveness of expression of an eTag as an elimination domain was tested in peripheral blood mononuclear cells (PBMCs). PBMCs were isolated and transduced with a lentiviral vector that included an eTag operably linked to a promoter. Control cells were not transduced. Cells were treated with Cetuximab at concentrations from 0-10 μg/ml and cell depletion was detected at 22 hours after Cetuximab administration at all concentrations tested (FIG. 7A). Using a 1 μg/ml Cetuximab concentration, depletion was observed at all ratios of transduced effector (E) vs control (T) cells with generally increasing depletion with increasing ratio of transduced effector (E) cells (FIG. 7B). Thus, T cells expressing an eTag can be effectively eliminated by antibody to the eTag in the presence of PBMCs. It is noteworthy that follow-up experiments were performed with a FLAG epitope in which the FLAG epitope was expressed as part of the CAB-CAR polypeptide positioned between a CD8 signal peptide and a conditionally active ASTR of CAB-CARs F1-1-15 and F1-2-15. Although slightly diminished in some cases with these initial non-optimized constructs, these CAB-CARs retained their ability to kill target cells expressing their cognate antigen and to elicit cytokine responses. Thus, an elimination domain can be expressed as part of a CAB-CAR without destroying its activity.

### Example 3. Conditionally Active Chimeric Antigen Receptor Induction of Cytokines

This example demonstrates pH-dependent cytokine induction by conditionally active chimeric antigen receptors targeting Axl or Ror2. The cytokine levels of IL-2 and IFN-γ in the media were measured after a pH 6.7 or pH 7.4 co-incubation of control CHO cells or CHO cells expressing Axl (CHO-AXL) or Ror2 cells (CHO-ROR2) with T cells expressing a CAB-CAR with an anti-Axl ASTR (F1-2-15) or T cells expressing a CAB-CAR with an anti-Ror2 ASTR (F1-1-15), respectively. The cytokine levels from these co-incubations were compared with cytokine levels in negative and positive controls.

Cryopreserved effector cells were produced in advance by transducing Pan T cells (AllCells) with lentiviral particles encoding the CAB-CARs F1-1-15 (Ror2), F1-2-15 (Axl), or eTag alone (F1-0-01), and expanded as described in Example 1. On Day 1, effector cells were thawed and allowed to recover from cryopreservation for 2 days by culture at 37° C. and 5% C02 in human T cell medium consisting of X-VIV015 (Lonza #04-418Q), 5% human AB serum (Valley Biomedical Inc., #HP1022), 10 mM N-acetyl L-Cysteine (Sigma-Aldrich #A9165), and supplemented with 100 IU/ml IL2. On Day 2, 3.0×10⁴ CHO, CHO-Ror2, or CHO-Axl target cells were seeded in the wells of a 96 well flat bottom tissue culture plate in 100 μl human T cell medium containing 40 mM HEPES/PIPES and adjusted to pH 6.7 or pH 7.4 and cultured overnight at 37° C. and 5% CO₂. On Day 3, rested effector cells were added to experimental wells at an effector cell/target cell ratio of 3:1 in 140 μl of human T cell medium at the appropriate pH. Effector and target cells were co-incubated overnight at 37° C. and 5% CO2 and the supernatant was collected the next day and assayed for cytokine levels by ELISA. Each experimental condition was run in triplicate.

### Results

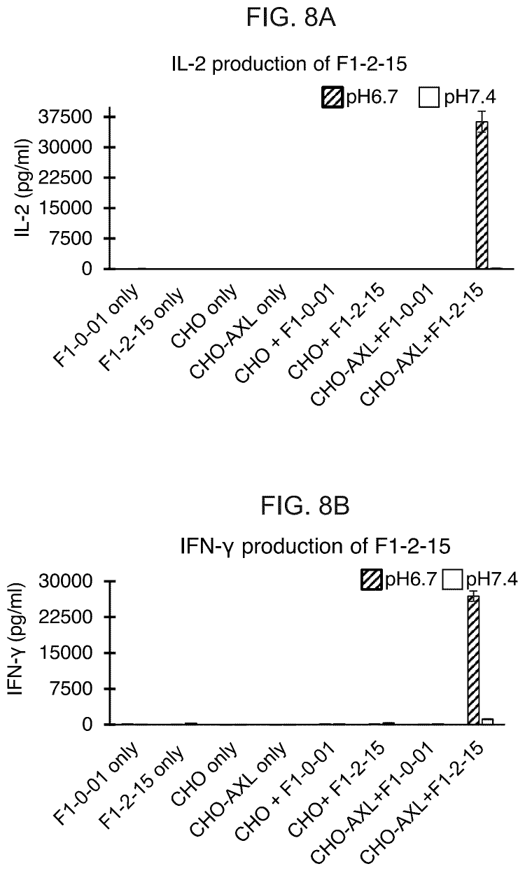

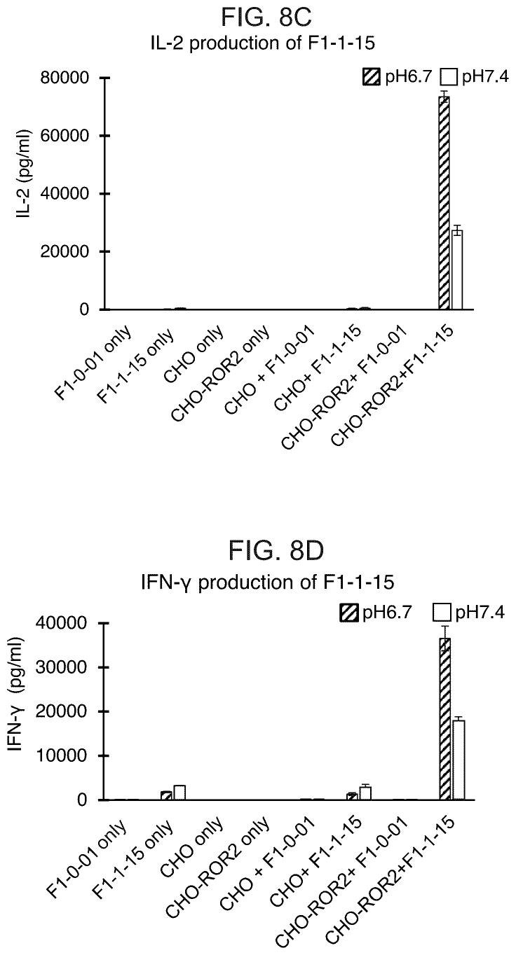

As shown in FIGS. 8A-8D, T cells transduced with a lentiviral particle containing a nucleic acid encoding the anti-Axl CAB-CAR F-2-15 or the anti-Ror2 CAB-CAR F-1-15 were activated to secrete cytokines in response to recognizing CHO cells expressing Axl or Ror2 respectively, in a pH dependent manner. As shown in FIG. 8A, T cells transduced with a lentiviral particle containing a nucleic acid encoding the anti-Axl CAB-CAR F1-2-15 secreted IL-2 when cultured at a pH of 6.7 but not at a pH of 7.4, and only in the presence of CHO cells expressing Axl. Similarly, as shown in FIG. 8B, T cells transduced with a lentiviral particle containing a nucleic acid encoding the anti-Axl CAB-CAR F1-2-15 secreted IFN-γ when cultured at a pH of 6.7 but not at a pH of 7.4, and only in the presence of CHO cells expressing Axl. As shown in FIG. 8C, T cells expressing the anti-Ror2 CAB-CAR F-1-15 secreted approximately 2.5-fold as much IL-2 when cultured at pH 6.7 as compared to pH 7.4, and only secreted appreciable amounts of IL-2 in the presence of CHO cells expressing Ror2. Similarly, as shown in FIG. 8D, T cells expressing the anti-Ror2 CAB-CAR F-1-15 secreted approximately 2-fold as much IFN-γ when cultured at pH 6.7 as compared to pH 7.4, and only in the presence of CHO cells expressing Ror2. Samples that included effector T cells that expressed a control EGFR polypeptide instead of the anti-Axl or anti-Ror2 CAB-CARs did not increase secretion of IL2 or IFN-γ in response to co-culture with any of the CHO cells at either pH. Thus, exemplary conditionally active CARs against Axl and Ror2 identified herein when expressed on T cells in vitro, elicited conditional cytokine secretion from these T cells when exposed to the CARs cognate antigen. Furthermore, these experiments provide non-limiting exemplary in vitro methods for activating T cells that express CAB-CARs provided herein.

### Example 4. Cytotoxic Activity of Conditionally Active Biologic Car-Expressing T Cells can be Controlled by Changing pH

The following example illustrates how the cytotoxic activity of transduced T cells (also referred to as effector cells) expressing CAB-CARs can be modulated by changes in the pH of the microenvironment. In this example, nucleic acids encoding a CAB-CAR capable of binding the cognate antigen Axl (anti-Axl) were used to generate replication incompetent recombinant lentiviral particles. Pan T cells were transduced with the lentiviral particles and the cytotoxic activity of the effector cells were compared using Real-Time Cell Analysis (RTCA) before and after changing the pH of the media.

### Production of CAB-CAR Effector T Cells

Lentiviral particles encoding a CAB-CAR directed to Axl (F1-2-15) and a negative control (C1) containing an eTag (SEQ ID NO:78) but lacking a CAR (F1-0-01) were produced as described in Example 1. These lentiviral particles were used to transduce Pan T cells (AllCells) and the transduced T cells were allowed to expand for 10 to 12 days as described in Example 1. These transduced T cells were cryopreserved for later use as effector cells.

### pH Shift Cytotoxicity Assay

The cytotoxic activity of transduced T cells before and after pH change by addition of NaHCO₃ or NaOH was measured using the xCELLigence System. Briefly, one day before the experiment, target cells (CHO cells stably transfected with a construct to express Axl on the cell surface (CHO-Axl cells)), were seeded into a 96-well E-plate (ACEA; San Diego, CA) at 10,000 cells/well with X-VIVO 15 media containing 40 mM HEPES and 40 mM PIPES, pH 6.7. Cryopreserved effector cells previously transduced with either lentiviral particles containing the nucleic acid encoding F1-2-15 or C1 (F1-2-15-VP and C1VP, respectively) produced as discussed above, were thawed and cultured for two days in X-VIVO 15 media containing 100 IU/mL of IL-2 (R&D Systems, Minneapolis, MN). On the day of the experiment, cells transduced with F1-2-15-VP or C1VP were washed and resuspended in X-VIVO 15 media containing 40 mM HEPES and 40 mM PIPES, pH 6.7 and then added into the experimental wells at effector cell/target cell ratios (E/T) of 1:1.

Impedance readings measured on the xCELLigence System (ACEA) were taken every 5 minutes and reported as the Cell Index (CI) to quantitate cell confluency as a measure of cell proliferation/cell lysis. Approximately 3 hours after effector cell addition, 8 μl of 7.5% NaHCO₃ or 14 μl of 0.5 M NaOH was added into the wells with X-VIVO 15 media containing 40 mM HEPES and 40 mM PIPES, pH 6.7 to increase the pH from 6.7 to 7.4. Impedance readings were continued for approximately 20 hours after effector cell addition. Percentage of specific cytolysis was calculated as follows ((CI Target+C1VP transduced effector T cells)−(CI Target+F1-2-15-VP transduced effector T cells))/(CI Target+C1VP transduced effector T cells)×100.

### HCl Switch on RTCA Killing Assay

The cytotoxic activity of transduced T cells before and after pH change by addition of HCl was measured using the xCELLigence System. Briefly, one day before the experiment, CHO-Axl cells were seeded into a 96 well E-plate at 10,000 cells/well with X-VIVO 15 media containing 40 mM HEPES and 40 mM PIPES, pH 7.4. Cryopreserved effector cells previously transduced with either C1VP or F1-2-15-VP, were thawed and cultured for two days in X-VIVO 15 media containing 100 IU/mL of IL-2. On the day of the experiment, cells transduced with F1-2-15-VP or C1VP were washed and resuspended in X-VIVO 15 media containing 40 mM HEPES and 40 mM PIPES, pH 7.4 and then added into experimental wells at effector cell/target cell ratios (E/T) of 1:1.

Impedance readings were taken every 5 minutes and reported as the Cell Index (CI). Approximately 3 hours after effector cell addition, 8 μl of 1 M HCl was added into the wells with X-VIVO 15 media containing 40 mM HEPES and 40 mM PIPES, pH 7.4 to switch the pH from 7.4 to 6.7. Impedance readings were continued for approximately 20 hours after effector cell addition. Percentage of specific cytolysis was calculated as follows ((CI Target+C1VP transduced effector T cells)−(CI Target+F1-2-15-VP transduced effector T cells))/(CI Target C1VP)×100.

### Results

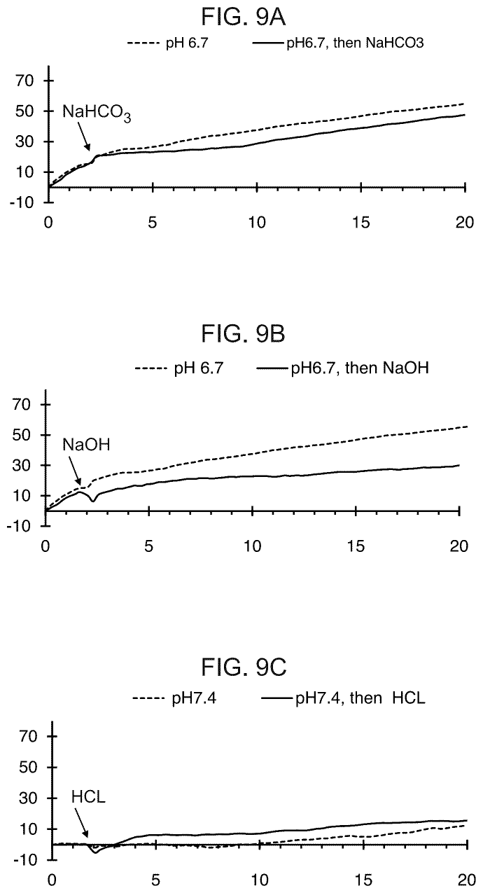

The cytotoxic activity of a CAB-CAR capable of binding cognate antigen Axl with increased activity at a reduced pH was compared in pH 6.7 and pH 7.4. T cells that were transduced with lentiviral particles containing a nucleic acid encoding F1-2-15, an anti-Axl CAB-CAR, were used to kill CHO cells expressing Axl, and then the pH was increased to determine whether the cytotoxic activity could be inhibited by a pH shift. As shown in FIGS. 9A and 9B, the addition of either NaHCO₃ or NaOH to the microenvironment of active CAR-T cells to increase the pH of the media inhibited the cytotoxic activity of the T cells expressing the CAB-CAR. These results show that active CAB-CAR expressing T cells can kill target-expressing cells and then this killing activity can be inhibited by increasing the pH of the microenvironment.

The ability of the cytotoxic activity of T cells expressing the CAB-CAR to be activated by a pH change was also determined. As shown in FIG. 9C, the cytotoxic activity of anti-Axl CAB-CAR expressing T cells on CHO-Axl cells was low at a pH of 7.4 and was increased by the addition of HCl to reduce the pH of the microenvironment.

Similar results were obtained when these experiments were repeated using F1-2-22 (CAB-CAR directed to Axl) and F1-1-15 (CAB-CAR directed to Ror2). Cumulatively, these results demonstrate the cytotoxic activity of T cells expressing CAB-CARs can be modulated by a shift in pH within the microenvironment, both by reducing cytotoxic activity after an increase in pH and increasing cytotoxic activity after a decrease in pH. In this non-limiting example, pH was increased from pH 6.7 and decreased from 7.4.

### Example 5. Bicarbonate Administration can Increase pH of the Tumor Microenvironment in Mice

The following example demonstrates the pH of an in vivo tumor microenvironment can be modulated by administering a pharmacologic agent. In this example, the pharmacologic agent is sodium bicarbonate and the tumor microenvironment is a CHO xenograft tumor in mice. The example includes two methods of measuring the pH of a tumor microenvironment, both in vivo and ex vivo.

The extracellular microenvironment of most solid tumors is acidic, with a pH typically between 6.5 and 6.9. On the contrary, normal tissue pH is basic, with a pH typically between 7.2 and 7.5. However, directly measuring the in vivo pH of a tumor microenvironment can be difficult. Fortunately, the relative protease activity of cathepsin is higher at lower pH and lower at higher pH. Therefore, the measurement of intratumoral cathepsin activity can serve as a surrogate measure of the pH of the tumor microenvironment. To measure in vivo activities of cathepsin B, L, S, K, V, and D, the near-infrared ProSense 750 FAST probe (PerkinElmer) was used. To further confirm modulation of the pH in the tumor microenvironment by administration of sodium bicarbonate, excised tumors were treated with phenol red and the color was noted. Phenol red is a pH indicator which undergoes a pH-dependent color transition. The sodium salt of phenol red is widely used in cell culture media to identify pH values. A solution of phenol red has a yellow color at a pH of 6.4 or below, an orange color around pH 7.0, a red color around pH 7.4, and a purple color above pH 7.8.

Mice were handled in accordance with Institutional Animal Care and Use Committee approved protocols. Subcutaneous (sc) Chinese Hamster Ovary (CHO) tumor xenografts were established in the hind flank of 12-14 week old female NOD-Prkdc^{scid}Il2rg^{tml}/Begen (B-NSG) mice (Beijing Biocytogen Co. Ltd.). Briefly, cultured CHO cells (ATCC, Manassas, VA) were washed in DPBS (Thermo Fisher), counted, resuspended in cold DPBS and mixed with an appropriate volume of Matrigel ECM (Corning; final concentration 5 mg/mL) at a concentration of 1.5×10⁶ cells/200 μl on ice.

Animals were prepared for injection using standard approved anesthesia with hair removal (Nair) prior to injection. 200 μl of the cell suspension in ECM was injected sc into the rear flanks of the mice. Once tumors were palpable, the tumors were measured using calipers 2 times/week. Tumor volume was calculated using the following equation: (longest diameter*shortest diameter²)/2. When average tumor volume reached 200 mm³, mice were randomly assigned to the respective treatment groups.

Two days before the administration of bicarbonate, the drinking water for the B-NSG mice was changed from acidic to regular pH autoclaved purified water. The following day, the 750 ProSense FAST probe was administered to 6 CHO-xenograft tumor bearing mice via 100 μl tail vein injections (4 nmol ProSense 750 FAST probe/100 μl PBS). A separate group of CHO-xenograft tumor bearing mice was left untreated. The following day, sodium bicarbonate was administered and imaging of the mice treated with the ProSense 750 FAST probe was performed using a Caliper IVIS Lumina XR. Briefly, mice were anesthetized using 3% O₂ 2 L/min isoflurane in 02 carrier gas at 2 L/min and then placed with nose cones supplying 1.5% isoflurane to anesthetized mice during imaging. Image acquisitions consisted of a 5 see exposure for near-infrared probes (745/810 nm excitation/emission wavelength). Fluorescence images were overlaid on normal light images of the mice. Time 0 (pretreatment) images were acquired before administration of either PBS (control) or sodium bicarbonate. The mice were then administered either 1 ml/mouse PBS (control, ThermoFisher) or 1 ml/mouse 1 M sodium bicarbonate (Shanghai Experiment Reagent Co., LTD) via intraperitoneal injection (ip). Mice were then imaged at 30 min post administration of PBS or bicarbonate. The collected fluorescence images were adjusted to have identical minimums, maximums, and threshold values. The photon counts were defined in this study as relative fluorescence units (RFU). RFU was calculated by normalizing the photon counts from the 30 min time point to the pretreatment time point (time 0; 100%) in each mouse. Due to variability between fluorescence values in each mouse at the time 0 pretreatment value, the observed fluorescence intensity values at different time points were normalized only to the individual mouse and not to a mean pretreatment value.

In a separate arm of the experiment, the 6 mice that did not receive the NIR cathepsin probe were euthanized by cervical dislocation at 1.5 hours post ip administration of PBS or sodium bicarbonate. The CHO xenograft tumor was excised from each mouse. The xenograft tumors were split into two halves with a scalpel and placed on a petri dish. The tumor tissue halves were then cut/sliced repeatedly using the scalpel. Water or 0.05% phenol red solution (50 mg phenol red/100 ml water) was added dropwise to each tumor half, respectively. The color was noted and images were taken of the treated tumor xenografts and of the phenol red solution remaining on the petri plate once the tumor xenograft samples were removed.

### Results

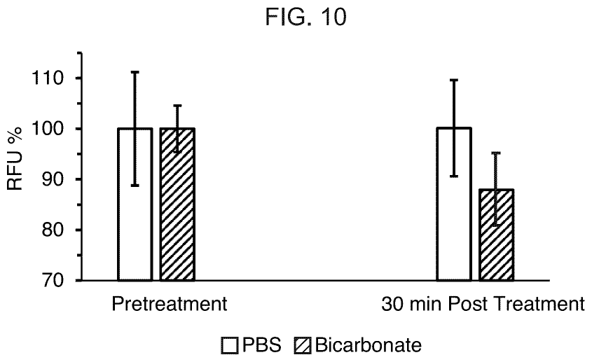

FIG. 10 shows the RFU results (mean with SEM) from imaging intratumoral cathepsin activity in CHO-xenograft tumor bearing mice before and after administration of PBS (control; n=3) or bicarbonate (n=3). These results suggest that sodium bicarbonate administration can increase the pH of the tumor microenvironment in vivo as evidenced by the decreased cathepsin activity observed following ip sodium bicarbonate administration.

A color change of the phenol red indicator from yellow/orange to red was observed using the tumor tissue excised from sodium bicarbonate-treated mice (n=3) relative to the PBS-treated mice (n=3). These results support that sodium bicarbonate administration increased the pH of the tumor microenvironment in vivo following ip administration as evidenced by the color change of the phenol red indicator from yellow/orange to red.

### Example 6. Target Dependent Killing of Tumors In Vivo by Cab-Cars Directed to Ror2 and Axl

Experiments were performed that demonstrated that CAB-CARs with conditional cytotoxic activity against Axl or Ror2 expressing cells in in vitro assays, had cytotoxic activity against Axl or Ror2 expressing tumor cells in vivo. A xenograft model using NSG, or NOD Scid Gamma mice was chosen that is non-reactive with anti-human scFv targets as a mechanism to probe the specificity and efficacy of CAB-CARs. NSG is a strain of mice that lack mature T cells, NK cells, and B cells and is among the most immunodeficient described to date. Removal of these cellular components is necessary to enable human peripheral blood derived mononuclear cells to engraft without innate, humoral or adaptive immune reactions from the host. Concentrations of homeostatic cytokines normally present only after radiation or lymphodepleting chemotherapy in humans is achieved due to the absence of the murine extracellular common gamma chain, which enables adoptively transferred human cells to receive such cytokines. At the same time, these animals can also be utilized to engraft tumor xenograft targets to examine the efficacy of CARs to kill target-expressing tumors. While the presence of xenoreactive T cell receptor antigens in the effector cellular product will eventually give rise to graft versus host disease, these models enable short term evaluation of animal pharmacology and acute tolerability.

Parental Chinese hamster ovary (CHO) cells and transgenic CHO cells stably transfected to express human ROR2 (CHO-ROR2) or human Axl (CHO-Axl) were utilized to generate a uniform target tumor to determine the specificity and efficacy of CAB-CAR effector cells to kill cognate antigen-expressing tumors. As all 3 cell lines have the same xenoreactive human leukocyte antigens (HLAs) and other non-specific antigens, the specificity of the CAB-CAR cells to tumor targets in the tumor microenvironment can be examined without consideration for variations in HLA reactivity of donor lymphocytes and target tumor cells. The parental and transgenic CHO variants grew rapidly with disseminated malignancy after subcutaneous administration into NSG mice in combination with Matrigel artificial basement membrane.

PBMCs were isolated from ACD-peripheral blood from healthy volunteers with informed consent by density gradient centrifugation with Ficoll-Pacque™ (General Electric) using a CS-900.2 kit (BioSafe; 1008) on a Sepax 2 S-100 device (Biosafe; 14000) according to the manufacturer's instructions. 5.0×10⁷ viable PBMCs were seeded in a 1 L G-Rex (Wilson-Wolf) and the volume was brought to 100 ml with Complete OpTmizer™ CTS™ T-Cell Expansion SFM supplemented with 100 IU/ml (IL-2) (Novoprotein), 10 ng/ml (IL-7) (Novoprotein), and 50 ng/ml anti-CD3 antibody (OKT3, Novoprotein) to activate the PBMCs for viral transduction. After incubation overnight at 37° C. and 5% CO₂, lentiviral particles encoding CAR constructs made as described in Example 1 were added directly to the activated PBMCs and incubated overnight at 37° C. and 5% CO₂. The following day, the media volume of each G-Rex brought to 1 L with Complete OpTmizer™ CTS™ T-Cell Expansion SFM supplemented with 10 mM NAC. Additionally, 100 IU/ml of IL-2 (Novoprotein) and 10 ng/ml IL-7 (Novoprotein) were added to each well on Day 2 and every 48 hours thereafter. The cells were allowed to expand up to Day 12 from the original blood collection (Day 0) before being harvested.

To examine CAB-CARs directed to ROR2, female NOD-Prkdc^{scid}Il2rg^{tm1}/Begen (B-NSG) mice (Beijing Biocytogen Co. Ltd.) 12-14 weeks old were inoculated subcutaneously with either parental CHO cells or CHO-ROR2 cells in Matrigel ECM at a concentration of 1.5×10⁶ cells/200 μl on ice as described in Example 5. Mice were dosed intravenously (IV) by tail vein injection with either 4×10⁶ T cells transduced with lentiviral particle containing a nucleic acid encoding F1-1-15 (n=6) or F1-0-01 (n=6) (See Examples 1 and 3 for construct details) prepared using the cell processing protocol provided in the above paragraph, or with PBS (n=6), when tumors had reached >200 mm³ to model heavy tumor burden therapy. Tumors were measured using calipers 3 times a week and tumor volume was calculated using the following equation: (longest diameter*shortest diameter²)/2. All animals reached necropsy guidelines from tumor burden by Day 13.

To examine CAB-CARs directed to AXL, female B-NSG mice 7-9 weeks old were inoculated subcutaneously with either parental CHO cells or CHO-AXL cells in Matrigel ECM (final concentration 5 mg/ml) at a concentration of 1.5×10⁶ cells/200 μl on ice as described in Example 5. IV or intratumoral (IT) administration of effector cells commenced when tumors reached >100 mm³ to model heavy tumor burden therapy. A first group of mice were dosed IV by tail vein injection with either 8×10⁶ T cells transduced with lentiviral particle containing a nucleic acid encoding F1-2-15 (n=6), F1-2-22 (n=6), or F1-0-01 (n=6) (See Examples 1 and 3 for construct details) prepared using the cell processing protocol provided in the above paragraph, or with PBS (n=6). A second group of mice were dosed IT with either 8×10⁶ T cells transduced with lentiviral particle containing a nucleic acid encoding F1-2-15 (n=6), F1-2-22 (n=6), or F1-0-01 (n=6), or with PBS (n=6). A third group of mice were dosed IV by tail vein injection with either 8×10⁶ T cells transduced with lentiviral particle containing a nucleic acid encoding F1-2-15 (n=6), F1-2-22 (n=6), or F1-0-01 (n=6), or with PBS ((n=6) and the mice received subcutaneous injections of IL-2 (50KIU) in the contralateral subcutaneous site away from the tumor every day for the first 3 days. Tumors were measured using calipers 3 times a week and tumor volume was calculated using the following equation: (longest diameter*shortest diameter²)/2. All animals were euthanized consistent with necropsy guidelines from tumor burden.

### Results

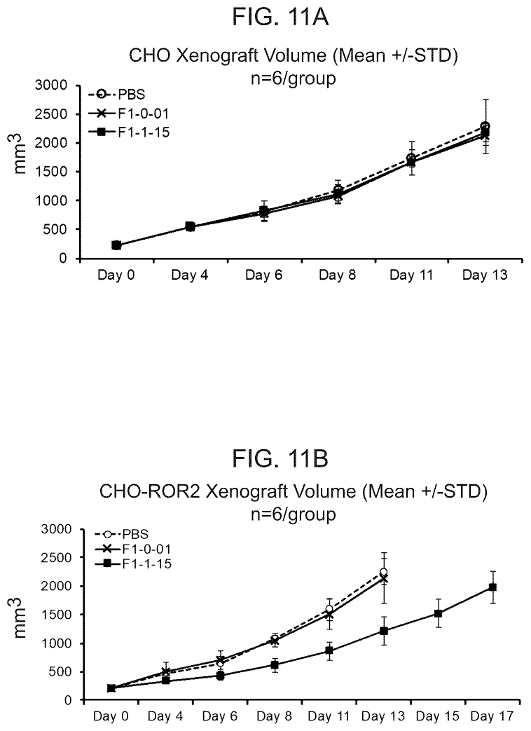

The in vivo tumor cell killing activity of some representative CAB-CARs disclosed in Example 1 was tested in a mouse model using engrafted tumor cells expressing Ror2, Axl, or neither. With respect to anti-Ror2 CAB-CARs, as shown in FIG. 11A, mice dosed IV with 4×10⁶ T cells transduced with a lentiviral particle containing a nucleic acid encoding F1-1-15 had no effect on ROR2 negative tumor growth compared to PBS controls or E-Tag only lentivirus transduced T cells (F1-0-01). In contrast, as shown in FIG. 11B, IV dosing of the same cell preparations into human Ror2-expressing tumors demonstrated significant tumor suppression of Ror2 positive tumors only by F1-1-15. These data demonstrate that the tumor microenvironment is reproduced in vivo in this engrafted tumor mouse model and that the genetically modified T cells expressing an exemplary anti-Ror2 CAB-CAR provided herein were capable of accessing the solid tumor and driving target cell killing in vivo despite the rapid doubling time of the target engrafted tumor cells.

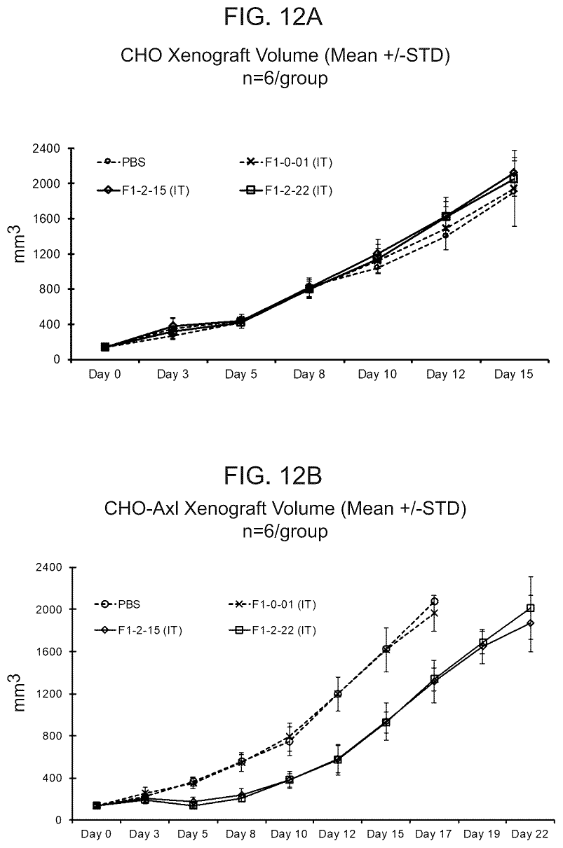

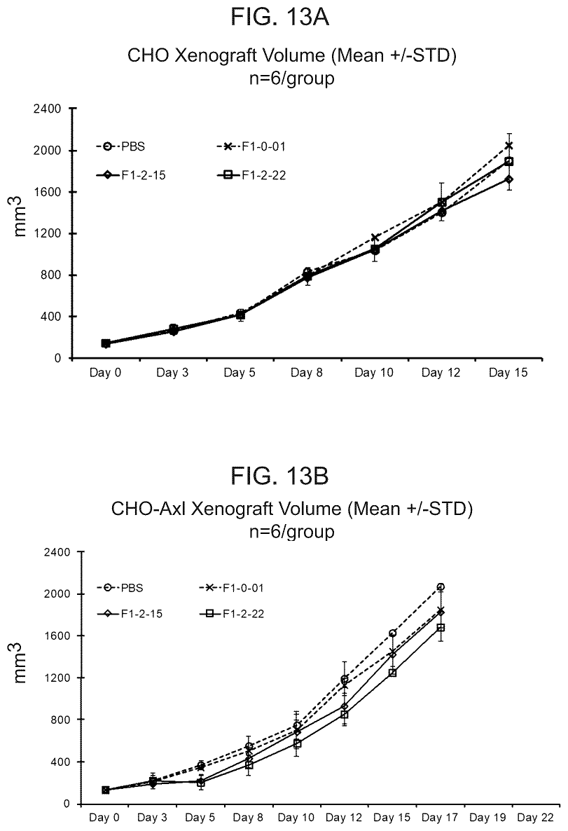

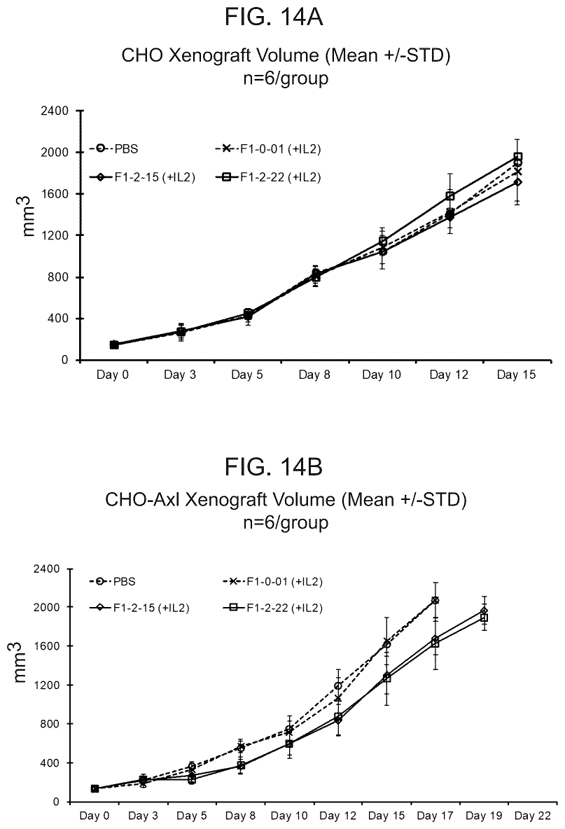

With respect to anti-Axl CAB-CARs, as shown in FIG. 12A, mice dosed intratumorally (IT) with 8×10⁶ T cells transduced with a lentiviral particle containing a nucleic acid encoding either F1-2-15 or F1-2-22 had no effect on Axl negative tumor growth compared to PBS controls or E-Tag only lentiviral particle transduced T cells (F1-0-01). In contrast, as shown in FIG. 12B, IT dosing of the same cell preparations into human Axl-expressing tumors demonstrated significant tumor suppression of Axl positive tumors by F1-2-15 and F1-2-22. While cells expressing F1-2-15 or F1-2-22 were equipotent in in vitro killing assays against tumors expressing Axl as compared to the ability of cells expressing F1-1-15 to kill tumors expressing Ror2, tumor growth inhibition in vivo was not as sustained over time following IV administration compared to local injection (FIG. 13A control vs FIG. 13B). Administration of systemic IL-2 into animals for 3 days (FIG. 14A control vs FIG. 14B) led to improved anti-tumor activity as compared to IV dosing without IL-2. These data demonstrate that the tumor microenvironment is again reproduced in vivo in this engrafted tumor mouse model and that the genetically modified T cells expressing exemplary anti-Ror2 CAB-CARs F1-2-15 and F1-2-22 were capable of accessing the solid tumor and cell killing in vivo despite the rapid doubling time of the target engrafted tumor cells. Furthermore, these experiments using exemplary anti-Ror2 and anti-Axl CAB-CARs provided herein, demonstrate that such CAB-CARs are effective in in vivo methods for activating T cells that express these CAB-CARs.

Those skilled in the art can devise many modifications and other embodiments within the scope and spirit of the present disclosure. Indeed, variations in the materials, methods, drawings, experiments, examples, and embodiments described may be made by skilled artisans without changing the fundamental aspects of the present disclosure. Any of the disclosed embodiments can be used in combination with any other disclosed embodiment.

In some instances, some concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

TABLE 3
Sequences of SEQ ID NOs.

| SEQ ID NO: | Name | Sequence |
|---|---|---|
| 1 | CD137 Co-stimulatory | KRGRKKLLYIFKQPFMRPVQTTQEEDGCSCRFPEEEEGGCEL |
| | domain | |
| 2 | CD28 Co-stimulatory | RSKRSRLLHSDYMNMTPRRPGPTRKHYQPYAPPRDFAAYRS |
| | domain | |
| 3 | ICΔ Co-stimulatory | RSKRSRLLHSDYMNMTPRRPGTRKHYQAYAAARDFAAYRS |
| | domain | |
| 4 | ICOS Co-stimulatory | TKKKYSSSVHDPNGEYMFMRAVNTAKKSRLTDVTL |
| | domain | |
| 5 | OX40 Co-stimulatory | RRDQRLPPDAHKPPGGGSFRTPIQEEQADAHSTLAKI |
| | domain | |
| 6 | CD27Co-stimulatory | HQRRKYRSNKGESPVEPAEPCRYSCPREEEGSTIPIQEDYRKPEPACSP |
| | domain | |
| 7 | BLTA Co-stimulatory | CCLRRHQGKQNELSDTAGREINLVDAHLKSEQTEASTRQNSQVLLSETGIYDNDPDLCFR |
| | domain | MQEGSEVYSNPCLEENKPGIVYASLNHSVIGPNSRLARNVKEAPTEYASICVRS |
| 8 | CD30 Co-stimulatory | RRACRKRIRQKLHLCYPVQTSQPKLELVDSRPRRSSTQLRSGASVTEPVAEERGLMSQPL |
| | domain | METCHSVGAAYLESLPLQDASPAGGPSSPRDLPEPRVSTEHTNNKIEKIYIMKADTVIVG |
| | | TVKAELPEGRGLAGPAEPELEEELEADHTPHYPEQETEPPLGSCSDVMLSVEEEGKEDPL |
| | | PTAASGK |
| 9 | GITR Co-stimulatory | HIWQLRSQCMWPRETQLLLEVPPSTEDARSCQFPEEERGERSAEEKGRLGDLWV |
| | domain | |
| 10 | HVEM Co-stimulatory | CVKRRKPRGDVVKVIVSVQRKRQEAEGEATVIEALQAPPDVTTVAVEETIPSFTGRSPNH |
| | domain | |
| 11 | CD3Z isoform 1 | MKWKALFTAAILQAQLPITEAQSFGLLDPKLCYLLDGILFIYGVILTALFLRVKFSRSAD |
| | Activating domain | APAYQQGQNQLYNELNLGRREEYDVLDKRRGRDPEMGGKPRRKNPQEGLYNELQKDKMAE |
| | | AYSEIGMKGERRRGKGHDGLYQGLSTATKDTYDALHMQALPPR |
| 12 | CD3Z isoform 2 | MKWKALFTAAILQAQLPITEAQSFGLLDPKLCYLLDGILFIYGVILTALFLRVKFSRSAD |
| | Activating domain | APAYQQGQNQLYNELNLGRREEYDVLDKRRGRDPEMGGKPQRRKNPQEGLYNELQKDKMA |
| | | EAYSEIGMKGERRRGKGHDGLYQGLSTATKDTYDALHMQALPPR |
| 13 | CD3Z 3 | RVKFSRSADAPAYQQGQNQLYNELNLGRREEYDVLDKRRGRDPEMGGKPRRKNPQEGLYN |
| | Activating domain | ELQKDKMAEAYSEIGMKGERRRGKGHDGLYQGLSTATKDTYDALHMQALPPR |
| 14 | CD3Z 4 | NQLYNELNLGRREEYDVLDKR |
| | Activating domain | |
| 15 | CD3Z 5 | EGLYNELQKDKMAEAYSEIGMK |
| | Activating domain | |
| 16 | CD3Z 6 | DGLYQGLSTATKDTYDALHMQ |
| | Activating domain | |
| 17 | CD3D 1 | MEHSTFLSGLVLATLLSQVSPFKIPIEELEDRVFVNCNTSITWVEGTVGTLLSDITRLDL |
| | Activating domain | GKRILDPRGIYRCNGTDIYKDKESTVQVHYRMCQSCVELDPATVAGIIVTDVIATLLLAL |
| | | GVFCFAGHETGRLSGAADTQALLRNDQVYQPLRDRDDAQYSHLGGNWARNK |
| 18 | CD3D 2 | MEHSTFLSGLVLATLLSQVSPFKIPIEELEDRVFVNCNTSITWVEGTVGILLSDITRLD |
| | Activating domain | LGKRILDPRGIYRCNGTDIYKDKESTVQVHYRTADTQALLRNDQVYQPLRDRDDAQYSH |
| | | LGGNWARNK |
| 19 | CD3D 3 | DQVYQPLRDRDDAQYSHLGGN |
| | Activating domain | |
| 20 | CD3E 1 | MQSGTHWRVLGLCLLSVGVWGQDGNEEMGGITQYPYKVSISGTTVILTCPQYPGSEILWQ |
| | Activating domain | HNDKNIGGEDDKNIGSDEDHLSLKEFSELEQSGYYVCYPRGSKPEDANFYLYLRARVCEN |
| | | CMEMDMSVATIVIVDICITGGLLLLVYYWSKNRKAKAKPVTRGAGAGGRQRGQNKERPPP |
| | | VPNPDYEPIRKGQRDLYSGLNQRRI |
| 21 | CD3E 2 | NPDYEPIRKGQRDLYSGLNQR |
| | Activating domain | |
| 22 | CD3G 1 | MEQGKGLAVLIKAIILLQGTLAQSIKGNHLVKYDYQEDGSVLLTCDAEAKNITWFKDGKM |
| | Activating domain | IGFLTEDKKKWNLGSNAKKPRGMYQCKGSQNKSKPLQVYYRMCQNCIELNAATISGFLFA |
| | | EIVSIFVLAVGVYFIAGQDGVRQSRASDKQTLLPNDQLYQPLKDREDDQYSHLQGNQLRR |
| | | N |
| 23 | CD3G 2 | DQLYQPLKDREDDQYSHLQGN |
| | Activating domain | |
| 24 | CD79A 1 | MPGGPGVLQALPATIFLLFLLSAVYLGPGCQALWMHKVPASLMVSLGEDAHFQCPHNSSN |
| | Activating domain | NANVTWWRVLHGNYTWPPEFLGPGEDPNGTLIIQNVNKSHGGIYVCRVQEGNESYQQSCG |
| | | TYLRVRQPPPRPFLDMGEGTKNRIITAEGIILLFCAVVPGTLLLFRKRWQNEKLGLDAGD |
| | | EYEDENLYEGLNLDDCSMYEDISRGLQGTYQDVGSLNIGDVQLEKP |
| 25 | CD79A 2 | MPGGPGVLQALPATIFLLFLLSAVYLGPGCQALWMHKVPSSLMVSLGEDAHFQCPHNSSN |
| | Activating domain | NANVTWWRVLHGNYTWPPEFLGPGEDPNEPPPRPFLDMGEGTKNRIITAEGIILLFCAVV |
| | | PGTLLLFRKRWQNEKLGLDAGDEYEDENLYEGLNLDDCSMYEDISRGLQGTYQDVGSLNI |
| | | GDVQLEKP |
| 26 | CD79A 3 | ENLYEGLNLDDCSMYEDISRG |
| | Activating domain | |
| 27 | DAP12 1 | MGGLEPCSRLLLLPLLLAVSGLRPVQAQAQSDCSCSTVSPGVLAGIVMGDLVLTVLIALA |
| | Activating domain | VYFLGRLVPRGRGAAEAATRKQRITETESPYQELQGQRSDVYSDLNTQRPYYK |
| 28 | DAP12 2 | MGGLEPCSRLLLLPLLLAVSGLRPVQAQAQSDCSCSTVSPGVLAGIVMGDLVLTVLIALA |
| | Activating domain | VYFLGRLVPRGRGAAEATRKQRITETESPYQELQGQRSDVYSDLNTQ |
| 29 | DAP12 3 | MGGLEPCSRLLLLPLLLAVSDCSCSTVSPGVLAGIVMGDLVLTVLIALAVYFLGRLVPRG |
| | Activating domain | RGAAEAATRKRQRITETESPYQELQGQRSDVYSDLNTQRPYYK |
| 30 | DAP12 4 | MGGLEPCSRLLLLPLLLAVSDCSCSTVSPGVLAGIVMGDLVLTVLIALAVYFLGRLVPRG |
| | Activating domain | RGAAEATRKQRITETESPYQELQGQRSDVYSDLNTQRPYYK |
| 31 | DAP12 5 | ESPYQELQGQRSDVYSDLNTQ |
| | Activating domain | |
| 32 | FCER1G 1 | MIPAVVLLLLLLVEQAAALGEPQLCYILDAILFLYGIVLTLLYCRLKIQVRKAAITSYEK |
| | Activating domain | SDGVYTGLSTRNQETYETLKHEKPPQ |
| 33 | FCER1G 2 | DGVYTGLSTRNQETYETLKHE |
| | Activating domain | |
| 34 | DAP10 | RPRRSPAQDGKVYINMPGRG |
| | Activating domain | |
| 35 | CD28 | FWVLVVVGGVLACYSLLVTVAFIIFWVRSKRSRLLHSDYMNMTPRRPGPTRKHYQPYAPP |
| | Activating domain | RDFAAYRS |
| 36 | ZAP70 | MPDPAAHLPFFYGSISRAEAEEHLKLAGMADGLFLLRQCLRSLGGYVLSLVHDVRFHHFP |
| | Activating domain | IERQLNGTYAIAGGKAHCGPAELCEFYSRDPDGLPCNLRKPCNRPSGLEPQPGVFDCLRD |
| | | AMVRDYVRQTWKLEGEALEQAIISQAPQVEKLIATTAHERMPWYHSSLTREEAERKLYSG |
| | | AQTDGKFLLRPRKEQGTYALSLIYGKTVYHYLISQDKAGKYCIPEGTKFDTLWQLVEYLK |
| | | LKADGLIYCLKEACPNSSASNASGAAAPTLPAHPSTLTHPQRRIDTLNSDGYTPEPARIT |
| | | SPDKPRPMPMDTSVYESPYSDPEELKDKKLFLKRDNLLIADIELGCGNFGSVRQGVYRMR |
| | | KKQIDVAIKVLKQGTEKADTEEMMREAQEIMQLDNPYIVRLIGVCQAEALMLVMEMAGGG |
| | | PLHKFLVGKREEIPVSNVAELLHQVSMGMKYLEEKNFVHRDLAARNVLLVNRHYAKISDF |
| | | GLSKALGADDSYYTARSAGKWPLKWYAPECINFRKFSSRSDVWSYGVTMWEALSYGQKPY |
| | | KKMKGPEVMAFIEQGKRMECPPECPPELYALMSDCWIYKWEDRPDFLTVEQRMRACYYSL |
| | | ASKVEGPPGSTQKAEAACA |
| 37 | HA Epitope | YPYDVPDYA |
| 38 | FLAG Epitope | DYKDDDDK |
| 39 | c-myc Epitope | EQKLISEEDL |
| 40 | His5 Affinity | HHHHH |
| 41 | HisX6 Affinity | HHHHHH |
| 42 | Strep Tag Affinity | WSHPQFEK |
| 43 | RYIRS Affinity | RYIRS |
| 44 | FHHT Affinity | FHHT |
| 45 | Affinity | WEAAAREACCRECCARA |
| 46 | CD8 alpha | IYIWAPLAGTCGVLLLSLVITLYC |
| | Transmembrane domain | |
| 47 | CD8 beta | LGLLVAGVLVLLVSLGVAIHLCC |
| | Transmembrane domain | |
| 48 | CD4 | ALIVLGGVAGLLLFIGLGIFFCVRC |
| | Transmembrane domain | |
| 49 | CD3 zeta | LCYLLDGILFIYGVILTALFLRV |
| | Transmembrane domain | |
| 50 | CD28 | FWVLVVVGGVLACYSLLVTVAFIIFWV |
| | Transmembrane domain | |
| 51 | OX40 | VAAILGLGLVLGLLGPLAILLALYLL |
| | Transmembrane domain | |
| 52 | CD7 | ALPAALAVISFLLGLGLGVACVLA |
| | Transmembrane domain | |
| 53 | Linker 1 | GGGGSGGGGSGGGGS |
| 54 | Linker 2 | GGGGSGGGGSGGGGSGGGGSGGGGSGGGGS |
| 55 | Linker 3 | GGGGSGGGGSGGGGS |
| 56 | Linker 4 | GGSG |
| 57 | Linker 5 | GGSGG |
| 58 | Linker 6 | GSGSG |
| 59 | Linker 7 | GSGGG |
| 60 | Linker 8 | GGGSG |
| 61 | Linker 9 | GSSSG |
| 62 | Hinge 1 | CPPC |
| 63 | Hinge 2 | DKTHT |
| 64 | Hinge 3 | CPEPKSCDTPPPCPR |
| 65 | Hinge 4 | ELKTPLGDTTHT |
| 66 | Hinge 5 | KSCDKTHTCP |
| 67 | Hinge 6 | KCCVDCP |
| 68 | Hinge 7 | KYGPPCP |
| 69 | Hinge 8 | ELPSCDKTHTCPPCP |
| 70 | Hinge 9 | ERKCCVECPPCP |
| 71 | Hinge 10 | ELKTPLGDTTHTCPRCP |
| 72 | Hinge 11 | SPNMVPHAHHAQ |
| 73 | Hinge 12 | TTTPAPRPPTPAPTIASQPLSLRPEACRPAAGGAVHTRGLDFACD |
| 74 | CD8 Signal peptide | MALPVTALLLPLALLLHAARP |
| 75 | CD8 Stalk-TM | TTTPAPRPPTPAPTIASQPLSLRPEACRPAAGGAVHTRGLDFACDIYWAPLAGTCGVLLL |
| | | SLVITLYC |
| 76 | CD28 Stalk-TM | IEVMYPPPYLDNEKSNGTIIHVKGKHLCPSPLFPGPSKPFWVLVVVGGVLACYSLLVTVA |
| | | FIIFWV |
| 77 | 2A-1 Cleavage signal | GSGEGRGSLLTCGDVEENPGP |
| 78 | eTAG | MLLLVTSLLLCELPHPAFLLIPRKVCNGIGIGEFKDSLSINATNIKHFKNCTSISGDLHI |
| | Elimination domain | LPVAFRGDSFTHTPPLDPQELDILKTVKEITGFLLIQAWPENRTDLHAFENLEIIRGRTK |
| | | QHGQFSLAVVSLNITSLGLRSLKEISDGDVIISGNKNLCYANTINWKKLFGTSGQKTKII |
| | | SNRGENSCKATGQVCHALCSPEGCWGPEPRDCVSCRNCSRGRECVDKCNLLEGEPREFVE |
| | | NSECIQCHPECLPQAMNITCTGRGPDNCIQCAHYIDGPHCVKTCPAGVMGENNTLVWKYA |
| | | DAGHVCHLCHPNCTYGCTGPGLEGCPTNGPKIPSIATGMVGALLLLLVVALGIGLFM |
| 79 | Axl 4007V VH of | EVQLVQSGAEVKKPGATVKISCKVSGYSFWGATMNWIRQPPGKGLEWIGLIKPSNGGTSY |
| | conditionally | NQKFKGRVTISADKSISTAYLQWSSLKASDTAMYYCAHGHYESYEAMDYWGQGTLVTVSS |
| | active scFv | |
| 80 | Axl 4007V VL of | DIQMTQSPSSLSASVGDRVTITCKASQDVVSAVAWYQQKPGQAPRLLIYWQDTRHTGVPS |
| | conditionally | RFSGSGSGTEFTLTISSLQPDDFATYYCQEHFSPPLTFGQGTKVEIK |
| | active scFv | |
| 81 | Ror2 VL 4 of | AIQLTQSPSSLSASVGDRVTITCSATSSVSYMHWYLQKPGQSPQLLIYGTSNLASGVPDR |
| | conditionally | FSGSGSGTDFTLKISRVEAEDVGVYYCQQRSSYPFTFGQGTKVEIK |
| | active scFv | |
| 82 | Ror2 R98H VH of | QVQLQESGPGLVKPSQTLSLTCTVSGYSITTGYYWNWVRQARGQRLEWIGYITYDGSKNY |
| | conditionally | NPSLKNRVTISVDTSKNQFSLKLSSVTAADTAVYYCSHFEGVWYGLDYGWQGTLVTVSS |
| | active scFv | |
| 83 | Ror2 YEE3 VH of | QVQLQESGPGLVKPSQTLSLTCTVSGYSITTGEYWNWVRQARGQRLEWIGYITYDGSKNY |
| | conditionally | NPSLKNRVTISVDTSKNQFSLKLSSVTAADTAVYYCSRFEGVWYGLDYWGQGTLVTVSS |
| | active scFv | |
| 84 | Ror2 VL of | AIQLTQSPSSLSASVGDRVTITCRASESVDRYGNSFIHWYQQKPGKAPKLLIYRTYNLES |
| | conditionally | GIPARFSGSGSGTEFTLTISSLQSEDFAVYYCQQTNEDPWTFGWGTKVEIK |
| | active scFv | |
| 85 | Ror2 L3 4 of | QQX₄₂NX₄₃DPX₄₄TX₄₅ (where X₄₂ is T, I, or P; X₄₃ is E or V; |
| | conditionally | X₄₄ is W or T; and X₄₅ is F or T) |
| | active scFv | |
| 86 | Ror2 VL 5 of | DIVLTQSPDSLAVSLGQRATISCRASESVDRYGNSFIHWYLQKPGQPPKLLIYRTYNLES |
| | conditionally | GIPARFSGTGSRTDFTLTINPVEADDVATYYCQQTNEDPWTFGQGTKVEIK |
| | active scFv | |
| 87 | Axl H1 1 of | X₁GX₂TMN (where X₁ is T or W; and X₂ is H or A) |
| | conditionally | |
| | active scFv | |
| 88 | Axl H2 1 of | LIKPSNGGTSYNQKFKG |
| | conditionally | |
| | active scFv | |
| 89 | Axl H3 1 of | GX₃YX₄SYX₅AMDY (where X₃ is H or D; X₄ is E or H; and X₅ is |
| | conditionally | E or F) |
| | active scFv | |
| 90 | Axl L1 1 of | KASQDVX₆SAVA (where X₆ is S or V) |
| | conditionally | |
| | active scFv | |
| 91 | Axl L2 1 of | WX₇X₈TRX₉T (where X₇ is A or Q; X₈ is S or D; and X₉ is |
| | conditionally | H or D) |
| | active scFv | |
| 92 | Axl L3 1 of | QEHFSX₁₀PLX₁₁ (where X₁₀ is T or P; and X₁₁ is T or R) |
| | conditionally | |
| | active scFv | |
| 93 | Axl WT VH of | EVQLVQSGAEVKKPGATVKISCKVSGYSFTGHTMNWIRQPPGKGLEWIGLIKPSNGGTSY |
| | conditionally | NQKFKGRVTISADKSISTAYLQWSSLKASDTAMYYCAHGHYESYFAMDYWGQGTLVTVSS |
| | active scFv | |
| 94 | Axl WT VL of | DIQMTQSPSSLSASVGDRVTITCKASQDVSSAVAWYQQKPGQAPRLLIYWASTRHTGVPS |
| | conditionally | RFSGSGSGTEFTLTISSLQPDDFATYYCQEHFSTPLTFGQGTKVEIK |
| | active scFv | |
| 95 | Ror2 H1 1 of | GYTX₁TEX₂TX₃H (where X₁ is F or E; X₂ is Y or D; and X₃ is |
| | conditionally | M or D) |
| | active scFv | |
| 96 | Ror2 H1 2 of | X₄GYSITTGYYWN (where X₄ is T or S) |
| | conditionally | |
| | active scFv | |
| 97 | Ror2 H2 1 of | GX₅NX₆NNGGTGYNQKFKG (where X₅ is E or I; and X₆ is T or D) |
| | conditionally | |
| | active scFv | |
| 98 | Ror2 H2 2 of | YITYDGSKNYNPSLKN |
| | conditionally | |
| | active scFv | |
| 99 | Ror2 H3 1 of | GSLYSYGNSYFDY |
| | conditionally | |
| | active scFv | |
| 100 | Ror2 H3 2 of | FEGVWX₇GLDY (where X₇ is Y or G) |
| | conditionally | |
| | active scFv | |
| 101 | Ror2 L1 1 of | SATSSX₈SYMH (where X₈ is E or V) |
| | conditionally | |
| | active scFv | |
| 102 | Ror2 L1 2 of | RASESVDRYGNSFIH |
| | conditionally | |
| | active scFv | |
| 103 | Ror2 L2 1 of | X₉TSNLAS (where X₉ is G or H) |
| | conditionally | |
| | active scFv | |
| 104 | Ror2 L2 2 of | RTYNLES |
| | conditionally | |
| | active scFv | |
| 105 | Ror2 L3 1 of | QQRSSYPFT |
| | conditionally | |
| | active scFv | |
| 106 | Ror2 L3 2 of | QQTNEDPWT |
| | conditionally | |
| | active scFv | |
| 107 | Ror2 WT VH of | QVQLQESGPGLVKPSQTLSLTCTVSGYSITTGYYWNSVRQARGQRLEWIGYITYDGSKNY |
| | conditionally | NPSLKNRVTISVDTSKNQFSLKLSSVTAADTAVYYCSRFEGVWYGLDYWGQGTLVTVSS |
| | active scFv | |
| 108 | Axl VL 1 of | DIQMTQSPSSLSASVGDRVTITCKASQDVVSAVAWYQQKPGQAPRLLIYWQDTRHTGVPS |
| | conditionally | RFSGSGSGTEFTLTISSLQPDDFATYYCQEHFSTPLTFGQGTKVEIK |
| | active scFv | |
| 109 | Axl VL 2 of | DIQMTQSPSSLSASVGDRVTITCKASQDVSSAVAWYQQKPGQAPRLLIYWQDTRHTGVPS |
| | conditionally | RFSGSGSGTEFTLTISSLQPDDFATYYCQEHFSPPLTFGQGTKVEIK |
| | active scFv | |
| 110 | Axl VL 3 of | DIQMTQSPSSLSASVGDRVTITCKASQDVVSAVAWYQQKPGQAPRLLIYWQDTRHTGVPS |
| | conditionally | RFSGSGSGTEFTLTISSLQPDDFATYYCQEHFSPPLTFGQGTKVEIK |
| | active scFv | |
| 111 | Axl VL 4 of | DIQMTQSPSSLSASVGDRVTITCKASQDVVSAVAWYQQKPGQAPRLLIYWQDTRHTGVPS |
| | conditionally | RFSGSGSGTEFTLTISSLQPDDFATYYCQEHFSPPLRFGQGTKVEIK |
| | active scFv | |
| 112 | Axl VH 1 of | EVQLVQSGAEVKKPGATVKISCKVSGYSFTGATMNWIRQPPGKGLEWIGLIKPSNGGTSY |
| | conditionally | NQKFKGRVTISADKSISTAYLQWSSLKASDTAMYYCAHGHYESYEAMDYWGQGTLVTVSS |
| | active scFv | |
| 113 | Axl VH 2 of | EVQLVQSAGEVKKPGATVKISCKVSGYSFWGATMNWIRQPPGKGLEWIGLIKPSNGGTSY |
| | conditionally | NQKFKGRVITSADKISSTAYLQWSSLKASDTAMYYCAHGHYESYEAMDYWGQGTLVTVSS |
| | active scFv | |
| 114 | Axl VH 3 of | EVQLVQSGAEVKKPGATVKISCKVSGYSFTGHTMNWIRQPPGKGLEWIGLIKPSNGGTSY |
| | conditionally | NQKFKGRVTISADKSISTAYLQWSSLKASDTAMYYCAHGHYESYEAMDYWGQGTLVTVSS |
| | active scFv | |
| 115 | Ror2 VH 1 of | EVQLQQSGPELVKPGASVKISCKTSGYTFTEYTMHWMKQSHRKSLEWIGGINTNNGGTGY |
| | conditionally | NQKFKGKATLTVDKSSSTAYMELRSLTSEDSAVYFCARGSLYSYGNSYFDYWGQGTLVTV |
| | active scFv | SS |
| 116 | Ror2 VH 2 of | EVQLQQSGPELVKPGASVKISCKTSGYTETEDTDHWMKQSHRKSLEWIGGENDNNGGTGY |
| | conditionally | NQKFKGKATLTVDKSSSTAYMELRSLTSEDSAVYFCAHGSLYSYGNSYFDYWGQGTLVTV |
| | active scFv | SS |
| 117 | Ror2 VH 3 of | QVQLVQSGAEVKKPGASVKVSCKASGYTFTEYTMHWVRQARGQRLEWIGGINTNNGGTGY |
| | conditionally | NQKFKGRVTISADKSISTAYLQWSSLKASDTAMYYCAHGSLYSYGNSYFDYWGQGTLVTV |
| | active scFv | SS |
| 118 | Ror2 VH 4 of | EVQLVQSGAEVKKPGESLRISCKGSGYTFTEYTMHWVRQAPGQGLEWMGGINTNNGGTGY |
| | conditionally | NQKFKGRVTISADKSISTAYLQWSSLKASDTAMYYCAHGSLYSYGNSYFDYWGQGTLVTV |
| | active scFv | SS |
| 119 | Ror2 VH 5 of | EVQLVQSGAEVKKPGESLRISCKGSGYTFTEYTMHWIRQSPSRGLEWLGGINTNNGGTGY |
| | conditionally | NQKFKGRVTISADKSISTAYLQWSSLKASDTAMYYCAHGSLYSYGNSYFDYWGQGTLVTV |
| | active scFv | SS |
| 120 | Ror2 VH 6 of | DVQLQESGPGLVKPSQSLSLTCSVTGYSITTGYYWNWIRQFPGNKLEWMAYITYDGSKNY |
| | conditionally | NPSLKNRISITRDTSKNKFFLKLNSVTSEDTATYYCSRFEGVWYGLDYWGQGTLVTVSS |
| | active scFv | |
| 121 | Ror2 VH 7 of | DVQLQESGPGLVKPSQSLSLTCSVTGYSITTGYYWNWIRQFPGNKLEWMAYITYDGSKNY |
| | conditionally | NPSLKNRISITRDTSKNKFFLKLNSVTSEDTATYYCSHFEGVWGGLDYWGQGTLVTVSS |
| | active scFv | |
| 122 | Ror2 VL 1 of | QIVLTQSPAIMSASPGEKVTITCSATSSVSYMHWFQQKPGTSPKLWIYGTSNLASGVPAR |
| | conditionally | FSGSGSGTSYSLTISRMEAEDAATYYCQQRSSYPFTFGQGTKVEIK |
| | active scFv | |
| 123 | Ror2 VL 2 of | QIVLTQSPAIMSASPGEKVTITCSATSSESYMHWFQQKPGTSPKLWIYHTSNLASGVPAR |
| | conditionally | FSGSGSGTSYSLTISRMEAEDAATYYCQQRSSYPFTFGQGTKVEIK |
| | active scFv | |
| 124 | Ror2 VL 3 of | AIQLTQSPSSLSASVGDRVTITCSATSSVSYMHWYQQKPGQAPRLLIYGTSNLASGVPDR |
| | conditionally | FSGSGSGTDFTLISRVEAEDVGVYYCQQRSSYPFTFGQGTKVEIK |
| | active scFv | |
| 125 | wild-type CD8 Stalk | TTTPAPRPPTPAPTIASQPLSLRPEACRPAAGGAVHTRGLDFA |
| 126 | wild-type CD28 Stalk | FCKIEVMYPPPYLDNEKSNGTIIHVKGKHLCPSPLFPGPSKP |
| 127 | CD3Z 7 | RVKFSRSADAPAYQQGQNQLYNELNLGRREEYDVLDKRRGRDPEMGGKPQRRKNPQEGLY |
| | Activating domain | NELQKDKMAEAYSEIGMKGERRRGKGHDGLYQGLSTATKDTYDALHMQALPPR |
| 128 | scFv from F1-2-15 | EVQLVQSGAEVKKPGATVKISCKVSGYSFWGATMNWIRQPPGKGLEWIGLIKPSNGGTSY |
| | | NQKFKGRVTISADKSISTAYLQWSSLKASDTAMYYCAHGHYESYEAMDYWGQGTLVTVSS |
| | | GGGGSGGGGSGGGGSDIQMTQSPSSLSASVGDRVTITCKASQDVVSAVAWYQQKPGQAPR |
| | | LLIYWQDTRHTGVPSRFSGSGSGTEFTLTISSLQPDDFATYYCQEHFSPPLTFGQGTKVE |
| | | IK |
| 129 | scFv from F1-2-13 | EVQLVQSGAEVKKPGATVKISCKVSGYSFWGATMNWIRQPPGKGLEWIGLIKPSNGGTSY |
| | | NQKFKGRVTISADKSISTAYLQWSSLKASDTAMYYCAHGHYESYEAMDYWGQGTLVTVSS |
| | | GGGGSGGGGSGGGGSDIQMTQSPSSLSASVGDRVTITCKASQDVVSAVAWYQQKPGQAPR |
| | | LLIYWQDTRHTGVPSRFSGSGSGTEFTLTISSLQPDDFATYYCQEHFSPPLTFGQGTKVE |
| | | IK |
| 130 | scFv from F1-1-19 | QVQLQESGPGLVKPSQTLSLTCTVSGYSITTGEYWMWVRQARGQRLEWIGYITYDGSKNY |
| | | NPSLKNRVTISVDTSKNQFSLKLSSVTAADTAVYYCSRFEGVWYGLDYWGQGTLVTVSSG |
| | | GGGSGGGGSGGGGSGGGGSGGGGSGGGGSAIQLTQSPSSLSASVGDRVTITCRASESVDR |
| | | YGNSFIHWYQQKPGKAPKLLIYRTYNLESGIPARFSGSGSGTEFTLTISSLQSEDFAVYY |
| | | CQQTNEDPWTFGQGTKVEIK |
| 131 | scFv from F1-1-23 | AIQLTQSPSSLSASVGDRVTITCRASESVDRYGNSFIHWYQQKPGKAPKLLIYRTYNLES |
| | | GIPARFSGSGSGTEFTLTISSLQSEDFAVYYCQQTNEDPWTFGQGTKVEIKGGGGSGGGG |
| | | SGGGGSGGGGSGGGGSGGGGSQVQLQESGPGLVKPSQTLSLTCTVSGYSITTGEYWNWVR |
| | | QARGQRLEWIGYITYDGSKNYNPSLKNRVTISVDTSKNQFSLKLSSVTAADTAVYYCSRF |
| | | EGVWYGLDYWGQGTLVTVSS |
| 132 | scFv from F1-1-15 | AIQLTQSPSSLSASVGDRVTITCRASESVDRYGNSFIHWYQQKPGKAPKLLIYRTYNLES |
| | | GIPARFSGSGSGTEFTLTISSLQSEDFAVYYCQQTNEDPWTFGQGTKVEIKGGGGSSSSS |
| | | SGGGGSGGGGSGGGGSGGGGSQVQLQESGPGLVKPSQTLSLTCTVSGYSITTGYYWNWVR |
| | | QARGQRLEWIGYITYDGSKNYNPSLKNRVTISVDTSKNQFSLKLSSVTAADTAVYYCSHF |
| | | EGVWYGLDYWGQGTLVTVSS |
| 133 | ICΔ CD137 Dual Co- | RSKRSRLLHSDYMNMTPRRPGPTRKHYQAYAAARDFAAYRSKRGRKKLLYIFKQPFMRPV |
| | stimulatory domain | QTTQEEDGCSCRFPEEEEGGCEL |
| 134 | Axl H1 2 of | X₁GX₂X₃MX₄ (where X₁ is T, A, or W; X₂ is H or A; X₃ is T or |
| | conditionally | I; and X₄ is N or I) |
| | active scFv | |
| 135 | Axl H2 2 of | LIKX₅SNGGTX₆YNQKFKG (where X₅ is P or N; and X₆ is S, I, or |
| | conditionally | T) |
| | active scFv | |
| 136 | Axl H3 2 of | GX₇X₈X₉X₁₀X₁₁X₁₂X₁₃X₁₄DYX₁₅X₁₆ (where X₇ is H, D, E, P, R, or W; |
| | conditionally | X₈ is Y or N; X₉ is E, A, D, F, G, H, I, L, M, N, R, V, or, |
| | active scFv | Y; X₁₀ is S, D, M, N, or Q; X₁₁ is Y, C, E, or P; X₁₂ is F, |
| | | E, N, S, T, or V; X₁₃ is A, D, G, L, or Y; X₁₄ is M, E, or F; |
| | | X₁₅ is W, A, D, H, L, N, P, R, or T; and X₁₆ is G or H) |
| 137 | Axl L1 2 of | KASQDX₁₇X₁₈SX₁₉VX₂₀ (where X₁₇ is V, D, G, N, or W; X₁₈ is S or |
| | conditionally | V; X₁₉ is A, L, or M; and X₂₀ is A, D, N, or Q) |
| | active scFv | |
| 138 | Axl L2 2 of | X₂₁X₂₂X₂₃TRX₂₄T (where X₂₁ is W or F; X₂₂ is A, I, N, P, or |
| | conditionally | Q; X₂₃ is S or D; and X₂₄ is H or D) |
| | active scFv | |
| 139 | Axl L3 2 of | QEX₂₅X₂₆SX₂₇X₂₈X₂₉X₃₀ (where X₂₅ is H, C, F, I, L, Q, S, T, |
| | conditionally | V, or Y; X₂₆ is F, C, D, E, G, N, or S; X₂₇ is T, C, or P; |
| | active scFv | X₂₈ is P, A, C, D, E, H, K, S, T, V, or W; X₂₉ is L, G, or |
| | | R; and X₃₀ is T, I, or R) |
| 140 | Ror2 H1 3 of | GYTXI₁TEX₂X₃X₄H (where X₁ is F or E; X₂ is Y or D; X₃ is T |
| | conditionally | or C; and X₄ is M, D, E, or Y) |
| | active scFv | |
| 141 | Ror2 H1 4 of | GYSITTGX₂₉YWN (where X₂₉ is Y, E, R, or T) |
| | conditionally | |
| | active scFv | |
| 142 | Ror2 H3 3 of | X₅X₆X₇X₈NNGGTGYNQKFKG (where X₅ is G or S; X₆ is I or E; X₇ |
| | conditionally | is N, C, L, or V; and X₈ is T, D or E) |
| | active scFv | |
| 143 | Ror2 H2 4 of | YITYDGSX₃₀NYNPSLKN (where X₃₀ is K or N) |
| | conditionally | |
| | active scFv | |
| 144 | Ror2 H3 3 of | X₉X₁₀X₁₁SX₁₂YX₁₃YX₁₄X₁₅SYFX₁₆X₁₇X₁₈ (where X₉ is A, M, or T; |
| | conditionally | X₁₀ is R or H; X₁₁ is G or E; X₁₂ is L or F; X₁₃ is S or G; |
| | active scFv | X₁₄ is G or D; X₁₅ is N or E; X₁₆ is D or L; X₁₇ is Y, C, or |
| | | T; and X₁₈ is W or L) |
| 145 | Ror2 H3 4 of | CSX₃₁X₃₂X₃₃X₃₄VX₃₅X₃₆X₃₇LDX₃₈ (where X₃₁ is R, G, H, W, or Y; |
| | conditionally | X₃₂ is F, C, N, or Q; X₃₃ is E or S; X₃₄ is G, E, F, H, M, Q, |
| | active scFv | or S; X₃₅ is W, A, I, P, Q, T, or V; X₃₆ is Y, G, N, or Q, |
| | | X₃₇ is G, S, or T; and X₃₈ is Y or I) |
| 146 | Ror2 L1 3 of | SATSSX₁₉X₂₀X₂₁MX₂₂ (where X₁₉ is V or E; X₂₀ is S or D; X₂₁ |
| | conditionally | is Y, C, or D; and X₂₂ is H, G, or L) |
| | active scFv | |
| 147 | Ror2 L1 4 of | RASESVDRYGNSX₃₉IH (where X₃₉ is F, S, or T) |
| | conditionally | |
| | active scFv | |
| 148 | Ror2 L2 3 of | X₂₃TSNLAS (where X₂₃ is G, C, H, or P) |
| | conditionally | |
| | active scFv | |
| 149 | Ror2 L2 4 of | X₄₀TYX₄₁LES (where X₄₀ is R, C, D, E, or W; and X₄₁ is N or |
| | conditionally | D) |
| | active scFv | |
| 150 | Ror2 L3 3 of | QX₂₄X₂₅SX₂₆YPFX₂₇X₂₈ (where X₂₄ is Q or E; X₂₅ is R or H; X₂₆ |
| | conditionally | is S, D, G, I, Q, or V; X₂₇ is T or D; and X₂₈ is F, D, or |
| | active scFv | E) |
| 151 | Ror2 CAB3 VH | QVQLVQSGAEVKKPGASVKVSCKASGYTFTEYTMHWIRQSPSRGLEWLGGINDNNGGTGY |
| | | NQKFKGRFTISRDNSKNTLYLQMNSLRAEDTAVYYCARGSLYSYGNSYFDYWGQGLTVTV |
| | | SS |
| 152 | Ror2 CAB VL | EIVLTQSPATLSLSPGERATLSCSATSSVSYMHWFQQRPGQSPRRLIYHTSNLASGVPSR |
| | | FSGSGSGTDFTLKISRVEAEDVGVYYCQQRSSYPFTFGQGTKVEIK |
| 153 | scFv from F1-1-11 | QVQLQESGPGLVKPSQTLSLTCTVSGYSITTGYYWNWVRQARGQRLEWIGYITYDGSKNY |
| | | NPSLKNRVTISVDTSKNQFSLKLSSVTAADTAVYYCSHFEGVWYGLDYQGQGTLVTVSSG |
| | | GGGSGGGGSGGGGSGGGGSGGGGSGGGGSAIQLTQSPSSLSASVGDRVTITCRASESVDR |
| | | YGNSFIHWYQQKPGKAPKLLIYRTYNLESGIPARFSGSGSGTEFTLTISSLQSEDFAVYY |
| | | CQQTNEDPWTFGQGTKVEIK |
| 154 | scFv from F1-1-17 | QVQLQESGPGLVKPSQTLSLTCTVSGYSITTGEYWNWVRQARGQRLEWIGYITYDGSKNY |
| | | NPSLKNRVTISVDTSKNQFSLKLSSVTAADTAVYYCSRFEGVWYGLDYWGQGTLVTVSSG |
| | | GGGSGGGGSGGGGSAIQLTQSPSSLSASVGDRVTITCRASESVDRYGNSFIHWYQQKPGK |
| | | APKLLIYRTYNLESGIPARFSGSGSGTEFTLTISSLQSEDFAVYYCQQTNEDPWTFGQGT |
| | | KVEIK |
| 155 | scFv from F1-1-25 | EIVLTQSPATLSLSPGERATLSCSATSSVSYMHWFQQRPGQSPRRLIYHTSNLASGVPDR |
| | | FSGSGSGTDFTLKISRVEAEDVGVYYCQQRSSYPFTFGQGTKVEIKGGGGSGGGGSGGGG |
| | | SQVQLVQSGAEVKKPGASVKVSCKASGYTFTEYTMHWIRQSPSRGLEWLGGINDNNGGTG |
| | | YNQKFKGRFTISRDNSKNTLYLQMNSLRAEDTAVYYCARGSLYSYGNSYFDYWGQGTLVT |
| | | VSS |
| 156 | scFv from F1-1-26 | LVQSGAEVKKPGASVKVSCKASGYTFTEYTMHWIRQSPSRGLEWLGGINDNNGGTGYNQK |
| | | FKGRFTISRDNSKNTLYLQMNSLRAEDTAVYYCARGSLYSYGNSYFDYWGQGTLVTVSS |
| 157 | scFv from F1-1-19 | QVQLQESGPGLVKPSQTLSLTCTVSGYSITTGYYWNWVRQARGQRLEWIGYITYDGSKNY |
| | | NPSLKNRVTISVDTSKNQFSLKLSSVTAADTAVYYCSHFEGVWYGLDYWGQGTLVTVSSG |
| | | GGGSGGGGSGGGGSAIQLTQSPSSLSASVGDRVTITCRASESVDRYGNSFIHWYQQKPGK |
| | | APKLLIYRTYNLESGIPARFSGSGSGTEFTLTISSLQSEDFAVYYCQQTNEDPWTFGQGT |
| | | KVEIK |
| 158 | scFv from F1-1-21 | AIQLTQSPSSLSASVGDRVTITCRASESVDRYGNSFIHWYQQKPGKAPKLLIYRTYNLES |
| | | GIPARFSGSGSGTEFTLTISSLQSEDFAVYYCQQTNEDPWTFGQGTKVEIKGGGGSGGGG |
| | | SGGGGSQVQLQESGPGLVKPSQTLSLTCTVSGYSITTGEYWNWVRQARGQRLEWIGYITY |
| | | DGSKNYNPSLKNRVTISVDTSKNQFSLKLSSVTAADTAVYYCSRFEGVWYGLDYWGQGTL |
| | | VTVSS |
| 159 | scFv from F1-2-3 | EVQLVQSGAEVKKPGATVKISCKVSGYSFWGATMNWIRQPPGKGLEWIGLIKPSNGGTSY |
| | | NQKFKGRVTISADKSISTAYLQWSSLKASDTAMYYCAHGHYESYEAMDYWGQGTLVTVSS |
| | | GGGGSGGGGSGGGGSGGGGSGGGGSGGGGSDIQMTQSPSSLSASVGDRVTITCKASQDVV |
| | | SAVAWYQQKPGQAPRLLIYWQDTRHTGVPSRFSGSGSGTEFTLTISSLQPDDFATYYCQE |
| | | HFSPPLTFGQGTKVEIK |
| 160 | scFv from F1-2-8 | DIQMTQSPSSLSASVGDRVTITCKASQDVVSAVAWYQQKPGQAPRLLIYWQDTRHTGVPS |
| | | RFSGSGSGTEFTLTISSLQPDDFATYYCQEHFSPPLTFGQGTKVEIKGGGGSGGGGSGGG |
| | | GSEVQLVQSGAEVKKPGATVKISCKVSGYSFWGATMNWIRQPPGKGLEWIGLIKPSNGGT |
| | | SYNQKFKGRVTISADKSISTAYLQWSSLKASDTAMYYCAHGHYESYEAMDYWGQGTLVTV |
| | | SS |
| 161 | scFv from F1-2-10 | DIQMTQSPSSLSASVGDRVTITCKASQDVVSAVAWYQQKPGQAPRLLIYWQDTRHTGVPS |
| | | RFSGSGSGTEFTLTISSLQPDDFATYYCQEHFSPPLTFGQGTKVEIKGGGGSGGGGSGGG |
| | | GSGGGGSGGGGSEVQLVQSGAEVKKPGATVKISCKVSGYSFWGATMNWIRQPPGKGLEWI |
| | | GLIKPSNGGTSYNQKFKGRVTISADKSISTAYLQWSSLKASDTAMYYCAHGHYESYEAMD |
| | | YWGQGTLVTVSS |

What is claimed is:

1. An isolated nucleic acid encoding a chimeric antigen receptor (CAR) for binding Axl, said CAR comprising:
a) a conditionally active antigen-specific targeting region (ASTR) that exhibits an increased binding to Axl at a pH of 6.7 compared to a pH of 7.4;
b) a transmembrane domain; and
c) an intracellular activating domain, wherein the transmembrane domain is located between the ASTR and the intracellular activating domain.

2. The isolated nucleic acid of claim 1, wherein the ASTR binds to the same epitope of Axl as a single-chain variable antibody fragment comprising an antibody heavy chain of SEQ ID NO:79 and an antibody light chain of SEQ ID NO:80.

3. The isolated nucleic acid of claim 2, wherein the ASTR is an antibody selected from a single-chain antibody, an Fab fragment, an Fab′ fragment, an (Fab′)2 fragment, an Fv fragment, and a divalent single-chain antibody or a diabody.

4. The isolated nucleic acid of claim 1, wherein the ASTR comprises a heavy chain variable region comprising three complementarity determining regions having H1, H2, and H3 sequences, wherein:
a) the H1 sequence is X₁GX₂TMN (SEQ ID NO:87);
b) the H2 sequence is LIKPSNGGTSYNQKFKG (SEQ ID NO:88); and
c) the H3 sequence is GX₃YX₄SYX₅AMDY (SEQ ID NO:89),
wherein X₁ is T or W; X₂ is H or A; X₃ is H or D; X₄ is E or H; and X₅ is E or F.

5. The isolated nucleic acid of claim 4, wherein the heavy chain variable region comprises an amino acid sequence of SEQ ID NO:79.

6. The isolated nucleic acid-of claim 1, wherein said ASTR comprises a light chain variable region comprising three complementarity determining regions having L1, L2, and L3 sequences, wherein:
a) the L1 sequence is KASQDVX₆SAVA (SEQ ID NO:90);
b) the L2 sequence is WX₇X₈TRX₉T (SEQ ID NO:91); and
c) the L3 sequence is QEHFSX₁₀PLX₁₁ (SEQ ID NO: 92),
wherein X₆ is S or V; X₇ is A or Q; X₈ is S or D; X₉ is H or D; X₁₀ is T or P; and X₁₁ is T or R.

7. The isolated nucleic acid of claim 6, wherein the light chain variable region comprises an amino acid sequence of SEQ ID NO:80.

8. The isolated nucleic acid of claim 7, wherein the ASTR comprises a heavy chain variable region comprising an amino acid sequence of SEQ ID NO:79.

9. The isolated nucleic acid of claim 2, wherein the ASTR is a single-chain variable fragment comprising a heavy chain and a light chain, wherein the heavy chain and the light chain are separated by a linker, wherein the linker is between 6 and 100 amino acids in length.

10. The isolated nucleic acid of claim 9, wherein the heavy chain and the light chain are separated by linker 1 (SEQ ID NO:53), linker 2 (SEQ ID NO:54), or linker 3 (SEQ ID NO:55).

11. The isolated nucleic acid of claim 1, wherein the ASTR comprises an amino acid sequence of any one of SEQ ID NOs:128-129 or 159-161.

12. An isolated recombinant T cell or NK cell, comprising a genome comprising one or more nucleic acid sequences operably linked to a promoter active in T cells and/or NK cells, wherein one of the one or more nucleic acid sequences comprises the isolated nucleic acid of claim 1.

13. The isolated recombinant T cell or NK cell of claim 12, wherein the isolated nucleic acid encoding the CAR further encodes a recognition domain, wherein nucleic acids encoding the recognition domain are separated from nucleic acids encoding the CAR by a ribosomal skip sequence.

14. A method for activating a T cell or NK cell, comprising contacting a target mammalian cell with the T cell or NK cell in a microenvironment at a pH of less than 7.0, wherein the target mammalian cell expresses Axl, and wherein the T cell or NK cell comprises the isolated nucleic acid of claim 1.

15. An expression vector comprising the isolated nucleic acid of claim 1 and a promoter that is active in T cells and/or NK cells, wherein the promoter is positioned on the expression vector to promote expression of the isolated nucleic acid encoding the CAR.

16. The expression vector of claim 15, wherein the expression vector is a replication incompetent retroviral particle.

17. A chimeric antigen receptor (CAR) for binding Axl, comprising:
a) a conditionally active antigen-specific targeting region (ASTR) that exhibits an increased binding to Axl at a pH of 6.7 compared to a pH of 7.4;
b) a transmembrane domain; and
c) an intracellular activating domain, wherein the transmembrane domain is located between the ASTR and the intracellular activating domain.

18. The CAR of claim 17, wherein the ASTR comprises a heavy chain variable region comprising three complementarity determining regions having H1, H2, and H3 sequences, wherein:
a) the H1 sequence is X₁GX₂TMN (SEQ ID NO:87);
b) the H2 sequence is LIKPSNGGTSYNQKFKG (SEQ ID NO:88); and
c) the H3 sequence is GX₃YX₄SYX₅AMDY (SEQ ID NO:89),
wherein X₁ is T or W; X₂ is H or A; X₃ is H or D; X₄ is E or H; and X₅ is E or F.

19. The CAR of claim 18, wherein the heavy chain variable region comprises an amino acid sequence of SEQ ID NO:79.

20. The CAR of claim 17, wherein the ASTR comprises a light chain variable region comprising three complementarity determining regions having L1, L2, and L3 sequences, wherein:
a) the L1 sequence is KASQDVX₆SAVA (SEQ ID NO:90);
b) the L2 sequence is WX₇X₈TRX₉T (SEQ ID NO:91); and
c) the L3 sequence is QEHFSX₁₀PLX₁₁ (SEQ ID NO:92),
wherein X₆ is S or V; X₇ is A or Q; X₈ is S or D; X₉ is H or D; X₁₀ is T or P; and X₁₁ is T or R.

21. The CAR of claim 20, wherein the light chain variable region comprises an amino acid sequence of SEQ ID NO:80.

22. The CAR of claim 21, wherein the ASTR comprises a heavy chain variable region comprising an amino acid sequence of SEQ ID NO:79.

23. A chimeric antigen receptor (CAR) for binding Ror2, comprising:
a) a conditionally active antigen-specific targeting region (ASTR) that exhibits an increased binding to Ror2 at a pH of 6.7 compared to a pH of 7.4;
b) a transmembrane domain; and
c) an intracellular activating domain, wherein the transmembrane domain is located between the ASTR and the intracellular activating domain.


SEQUENCE LISTING (161 sequences)

SEQ ID NO: 1 - protein, 42 aa, synthetic construct
  REGION 1..42: note = synthetic CD137 Co-stimulatory domain
KRGRKKLLYIFKQPFMRPVQTTQEEDGCSCRFPEEEEGGCEL

SEQ ID NO: 2 - protein, 41 aa, synthetic construct
  REGION 1..41: note = synthetic CD28 Co-stimulatory domain
RSKRSRLLHSDYMNMTPRRPGPTRKHYQPYAPPRDFAAYRS

SEQ ID NO: 3 - protein, 41 aa, synthetic construct
  REGION 1..41: note = synthetic IC-delta Co-stimulatory domain
RSKRSRLLHSDYMNMTPRRPGPTRKHYQAYAAARDFAAYRS

SEQ ID NO: 4 - protein, 35 aa, synthetic construct
  REGION 1..35: note = synthetic ICOS Co-stimulatory domain
TKKKYSSSVHDPNGEYMFMRAVNTAKKSRLTDVTL

SEQ ID NO: 5 - protein, 37 aa, synthetic construct
  REGION 1..37: note = synthetic OX40 Co-stimulatory domain
RRDQRLPPDAHKPPGGGSFRTPIQEEQADAHSTLAKI

SEQ ID NO: 6 - protein, 49 aa, synthetic construct
  REGION 1..49: note = synthetic CD27 Co-stimulatory domain
HQRRKYRSNKGESPVEPAEPCRYSCPREEEGSTIPIQEDYRKPEPACSP

SEQ ID NO: 7 - protein, 114 aa, synthetic construct
  REGION 1..114: note = synthetic BLTA Co-stimulatory domain
CCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 8 - protein, 187 aa, synthetic construct
  REGION 1..187: note = synthetic CD30 Co-stimulatory domain
RRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 9 - protein, 54 aa, synthetic construct
  REGION 1..54: note = synthetic GITR Co-stimulatory domain
HIWQLRSQCMWPRETQLLLEVPPSTEDARSCQFPEEERGERSAEEKGRLGDLWV

SEQ ID NO: 10 - protein, 60 aa, synthetic construct
  REGION 1..60: note = synthetic HVEM Co-stimulatory domain
CVKRRKPRGDVVKVIVSVQRKRQEAEGEATVIEALQAPPDVTTVAVEETIPSFTGRSPNH

SEQ ID NO: 11 - protein, 163 aa, synthetic construct
  REGION 1..163: note = synthetic CD3Z isoform 1 Activating domain
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 164 aa, synthetic construct
  REGION 1..164: note = synthetic CD3Z isoform 2 Activating domain
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 13 - protein, 112 aa, synthetic construct
  REGION 1..112: note = synthetic CD3Z 3 Activating domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 21 aa, synthetic construct
  REGION 1..21: note = synthetic CD3Z 4 Activating domain
NQLYNELNLGRREEYDVLDKR

SEQ ID NO: 15 - protein, 22 aa, synthetic construct
  REGION 1..22: note = synthetic CD3Z 5 Activating domain
EGLYNELQKDKMAEAYSEIGMK

SEQ ID NO: 16 - protein, 21 aa, synthetic construct
  REGION 1..21: note = synthetic CD3Z 6 Activating domain
DGLYQGLSTATKDTYDALHMQ

SEQ ID NO: 17 - protein, 171 aa, synthetic construct
  REGION 1..171: note = synthetic CD3D 1 Activating domain
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 18 - protein, 127 aa, synthetic construct
  REGION 1..127: note = synthetic CD3D 2 Activating domain
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 19 - protein, 21 aa, synthetic construct
  REGION 1..21: note = synthetic CD3D 3 Activating domain
DQVYQPLRDRDDAQYSHLGGN

SEQ ID NO: 20 - protein, 206 aa, synthetic construct
  REGION 1..206: note = synthetic CD3E 1 Activating domain
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDMSVATIVIVDICITGGLLLLVYYWSKNRKAKAKPVTRGAGAGGRQRGQNKERPPPVPNPDYEPIRKGQRDLYSGLNQRRI

SEQ ID NO: 21 - protein, 21 aa, synthetic construct
  REGION 1..21: note = synthetic CD3E 2 Activating domain
NPDYEPIRKGQRDLYSGLNQR

SEQ ID NO: 22 - protein, 182 aa, synthetic construct
  REGION 1..182: note = synthetic CD3G 1 Activating domain
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 23 - protein, 21 aa, synthetic construct
  REGION 1..21: note = synthetic CD3G 2 Activating domain
DQLYQPLKDREDDQYSHLQGN

SEQ ID NO: 24 - protein, 226 aa, synthetic construct
  REGION 1..226: note = synthetic CD79A 1 Activating domain
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 25 - protein, 188 aa, synthetic construct
  REGION 1..188: note = synthetic CD79A 2 Activating domain
MPGGPGVLQALPATIFLLFLLSAVYLGPGCQALWMHKVPASLMVSLGEDAHFQCPHNSSNNANVTWWRVLHGNYTWPPEFLGPGEDPNEPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 26 - protein, 21 aa, synthetic construct
  REGION 1..21: note = synthetic CD79A 3 Activating domain
ENLYEGLNLDDCSMYEDISRG

SEQ ID NO: 27 - protein, 113 aa, synthetic construct
  REGION 1..113: note = synthetic DAP12 1 Activating domain
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 28 - protein, 107 aa, synthetic construct
  REGION 1..107: note = synthetic DAP12 2 Activating domain
MGGLEPCSRLLLLPLLLAVSGLRPVQAQAQSDCSCSTVSPGVLAGIVMGDLVLTVLIALAVYFLGRLVPRGRGAAEATRKQRITETESPYQELQGQRSDVYSDLNTQ

SEQ ID NO: 29 - protein, 102 aa, synthetic construct
  REGION 1..102: note = synthetic DAP12 3 Activating domain
MGGLEPCSRLLLLPLLLAVSDCSCSTVSPGVLAGIVMGDLVLTVLIALAVYFLGRLVPRGRGAAEAATRKQRITETESPYQELQGQRSDVYSDLNTQRPYYK

SEQ ID NO: 30 - protein, 101 aa, synthetic construct
  REGION 1..101: note = synthetic DAP12 4 Activating domain
MGGLEPCSRLLLLPLLLAVSDCSCSTVSPGVLAGIVMGDLVLTVLIALAVYFLGRLVPRGRGAAEATRKQRITETESPYQELQGQRSDVYSDLNTQRPYYK

SEQ ID NO: 31 - protein, 21 aa, synthetic construct
  REGION 1..21: note = synthetic DAP12 5 Activating domain
ESPYQELQGQRSDVYSDLNTQ

SEQ ID NO: 32 - protein, 86 aa, synthetic construct
  REGION 1..86: note = synthetic FCERlG 1 Activating domain
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 33 - protein, 21 aa, synthetic construct
  REGION 1..21: note = synthetic FCERlG 2 Activating domain
DGVYTGLSTRNQETYETLKHE

SEQ ID NO: 34 - protein, 20 aa, synthetic construct
  REGION 1..20: note = synthetic DAP10 Activating domain
RPRRSPAQDGKVYINMPGRG

SEQ ID NO: 35 - protein, 68 aa, synthetic construct
  REGION 1..68: note = synthetic CD28 Activating domain
FWVLVVVGGVLACYSLLVTVAFIIFWVRSKRSRLLHSDYMNMTPRRPGPTRKHYQPYAPPRDFAAYRS

SEQ ID NO: 36 - protein, 619 aa, synthetic construct
  REGION 1..619: note = synthetic ZAP70 Activating domain
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 37 - protein, 9 aa, synthetic construct
  REGION 1..9: note = synthetic HA Epitope
YPYDVPDYA

SEQ ID NO: 38 - protein, 8 aa, synthetic construct
  REGION 1..8: note = synthetic FLAG Epitope
DYKDDDDK

SEQ ID NO: 39 - protein, 10 aa, synthetic construct
  REGION 1..10: note = synthetic c-myc Epitope
EQKLISEEDL

SEQ ID NO: 40 - protein, 5 aa, synthetic construct
  REGION 1..5: note = synthetic His5 Affinity
HHHHH

SEQ ID NO: 41 - protein, 6 aa, synthetic construct
  REGION 1..6: note = synthetic HisX6 Affinity
HHHHHH

SEQ ID NO: 42 - protein, 8 aa, synthetic construct
  REGION 1..8: note = synthetic Strep Tag Affinity
WSHPQFEK

SEQ ID NO: 43 - protein, 5 aa, synthetic construct
  REGION 1..5: note = synthetic RYIRS Affinity
RYIRS

SEQ ID NO: 44 - protein, 4 aa, synthetic construct
  REGION 1..4: note = synthetic FHHT Affinity
FHHT

SEQ ID NO: 45 - protein, 17 aa, synthetic construct
  REGION 1..17: note = synthetic Affinity
WEAAAREACCRECCARA

SEQ ID NO: 46 - protein, 24 aa, synthetic construct
  REGION 1..24: note = synthetic CD8 alpha Transmembrane domain
IYIWAPLAGTCGVLLLSLVITLYC

SEQ ID NO: 47 - protein, 23 aa, synthetic construct
  REGION 1..23: note = synthetic CD8 beta Transmembrane domain
LGLLVAGVLVLLVSLGVAIHLCC

SEQ ID NO: 48 - protein, 25 aa, synthetic construct
  REGION 1..25: note = synthetic CD4 Transmembrane domain
ALIVLGGVAGLLLFIGLGIFFCVRC

SEQ ID NO: 49 - protein, 23 aa, synthetic construct
  REGION 1..23: note = synthetic CD3 zeta Transmembrane domain
LCYLLDGILFIYGVILTALFLRV

SEQ ID NO: 50 - protein, 27 aa, synthetic construct
  REGION 1..27: note = synthetic CD28 Transmembrane domain
FWVLVVVGGVLACYSLLVTVAFIIFWV

SEQ ID NO: 51 - protein, 26 aa, synthetic construct
  REGION 1..26: note = synthetic OX40 Transmembrane domain
VAAILGLGLVLGLLGPLAILLALYLL

SEQ ID NO: 52 - protein, 24 aa, synthetic construct
  REGION 1..24: note = synthetic CD7 Transmembrane domain
ALPAALAVISFLLGLGLGVACVLA

SEQ ID NO: 53 - protein, 15 aa, synthetic construct
  REGION 1..15: note = synthetic Linker 1
GGGGSGGGGSGGGGS

SEQ ID NO: 54 - protein, 30 aa, synthetic construct
  REGION 1..30: note = synthetic Linker 2
GGGGSGGGGSGGGGSGGGGSGGGGSGGGGS

SEQ ID NO: 55 - protein, 14 aa, synthetic construct
  REGION 1..14: note = synthetic Linker 3
GGGGSGGGSGGGGS

SEQ ID NO: 56 - protein, 4 aa, synthetic construct
  REGION 1..4: note = synthetic Linker 4
GGSG

SEQ ID NO: 57 - protein, 5 aa, synthetic construct
  REGION 1..5: note = synthetic Linker 5
GGSGG

SEQ ID NO: 58 - protein, 5 aa, synthetic construct
  REGION 1..5: note = synthetic Linker 6
GSGSG

SEQ ID NO: 59 - protein, 5 aa, synthetic construct
  REGION 1..5: note = synthetic Linker 7
GSGGG

SEQ ID NO: 60 - protein, 5 aa, synthetic construct
  REGION 1..5: note = synthetic Linker 8
GGGSG

SEQ ID NO: 61 - protein, 5 aa, synthetic construct
  REGION 1..5: note = synthetic Linker 9
GSSSG

SEQ ID NO: 62 - protein, 4 aa, synthetic construct
  REGION 1..4: note = synthetic Hinge 1
CPPC

SEQ ID NO: 63 - protein, 5 aa, synthetic construct
  REGION 1..5: note = synthetic Hinge 2
DKTHT

SEQ ID NO: 64 - protein, 15 aa, synthetic construct
  REGION 1..15: note = synthetic Hinge 3
CPEPKSCDTPPPCPR

SEQ ID NO: 65 - protein, 12 aa, synthetic construct
  REGION 1..12: note = synthetic Hinge 4
ELKTPLGDTTHT

SEQ ID NO: 66 - protein, 10 aa, synthetic construct
  REGION 1..10: note = synthetic Hinge 5
KSCDKTHTCP

SEQ ID NO: 67 - protein, 7 aa, synthetic construct
  REGION 1..7: note = synthetic Hinge 6
KCCVDCP

SEQ ID NO: 68 - protein, 7 aa, synthetic construct
  REGION 1..7: note = synthetic Hinge 7
KYGPPCP

SEQ ID NO: 69 - protein, 15 aa, synthetic construct
  REGION 1..15: note = synthetic Hinge 8
EPKSCDKTHTCPPCP

SEQ ID NO: 70 - protein, 12 aa, synthetic construct
  REGION 1..12: note = synthetic Hinge 9
ERKCCVECPPCP

SEQ ID NO: 71 - protein, 17 aa, synthetic construct
  REGION 1..17: note = synthetic Hinge 10
ELKTPLGDTTHTCPRCP

SEQ ID NO: 72 - protein, 12 aa, synthetic construct
  REGION 1..12: note = synthetic Hinge 11
SPNMVPHAHHAQ

SEQ ID NO: 73 - protein, 45 aa, synthetic construct
  REGION 1..45: note = synthetic Hinge 12
TTTPAPRPPTPAPTIASQPLSLRPEACRPAAGGAVHTRGLDFACD

SEQ ID NO: 74 - protein, 21 aa, synthetic construct
  REGION 1..21: note = synthetic CD8 Signal peptide
MALPVTALLLPLALLLHAARP

SEQ ID NO: 75 - protein, 69 aa, synthetic construct
  REGION 1..69: note = synthetic CD8 Stalk-TM
TTTPAPRPPTPAPTIASQPLSLRPEACRPAAGGAVHTRGLDFACDIYIWAPLAGTCGVLLLSLVITLYC

SEQ ID NO: 76 - protein, 66 aa, synthetic construct
  REGION 1..66: note = synthetic CD28 Stalk-TM
IEVMYPPPYLDNEKSNGTIIHVKGKHLCPSPLFPGPSKPFWVLVVVGGVLACYSLLVTVAFIIFWV

SEQ ID NO: 77 - protein, 21 aa, synthetic construct
  REGION 1..21: note = synthetic 2A-1 Cleavage signal
GSGEGRGSLLTCGDVEENPGP

SEQ ID NO: 78 - protein, 357 aa, synthetic construct
  REGION 1..357: note = synthetic eTAG Elimination domain
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 79 - protein, 120 aa, synthetic construct
  REGION 1..120: note = synthetic Axl 4007V VH Conditionally active scFv
EVQLVQSGAEVKKPGATVKISCKVSGYSFWGATMNWIRQPPGKGLEWIGLIKPSNGGTSYNQKFKGRVTISADKSISTAYLQWSSLKASDTAMYYCAHGHYESYEAMDYWGQGTLVTVSS

SEQ ID NO: 80 - protein, 107 aa, synthetic construct
  REGION 1..107: note = synthetic Axl 4007V VL Conditionally active scFv
DIQMTQSPSSLSASVGDRVTITCKASQDVVSAVAWYQQKPGQAPRLLIYWQDTRHTGVPSRFSGSGSGTEFTLTISSLQPDDFATYYCQEHFSPPLTFGQGTKVEIK

SEQ ID NO: 81 - protein, 106 aa, synthetic construct
  REGION 1..106: note = synthetic Ror2 VL 4 Conditionally active scFv
AIQLTQSPSSLSASVGDRVTITCSATSSVSYMHWYLQKPGQSPQLLIYGTSNLASGVPDRFSGSGSGTDFTLKISRVEAEDVGVYYCQQRSSYPFTFGQGTKVEIK

SEQ ID NO: 82 - protein, 119 aa, synthetic construct
  REGION 1..119: note = synthetic Ror2 R98H VH Conditionally active scFv
QVQLQESGPGLVKPSQTLSLTCTVSGYSITTGYYWNWVRQARGQRLEWIGYITYDGSKNYNPSLKNRVTISVDTSKNQFSLKLSSVTAADTAVYYCSHFEGVWYGLDYWGQGTLVTVSS

SEQ ID NO: 83 - protein, 119 aa, synthetic construct
  REGION 1..119: note = synthetic Ror2 Y33E VH Conditionally active scFv
QVQLQESGPGLVKPSQTLSLTCTVSGYSITTGEYWNWVRQARGQRLEWIGYITYDGSKNYNPSLKNRVTISVDTSKNQFSLKLSSVTAADTAVYYCSRFEGVWYGLDYWGQGTLVTVSS

SEQ ID NO: 84 - protein, 111 aa, synthetic construct
  REGION 1..111: note = synthetic Ror2 VL Conditionally active scFv
AIQLTQSPSSLSASVGDRVTITCRASESVDRYGNSFIHWYQQKPGKAPKLLIYRTYNLESGIPARFSGSGSGTEFTLTISSLQSEDFAVYYCQQTNEDPWTFGQGTKVEIK

SEQ ID NO: 85 - protein, 10 aa, synthetic construct
  VARIANT 3: note = X is T, I, or P
  VARIANT 5: note = X is E or V
  VARIANT 8: note = X is F or T
  VARIANT 10: note = X is F or T
QQXNXDPXTX

SEQ ID NO: 86 - protein, 111 aa, synthetic construct
  REGION 1..111: note = synthetic Ror2 VL 5 Conditionally active scFv
DIVLTQSPDSLAVSLGQRATISCRASESVDRYGNSFIHWYLQKPGQPPKLLIYRTYNLESGIPARFSGTGSRTDFTLTINPVEADDVATYYCQQTNEDPWTFGQGTKVEIK

SEQ ID NO: 87 - protein, 6 aa, synthetic construct
  REGION 1..6: note = synthetic Axl H1 1 Conditionally active scFv
  VARIANT 1: note = X is T or W
  VARIANT 3: note = X is H or A
XGXTMN

SEQ ID NO: 88 - protein, 17 aa, synthetic construct
  REGION 1..17: note = synthetic Axl H2 1 Conditionally active scFv
LIKPSNGGTSYNQKFKG

SEQ ID NO: 89 - protein, 11 aa, synthetic construct
  REGION 1..11: note = synthetic Axl H31 Conditionally active scFv
  VARIANT 2: note = X is H or D
  VARIANT 4: note = X is E or H
  VARIANT 7: note = X is E or F
GXYXSYXAMDY

SEQ ID NO: 90 - protein, 11 aa, synthetic construct
  REGION 1..11: note = synthetic Axl L1 1 Conditionally active scFv
  VARIANT 7: note = X is S or V
KASQDVXSAVA

SEQ ID NO: 91 - protein, 7 aa, synthetic construct
  REGION 1..7: note = synthetic Axl L2 1 Conditionally active scFv
  MUTAGEN 2: note = X is A or Q
  VARIANT 3: note = X is S or D
  VARIANT 6: note = X is H or D
WXXTRXT

SEQ ID NO: 92 - protein, 9 aa, synthetic construct
  REGION 1..9: note = synthetic Axl L3 1 Conditionally active scFv
  VARIANT 6: note = X is T or P
  VARIANT 9: note = X is T or R
QEHFSXPLX

SEQ ID NO: 93 - protein, 120 aa, synthetic construct
  REGION 1..120: note = synthetic Axl WT VH Conditionally active scFv
EVQLVQSGAEVKKPGATVKISCKVSGYSFTGHTMNWIRQPPGKGLEWIGLIKPSNGGTSYNQKFKGRVTISADKSISTAYLQWSSLKASDTAMYYCAHGHYESYFAMDYWGQGTLVTVSS

SEQ ID NO: 94 - protein, 107 aa, synthetic construct
  REGION 1..107: note = synthetic Axl WT VL Conditionally active scFv
DIQMTQSPSSLSASVGDRVTITCKASQDVSSAVAWYQQKPGQAPRLLIYWASTRHTGVPSRFSGSGSGTEFTLTISSLQPDDFATYYCQEHFSTPLTFGQGTKVEIK

SEQ ID NO: 95 - protein, 10 aa, synthetic construct
  REGION 1..10: note = synthetic Ror2 H1 1 Conditionally active scFv
  VARIANT 4: note = X is F or E
  VARIANT 7: note = X is Y or D
  VARIANT 9: note = X is M or D
GYTXTEXTXH

SEQ ID NO: 96 - protein, 12 aa, synthetic construct
  REGION 1..12: note = synthetic Ror2 H1 2 Conditionally active scFv
  VARIANT 1: note = X is T or S
XGYSITTGYYWN

SEQ ID NO: 97 - protein, 17 aa, synthetic construct
  REGION 1..17: note = synthetic Ror2 H1 2 Conditionally active scFv
  VARIANT 2: note = X is E or I
  VARIANT 4: note = X is T or D
GXNXNNGGTGYNQKFKG

SEQ ID NO: 98 - protein, 16 aa, synthetic construct
  REGION 1..16: note = synthetic Ror2 H2 2 Conditionally active scFv
YITYDGSKNYNPSLKN

SEQ ID NO: 99 - protein, 13 aa, synthetic construct
  REGION 1..13: note = synthetic Ror2 H3 1 Conditionally active scFv
GSLYSYGNSYFDY

SEQ ID NO: 100 - protein, 10 aa, synthetic construct
  REGION 1..10: note = synthetic Ror2 H3 2 Conditionally active scFv
  VARIANT 6: note = X is Y or G
FEGVWXGLDY

SEQ ID NO: 101 - protein, 10 aa, synthetic construct
  REGION 1..10: note = synthetic Ror2 L1 1 Conditionally active scFv
  VARIANT 6: note = X is E or V
SATSSXSYMH

SEQ ID NO: 102 - protein, 15 aa, synthetic construct
  REGION 1..15: note = synthetic Ror2 L1 2 Conditionally active scFv
RASESVDRYGNSFIH

SEQ ID NO: 103 - protein, 7 aa, synthetic construct
  REGION 1..7: note = synthetic Ror2 L2 1 Conditionally active scFv
  VARIANT 1: note = X is G or H
XTSNLAS

SEQ ID NO: 104 - protein, 7 aa, synthetic construct
  REGION 1..7: note = synthetic Ror2 L2 2 Conditionally active scFv
RTYNLES

SEQ ID NO: 105 - protein, 9 aa, synthetic construct
  REGION 1..9: note = synthetic Ror2 L3 1 Conditionally active scFv
QQRSSYPFT

SEQ ID NO: 106 - protein, 9 aa, synthetic construct
  REGION 1..9: note = synthetic Ror2 L3 2 Conditionally active scFv
QQTNEDPWT

SEQ ID NO: 107 - protein, 119 aa, synthetic construct
  REGION 1..119: note = synthetic Ror2 WT VH Conditionally active scFv
QVQLQESGPGLVKPSQTLSLTCTVSGYSITTGYYWNWVRQARGQRLEWIGYITYDGSKNYNPSLKNRVTISVDTSKNQFSLKLSSVTAADTAVYYCSRFEGVWYGLDYWGQGTLVTVSS

SEQ ID NO: 108 - protein, 107 aa, synthetic construct
  REGION 1..107: note = synthetic Axl VL 1 Conditionally active scFv
DIQMTQSPSSLSASVGDRVTITCKASQDVVSAVAWYQQKPGQAPRLLIYWQDTRHTGVPSRFSGSGSGTEFTLTISSLQPDDFATYYCQEHFSTPLTFGQGTKVEIK

SEQ ID NO: 109 - protein, 107 aa, synthetic construct
  REGION 1..107: note = synthetic Axl VL 2 Conditionally active scFv
DIQMTQSPSSLSASVGDRVTITCKASQDVSSAVAWYQQKPGQAPRLLIYWQDTRHTGVPSRFSGSGSGTEFTLTISSLQPDDFATYYCQEHFSPPLTFGQGTKVEIK

SEQ ID NO: 110 - protein, 107 aa, synthetic construct; identical to SEQ ID NO: 80
  REGION 1..107: note = synthetic Axl VL 3 Conditionally active scFv

SEQ ID NO: 111 - protein, 107 aa, synthetic construct
  REGION 1..107: note = synthetic Axl VL 4 Conditionally active scFv
DIQMTQSPSSLSASVGDRVTITCKASQDVVSAVAWYQQKPGQAPRLLIYWQDTRHTGVPSRFSGSGSGTEFTLTISSLQPDDFATYYCQEHFSPPLRFGQGTKVEIK

SEQ ID NO: 112 - protein, 120 aa, synthetic construct
  REGION 1..120: note = synthetic Axl VH 1 Conditionally active scFv
EVQLVQSGAEVKKPGATVKISCKVSGYSFTGATMNWIRQPPGKGLEWIGLIKPSNGGTSYNQKFKGRVTISADKSISTAYLQWSSLKASDTAMYYCAHGHYESYEAMDYWGQGTLVTVSS

SEQ ID NO: 113 - protein, 120 aa, synthetic construct; identical to SEQ ID NO: 79
  REGION 1..120: note = synthetic Axl VH 2 Conditionally active scFv

SEQ ID NO: 114 - protein, 120 aa, synthetic construct
  REGION 1..120: note = synthetic Axl VH 3 Conditionally active scFv
EVQLVQSGAEVKKPGATVKISCKVSGYSFTGHTMNWIRQPPGKGLEWIGLIKPSNGGTSYNQKFKGRVTISADKSISTAYLQWSSLKASDTAMYYCAHGHYESYEAMDYWGQGTLVTVSS

SEQ ID NO: 115 - protein, 122 aa, synthetic construct
  REGION 1..122: note = synthetic Ror2 VH 1 Conditionally active scFv
EVQLQQSGPELVKPGASVKISCKTSGYTFTEYTMHWMKQSHRKSLEWIGGINTNNGGTGYNQKFKGKATLTVDKSSSTAYMELRSLTSEDSAVYFCARGSLYSYGNSYFDYWGQGTLVTVSS

SEQ ID NO: 116 - protein, 122 aa, synthetic construct
  REGION 1..122: note = synthetic Ror2 VH 1 Conditionally active scFv
EVQLQQSGPELVKPGASVKISCKTSGYTETEDTDHWMKQSHRKSLEWIGGENDNNGGTGYNQKFKGKATLTVDKSSSTAYMELRSLTSEDSAVYFCAHGSLYSYGNSYFDYWGQGTLVTVSS

SEQ ID NO: 117 - protein, 122 aa, synthetic construct
  REGION 1..122: note = synthetic Ror2 VH 3 Conditionally active scFv
QVQLVQSGAEVKKPGASVKVSCKASGYTFTEYTMHWVRQARGQRLEWIGGINTNNGGTGYNQKFKGRVTISADKSISTAYLQWSSLKASDTAMYYCAHGSLYSYGNSYFDYWGQGTLVTVSS

SEQ ID NO: 118 - protein, 122 aa, synthetic construct
  REGION 1..122: note = synthetic Ror2 VH 4 Conditionally active scFv
EVQLVQSGAEVKKPGESLRISCKGSGYTFTEYTMHWVRQAPGQGLEWMGGINTNNGGTGYNQKFKGRVTISADKSISTAYLQWSSLKASDTAMYYCAHGSLYSYGNSYFDYWGQGTLVTVSS

SEQ ID NO: 119 - protein, 122 aa, synthetic construct
  REGION 1..122: note = synthetic Ror2 VH 5 Conditionally active scFv
EVQLVQSGAEVKKPGESLRISCKGSGYTFTEYTMHWIRQSPSRGLEWLGGINTNNGGTGYNQKFKGRVTISADKSISTAYLQWSSLKASDTAMYYCAHGSLYSYGNSYFDYWGQGTLVTVSS

SEQ ID NO: 120 - protein, 119 aa, synthetic construct
  REGION 1..119: note = synthetic Ror2 VH 6 Conditionally active scFv
DVQLQESGPGLVKPSQSLSLTCSVTGYSITTGYYWNWIRQFPGNKLEWMAYITYDGSKNYNPSLKNRISITRDTSKNKFFLKLNSVTSEDTATYYCSRFEGVWYGLDYWGQGTLVTVSS

SEQ ID NO: 121 - protein, 119 aa, synthetic construct
  REGION 1..119: note = synthetic Ror2 VH 7 Conditionally active scFv
DVQLQESGPGLVKPSQSLSLTCSVTGYSITTGYYWNWIRQFPGNKLEWMAYITYDGSKNYNPSLKNRISITRDTSKNKFFLKLNSVTSEDTATYYCSHFEGVWGGLDYWGQGTLVTVSS

SEQ ID NO: 122 - protein, 106 aa, synthetic construct
  REGION 1..106: note = synthetic Ror2 VL 1 Conditionally active scFv
QIVLTQSPAIMSASPGEKVTITCSATSSVSYMHWFQQKPGTSPKLWIYGTSNLASGVPARFSGSGSGTSYSLTISRMEAEDAATYYCQQRSSYPFTFGQGTKVEIK

SEQ ID NO: 123 - protein, 106 aa, synthetic construct
  REGION 1..106: note = synthetic Ror2 VL 2 Conditionally active scFv
QIVLTQSPAIMSASPGEKVTITCSATSSESYMHWFQQKPGTSPKLWIYHTSNLASGVPARFSGSGSGTSYSLTISRMEAEDAATYYCQQRSSYPFTFGQGTKVEIK

SEQ ID NO: 124 - protein, 106 aa, synthetic construct
  REGION 1..106: note = synthetic Ror2 VL 3 Conditionally active scFv
AIQLTQSPSSLSASVGDRVTITCSATSSVSYMHWYQQKPGQAPRLLIYGTSNLASGVPDRFSGSGSGTDFTLKISRVEAEDVGVYYCQQRSSYPFTFGQGTKVEIK

SEQ ID NO: 125 - protein, 43 aa, synthetic construct
  REGION 1..43: note = synthetic wild-type CD8 Stalk
TTTPAPRPPTPAPTIASQPLSLRPEACRPAAGGAVHTRGLDFA

SEQ ID NO: 126 - protein, 42 aa, synthetic construct
  REGION 1..42: note = synthetic wild-type CD28 Stalk
FCKIEVMYPPPYLDNEKSNGTIIHVKGKHLCPSPLFPGPSKP

SEQ ID NO: 127 - protein, 113 aa, synthetic construct
  REGION 1..113: note = synthetic CD3Z 7 Activating domain
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 128 - protein, 241 aa, synthetic construct
  REGION 1..241: note = synthetic scFv from F1-2-15
EVQLVQSGAEVKKPGATVKISCKVSGYSFWGATMNWIRQPPGKGLEWIGLIKPSNGGTSYNQKFKGRVTISADKSISTAYLQWSSLKASDTAMYYCAHGHYESYEAMDYWGQGTLVTVSSGGGGSGGGSGGGGSDIQMTQSPSSLSASVGDRVTITCKASQDVVSAVAWYQQKPGQAPRLLIYWQDTRHTGVPSRFSGSGSGTEFTLTISSLQPDDFATYYCQEHFSPPLTFGQGTKVEIK

SEQ ID NO: 129 - protein, 242 aa, synthetic construct
  REGION 1..242: note = synthetic scFv from F1-2-13
EVQLVQSGAEVKKPGATVKISCKVSGYSFWGATMNWIRQPPGKGLEWIGLIKPSNGGTSYNQKFKGRVTISADKSISTAYLQWSSLKASDTAMYYCAHGHYESYEAMDYWGQGTLVTVSSGGGGSGGGGSGGGGSDIQMTQSPSSLSASVGDRVTITCKASQDVVSAVAWYQQKPGQAPRLLIYWQDTRHTGVPSRFSGSGSGTEFTLTISSLQPDDFATYYCQEHFSPPLTFGQGTKVEIK

SEQ ID NO: 130 - protein, 260 aa, synthetic construct
  REGION 1..260: note = synthetic scFv from F1-1-19
QVQLQESGPGLVKPSQTLSLTCTVSGYSITTGEYWNWVRQARGQRLEWIGYITYDGSKNYNPSLKNRVTISVDTSKNQFSLKLSSVTAADTAVYYCSRFEGVWYGLDYWGQGTLVTVSSGGGGSGGGGSGGGGSGGGGSGGGGSGGGGSAIQLTQSPSSLSASVGDRVTITCRASESVDRYGNSFIHWYQQKPGKAPKLLIYRTYNLESGIPARFSGSGSGTEFTLTISSLQSEDFAVYYCQQTNEDPWTFGQGTKVEIK

SEQ ID NO: 131 - protein, 260 aa, synthetic construct
  REGION 1..260: note = synthetic scFv from F1-1-23
AIQLTQSPSSLSASVGDRVTITCRASESVDRYGNSFIHWYQQKPGKAPKLLIYRTYNLESGIPARFSGSGSGTEFTLTISSLQSEDFAVYYCQQTNEDPWTFGQGTKVEIKGGGGSGGGGSGGGGSGGGGSGGGGSGGGGSQVQLQESGPGLVKPSQTLSLTCTVSGYSITTGEYWNWVRQARGQRLEWIGYITYDGSKNYNPSLKNRVTISVDTSKNQFSLKLSSVTAADTAVYYCSRFEGVWYGLDYWGQGTLVTVSS

SEQ ID NO: 132 - protein, 260 aa, synthetic construct
  REGION 1..260: note = synthetic scFV from F1-1-15
AIQLTQSPSSLSASVGDRVTITCRASESVDRYGNSFIHWYQQKPGKAPKLLIYRTYNLESGIPARFSGSGSGTEFTLTISSLQSEDFAVYYCQQTNEDPWTFGQGTKVEIKGGGGSGGGGSGGGGSGGGGSGGGGSGGGGSQVQLQESGPGLVKPSQTLSLTCTVSGYSITTGYYWNWVRQARGQRLEWIGYITYDGSKNYNPSLKNRVTISVDTSKNQFSLKLSSVTAADTAVYYCSHFEGVWYGLDYWGQGTLVTVSS

SEQ ID NO: 133 - protein, 83 aa, synthetic construct
  REGION 1..83: note = synthetic IC-delta CD137 Dual Co-stimulatory domain
RSKRSRLLHSDYMNMTPRRPGPTRKHYQAYAAARDFAAYRSKRGRKKLLYIFKQPFMRPVQTTQEEDGCSCRFPEEEEGGCEL

SEQ ID NO: 134 - skipped

SEQ ID NO: 135 - protein, 17 aa, synthetic construct
  REGION 1..17: note = synthetic Axl H1 2 Conditionally active scFv
  VARIANT 10: note = X is S, I, or T
  VARIANT 4: note = X is P or N
LIKXSNGGTXYNQKFKG

SEQ ID NO: 136 - skipped

SEQ ID NO: 137 - protein, 11 aa, synthetic construct
  REGION 1..11: note = synthetic Axl L1 2 Conditionally active scFv
  VARIANT 6: note = X is V, D, G, N, or W
  VARIANT 7: note = X is S or V
  VARIANT 9: note = X is A, L, or M
  VARIANT 11: note = X is A, D, N, or Q
KASQDXXSXVX

SEQ ID NO: 138-139 - skipped (2 entries)

SEQ ID NO: 140 - protein, 10 aa, synthetic construct
  REGION 1..10: note = synthetic Ror2 H1 3 Conditionally active scFv
  VARIANT 4: note = X is F or E
  VARIANT 7: note = X is Y or D
  VARIANT 8: note = X is T or C
  VARIANT 9: note = X is M, D, E, or Y
GYTXTEXXXH

SEQ ID NO: 141 - protein, 11 aa, synthetic construct
  REGION 1..11: note = synthetic Ror2 H1 4 Conditionally active scFv
  VARIANT 8: note = X is Y, E, R, or T
GYSITTGXYWN

SEQ ID NO: 142 - protein, 17 aa, synthetic construct
  REGION 1..17: note = synthetic Ror2 H2 3 Conditionally active scFv
  VARIANT 1: note = X is G or S
  VARIANT 2: note = X is I or E
  VARIANT 3: note = X is N, C, L, or V
  VARIANT 4: note = X is T, D, or E
XXXXNNGGTGYNQKFKG

SEQ ID NO: 143 - protein, 16 aa, synthetic construct
  REGION 1..16: note = synthetic Ror2 H2 4 Conditionally active scFv
  VARIANT 8: note = X is K or N
YITYDGSXNYNPSLKN

SEQ ID NO: 144 - protein, 16 aa, synthetic construct
  REGION 1..16: note = synthetic Ror2 H3 3 Conditionally active scFv
  VARIANT 1: note = X is A, M, or T
  VARIANT 2: note = X is R or H
  VARIANT 3: note = X is G or E
  VARIANT 5: note = X is L or F
  VARIANT 7: note = X is S or G
  VARIANT 9: note = X is G or D
  VARIANT 10: note = X is N or E
  VARIANT 14: note = X is D or L
  VARIANT 15: note = X is Y, C, or T
  VARIANT 16: note = X is W or L
XXXSXYXYXXSYFXXX

SEQ ID NO: 145 - protein, 13 aa, synthetic construct
  REGION 1..13: note = synthetic Ror2 H3 4 Conditionally active scFv
  VARIANT 4: note = X is F, C, N, or Q
  VARIANT 5: note = X is E or S
  VARIANT 6: note = X is G, E, F, H, M, Q, or S
  VARIANT 8: note = X is W, A, I, P, Q, T, or V
  VARIANT 9: note = X is Y, G, N, or Q
  VARIANT 10: note = X is G, S, or T
  VARIANT 13: note = X is Y or I
  VARIANT 3: note = X is R, G, H, W, or Y
CSXXXXVXXXLDX

SEQ ID NO: 146 - protein, 10 aa, synthetic construct
  REGION 1..10: note = synthetic Ror2 L1 3 Conditionally active scFv
  VARIANT 6: note = X is V or E
  VARIANT 7: note = X is S or D
  VARIANT 8: note = X is Y, C, or D
  VARIANT 10: note = X is H, G, or L
SATSSXXXMX

SEQ ID NO: 147 - protein, 15 aa, synthetic construct
  REGION 1..15: note = synthetic Ror2 L1 4 Conditionally active scFv
  VARIANT 13: note = X is F, S, or T
RASESVDRYGNSXIH

SEQ ID NO: 148 - protein, 7 aa, synthetic construct
  REGION 1..7: note = synthetic Ror2 L2 3 Conditionally active scFv
  VARIANT 1: note = X is G, C, H, or P
XTSNLAS

SEQ ID NO: 149 - protein, 7 aa, synthetic construct
  REGION 1..7: note = synthetic Ror2 L2 4 Conditionally active scFv
  VARIANT 4: note = X is N or D
  VARIANT 1: note = X is R, C, D, E, or W
XTYXLES

SEQ ID NO: 150 - protein, 10 aa, synthetic construct
  REGION 1..10: note = synthetic Ror2 L3 3 Conditionally active scFv
  VARIANT 2: note = X is Q or E
  VARIANT 3: note = X is R or H
  VARIANT 5: note = X is S, D, G, I, Q, or V
  VARIANT 9: note = X is T or D
  VARIANT 10: note = X is F, D, or E
QXXSXYPFXX

SEQ ID NO: 151 - protein, 122 aa, synthetic construct
  REGION 1..122: note = synthetic Ror2 CAB3 VH
QVQLVQSGAEVKKPGASVKVSCKASGYTFTEYTMHWIRQSPSRGLEWLGGINDNNGGTGYNQKFKGRFTISRDNSKNTLYLQMNSLRAEDTAVYYCARGSLYSYGNSYFDYWGQGTLVTVSS

SEQ ID NO: 152 - protein, 106 aa, synthetic construct
  REGION 1..106: note = synthetic Ror2 CAB VL
EIVLTQSPATLSLSPGERATLSCSATSSVSYMHWFQQRPGQSPRRLIYHTSNLASGVPDRFSGSGSGTDFTLKISRVEAEDVGVYYCQQRSSYPFTFGQGTKVEIK

SEQ ID NO: 153 - protein, 260 aa, synthetic construct
  REGION 1..260: note = synthetic scFV from F1-1-11
QVQLQESGPGLVKPSQTLSLTCTVSGYSITTGYYWNWVRQARGQRLEWIGYITYDGSKNYNPSLKNRVTISVDTSKNQFSLKLSSVTAADTAVYYCSHFEGVWYGLDYWGQGTLVTVSSGGGGSGGGGSGGGGSGGGGSGGGGSGGGGSAIQLTQSPSSLSASVGDRVTITCRASESVDRYGNSFIHWYQQKPGKAPKLLIYRTYNLESGIPARFSGSGSGTEFTLTISSLQSEDFAVYYCQQTNEDPWTFGQGTKVEIK

SEQ ID NO: 154 - protein, 245 aa, synthetic construct
  REGION 1..245: note = synthetic scFV from F1-1-17
QVQLQESGPGLVKPSQTLSLTCTVSGYSITTGEYWNWVRQARGQRLEWIGYITYDGSKNYNPSLKNRVTISVDTSKNQFSLKLSSVTAADTAVYYCSRFEGVWYGLDYWGQGTLVTVSSGGGGSGGGGSGGGGSAIQLTQSPSSLSASVGDRVTITCRASESVDRYGNSFIHWYQQKPGKAPKLLIYRTYNLESGIPARFSGSGSGTEFTLTISSLQSEDFAVYYCQQTNEDPWTFGQGTKVEIK

SEQ ID NO: 155 - protein, 243 aa, synthetic construct
  REGION 1..243: note = synthetic scFV from F1-1-25
EIVLTQSPATLSLSPGERATLSCSATSSVSYMHWFQQRPGQSPRRLIYHTSNLASGVPDRFSGSGSGTDFTLKISRVEAEDVGVYYCQQRSSYPFTFGQGTKVEIKGGGGSGGGGSGGGGSQVQLVQSGAEVKKPGASVKVSCKASGYTFTEYTMHWIRQSPSRGLEWLGGINDNNGGTGYNQKFKGRFTISRDNSKNTLYLQMNSLRAEDTAVYYCARGSLYSYGNSYFDYWGQGTLVTVSS

SEQ ID NO: 156 - protein, 258 aa, synthetic construct
  REGION 1..258: note = synthetic scFv from F1-1-26
EIVLTQSPATLSLSPGERATLSCSATSSVSYMHWFQQRPGQSPRRLIYHTSNLASGVPDRFSGSGSGTDFTLKISRVEAEDVGVYYCQQRSSYPFTFGQGTKVEIKGGGGSGGGGSGGGGSGGGGSGGGGSGGGGSQVQLVQSGAEVKKPGASVKVSCKASGYTFTEYTMHWIRQSPSRGLEWLGGINDNNGGTGYNQKFKGRFTISRDNSKNTLYLQMNSLRAEDTAVYYCARGSLYSYGNSYFDYWGQGTLVTVSS

SEQ ID NO: 157 - protein, 245 aa, synthetic construct
  REGION 1..245: note = synthetic scFV from F1-1-9
QVQLQESGPGLVKPSQTLSLTCTVSGYSITTGYYWNWVRQARGQRLEWIGYITYDGSKNYNPSLKNRVTISVDTSKNQFSLKLSSVTAADTAVYYCSHFEGVWYGLDYWGQGTLVTVSSGGGGSGGGGSGGGGSAIQLTQSPSSLSASVGDRVTITCRASESVDRYGNSFIHWYQQKPGKAPKLLIYRTYNLESGIPARFSGSGSGTEFTLTISSLQSEDFAVYYCQQTNEDPWTFGQGTKVEIK

SEQ ID NO: 158 - protein, 245 aa, synthetic construct
  REGION 1..245: note = synthetic scFV from F1-1-21
AIQLTQSPSSLSASVGDRVTITCRASESVDRYGNSFIHWYQQKPGKAPKLLIYRTYNLESGIPARFSGSGSGTEFTLTISSLQSEDFAVYYCQQTNEDPWTFGQGTKVEIKGGGGSGGGGSGGGGSQVQLQESGPGLVKPSQTLSLTCTVSGYSITTGEYWNWVRQARGQRLEWIGYITYDGSKNYNPSLKNRVTISVDTSKNQFSLKLSSVTAADTAVYYCSRFEGVWYGLDYWGQGTLVTVSS

SEQ ID NO: 159 - protein, 257 aa, synthetic construct
  REGION 1..257: note = synthetic scFV from F1-2-3
EVQLVQSGAEVKKPGATVKISCKVSGYSFWGATMNWIRQPPGKGLEWIGLIKPSNGGTSYNQKFKGRVTISADKSISTAYLQWSSLKASDTAMYYCAHGHYESYEAMDYWGQGTLVTVSSGGGGSGGGGSGGGGSGGGGSGGGGSGGGGSDIQMTQSPSSLSASVGDRVTITCKASQDVVSAVAWYQQKPGQAPRLLIYWQDTRHTGVPSRFSGSGSGTEFTLTISSLQPDDFATYYCQEHFSPPLTFGQGTKVEIK

SEQ ID NO: 160 - protein, 242 aa, synthetic construct
  REGION 1..242: note = synthetic scFV from F1-2-8
DIQMTQSPSSLSASVGDRVTITCKASQDVVSAVAWYQQKPGQAPRLLIYWQDTRHTGVPSRFSGSGSGTEFTLTISSLQPDDFATYYCQEHFSPPLTFGQGTKVEIKGGGGSGGGGSGGGGSEVQLVQSGAEVKKPGATVKISCKVSGYSFWGATMNWIRQPPGKGLEWIGLIKPSNGGTSYNQKFKGRVTISADKSISTAYLQWSSLKASDTAMYYCAHGHYESYEAMDYWGQGTLVTVSS

SEQ ID NO: 161 - protein, 257 aa, synthetic construct
  REGION 1..257: note = synthetic scFV from F1-2-10
DIQMTQSPSSLSASVGDRVTITCKASQDVVSAVAWYQQKPGQAPRLLIYWQDTRHTGVPSRFSGSGSGTEFTLTISSLQPDDFATYYCQEHFSPPLTFGQGTKVEIKGGGGSGGGGSGGGGSGGGGSGGGGSGGGGSEVQLVQSGAEVKKPGATVKISCKVSGYSFWGATMNWIRQPPGKGLEWIGLIKPSNGGTSYNQKFKGRVTISADKSISTAYLQWSSLKASDTAMYYCAHGHYESYEAMDYWGQGTLVTVSS